(19) United States
(12) Patent Application Publication
(10) Pub. No.: US 2014/0308714 A1
(43) Pub. Date: Oct. 16, 2014

(54) DECREASED POLYSACCHARIDE O-ACETYLATION

(71) Applicant: The Regents of the University of California, Oakland, CA (US)

(72) Inventors: Markus Pauly, Berkeley, CA (US); Sascha Gille, Berkeley, CA (US); Alex Schultink, Berkeley, CA (US)

(73) Assignee: The Regents of the University of California, Oakland, CA (US)

(21) Appl. No.: 14/053,505

(22) Filed: Oct. 14, 2013

Related U.S. Application Data
(63) Continuation-in-part of application No. PCT/US12/33656, filed on Apr. 13, 2012.
(60) Provisional application No. 61/476,155, filed on Apr. 15, 2011.
(60) Provisional application No. 61/715,192, filed on Oct. 17, 2012.

(51) Int. Cl. C12P 19/02

(52) U.S. Cl. CPC C12P 19/02
USPC 435/105

(57) ABSTRACT

The disclosure relates to polypeptides and polynucleotides from multiple species related to the O-acetylation of polysaccharides in plants. The disclosure also describes plants with reduced polysaccharide O-acetylation, methods related to the generation of plants with reduced polysaccharide O-acetylation, polysaccharides with reduced O-acetylation, and methods of using plants and polysaccharides having reduced O-acetylation.

### CROSS-REFERENCE TO RELATED APPLICATIONS

This application is a continuation-in-part of International Application No. PCT/US12/033,656, filed Apr. 13, 2012, which claims priority to U.S. Provisional Application No. 61/476,155, filed Apr. 15, 2011, which is hereby incorporated by reference, in its entirety. This application also claims priority to U.S. Provisional Application No. 61/715,192, filed Oct. 17, 2012, which is hereby incorporated by reference, in its entirety.

### STATEMENT REGARDING FEDERALLY-SPONSORED RESEARCH

This invention was made with government support under GM007127 awarded by the National Institutes of Health. The government has certain rights in the invention.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 416272009820SeqList.TXT, date recorded: Oct. 10, 2013, size: 323 KB).

### FIELD

The present disclosure relates to the O-acetylation of polysaccharides in plants. In particular, the disclosure relates to polypeptides and polynucleotides related to the O-acetylation of polysaccharides in plants, plants with reduced polysaccharide O-acetylation, and methods related to the generation of plants with reduced polysaccharide O-acetylation.

### BACKGROUND

Lignocellulosic plant materials are considered to be valuable feedstocks for the biorefinery industry, in particular for the production of biofuels and other commodity chemicals. Lignocellulosic plant materials are composed primarily of cellulose, hemicellulose and lignin. Cellulose is a polysaccharide composed of long chains of 13(1-4) linked D-glucose molecules. Hemicelluloses are various carbohydrate polymer chains including xylans, xyloglucans, glucuronoxylans, and glucomannans, that are composed of various different sugars, including xylose, arabinose, glucose, galactose, glucuronic acid, and mannose. Ligins are a diverse group of phenolic polymers that can be covalently linked to hemicelluloses. Lignin provides mechanical strength to plant cell walls.

Due to the high polysaccharide content of lignocellulosic plant materials, they are a rich potential source material for the production of biofuels and other sugar-derived products. One way to unlock the energy in lignocellulosic feedstocks is to degrade the material chemically and/or enzymatically to its component monosaccharides, which can then be fermented by microbes to ethanol. However, many of the polysaccharides in plant cell walls contain O-acetyl substituents. The acetyl groups inhibit the enzymatic breakdown of the polysaccharides (saccharification). Thus, the presence of acetyl groups on polysaccharides reduces the yield of monosaccharides generated from lignocellulosic material, and/or increases the amount of time and reagents necessary to release monosaccharides from lignocellulosic material.

In addition, degradation of acetylated polysaccharides causes the release of acetate groups, and the released acetate is an inhibitor of the subsequent fermentation of monosaccharides by microorganisms. The acetic acid contained in a biomass mixture for fermentation can be in the order of 0.1 M or 6 g/l, which is a highly inhibitory level. Reduction in the level of acetic acid would therefore be highly beneficial for fermentation.

In addition to affecting the degradation of lignocellulosic material, O-acetylation of polysaccharides is also known to be relevant to other applications of plant polysaccharides. The physicochemical properties of polysaccharides are commonly affected by their degree of O-acetylation. For example, the galacturonic acid residues of pectin can be O-acetylated, and the degree of a pectin chain's acetylation affects its gelling properties.

Another plant-derived polysaccharide that is acetylated is glucomannan. Glucomannan is a hemicellulose, and it also occurs in plant storage tissues. Glucomannan has main chains that are composed of β (1-4) linked D-glucose and D-mannose, and it has some branching of chains through β-1,6-glucosyl units. Purified glucomannan has been used as a food source and traditional medicine for thousands of years in Asia, and it is now used worldwide for various purposes.

Glucomannan is commonly obtained from plants of the genus Amorphophallus, and in particular, plants of the species Amorphophallus konjac (“A. konjac”), which develop underground corms (underground storage stems) that contain a high glucomannan content. A. konjac corms can be processed to extract purified glucomannan. Glucomannan is water soluble and highly viscous, and is used as a gelling agent and stabilizer for various products including food, drinks, pharmaceuticals, and cosmetics. The physicochemical properties, in particular the gelling properties, of glucomannan are affected by the degree of O-acetylation. For example, increasing the degree of O-acetylation of glucomannans by chemical means increases solubility of the polymers and reduces the viscosity of its respective aqueous solution.

Thus, to improve polysaccharides for multiple applications, including for use as a feedstock for the biorefinery industry and for use as an ingredient and in food and other products, there is a great need for plants with tailored polysaccharide O-acetylation.

### BRIEF SUMMARY

Provided herein are methods and compositions related to increasing the saccharification, fermentation, and chemical properties of plant polysaccharides by reducing the O-acetylation of the polysaccharides in plants.

Accordingly, certain aspects of the present disclosure provide a method of increasing biomass degradation, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene encoding a polypeptide having a polypeptide sequence selected from the group consisting of SEQ ID NOs: 113, 114, 115, 116, 117, 118, 119, 120, 121, and 124, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; and C) subjecting the biomass to a degradation procedure, thereby yielding degraded biomass, where the reduced O-acetylation of one or more plant cell wall polysaccharides in the mutant or transgenic plant increases the amount of degraded biomass compared to the amount of degraded biomass generated from the degradation of biomass obtained from the corresponding non-mutant or non-transgenic plant.

Other aspects of the present disclosure provide a method of increasing biomass saccharification, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene encoding a polypeptide having a polypeptide sequence selected from SEQ ID NOs: 113, 114, 115, 116, 117, 118, 119, 120, 121, and 124, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; and C) subjecting the biomass to a saccharification procedure, thereby yielding degraded biomass, where the reduced O-acetylation of one or more plant cell wall polysaccharides in the mutant or transgenic plant increases the amount of degraded biomass compared to the amount of degraded biomass generated from the saccharification of biomass obtained from the corresponding non-mutant or non-transgenic plant.

Other aspects of the present disclosure provide a method of increasing the yield of fermentation product from a fermentation reaction, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene encoding a polypeptide having a polypeptide sequence selected from SEQ ID NOs: 113, 114, 115, 116, 117, 118, 119, 120, 121, and 124, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; C) subjecting the biomass to a degradation procedure, thereby yielding degraded biomass, where the reduced O-acetylation of one or more plant cell wall polysaccharides in the mutant or transgenic plant increases the amount of degraded biomass compared to the amount of degraded biomass generated from the degradation of biomass obtained from the corresponding non-mutant or non-transgenic plant; and D) incubating the degraded biomass with a fermentative organism under conditions suitable to yield a fermentation product, where an increased yield of fermentation product from the fermentation reaction is obtained, as compared to the yield of fermentation product obtained from a fermentation reaction using degraded biomass from the corresponding non-mutant or non-transgenic plant.

Other aspects of the present disclosure provide a method of reducing O-acetylation of one or more plant cell wall polysaccharides in a non-Arabidopsis plant, by reducing the expression in a non-Arabidopsis plant of a gene encoding a polypeptide containing a polypeptide sequence selected from SEQ ID NOs: 113, 114, 115, 116, 117, 118, 119, 120, 121, and 124.

Other aspects of the present disclosure provide a method of altering the physicochemical properties of a polysaccharide derived from a non-Arabidopsis plant, by reducing the expression in a non-Arabidopsis plant of a gene encoding a polypeptide containing a polypeptide sequence selected from SEQ ID NOs: 113, 114, 115, 116, 117, 118, 119, 120, 121, and 124.

In certain embodiments that may be combined with any of the preceding aspects of the present disclosure, the gene is orthologous to an Arabidopsis thaliana gene selected from SEQ ID NOs: 5, 33, 35, 37, 39, 41, 43, 45, 47, 49, 51, 53, 93, 95, 97, 99, 101, 103, and 105. In certain embodiments that may be combined with any of the preceding embodiments, the gene is an ortholog of A. thaliana SEQ ID NO: 5. In certain embodiments that may be combined with any of the preceding aspects of the present disclosure, the gene is an ortholog of A. thaliana SEQ ID NO: 53. In certain embodiments that may be combined with any of the preceding aspects of the present disclosure, the gene is an ortholog of A. thaliana SEQ ID NO: 49. In certain embodiments that may be combined with any of the preceding embodiments, the polysaccharide is glucomannan and the expression of a gene orthologous to SEQ ID NO: 49 is reduced. In certain embodiments, the mutant or transgenic plant is Amorphophallus konjac and the gene is SEQ ID NO: 109. In certain embodiments that may be combined with any of the preceding embodiments, the mutant or transgenic plant is a mutant plant. In certain embodiments, the reduced expression of the gene or reduced activity of the protein encoded by the gene in the mutant plant is a result of a mutation in the gene. In certain embodiments, the mutation in the gene was the result of TILLING or T-DNA insertion. In certain embodiments that may be combined with any of the preceding embodiments, the mutant or transgenic plant is a transgenic plant. In certain embodiments, the transgenic plant further contains an RNAi-inducing vector or an antisense RNA construct. In certain embodiments, the reduced expression of the gene or reduced activity of the protein encoded by the gene in the transgenic plant is a result of the RNAi-inducing vector or the antisense RNA construct. In certain embodiments that may be combined with any of the preceding embodiments, the plant is selected from Zea mays, Oryza sativa, Sorghum bicolor, Populus trichocarpa, Picea sitchensis, Panicum virgatum, Miscanthus giganteus, Brachypodium distanchyon, and Amorphophallus konjac.

Other aspects of the present disclosure provide a non-Arabidopsis mutant plant, containing a mutation in a gene encoding a polypeptide containing a polypeptide sequence selected from SEQ ID NOs: 113, 114, 115, 116, 117, 118, 119, 120, 121, and 124, where the non-Arabidopsis plant has reduced O-acetylation activity as compared to a corresponding non-Arabidopsis plant lacking the mutation. In certain embodiments that may be combined with any of the preceding embodiments, the gene is orthologous to an Arabidopsis thaliana gene selected from SEQ ID NOs: 5, 33, 35, 37, 39, 41, 43, 45, 47, 49, 51, 53, 93, 95, 97, 99, 101, 103, and 105. In certain embodiments, the gene is an ortholog of A. thaliana SEQ ID NO: 5. In certain embodiments, the gene is an ortholog of A. thaliana SEQ ID NO: 53. In certain embodiments, the gene is an ortholog of A. thaliana SEQ ID NO: 49. In certain embodiments that may be combined with any of the preceding embodiments, the plant is selected from Zea mays, Oryza sativa, Sorghum bicolor, Populus trichocarpa, Picea sitchensis, Panicum virgatum, Miscanthus giganteus, Brachypodium distanchyon, and Amorphophallus konjac. Other aspects of the present disclosure provide a seed of the mutant plant of any of the preceding embodiments.

Other aspects of the present disclosure provide a plant containing an RNAi-inducing vector, where the vector generates RNAi against a gene encoding a polypeptide containing a polypeptide sequence selected from SEQ ID NOs: 113, 114, 115, 116, 117, 118, 119, 120, 121, and 124. In certain embodiments that may be combined with any of the preceding embodiments, the gene is orthologous to an Arabidopsis thaliana gene selected from SEQ ID NOs: 5, 33, 35, 37, 39, 41, 43, 45, 47, 49, 51, 53, 93, 95, 97, 99, 101, 103, and 105. In certain embodiments, the gene is an ortholog of A. thaliana SEQ ID NO: 5. In certain embodiments, the gene is an ortholog of A. thaliana SEQ ID NO: 53. In certain embodiments, the gene is an ortholog of A. thaliana SEQ ID NO: 49. In certain embodiments that may be combined with any of the preceding embodiments, the plant is selected from Zea mays, Oryza sativa, Sorghum bicolor, Populus trichocarpa, Picea sitchensis, Panicum virgatum, Miscanthus giganteus, Brachypodium distanchyon, and Amorphophallus konjac. In certain embodiments that may be combined with any of the preceding embodiments, the RNAi-inducing vector is stably transformed in the plant. Other aspects of the present disclosure provide a seed of the plant of any of the preceding embodiments.

Other aspects of the present disclosure provide a transgenic plant, containing a construct containing an acetylesterase gene having the polynucleotide sequence of SEQ ID NO: 107, SEQ ID NO: 111, an ortholog of SEQ ID NO: 107 or an ortholog of SEQ ID NO: 111, where the transgenic plant has reduced O-acetylation of one or more polysaccharides as compared to a corresponding plant lacking the construct. In certain embodiments, the plant is Amorphophallus konjac and the construct contains the polynucleotide sequence of SEQ ID NO: 111.

Other aspects of the present disclosure provide a non-Arabidopsis mutant plant, containing a mutation in a gene orthologous to an Arabidopsis thaliana gene selected from SEQ ID NOs: 5, 53, 49, 93, 95, 97, 99, 101, 103, and 105, where the non-Arabidopsis plant has reduced O-acetylation activity as compared to a corresponding non-Arabidopsis plant lacking the mutation.

Also provided herein is a seed of a non-Arabidopsis mutant plant, containing a mutation in a gene orthologous to an Arabidopsis thaliana gene selected from SEQ ID NOs: 5, 53, 49, 93, 95, 97, 99, 101, 103, and 105, where the non-Arabidopsis plant has reduced O-acetylation activity as compared to a corresponding non-Arabidopsis plant lacking the mutation.

Other aspects of the present disclosure provide a plant containing an RNAi-inducing vector, where the vector generates RNAi against a gene orthologous to an Arabidopsis thaliana gene selected from SEQ ID NOs: 5, 53, 49, 93, 95, 97, 99, 101, 103, and 105.

Also provided herein is a plant containing an RNAi-inducing vector, where the vector generates RNAi against a gene orthologous to an Arabidopsis thaliana gene selected from SEQ ID NOs: 5, 53, 49, 93, 95, 97, 99, 101, 103, and 105, and where the RNAi-inducing vector is stably transformed in the plant.

Also provided herein is the seed of a plant containing an RNAi-inducing vector, where the vector generates RNAi against a gene orthologous to an Arabidopsis thaliana gene selected from SEQ ID NOs: 5, 53, 49, 93, 95, 97, 99, 101, 103, and 105, and where the RNAi-inducing vector is stably transformed in the plant.

Other aspects of the present disclosure provide a method of reducing O-acetylation of one or more plant cell wall polysaccharides in a non-Arabidopsis plant, the method including reducing the expression in a non-Arabidopsis plant of a gene orthologous to an Arabidopsis thaliana gene selected from SEQ ID NOs: 5, 53, 49, 93, 95, 97, 99, 101, 103, and 105.

Also provided herein is a method of reducing O-acetylation of one or more plant cell wall polysaccharides in a non-Arabidopsis plant, the method including reducing the expression in a non-Arabidopsis plant of a gene orthologous to an Arabidopsis thaliana gene selected from SEQ ID NOs: 5, 53, 49, 93, 95, 97, 99, 101, 103, and 105, where reducing the expression of a gene includes one or more techniques selected from RNAi; antisense RNA; T-DNA insertion; and TILLING.

Other aspects of the present disclosure provide a method of altering the physicochemical properties of a polysaccharide derived from a non-Arabidopsis plant, the method including reducing the expression in a non-Arabidopsis plant of a gene orthologous to an Arabidopsis thaliana gene selected from SEQ ID NOs: 5, 33, 35, 37, 39, 41, 43, 45, 47, 49, 51, and 53.

Also provided herein is a method of altering the physicochemical properties of a polysaccharide derived from a non-Arabidopsis plant, the method including reducing the expression in a non-Arabidopsis plant of a gene orthologous to an Arabidopsis thaliana gene selected from SEQ ID NOs: 5, 33, 35, 37, 39, 41, 43, 45, 47, 49, 51, and 53, where the polysaccharide is glucomannan and the expression of a gene orthologous to SEQ ID NO: 49 is reduced.

Also provided herein is a method of altering the physicochemical properties of a polysaccharide derived from a non-Arabidopsis plant, the method including reducing the expression in a non-Arabidopsis plant of a gene orthologous to an Arabidopsis thaliana gene selected from SEQ ID NOs: 5, 33, 35, 37, 39, 41, 43, 45, 47, 49, 51, and 53, where the polysaccharide is glucomannan and the expression of a gene orthologous to SEQ ID NO: 49 is reduced, and where the non-Arabidopsis plant is Amorphophallus konjac and the gene is SEQ ID NO: 109.

Other aspects of the present disclosure provide a method of increasing biomass degradation, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene orthologous to an Arabidopsis thaliana gene selected from SEQ ID NOs: 5, 53, 49, 93, 95, 97, 99, 101, 103, and 105, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; and C) subjecting the biomass to a degradation procedure, thereby yielding degraded biomass, where the reduced O-acetylation of one or more plant cell wall polysaccharides in the mutant or transgenic plant increases the amount of degraded biomass compared to the amount of degraded biomass generated from the degradation of biomass obtained from the corresponding non-mutant or non-transgenic plant.

Other aspects of the present disclosure provide a method of increasing the yield of fermentation product from a fermentation reaction, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene orthologous to an Arabidopsis thaliana gene selected from SEQ ID NOs: 5, 53, 49, 93, 95, 97, 99, 101, 103, and 105, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; C) subjecting the biomass to a degradation procedure, thereby yielding degraded biomass, where the reduced O-acetylation of one or more plant cell wall polysaccharides in the mutant or transgenic plant increases the amount of degraded biomass compared to the amount of degraded biomass generated from the degradation of biomass obtained from the corresponding non-mutant or non-transgenic plant; and D) incubating the degraded biomass with a fermentative organism under conditions suitable to yield a fermentation product, where an increased yield of fermentation product from the fermentation reaction is obtained, as compared to the yield of fermentation product obtained from a fermentation reaction using degraded biomass from the corresponding non-mutant or non-transgenic plant.

Also provided herein is a method of increasing biomass degradation, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene orthologous to an Arabidopsis thaliana gene selected from SEQ ID NOs: 5, 53, 49, 93, 95, 97, 99, 101, 103, and 105, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; and C) subjecting the biomass to a degradation procedure, thereby yielding degraded biomass, where the reduced O-acetylation of one or more plant cell wall polysaccharides in the mutant or transgenic plant increases the amount of degraded biomass compared to the amount of degraded biomass generated from the degradation of biomass obtained from the corresponding non-mutant or non-transgenic plant, where the plant is a mutant plant, and where the reduced expression of the gene is a result of mutagenesis of the gene.

Also provided herein is a method of increasing the yield of fermentation product from a fermentation reaction, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene orthologous to an Arabidopsis thaliana gene selected from SEQ ID NOs: 5, 53, 49, 93, 95, 97, 99, 101, 103, and 105, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; C) subjecting the biomass to a degradation procedure, thereby yielding degraded biomass, where the reduced O-acetylation of one or more plant cell wall polysaccharides in the mutant or transgenic plant increases the amount of degraded biomass compared to the amount of degraded biomass generated from the degradation of biomass obtained from the corresponding non-mutant or non-transgenic plant; and D) incubating the degraded biomass with a fermentative organism under conditions suitable to yield a fermentation product, where an increased yield of fermentation product from the fermentation reaction is obtained, as compared to the yield of fermentation product obtained from a fermentation reaction using degraded biomass from the corresponding non-mutant or non-transgenic plant, where the plant is a mutant plant, and where the reduced expression of the gene is a result of mutagenesis of the gene.

Also provided herein is a method of increasing biomass degradation, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene orthologous to an Arabidopsis thaliana gene selected from SEQ ID NOs: 5, 53, 49, 93, 95, 97, 99, 101, 103, and 105, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; and C) subjecting the biomass to a degradation procedure, thereby yielding degraded biomass, where the reduced O-acetylation of one or more plant cell wall polysaccharides in the mutant or transgenic plant increases the amount of degraded biomass compared to the amount of degraded biomass generated from the degradation of biomass obtained from the corresponding non-mutant or non-transgenic plant, where the plant is a mutant plant, where the reduced expression of the gene is a result of mutagenesis of the gene, wand here the mutagenesis of the gene is by TILLING or T-DNA insertion.

Also provided herein is a method of increasing the yield of fermentation product from a fermentation reaction, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene orthologous to an Arabidopsis thaliana gene selected from SEQ ID NOs: 5, 53, 49, 93, 95, 97, 99, 101, 103, and 105, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; C) subjecting the biomass to a degradation procedure, thereby yielding degraded biomass, where the reduced O-acetylation of one or more plant cell wall polysaccharides in the mutant or transgenic plant increases the amount of degraded biomass compared to the amount of degraded biomass generated from the degradation of biomass obtained from the corresponding non-mutant or non-transgenic plant; and D) incubating the degraded biomass with a fermentative organism under conditions suitable to yield a fermentation product, where an increased yield of fermentation product from the fermentation reaction is obtained, as compared to the yield of fermentation product obtained from a fermentation reaction using degraded biomass from the corresponding non-mutant or non-transgenic plant, where the plant is a mutant plant, where the reduced expression of the gene is a result of mutagenesis of the gene, and where the mutagenesis of the gene is by TILLING or T-DNA insertion.

Other aspects of the present disclosure provide a non-Arabidopsis plant having reduced expression of a gene encoding a polypeptide containing a polypeptide sequence of SEQ ID NOs: 113, 114, 115, or 116.

Also provided herein is a seed of a non-Arabidopsis plant having reduced expression of a gene encoding a polypeptide containing a polypeptide sequence of SEQ ID NOs: 113, 114, 115, or 116.

Other aspects of the present disclosure provide a non-Arabidopsis plant having reduced expression of a gene encoding a polypeptide containing a polypeptide sequence of SEQ ID NOs: 117, 118, 119, 120, 121, or 124.

Also provided herein is a non-Arabidopsis plant having reduced expression of a gene encoding a polypeptide containing a polypeptide sequence of SEQ ID NOs: 117, 118, 119, 120, 121, or 124.

Also provided herein is a non-Arabidopsis mutant plant, containing a mutation in a gene orthologous to an Arabidopsis thaliana gene selected from SEQ ID NOs: 5, 53, 49, 93, 95, 97, 99, 101, 103, and 105, where the non-Arabidopsis plant has reduced O-acetylation activity as compared to a corresponding non-Arabidopsis plant lacking the mutation, and where the plant is selected from Zea mays, Oryza sativa, Sorghum bicolor, Populus trichocarpa, Picea sitchensis, Panicum virgatum, Miscanthus giganteus, Brachypodium distanchyon, or Amorphophallus konjac.

Also provided herein is a plant containing an RNAi-inducing vector, where the vector generates RNAi against a gene orthologous to an Arabidopsis thaliana gene selected from SEQ ID NOs: 5, 53, 49, 93, 95, 97, 99, 101, 103, and 105, and where the plant is selected from Zea mays, Oryza sativa, Sorghum bicolor, Populus trichocarpa, Picea sitchensis, Panicum virgatum, Miscanthus giganteus, Brachypodium distanchyon, or Amorphophallus konjac.

Also provided herein is a non-Arabidopsis plant having reduced expression of a gene encoding a polypeptide containing a polypeptide sequence of SEQ ID NOs: 113, 114, 115, or 116, where the plant is selected from Zea mays, Oryza sativa, Sorghum bicolor, Populus trichocarpa, Picea sitchensis, Panicum virgatum, Miscanthus giganteus, Brachypodium distanchyon, or Amorphophallus konjac.

Also provided herein is a non-Arabidopsis mutant plant, containing a mutation in a gene orthologous to an Arabidopsis thaliana gene selected from SEQ ID NOs: 5, 53, 49, 93, 95, 97, 99, 101, 103, and 105, where the non-Arabidopsis plant has reduced O-acetylation activity as compared to a corresponding non-Arabidopsis plant lacking the mutation, where the plant is selected from Zea mays, Oryza sativa, Sorghum bicolor, Populus trichocarpa, Picea sitchensis, Panicum virgatum, Miscanthus giganteus, Brachypodium distanchyon, or Amorphophallus konjac, and where the gene is an ortholog of A. thaliana SEQ ID NO: 5, 53, or 49.

Also provided herein is a plant containing an RNAi-inducing vector, where the vector generates RNAi against a gene orthologous to an Arabidopsis thaliana gene selected from SEQ ID NOs: 5, 53, 49, 93, 95, 97, 99, 101, 103, and 105, where the plant is selected from Zea mays, Oryza sativa, Sorghum bicolor, Populus trichocarpa, Picea sitchensis, Panicum virgatum, Miscanthus giganteus, Brachypodium distanchyon, or Amorphophallus konjac, and where the gene is an ortholog of A. thaliana SEQ ID NO: 5, 53, or 49.

Also provided herein is a non-Arabidopsis plant having reduced expression of a gene encoding a polypeptide containing a polypeptide sequence of SEQ ID NOs: 113, 114, 115, or 116, where the plant is selected from Zea mays, Oryza sativa, Sorghum bicolor, Populus trichocarpa, Picea sitchensis, Panicum virgatum, Miscanthus giganteus, Brachypodium distanchyon, or Amorphophallus konjac, and where the gene is an ortholog of A. thaliana SEQ ID NO: 5, 53, or 49.

Certain aspects of the present disclosure also provide a method for decreasing the acetylation level of biomass, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene encoding a polypeptide where the gene has the nucleotide sequence of SEQ ID NO: 127 or a homolog of this sequence, and where the mutant or transgenic plant has reduced expression of the gene or reduced activity of a protein encoded by the gene compared to the expression of the gene or activity of the protein encoded by the gene in the corresponding non-mutant or non-transgenic plant; and B) obtaining biomass from the mutant or transgenic plant.

Other aspects of the present disclosure provide a method of increasing the yield of fermentation product from a fermentation reaction, by: A) providing a mutant or transgenic plant having reduced O-acetylation of one or more plant cell wall polysaccharides in the mutant or transgenic plant compared to the O-acetylation of one or more plant cell wall polysaccharides of a corresponding non-mutant or non-transgenic plant, where the mutant or transgenic plant contains a gene encoding a polypeptide where the gene has the nucleotide sequence of SEQ ID NO: 127 or a homolog of this sequence, and where the mutant or transgenic plant has reduced expression of the gene or reduced activity of a protein encoded by the gene compared to the expression of the gene or activity of the protein encoded by the gene in the corresponding non-mutant or non-transgenic plant; B) obtaining biomass from the mutant or transgenic plant; C) subjecting the biomass to a degradation procedure, thereby yielding degraded biomass; and D) incubating the degraded biomass with a fermentative organism under conditions suitable to yield a fermentation product, where an increased yield of fermentation product from the fermentation reaction is obtained, as compared to the yield of fermentation product obtained from a fermentation reaction using degraded biomass from the corresponding non-mutant or non-transgenic plant.

Other aspects of the present disclosure provide a method of reducing O-acetylation of one or more plant cell wall polysaccharides in a non-Arabidopsis plant, by reducing the expression in a non-Arabidopsis plant of a gene encoding a polypeptide where the gene has the nucleotide sequence of SEQ ID NO: 127 or a homolog of this sequence.

Other aspects of the present disclosure provide a method of altering the physicochemical properties of a polysaccharide derived from a non-Arabidopsis plant, by reducing the expression in a non-Arabidopsis plant of a gene encoding a polypeptide where the gene has the nucleotide sequence of SEQ ID NO: 127 or a homolog of this sequence.

In certain embodiments that may be combined with any of the preceding embodiments, the polysaccharide is xylan and the expression of a gene homologous to SEQ ID NO: 127 is reduced. In certain embodiments that may be combined with any of the preceding embodiments, the polysaccharide is mannan and the expression of a gene homologous to SEQ ID NO: 127 is reduced. In certain embodiments that may be combined with any of the preceding embodiments, the polysaccharide is xylan and concomitantly mannan and the expression of a gene homologous to SEQ ID NO: 127 is reduced. In certain embodiments that may be combined with any of the preceding embodiments, the mutant or transgenic plant is a mutant plant. In certain embodiments, the reduced expression of the gene or reduced activity of the protein encoded by the gene in the mutant plant is a result of a mutation in the gene. In certain embodiments, the mutation in the gene was the result of a single base pair change or a T-DNA insertion. In certain embodiments that may be combined with any of the preceding embodiments, the mutant or transgenic plant is a transgenic plant. In certain embodiments, the transgenic plant further contains an RNAi-inducing vector or an antisense RNA construct. In certain embodiments, the reduced expression of the gene or reduced activity of the protein encoded by the gene in the transgenic plant is a result of the RNAi-inducing vector or the antisense RNA construct. In certain embodiments that may be combined with any of the preceding embodiments, the plant is selected from Zea mays, Oryza sativa, Sorghum bicolor, Populus trichocarpa, Picea sitchensis, Panicum virgatum, Miscanthus giganteus, Brachypodium distanchyon, and Amorphophallus konjac.

Other aspects of the present disclosure provide a non-Arabidopsis mutant plant, containing a mutation in a gene encoding a polypeptide where the gene has the nucleotide sequence of SEQ ID NO: 127 or a homolog of this sequence, where the non-Arabidopsis plant has reduced O-acetylation activity as compared to a corresponding non-Arabidopsis plant lacking the mutation. In certain embodiments that may be combined with any of the preceding embodiments, the plant is selected from Zea mays, Oryza sativa, Sorghum bicolor, Populus trichocarpa, Picea sitchensis, Panicum virgatum, Miscanthus giganteus, Brachypodium distanchyon, and Amorphophallus konjac. Other aspects of the present disclosure provide a seed of the mutant plant of any of the preceding embodiments.

Other aspects of the present disclosure provide a plant containing an RNAi-inducing vector, where the vector generates RNAi against a gene encoding a polypeptide where the gene has the nucleotide sequence of SEQ ID NO: 127 or a homolog of this sequence. In certain embodiments that may be combined with any of the preceding embodiments, the plant is selected from Zea mays, Oryza sativa, Sorghum bicolor, Populus trichocarpa, Picea sitchensis, Panicum virgatum, Miscanthus giganteus, Brachypodium distanchyon, and Amorphophallus konjac. In certain embodiments that may be combined with any of the preceding embodiments, the RNAi-inducing vector is stably transformed in the plant. Other aspects of the present disclosure provide a seed of the plant of any of the preceding embodiments.

### BRIEF DESCRIPTION OF THE DRAWINGS

FIG. 1A shows a graphic presentation of the genomic sequence of At1g70230 (TBL27). Shown are the T-DNA insertion positions as well as the position of the axy4-1 point mutation for the analyzed mutant lines. FIG. 1B shows a representative MALDI-TOF MS spectra of xyloglucan oligosaccharides derived by digestion of root material from wild type and TBL27 mutant lines with a xyloglucanase (XEG). The detectable O-acetylated oligosaccharide mass signals (or lack thereof) are highlighted in light gray.

FIG. 2 shows the genomic DNA sequence (SEQ ID NO: 126) of At1g70230 (TBL27). The exon sequences are in bold and underlines. Start and stop codon are highlighted in light gray.

FIG. 3 shows the protein sequence of A. thaliana TBL27 (SEQ ID NO: 54) with marked predicted transmembrane domain (dotted line box), conserved TBL domain (solid line box) and DUF231 domain (dashed line box). Highlighted in gray—Amino acid mutated in the axy4 mutant.

FIG. 4 shows an alignment of the conserved TBL domain of 46 TBR/TBL family members in Arabidopsis.

FIG. 5 shows a protein alignment of TBL25 from Amorphophallus konjac (AkTBL25) (SEQ ID NO: 110) and TBL25 (SEQ ID NO: 50) from Arabidopsis thaliana.

FIG. 6 shows results of wall bound acetate content and saccharification yields of various Arabidopsis mutant lines with a lack of expression of various gene candidates affecting cell wall polymer O-acetylation. All values are presented as percent wild type of the respective tissues.

FIG. 7 shows an A. thaliana TBL27 protein model. Predicted and conserved domains are given in black.

FIG. 8 shows overall xyloglucan O-acetylation determined by MALDI-TOF MS of xyloglucan oligosaccharides derived from digestion of wall material with a xyloglucanase (XEG) in different tissues of axy4-1.

FIG. 9 shows total wall bound acetate of root tissues as determined by acetic acid assay.

FIG. 10 shows the evolutionary relationships of 231 TBR/TBL genes in various plant taxa. The evolutionary history was inferred using the Neighbor-Joining method (Saitou & Nei, Mol. Biol. & Evo. 4:406-425 (1987)). The optimal tree with the sum of branch length=31.63210554 is shown. The percentage of replicate trees in which the associated taxa clustered together in the bootstrap test (500 replicates) are shown next to the branches (Felsenstein, Evo. 39:783-791 (1985)). The tree is drawn to scale, with branch lengths in the same units as those of the evolutionary distances used to infer the phylogenetic tree. The evolutionary distances were computed using the Poisson correction method (Zuckerkandl & Pauling, pp. 97-166 in Evolving Genes and Proteins, edited by V. Bryson and H. J. Vogel. Academic Press, New York (1965)) and are in the units of the number of amino acid substitutions per site. All positions containing gaps and missing data were eliminated from the dataset (Complete deletion option). There were a total of 58 positions in the final dataset. Phylogenetic analyses were conducted in MEGA4 (Tamura et al. Mol. Biol. & Evo. 24:1596-1599 (2007)).

FIG. 11 shows an alignment of A. thaliana sequences of the TBL17-27 clade.

FIG. 12 shows total wall bound acetate of stem tissue as determined by acetic acid assay.

FIG. 13 illustrates axy9 mutants. (A) The gene model of AXY9 and associated mutations in A. thaliana. The start codon (ATG) and stop codon (TGA) are shown along with the axy9.1 EMS mutation (W276Stop) and the axy9.2 T-DNA mutation. (B) MALDI-TOF MS spectra of xyloglucan oligosaccharides (for one letter code nomenclature, see Fry et al., 1993, Physiologia Plantarum, 89 (1) 1-3) released from wild-type, axy9.1 and axy9.2 hypocotyls in Arabidopsis thaliana. The gray box highlights the ion signal corresponding to acetylated xyloglucan oligosaccharides.

FIG. 14 illustrates the sequence and structural features of A. thaliana gene At3g03210 (AXY9). (A) The genomic DNA sequence (SEQ ID NO: 127) of AXY9. Untranslated regions (UTRs) are shown in lowercase and the coding sequence is in upper case. Start and stop codons shown in bold. (B) Model of AXY9 protein. AXY9 protein is 369 amino acids in length and is predicted to have two transmembrane domains near the N-terminus.

FIG. 15 illustrates total acetic acid content in lignocellulosic material (alcohol insoluble residue (AIR) of wild-type (Col-0) and axy9.1 stem (A), leaf (B) and leaf cell wall fractions (C) including buffer soluble polymers, pectic polysaccharides, xyloglucan (XyG) and the remaining pellet containing mainly xylans and mannans.

FIG. 16 illustrates the percent of xyloglucan acetylation for wild type, axy9.1 and axy9.2 in hypocotyls as well as percent xyloglucan acetylation for wild type and axy9.1 in leaves.

FIG. 17 illustrates quantification of xylan and mannan acetylation in the stem of wild type and axy9.1 using NMR (nuclear magnetic resonance).

FIG. 18 illustrates phylogenetic analysis of AXY9. Shown is a maximum likelihood tree of identified AXY9 putative orthologs. Putative orthologs of AXY9 were not identified outside of land plants. A single copy of AXY9 was found in most species while some species have multiple copies. Abbreviations: Acoe: Aquilegia coerulea, Alyr: Arabidopsis lyrata, Atha: Arabidopsis thaliana, Atri: Amborella trichopoda, Bdis: Brachipodium distachyon, Brap: Brassica rapa, Ccle: Citrus clementine, Cpap: Carica papaya, Cpur: Ceratodon purpureus, Crub: Capsella rubella, Csat: Cucumis sativus, Csin: Citrus sinensis, Egra: Eucalyptus grandis, Gmax: Glycine max, Grai: Gossypium raimondii, Lusi: Linum usitatissimum, Mdom: Malus domestica, Mesc: Manihot esculenta, Mgut: Mimulus guttatus, Mtru: Medicago truncatula, Nadv: Nuphar advena, Osat: Oryza sativa, Pdac: Phoenix dactilofera, Pgla: Picea glauca, Ppat: Physcomitrella patens, Pper: Prunus persica, Ppin: Pinus pinaster, Ptid: Pinus taeda, Ptri: Populus trichocarpa, Pvir: Panicum virgatum, Pvul: Phaseolus vulgaris, Rcom: Ricinus communis, Sbic: Sorghum bicolor, Sita: Setaria italic, Slyc: Solanum lycopersicum, Some: Selaginella moellendorffii, Stub: Solanum tuberosum, Thal: Thellungiella halophile, Tmaj: Tropaeolum majus, Vvin: Vitis vinifera, Zmay: Zea mays.

### DETAILED DESCRIPTION OF EMBODIMENTS

The present disclosure is based, at least in part, on the surprising discovery that plants can be engineered to have reduced O-acetylation of polysaccharides present in the plant material by reducing the expression of one or more O-acetylation related-genes or reducing the activity of one or more proteins encoded by one or more O-acetylation related-genes. In some embodiments, gene expression of various polysaccharide O-acetylation related-genes may be reduced to reduce the amount of acetate, i.e., polymer-bound acetyl esters, present in the plant material. In some embodiments, gene expression of various polysaccharide O-acetylation related-genes may be increased to reduce the amount of acetate, i.e., polymer-bound acetyl esters, present in the plant material.

In some embodiments, plant polysaccharides having decreased O-acetylation are more readily degraded into monosaccharides and/or oligosaccharides than polysaccharides from corresponding non-modified plants. In some embodiments, sugars derived from plant polysaccharides having decreased O-acetylation provide enhanced yield of a fermentation product in a fermentation reaction over sugars derived from plant polysaccharides from non-modified plants. Commonly, fermentation reactions employ a microorganism, such as a yeast or bacteria, that is sensitive to acetic acid levels in the fermentation reaction. In some embodiments, plant polysaccharides such as glucomannan or pectin that have reduced acetylation have improved properties as an ingredient for food or other products.

The disclosure provides methods of engineering plants to reduce acetate content by reducing expression of at least one, often two or three, polysaccharide O-acetylation related-genes in the plant. In some embodiments, the disclosure provides methods of engineering plants to reduce acetate content by increasing expression of at least one polysaccharide O-acetylation related-gene in the plant. The disclosure further provides plants that have been engineered to alter the expression of one or more O-acetylation related-genes, as well as methods of using such plants, e.g., to enhance biofuel yield from plant material, or to improve the physicochemical properties of sugars derived from the plant material.

The yield of a fermentation product from a fermentation reaction is generally increased due to the reduced polymer acetylation in a plant. The increased yield may result from having less acetate in the plant products that are used in the fermentation. To obtain sugars for the fermentation reaction, one or both of enzymatic or chemical degradation of the polysaccharides from the plant material can be used.

In some aspects, enzymatic degradation of plant polysaccharides is employed. In such aspects, the enzymatic degradation reaction can itself be improved due to the lowered acetate content in mutant plants having reduced gene expression of one or more genes related to polysaccharide O-acetylation. Typically, this results in increased yield in a fermentation reaction (i.e., either in the rate of fermentation and/or the total amount of fermentation product generated).

Improved degradation can also be advantageous without an effect on the final yield in fermentation. For example, in some embodiments, a reaction may employ less enzymes in order to degrade the biomass obtained from plants having reduced expression of one or more genes related to polysaccharide O-acetylation compared to the amount of enzyme required to degrade the biomass from non-modified plants. Accordingly, in some embodiments, improved yield from biomass from plants having reduced expression of one or more genes related to polysaccharide O-acetylation results from an increase in the amount of degradation product generated per enzyme unit per unit of time relative to the yield from corresponding biomass from a non-modified plant.

The degradation and fermentation of the biomass from the plant can be performed in one reaction mixture or using separate reaction mixtures. Plant material from a plant having reduced expression of one or more genes related to O-acetylation, e.g., cell wall material from shoots, stems, etc., can be degraded either enzymatically or chemically in one reaction and the degradation products then fermented in a separate reaction mixture. In other aspects, the degradation reaction and the fermentation reaction are conducted in the same reaction mixture such that the degradation products generated from enzymatic or chemical degradation of the plant biomass is fermented in the same mixture in which the biomass is degraded.

An “improved yield” from a fermentation reaction can thus arise from an improvement in the overall amount of product obtained from a reaction or from an increased efficiency of the overall reaction.

O-Acetylation

As used herein, “acetylation” refers to the covalent addition of an acetyl group (chemical formula COCH₃) to a molecule. O-acetylation refers to the addition of an acetyl group to an oxygen atom. The release of an O-acetylated acetyl group from a molecule may result in the release of an acetate anion [CH₃OO]⁻, which is the conjugate base of acetic acid. Accordingly, release of O-acetyl groups may result in the formation of acetic acid. An “acetyltransferase” is an enzyme which transfers an acetyl group onto (acetylates) a molecule.

Acetyl groups may be bound to polysaccharides and to glycan structures on glycoproteins and proteoglycans. The acetyl group can be bound to different OH-groups on sugars, and individual sugar residues can contain more than one acetyl ester group. Many different plant polysaccharides are known to be acetylated, including xylan, arabinoxylan, glucuronoarabinoxylan, xyloglucan, mannan, glucomannan, glucogalactomannan, and pectin.

Sequence Homologs/Orthologs/Paralogs

As used herein, “homologs” are polypeptide or polynucleotide sequences that share a significant degree of sequence identity or similarity. Sequences that are homologs are referred to as being “homologous” to each other. Homologs include sequences that are orthologs or paralogs.

As used herein, “orthologs” are evolutionarily related polypeptide or polynucleotide sequences in different species that have similar sequences and functions, and that develop through a speciation event. Sequences that are orthologs are referred to as being “orthologous” to each other.

As used herein, “paralogs” are evolutionarily related polypeptide or polynucleotide sequences in the same organism that have similar sequences and functions, and that develop through a gene duplication event. Sequences that are paralogs are referred to as being “paralogous” to each other.

### Methods of Identification of Homologous Sequences/Sequence Identity and Similarity

Several different methods are known to those of skill in the art for identifying homologous sequences, including phylogenetic methods, sequence similarity analysis, and hybridization methods.

Phylogenetic Methods

Phylogenetic trees may be created for a gene family by using a program such as CLUSTAL (Thompson et al. Nucleic Acids Res. 22: 4673-4680 (1994); Higgins et al. Methods Enzymol 266: 383-402 (1996)) or MEGA (Tamura et al. Mol. Biol. & Evo. 24:1596-1599 (2007)). Once an initial tree for genes from one species is created, potential orthologous sequences can be placed in the phylogenetic tree and their relationships to genes from the species of interest can be determined. Evolutionary relationships may also be inferred using the Neighbor-Joining method (Saitou & Nei, Mol. Biol. & Evo. 4:406-425 (1987)). Homologous sequences may also be identified by a reciprocal BLAST strategy. Evolutionary distances may be computed using the Poisson correction method (Zuckerkandl & Pauling, pp. 97-166 in Evolving Genes and Proteins, edited by V. Bryson and H. J. Vogel. Academic Press, New York (1965)).

In addition, evolutionary information may be used to predict gene function. Functional predictions of genes can be greatly improved by focusing on how genes became similar in sequence (i.e. by evolutionary processes) rather than on the sequence similarity itself (Eisen, Genome Res. 8: 163-167 (1998)). Many specific examples exist in which gene function has been shown to correlate well with gene phylogeny (Eisen, Genome Res. 8: 163-167 (1998)). By using a phylogenetic analysis, one skilled in the art would recognize that the ability to deduce similar functions conferred by closely-related polypeptides is predictable.

When a group of related sequences are analyzed using a phylogenetic program such as CLUSTAL, closely related sequences typically cluster together or in the same clade (a group of similar genes) Groups of similar genes can also be identified with pair-wise BLAST analysis (Feng and Doolittle, J. Mol. Evol. 25: 351-360 (1987)). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount, Bioinformatics: Sequence and Genome Analysis Cold Spring Harbor Laboratory Press, Cold Spring Harbor, N.Y., page 543 (2001)).

To find sequences that are homologous to a reference sequence, BLAST nucleotide searches can be performed with the BLASTN program, score=100, wordlength=12, to obtain nucleotide sequences homologous to a nucleotide sequence encoding a protein of the disclosure. BLAST protein searches can be performed with the BLASTX program, score=50, wordlength=3, to obtain amino acid sequences homologous to a protein or polypeptide of the disclosure. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used.

Sequence Alignment/Sequence Similarity and Identity Analysis

Methods for the alignment of sequences and for the analysis of similarity and identity of polypeptide and polynucleotide sequences are well known in the art.

As used herein “sequence identity” refers to the percentage of residues that are identical in the same positions in the sequences being analyzed. As used herein “sequence similarity” refers to the percentage of residues that have similar biophysical/biochemical characteristics in the same positions (e.g. charge, size, hydrophobicity) in the sequences being analyzed.

Methods of alignment of sequences for comparison are well-known in the art, including manual alignment and computer assisted sequence alignment and analysis. This latter approach is a preferred approach in the present disclosure, due to the increased throughput afforded by computer assisted methods. As noted below, a variety of computer programs for performing sequence alignment are available, or can be produced by one of skill.

The determination of percent sequence identity and/or similarity between any two sequences can be accomplished using a mathematical algorithm. Non-limiting examples of such mathematical algorithms are the algorithm of Myers and Miller, CABIOS 4:11-17 (1988); the local homology algorithm of Smith et al., Adv. Appl. Math. 2:482 (1981); the homology alignment algorithm of Needleman and Wunsch, J. Mol. Biol. 48:443-453 (1970); the search-for-similarity-method of Pearson and Lipman, Proc. Natl. Acad. Sci. 85:2444-2448 (1988); the algorithm of Karlin and Altschul, Proc. Natl. Acad. Sci. USA 87:2264-2268 (1990), modified as in Karlin and Altschul, Proc. Natl. Acad. Sci. USA 90:5873-5877 (1993).

Computer implementations of these mathematical algorithms can be utilized for comparison of sequences to determine sequence identity and/or similarity. Such implementations include, but are not limited to: CLUSTAL in the PC/Gene program (available from Intelligenetics, Mountain View, Calif.); the AlignX program, version10.3.0 (Invitrogen, Carlsbad, Calif.) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. Gene 73:237-244 (1988); Higgins et al. CABIOS 5:151-153 (1989); Corpet et al. Nucleic Acids Res. 16:10881-90 (1988); Huang et al. CABIOS 8:155-65 (1992); and Pearson et al. Meth. Mol. Biol. 24:307-331 (1994). The BLAST programs of Altschul et al. J. Mol. Biol. 215:403-410 (1990) are based on the algorithm of Karlin and Altschul (1990) supra.

Hybridization Methods

Polynucleotides homologous to a reference sequence can be identified by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in references cited below (e.g., Sambrook et al., Molecular Cloning: A Laboratory Manual, 2nd Ed., Vol. 1-3, Cold Spring Harbor Laboratory, Cold Spring Harbor, N.Y. (“Sambrook”) (1989); Berger and Kimmel, Guide to Molecular Cloning Techniques, Methods in Enzymology, vol. 152 Academic Press, Inc., San Diego, Calif. (“Berger and Kimmel”) (1987); and Anderson and Young, “Quantitative Filter Hybridisation.” In: Hames and Higgins, ed., Nucleic Acid Hybridisation, A Practical Approach. Oxford, TRL Press, 73-111 (1985)).

Encompassed by the disclosure are polynucleotide sequences that are capable of hybridizing to the disclosed polynucleotide sequences, including any polynucleotide within the Sequence Listing, and fragments thereof under various conditions of stringency (see, for example, Wahl and Berger, Methods Enzymol. 152: 399-407 (1987); and Kimmel, Methods Enzymo. 152: 507-511, (1987)). In addition to the nucleotide sequences in the Sequence Listing, full length cDNA, orthologs, and paralogs of the present nucleotide sequences may be identified and isolated using well-known polynucleotide hybrization methods.

With regard to hybridization, conditions that are highly stringent, and means for achieving them, are well known in the art. See, for example, Sambrook et al. (1989) (supra); Berger and Kimmel (1987) pp. 467-469 (supra); and Anderson and Young (1985)(supra).

Hybridization experiments are generally conducted in a buffer of pH between 6.8 to 7.4, although the rate of hybridization is nearly independent of pH at ionic strengths likely to be used in the hybridization buffer (Anderson and Young (1985)(supra)). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency. As a general guidelines high stringency is typically performed at Tₘ-5° C. to Tₘ-20° C., moderate stringency at Tₘ-20° C. to Tₘ-35° C. and low stringency at Tₘ-35° C. to Tₘ-50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ-25° C. for DNA-DNA duplex and Tₘ-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tₘ) for the specific sequence at a defined ionic strength and pH.

Hybridization and wash conditions that may be used to bind and remove polynucleotides with less than the desired homology to the nucleic acid sequences or their complements that encode the present transcription factors include, for example: 6×SSC and 1% SDS at 65° C.; 50% formamide, 4×SSC at 42° C.; 0.5×SSC to 2.0×SSC, 0.1% SDS at 50° C. to 65° C.; or 0.1×SSC to 2×SSC, 0.1% SDS at 50° C.-65° C.; with a first wash step of, for example, 10 minutes at about 42° C. with about 20% (v/v) formamide in 0.1×SSC, and with, for example, a subsequent wash step with 0.2×SSC and 0.1% SDS at 65° C. for 10, 20 or 30 minutes.

For identification of less closely related homologs, wash steps may be performed at a lower temperature, e.g., 50° C. An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Wash procedures will generally employ at least two final wash steps. Additional variations on these conditions will be readily apparent to those skilled in the art (see, for example, US Patent Application No. 20010010913).

If desired, one may employ wash steps of even greater stringency, including conditions of 65° C.-68° C. in a solution of 15 mM NaCl, 1.5 mM trisodium citrate, and 0.1% SDS, or about 0.2×SSC, 0.1% SDS at 65° C. and washing twice, each wash step of 10, 20 or 30 min in duration, or about 0.1×SSC, 0.1% SDS at 65° C. and washing twice for 10, 20 or 30 min. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C.

Polynucleotide probes may be prepared with any suitable label, including a fluorescent label, a colorimetric label, a radioactive label, or the like. Labeled hybridization probes for detecting related polynucleotide sequences may be produced, for example, by oligolabeling, nick translation, end-labeling, or PCR amplification using a labeled nucleotide.

Polypeptides of the Disclosure

The present disclosure relates to polypeptides that affect the acetylation of polysaccharides in plants. In some aspects, the disclosure relates to polypeptides that promote the O-acetylation of polysaccharides in plants. In some aspects, the disclosure relates to AXY9 polypeptides and homologs thereof. In some aspects, the disclosure relates to polypeptides in the Trichome Birefringence (TBR)/Trichome Birefringence-Like (TBL) family. In some aspects, the disclosure relates to polypeptides in the Membrane-Bound O-Acyl Transferase (MBOAT) family. In some aspects, the disclosure relates to polypeptides in the BAHD acyltransferase family. In some aspects, the disclosure relates to acetylesterases.

As used herein, a “polypeptide” is an amino acid sequence including a plurality of consecutive polymerized amino acid residues (e.g., at least about 15 consecutive polymerized amino acid residues). As used herein, “polypeptide” refers to an amino acid sequence, oligopeptide, peptide, protein, or portions thereof.

As used herein with reference to polypeptides encoded by polynucleotides, “encode” refers to the ability of a polynucleotide to be translated into a polypeptide. Therefore, a polynucleotide may encode a polypeptide. Polypeptides encoded by a polynucleotide arise as a result of translation of the polynucleotide. A polynucleotide may include nucleotide sequences that are not translated into amino acids of the final translated polypeptide, such as untranslated regions (UTRs). Where such nucleotide sequences are known or labeled, one of skill in the art would understand that such nucleotide sequences, while part of the gene, are not translated into amino acids in the translated polypeptide.

Polypeptides that Promote the O-Acetylation of Polysaccharides in Plants

The present disclosure also relates to polypeptides that promote the O-acetylation of polysaccharides in plants. As provided herein, polypeptides of the disclosure that promote O-acetylation in plants include polypeptides in the TBR/TBL family, polypeptides in the TBL17-TBL27 clade including TBL25 and TBL27, polypeptides in the MBOAT family, polypeptides in the BAHD acyltransferase family, and AXY9 polypeptides and homologs thereof.

In some aspects, polypeptides that promote the O-acetylation of polysaccharides in plants are O-acetyltransferases. Methods of assessing a polypeptide for O-acetyltransferase activity are well-known in the art, and are further described in Example 6.

Polypeptides that Reduce the O-Acetylation of Polysaccharides in Plants

The present disclosure also relates to polypeptides that reduce the O-acetylation of polysaccharides in plants. As provided herein, polypeptides of the disclosure that reduce O-acetylation in plants include acetylesterases.

TBR/TBL Family Polypeptides

The present disclosure also relates to decreasing the expression of a gene encoding a polypeptide of the Trichome Birefringence (TBR)/Trichome Birefringence-Like (TBL) family, or to decreasing the activity of a polypeptide of the TBR/TBL family. The TBR/TBL family contains 46 members in Arabidoposis thaliana (“A. thaliana”). The 46 members of the TBR/TBL family in Arabidopsis are the A. thaliana genes TBR, TBL1, TBL2, TBL3, TBL4, TBL5, TBL6, TBL7, TBL8, TBL9, TBL10, TBL11, TBL12, TBL13, TBL14, TBL15, TBL16, TBL17 (YLS7), TBL18, TBL19, TBL20, TBL21, TBL22, TBL23, TBL24, TBL25, TBL26, TBL27, TBL28, TBL29 (ESK1), TBL30, TBL31, TBL32, TBL33, TBL34, TBL35, TBL36, TBL37, TBL38, TBL39, TBL40, TBL41, TBL42, TBL43, TBL44 (PMR5), and TBL45. The polypeptide sequences for the TBR/TBL family members are provided in SEQ ID NOs: 92 (TBR), 2 (TBL1), 4 (TBL2), 6 (TBL3), 8 (TBL4), 10 (TBL5), 12 (TBL6), 14 (TBL7), 16 (TBL8), 18 (TBL9), 20 (TBL10), 22 (TBL11), 24 (TBL12), 26 (TBL13), 28 (TBL14), 30 (TBL15), 32 (TBL16), 34 (TBL17) (YLS7), 36 (TBL18), 38 (TBL19), 40 (TBL20), 42 (TBL21), 44 (TBL22), 46 (TBL23), 48 (TBL24), 50 (TBL25), 52 (TBL26), 54 (TBL27), 56 (TBL28), 58 (TBL29) (ESK1), 60 (TBL30), 62 (TBL31), 64 (TBL32), 66 (TBL33), 68 (TBL34), 70 (TBL35), 72 (TBL36), 74 (TBL37), 76 (TBL38), 78 (TBL39), 80 (TBL40), 82 (TBL41), 84 (TBL42), 86 (TBL43), 88 (TBL44) (PMR5), and 90 (TBL45).

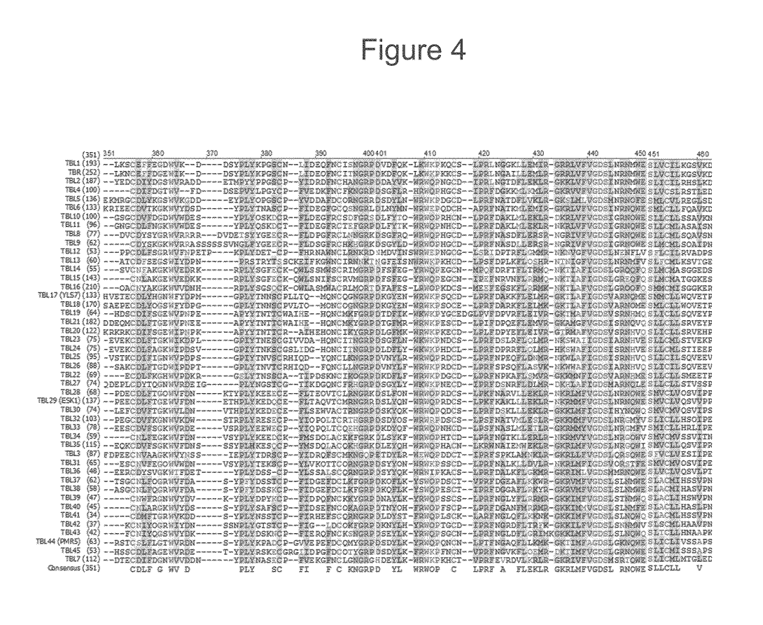

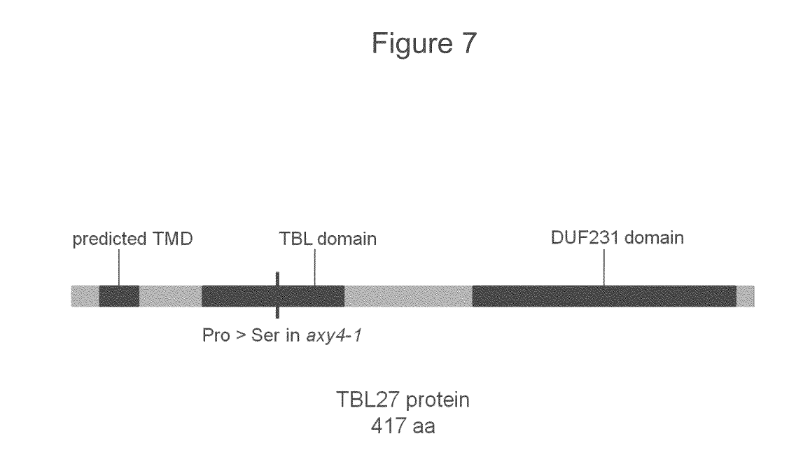

All TBR/TBL proteins have a transmembrane domain at the N-terminus, a plant kingdom specific DUF231 domain at the C-terminus, and a highly conserved TBL domain (see FIG. 7). The TBL domain is unique to members of the TBR/TBL family of proteins. An alignment of the TBL domain from A. thaliana TBR/TBL family proteins is provided in FIG. 4, and consensus sequence of the TBL domain across 46 members of A. thaliana TBR/TBL members is:

(SEQ ID NO: 113)
C-[D/N/S/E]-[L/I/W/Y/V/F]-[F/Y/T/S/A]-X-G-X-W-
[V/I/F/T]-X-[D/R/N]-X-X-X-X-X-X-X-X-X-[P/T/G/S/]-
[L/Y/V/S/R/I/F]-[Y/F/S/H]-X-X-X-[S/Y/D/E/T/Q/K]-
[C/S]-X-X-X-[F/L/Y/E/W/T/H/A/I/Q]-
[I/V/L/H/Q/E/D/M]-X-X-X-[F/W/Q/K/V/L/T/]-X-C-
X-[K/S/T/A/R/D/E/N/I/V/L/G/M]-[N/F/H/M/Y/Q/A]-
[G/K/N/Q]-[R/K/Q]-[P/D/R/L/S/K/D/T/G]-[D/N/H]-
X-X-[Y/F/V/E]-[L/Q/V/M/T/R/I/E/Q/K/S]-X-X-
[W/L/H/Y]-[R/K/E/S]-W-[Q/K/E/R/I]-P-X-X-C-X-
X-X-[L/I/A/M/V]-[P/S/E/K/A]-[R/S/Q/E/V/I/L/K/T]-
[F/L/I/W]-X-[A/G/P/R/S/V]-X-X-[F/L/M/A/V]-
[L/W/M]-[E/K/V/G/Q/T/R/S/A/N/L]-
[K/M/R/S/L/I/N/V/E]-[L/I/S/M/H/V/W/Y/N/F]-
[R/Q/K/M]-X-[G/D/N/H]-[K/R/G/T]-[R/S/N/T/A/W/K/H]-
[L/V/I/M/W]-[M/V/G/A/N/L]-[F/L/I/Y/M]-[V/I/A]-G-
D-S-[L/I/M/V]-X-[R/E/Y/L/T/N/K]-[N/Q/E/G/S/T]-
[Q/M/F/H/T]-[W/F/L/M/V/Y]-[E/V/Q/I/]-S-[L/M/F]-
[L/V/F/M/I/A/S/T]-C-[L/I/S/V/M]-[L/A/I/V]-X-X-X-
[V/L/I/D/T/E/K/S/A];
where X is any amino acid, [D/N/S/E] is D, N, S,
or E, [L/I/W/Y/V/F] is L, I, W, Y, V, or F,
[F/Y/T/S/A] is F, Y, T, S, or A, [V/I/F/T] is V,
I, F, or T, [D/R/N] is D, R, or N, [P/T/G/S] is
P, T, G, or S, [L/Y/V/S/R/I/F] is L, Y, V, S, R,
I, or F, [Y/F/S/H] is Y, F, S, or H,
[S/Y/D/E/T/Q/K] is S, Y, D, E, T, Q, or K, [C/S]
is C or S, [F/L/Y/E/W/T/H/A/I/Q] is F, L, Y,
E, W, T, H, A, I, or Q, [I/V/L/H/Q/E/D/M] is I,
V, L, H,Q, E, D, or M, [F/W/Q/K/V/L/T]
is F, W, Q, K, V, L, or T, S, T,
[K/S/T/A/R/D/E/N/I/V/L/G/M] is K,
A, R, D, E, N, I, V, L, G, or M,
[N/F/H/M/Y/Q/A] is N, F, H, M, Y, Q, or A,
[G/K/N/Q] is G, K, N, or Q, [R/K/Q] is R, K
or Q, [P/D/R/L/S/K/D/T/G] is P, D, R, L, S,
K, D, T, or G, is D, N, or H, [Y/F/V/E] is
Y, F, V, or E, [L/Q/V/M/T/R/I/E/Q/K/S] is L,
Q, V, M, T, R, I, E, Q, K, or S, [W/L/H/Y] is
W, L, H, or Y, [R/K/E/S] is R, K, E, or S,
[Q/K/E/R/I] is Q, K, E, R, or I, [L/I/A/M/V]
is L, I, A, M, or V, [P/S/E/K/A] is P, S,
E, K, or A, [R/S/Q/E/V/I/L/K/T] is R, S, Q,
E, V, I, L, K, or T, [F/L/I/W] is F, L, I,
or W, [A/G/P/R/S/V] is A, G, P, R, S, or V,
[F/L/M/A/V] is F, L, M, A, or V, [L/W/M] is
L, W, or M, [E/K/V/G/Q/T/R/S/A/N/L] is E, K,
V, G, Q, T, R, S, A, N, or L, [K/M/R/S/L/I/N/V/E]
is K, M, R, S, L, I, N, V, or E,
[L/I/S/M/H/V/W/Y/N/F] is L, I, S, M, H, V,
W, Y, N, or F, [R/Q/K/M] is R, Q, K, or M,
[G/D/N/H] G, D, N, or H, [K/R/G/T] is K, R, G,
or T, [R/S/N/T/A/W/K/H] is R, S, N, T, A, W,
K, or H, [L/V/I/M/W] is L, V, I, M, or W,
[M/V/G/A/N/L] is M, V, G, A, N, or L, [F/L/I/Y/M]
is F, L, I, Y, or M, [V/I/A] is V, I, or A,
[L/I/M/V] is L, I, M, or V, [R/E/Y/L/T/N/K] is
R, E, Y, L, T, N, or K, [N/Q/E/G/S/T] is N, Q,
E, G, S, or T, [Q/M/F/H/T] is Q, M, F, H, or T,
[W/F/L/M/V/Y] is W, F, L, M, V, or Y, [E/V/Q/I]
is E, V, Q, or I, [L/M/F] is L, M, or F,
[L/V/F/M/I/A/S/T] is L, V, F, M, I, A, S, or T,
[L/I/S/V/M] is L, I, S, V, or M, [L/A/I/V] is
L, A, I, or V,
and
[V/L/I/D/T/E/K/S/A] is V, L, I, D, T, E, K, S,
or A.

In the above motif and all other motifs provided herein, the accepted IUPAC single letter amino acid abbreviation is employed.

A consensus sequence of the TBL domain from TBR/TBL proteins across multiple different species is also provided herein. The consensus TBL domain from TBR/TBL proteins across multiple species provided herein has 60.2% similarity across TBR/TBL proteins from Oryza sativa (rice), Zea mays (corn), Populus trichocarpa (poplar), Sorghum bicolor (sorghum), Pichea sitchensis (Sitka spruce), and Arabidopsis thaliana. The consensus sequence of the TBL domain from TBR/TBL proteins across multiple different species provided herein is:

(SEQ ID NO: 114)
C-D-L-F-X-X-X-X-X-G-X-W-V-X-D-X-X-X-X-X-X-X-X-X-
P-L-Y-X-X-X-X-C-X-X-X-F-I-D-X-X-X-X-C-X-K-N-G-R-
P-D-X-X-Y-L-K-W-R-W-Q-P-X-X-X-X-X-X-X-C-X-L-P-R-
X-F-D-A-X-X-F-L-E-R-L-R-X-G-K-R-L-M-F-V-G-D-S-L-
X-R-N-Q-W-E-S-L-V-C-L-L-;
where X is any amino acid.

In some aspects, the present disclosure relates to polypeptides that are orthologs of Arabidoposis polypeptides of the TBR/TBL family. Methods for identification of polypeptides that are homologs/orthologs of a polypeptide of interest are well known to one of skill in the art, as described above.

Polypeptides of the disclosure include polypeptides having the sequence motif of SEQ ID NO: 113 or SEQ ID NO: 114. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 113. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 114. Polypeptides of the disclosure also include polypeptides having at least 10, at least 12, at least 14, at least 16, at least 18, or at least 20 consecutive amino acids of SEQ ID NOs: 113 or 114.

Polypeptides of the disclosure further include polypeptides that are orthologous to any member of the A. thaliana TBR/TBL family, including, without limitation, orthologous polypeptides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Examples of polypeptides that are orthologous to A. thaliana TBR/TBL family proteins include, without limitation the polypeptides of the sequences of Table 1.

TABLE 1

| A. thaliana | |
|---|---|
| Gene(s) | Orthologs in Other Plant Species |
| TBR/TBL1/ | rice: NP_001057100; NP_001053267 |
| TBL2/TBL4 | sorghum: XP_002436669; XP_002454683; XP_002448144 |
| | corn: NP_001141610 |
| | poplar: XP_002297655; XP_002308359; XP_002323329; XP_002311402 |
| TBL12/TBL13 | sorghum: XP_002453188; XP_002462885 |
| | corn: NP_001136835 |
| | poplar: XP_002300208; XP_002313938 |
| TBL7 | rice: NP_001044993; NP_001055601 |
| | sorghum: XP_002458862; XP_002439830; XP_002439831 |
| | corn: NP_001143090 |
| | poplar: XP_002300940; XP_002307018 |
| TBL6 | rice: NP_001049898 |
| | sorghum: XP_002467977; XP_002467978 |
| | poplar: XP_002301513 |
| TBL8/TBL9/ | rice: NP_001066145 |
| TBL10/TBL11 | sorghum: XP_002463295; XP_002448984; XP_002441819; XP_002440930 |
| | corn: NP_001169705; NP_001142167 |
| | poplar: XP_002322809; XP_002302119; XP_002311838; XP_002326021 |
| | pine: ABR16389 |
| TBL5 | rice: NP_001058264 |
| | sorghum: XP_002437399 |
| | poplar: XP_002308095; XP_002324663 |
| | pine: ACN40917; ADE76095 |
| TBL34/TBL35 | rice: NP_001051701 |
| | sorghum: XP_002441670; XP_002448865; XP_002448866; XP_002441671 |
| | corn: NP_001145493; NP_001169905 |
| | poplar: XP_002323025; XP_002323026 |
| TBL32/TBL33 | sorghum: XP_002440936; XP_002463646; XP_002463647 |
| | corn: NP_001130809; NP_001140631 |
| | poplar: XP_002311248; XP_002316189 |
| TBL28/ | rice: NP_001060731; NP_001049800; NP_001055286; NP_001044698; |
| TBL29/TBL30 | NP_001055807; NP_001065927 |
| | sorghum: XP_002463443; XP_002468038; XP_002456582; XP_002442440; |
| | XP_002441664 |
| | corn: NP_001142969; NP_001130739; NP_001183316; NP_001168900; |
| | NP_001131868; NP_001168546 |
| | poplar: XP_002316205; XP_002316206; XP_002311231; XP_002311232; |
| | XP_002316204; XP_002311234; XP_002316203 |
| TBL3/ | sorghum: XP_002468039 |
| TBL31/TBL36 | corn: NP_001143927 |
| | poplar: XP_002311947; XP_002323595; XP_002298909 |
| TBL44/TBL45 | rice: NP_001049797 |
| | sorghum: XP_002468040 |
| | corn: NP_001142288 |
| | poplar: XP_002298483; XP_002313988 |
| | pine: ABK22287; ABK25068; ABK26918 |
| TBL37/TBL38/ | rice: NP_001057784 |
| TBL39/TBL40 | sorghum: XP_002437078; XP_002457753 |
| | corn: NP_001145271 |
| | poplar: XP_002305083; XP_002316834; XP_002325581; XP_002327316 |
| TBL41/ | rice: NP_001043736 |
| TBL42/TBL43 | sorghum: XP_002458239 |
| | corn: NP_001144145 |
| | poplar: XP_002317443; XP_002332481; XP_002306016; XP_002304780; |
| | XP_002337457; XP_002304779; XP_002317442 |
| TBL14/ | sorghum: XP_002444474 |
| TBL15/TBL16 | corn: NP_001130877; NP_001144812 |
| | poplar: XP_002307039; XP_002334300; XP_002310522; XP_002326131 |
| | pine: ABK24354 |
| TBL17/TBL18 | rice: NP_001045183 |
| | corn: NP_001168473 |
| | poplar: XP_002318271; XP_002322423 |
| TBL27 | rice: NP_001057240; NP_001063036; NP_001059311; NP_001175123 |
| | sorghum: XP_002436768; XP_002450061; XP_002460152; XP_002460153; |
| | XP_002438121; XP_002438125; XP_002438123 |
| | poplar: XP_002312510; XP_002314730 |
| TBL22/ | rice: NP_001057237; NP_001173972; NP_001054930; NP_001057758 |
| TBL23/TBL24/ | sorghum: XP_002436766; XP_002446633; XP_002440799 |
| TBL25/TBL26 | corn: NP_001131156; NP_001140539 |
| | poplar: XP_002299487; XP_002303634; XP_002301401; XP_002320186; |
| | XP_002324017; XP_002317022 |
| TBL19/ | rice: NP_001048004; NP_001057365; NP_001172486; NP_001043741; |
| TBL20/TBL21 | NP_001057369; NP_001057366; NP_001057370; NP_001174709 |
| | sorghum: XP_002454344; XP_002454343; XP_002467212; XP_002348226; |
| | XP_002458240; XP_002438227 |
| | corn: NP_001136770; NP_001143774; NP_001144354 |
| | poplar: XP_002305967; XP_002329258; XP_002305969; XP_002305968 |

TBL3

The disclosure also relates to the A. thaliana TBL3, and to orthologous polypeptides. Polypeptides of the disclosure include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 6. Polypeptides of the disclosure also include polypeptides having at least 10, at least 12, at least 14, at least 16, at least 18, or at least 20 consecutive amino acids of SEQ ID NO: 6.
A. thaliana having a knock out mutation of TBL3 have reduced levels of O-acetylation of xylan and mannan (Example 7).

Polypeptides of the disclosure also include polypeptides that are orthologs of A. thaliana TBL3. Methods for identification of polypeptides that are homologs and orthologs of a polypeptide of interest are well known to one of skill in the art, as described above. The disclosure relates to polypeptides that are orthologous to A. thaliana TBL3, including, without limitation, orthologous polypeptides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Examples of polypeptides that are orthologous to A. thaliana TBL3 include, without limitation, the polypeptides orthologous to A. thaliana TBL3 provided in Table 1.

TBL17-TBL27 Clade

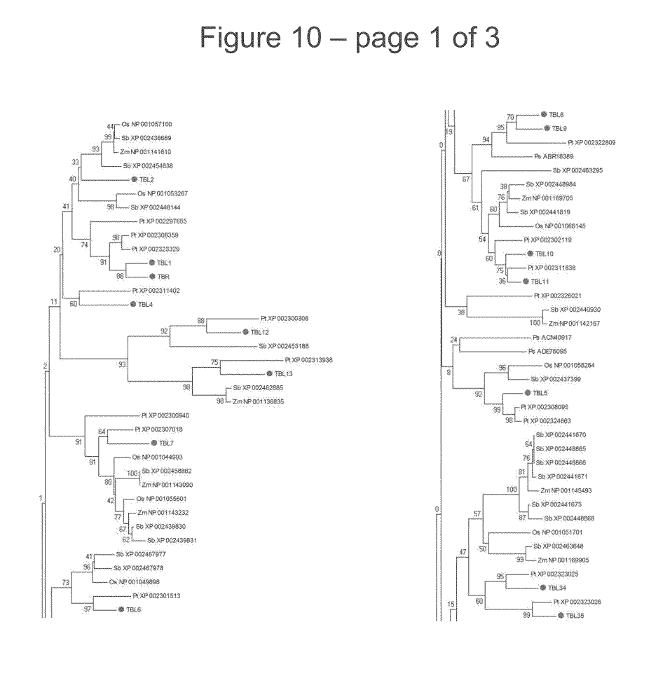

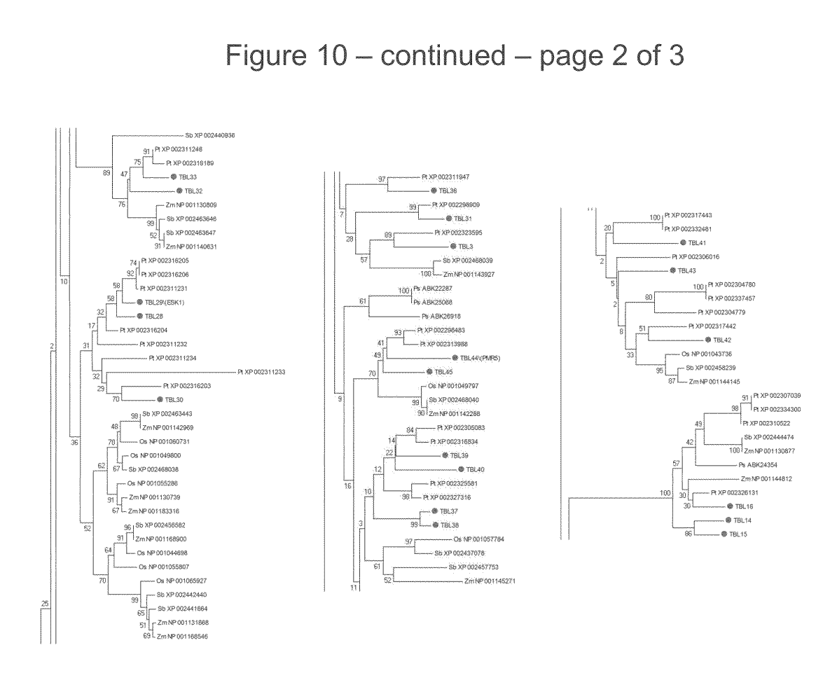

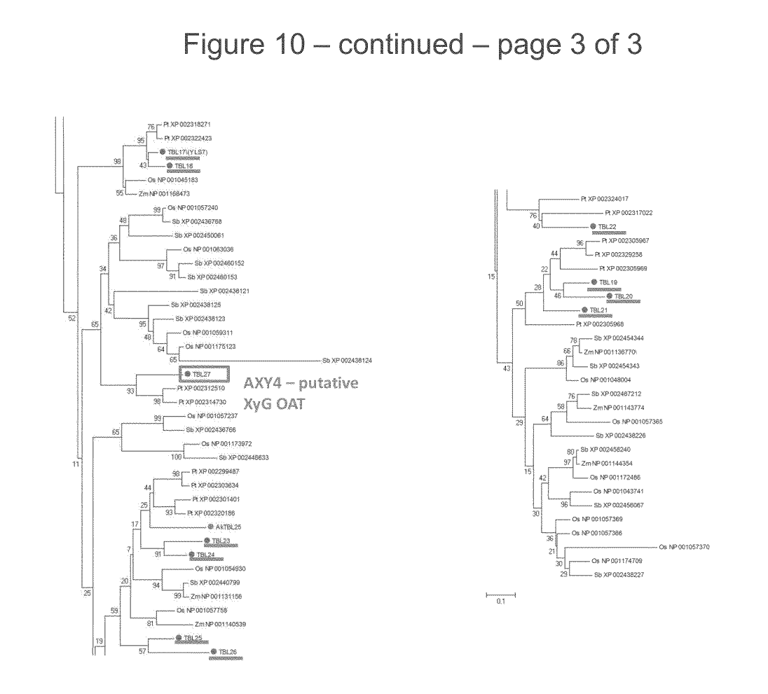

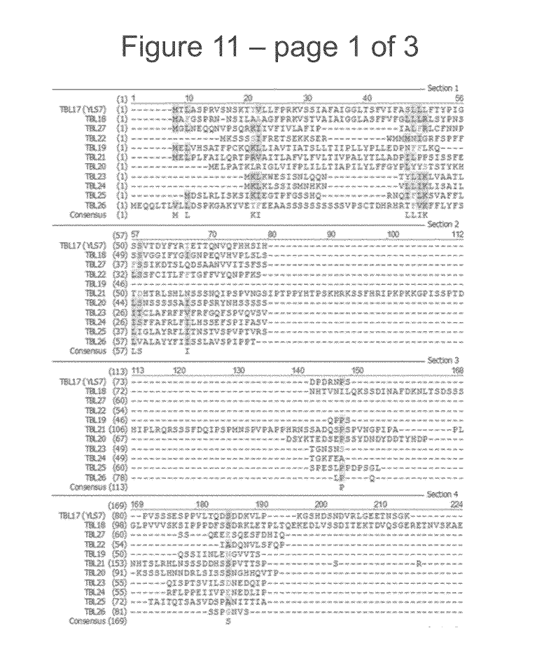

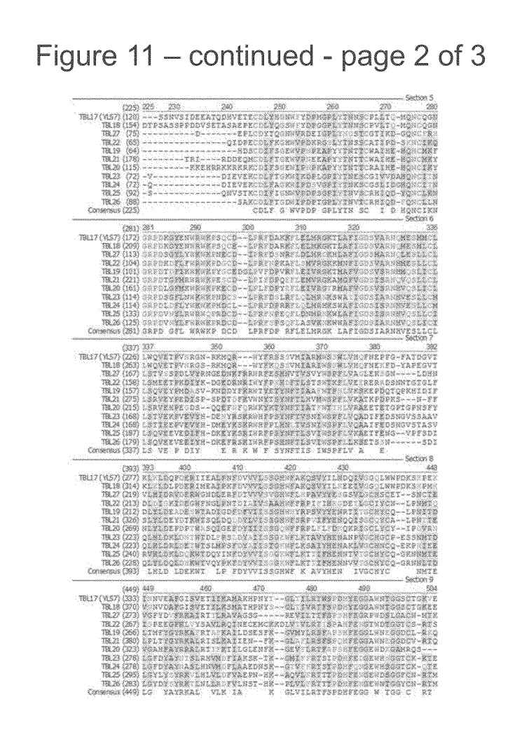

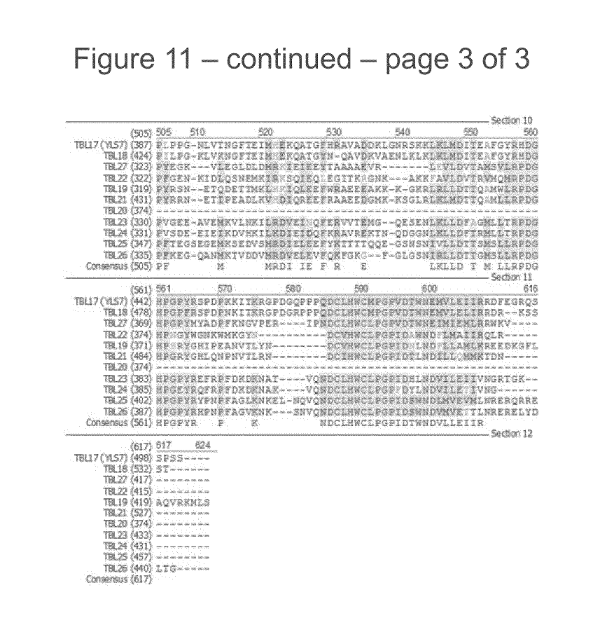

In some aspects, the present disclosure relates to polypeptides that are members of the TBL17-TBL27 clade. A. thaliana TBL17, TBL18, TBL19, TBL20, TBL21, TBL22, TBL23, TBL24, TBL25, TBL26, and TBL27 are particularly closely related phylogenetically, and form a distinct clade within the larger TBR/TBL family (FIGS. 10 and 11). This clade is referred to herein as the “TBL17-27 clade.”

Polypeptides of the TBL17-TBL27 clade have a highly conserved TBL domain. The TBL domains from TBL17-TBL27 polypeptides of A. thaliana have 83.9% similarity and 30.1% identity. A consensus sequence of the TBL domain from A. thaliana TBL17-TBL27 polypeptides is:

(SEQ ID NO: 115)
C-D-[L/Y/I]-[Y/T/F]-X-G-X-[V/F/I]-[P/Y/R/K]-
[D/N]-[P/E/K/S]-X-[G/A]-[P/S]-[L/I/Y]-Y-[T/N]-
[N/G]-X-[S/T]-C-X-X-[I/L/V]-X-[D/Q/E]-X-
[H/M/G/S/F]-[Q/K]-N-C-[I/Q/F/M/L]-[K/G/R/T/L]-
[N/H/Q/F/Y/N]-G-R-P-D-X-[G/D/N]-[Y/F]-[L/E/I/M]-
X-W-[R/K]-W-[K/Q]-P-X-[D/Q/E/G/S]-C-[D/E/S/L]-
X-X-[L/I]-P-[R/V/I/L]-F-[D/N/S]-[P/A/S]-X-
[R/K/A/E/Q]-F-L-[E/S/D/Q/A]-[L/M/I/N/S]-[M/V]-
[R/K]-[G/D/N/H]-[K/T]-X-[L/M/W]-[A/N/G]-
[F/I]-[I/V]-G-D-S-[V/M/I]-[A/S]-R-N-[H/Q]-
[V/M/L]-[E/Q]-S-[L/M]-[L/M/I]-C-[L/M/I]-L-
[S/W],
where X is any amino acid, [L/Y/I] is L, Y, or
I, [Y/T/F] is Y, T, or F, [V/F/I] is V, F, or I,
[P/Y/R/K] is P, Y, R, or K, [D/N] is D or N,
[P/E/K/S] is P, E, K, or S, [G/A] is G or A,
[P/S] is P or S, [L/I/Y] is L, I, or Y, [T/N] is
T or N, [N/G] is N or G, [S/T] is S or T,
[I/L/V] is I, L, or V, [D/Q/E] is D, Q, or E,
[H/M/G/S/F] is H, M, G, S, or F, [Q/K] is Q or
K, [I/Q/F/M/L]is I, Q, F, M, or L, [K/G/R/T/L]
is K, G, R, T, or L, [N/H/Q/F/Y/N] is N, H, Q,
F, Y, or N, [G/D/N] is G, D, or N, [Y/F] is Y or
F, [L/E/I/M] is L, E, I,or M, [R/K] is R or K,
[K/Q] is K or Q, [D/Q/E/G/S] is D, Q, E, G, or
S, [D/E/S/L] is D, E,S, or L, [L/I] is L or I,
[R/V/I/L] is R, V, I, or L, [D/N/S]is D, N, or
S, [P/A/S] is P, A, or S, [R/K/A/E/Q] is R, K,
A, E, or Q, [E/S/D/Q/A] is E, S, D, Q, or A,
[L/M/I/N/S] is L, M, I, N, or S, [M/V] is M
or V, [R/K] is R or K, [G/D/N/H] is G, D,
N, or H, [K/T] is K or T, [L/M/W] is
L, M, or W, [A/N/G] is A, N, or G, [F/I] is F or
I, [I/V] is I or V, [V/M/I] is V, M, or I,
[A/S] is A or S, [H/Q] is H or Q, [V/M/L] is V,
M, or L, [E/Q] is E or Q, [L/M] is L or M,
[L/M/I] is L, M, or I, [L/M/I] is L, M, or I,
and
[S/W] is S or W.

A consensus sequence of the TBL domain from TBL17-TBL27 clade proteins across multiple different species is also provided herein. The consensus TBL domain from TBL17-TBL27 clade proteins across multiple species provided herein has 69.5% similarity and 8.6% identity across TBL17-TBL27 clade proteins from Oryza sativa (rice), Zea mays (corn), Populus trichocarpa (poplar), Sorghum bicolor (sorghum), and Arabidopsis thaliana. A consensus sequence of the TBL domain from TBL17-TBL27 clade proteins across multiple different species provide herein is:

(SEQ ID NO: 116)
C-D-L-F-X-G-X-X-X-X-E-W-V-P-D-X-X-G-P-X-X-X-X-
X-X-Y-Y-T-N-X-T-C-X-X-I-X-X-X-Q-N-C-M-K-X-G-R-
P-D-X-G-Y-L-X-W-R-W-K-P-X-G-C-D-X-X-X-X-X-L-P-
R-F-D-X-X-R-F-L-X-L-V-R-G-K-S-L-A-F-V-G-D-S-L-
A-R-N-Q-M-X-S-L-L-C-L-L-S,
where X is any amino acid.

The present disclosure further relates to polypeptides that are orthologs of A. thaliana polypeptides of the TBL17-TBL27 clade. Methods for identification of polypeptides that are homologs and orthologs of a polypeptide of interest are well known to one of skill in the art, as described above.

Polypeptides of the disclosure include polypeptides having the sequence motif of SEQ ID NO: 115 or SEQ ID NO: 116. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 115. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 116. Polypeptides of the disclosure also include polypeptides having at least 10, at least 12, at least 14, at least 16, at least 18, or at least 20 consecutive amino acids of SEQ ID NOs: 115 or 116.

Polypeptides of the disclosure further include polypeptides that are orthologous to any member of the 11 member A. thaliana TBL17-TBL27 clade, including orthologous polypeptides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Examples of polypeptides that are orthologous to A. thaliana TBL17-TBL27 clade proteins include, without limitation, the polypeptides orthologous to A. thaliana TBL17-TBL27 polypeptides provided in Table 1.

TBL27

The disclosure further relates to the A. thaliana TBL27, and to orthologous polypeptides. Polypeptides of the disclosure include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 54. Polypeptides of the disclosure also include polypeptides having at least 10, at least 12, at least 14, at least 16, at least 18, or at least 20 consecutive amino acids of SEQ ID NO: 54.

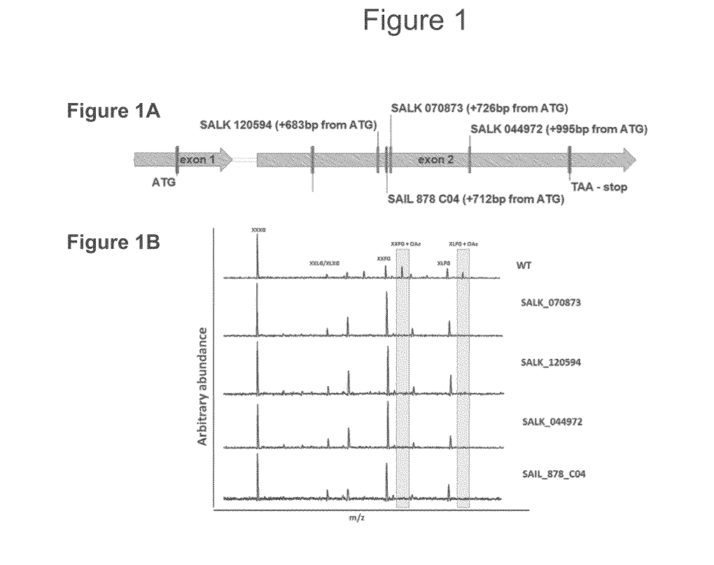

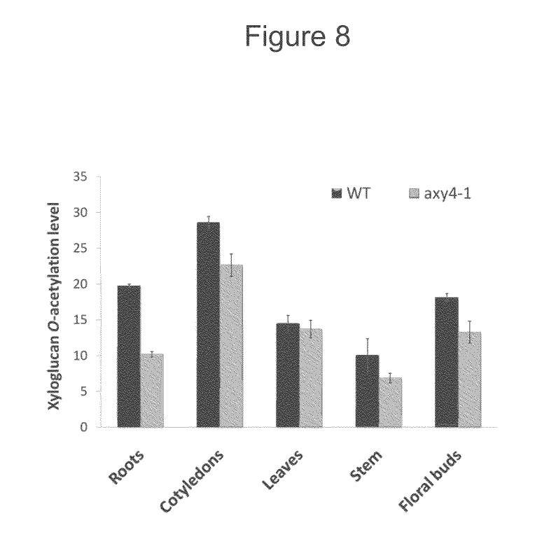

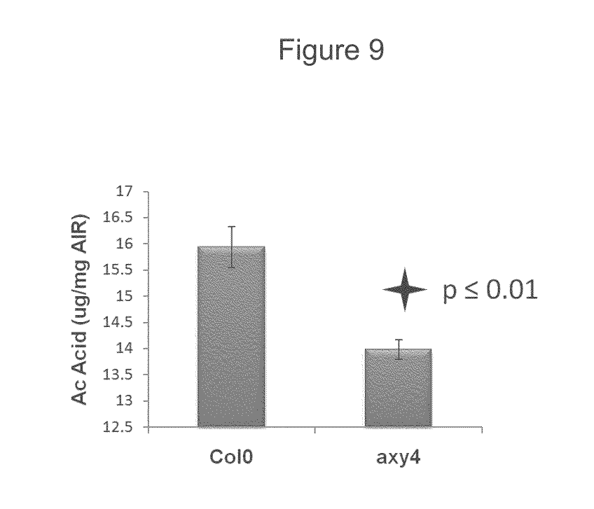

B. thaliana having a TBL27 gene with a proline to serine mutation in residue #126 of TBL27 (strain “axy4-1”) have reduced levels of O-acetylation of the hemicellulose xyloglucan in various tissues (FIGS. 8 and 9), and A. thaliana with TBL27 knocked out (through T-DNA insertion) have total loss of O-acetylation of xyloglucan (FIG. 1B).

Polypeptides of the disclosure also include polypeptides that are orthologs of A. thaliana TBL27. Methods for identification of polypeptides that are homologs and orthologs of a polypeptide of interest are well known to one of skill in the art, as described above. The disclosure relates to polypeptides that are orthologous to A. thaliana TBL27, including, without limitation, orthologous polypeptides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Examples of polypeptides that are orthologous to A. thaliana TBL27 include, without limitation, the polypeptides orthologous to A. thaliana TBL27 provided in Table 1.

TBL25

The disclosure also relates to the A. thaliana TBL25, and to orthologous polypeptides. Polypeptides of the disclosure include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 50. Polypeptides of the disclosure also include polypeptides having at least 10, at least 12, at least 14, at least 16, at least 18, or at least 20 consecutive amino acids of SEQ ID NO: 50.

A polypeptide orthologous to A. thaliana TBL25 in Amorphophallus konjac (“A. konjac”), AkTBL25 (SEQ ID NO: 110), is highly expressed in the developing corm of A. konjac, which produces a high level of acetylated glucomannan (Example 2). Accordingly, AkTBL25 is believed to promote the O-acetylation of glucomannan.

The disclosure also relates to A. konjac AkTBL25, and to orthologous polypeptides. Polypeptides of the disclosure include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 110. Polypeptides of the disclosure also include polypeptides having at least 10, at least 12, at least 14, at least 16, at least 18, or at least 20 consecutive amino acids of SEQ ID NO: 110.

Polypeptides of the disclosure also include polypeptides that are orthologs of A. thaliana TBL25. Methods for identification of polypeptides that are homologs and orthologs of a polypeptide of interest are well known to one of skill in the art, as described above. The disclosure relates to polypeptides that are orthologous to A. thaliana TBL25, including orthologous polypeptides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Examples of polypeptides that are orthologous to A. thaliana TBL25 include, without limitation, the polypeptides orthologous to A. thaliana TBL25 provided in Table 1.

MBOAT Polypeptides

The disclosure further relates to polypeptides that are members of the Membrane Bound O-Acyl Transferases (MBOAT) family. Members of the MBOAT superfamily contain several membrane spanning domains, usually between 8 and 10, share a certain detectable sequence similarity and transfer acyl residues onto hydroxyl groups of target components. (Hofmann, Trends Bio. Sci. 25(3): 111-112 (2000)) One such acyl residue might be an acetyl group that is being transferred onto a carbohydrate.

The disclosure also relates to the A. thaliana MBOAT family members At1g12640 (SEQ ID NO: 94), At1g34490 (SEQ ID NO: 96), At1g57600 (SEQ ID NO: 98), At1g63050 (SEQ ID NO: 100), At5g55320 (SEQ ID NO: 102), and At5g55350 (SEQ ID NO: 104). These and other MBOATs may be grouped into different MBOAT clades based on sequence homology. At1g12640, At1g34490, At1g57600, At1g63050, At5g55320, and At5g55350 may be grouped into three different clades based on homology, referred to herein as MBOAT Clade A, MBOAT Clade B, and MBOAT Clade C.

Also provided herein are MBOATs of Clade A. Examples of MBOATs of Clade A include, without limitation, A. thaliana At1g12640 (MBOAT1) and At1g63050 (MBOAT5). The polypeptides of MBOAT Clade A of A. thaliana share a consensus sequence that has 93.5% similarity and 87.0% identity across At1g12640 (MBOAT1) and At1g63050 (MBOAT5). In some aspects, a consensus sequence shared by A. thaliana MBOAT Clade A polypeptides is:

(SEQ ID NO: 117)
L-D-M-X-S-M-A-A-S-I-G-V-S-V-A-V-L-R-F-L-L-C-F-
V-A-T-I-P-I-S-F-X-X-R-X-I-P-S-R-L-G-K-H-I-Y-A-
A-A-S-G-A-F-L-S-Y-L-S-F-G-F-S-S-N-L-H-F-L-V-P-
M-T-I-G-Y-A-S-M-A-I-Y-R-P-X-X-G-X-I-T-F-F-L-G-
F-A-Y-L-I-G-C-H-V-F-Y-M-S-G-D-A-W-K-E-G-G-I-D-
S-T-G-A-L-M-V-L-T-L-K-V-I-S-C-S-I-N-Y-N-D-G-M-
L-K-E-E-G-L-R-E-A-Q-K-K-N-R-L-I-Q-M-P-S-L-I-E-
Y-F-G-Y-C-L-C-C-G-S-H-F-A-G-P-V-F-E-M-K-D-Y-L-
E-W-T-E-X-K-G-I-W-X-X-S-E-K-X-K-K-P-S-P-Y-G-A-
X-I-R-A-I-X-Q-A-A-I-C-M-A-L-Y-L-Y-L-V-P-Q-F-P-
L-T-R-F-T-E-P-V-Y-Q-E-W-G-F-L-K-K-F-X-Y-Q-Y-M-
A-G-F-T-A-R-W-K-Y-Y-F-I-W-S-I-S-E-A-S-I-I-I-S-
G-L-G-F-S-G-W-T-D-D-X-X-X-K-X-K-W-D-R-A-K-N-V-
D-I-L-G-V-E-L-A-K-S-A-V-Q-I-P-L-X-W-N-I-Q-V-S-
T-W-L-R-H-Y-V-Y-E-R-I-V-X-X-G-K-K-A-G-F-F-Q-L-
L-A-T-Q-T-V-S-A-V-W-H-G-L-Y-P-G-Y-I-I-F-F-V-Q-
S-A-L-M-I-X-G-S-K-X-I-Y-R-W-Q-Q-A-I-X-P-K-M-A-
M-L-R-N-I-L-V-X-I-N-F-L-Y-T-V-L-V-L-N-Y-S-A-V-
G-F-M-V-L-S-L-H-E-T-L-X-A-F-X-S-V-Y-Y-I-G-T-I-
I-P-I-A-L-I-L-L-S-Y-L-V-P-X-K-P-X-R-P-K-X-R-K-
E-E,
where X is any amino acid.

A consensus sequence shared by MBOAT Clade A proteins across multiple different species is also provided herein. The MBOAT Clade A proteins across multiple species share a consensus sequence provided herein that has 99.2% similarity and 61.8% identity across MBOAT Clade A proteins from Oryza sativa (rice), Zea mays (corn), Populus trichocarpa (poplar), Sorghum bicolor (sorghum), and Arabidopsis thaliana. In some aspects, a consensus sequence shared by MBOAT Clade A proteins across multiple different species is:

(SEQ ID NO: 118)
M-E-S-M-A-A-A-I-G-V-S-V-P-V-L-R-F-L-L-C-F-V-A-T-
I-P-V-S-F-L-W-R-F-V-P-S-A-X-G-K-H-L-Y-A-A-L-S-G-
A-F-L-S-Y-L-S-F-G-F-S-S-N-L-H-F-L-V-P-M-T-L-G-Y-
L-S-M-L-L-F-R-P-Y-C-G-I-I-T-F-L-X-G-F-G-Y-L-I-G-
C-H-V-Y-Y-M-S-G-D-A-W-K-E-G-G-I-D-A-T-G-A-L-M-V-
L-T-L-K-V-I-S-C-A-I-N-Y-N-D-G-M-L-K-E-E-G-L-R-E-
A-Q-K-K-N-R-L-I-K-L-P-S-L-I-E-Y-F-G-Y-C-L-C-C-G-
S-H-F-A-G-P-V-Y-E-M-K-D-Y-L-E-W-T-E-R-K-G-I-W-A-
S-S-E-K-G-P-T-P-S-P-F-G-A-T-I-R-A-L-L-Q-A-A-V-C-
M-A-L-Y-L-Y-L-I-P-Q-F-P-L-S-R-F-S-E-P-L-Y-Q-E-W-
G-F-W-K-R-L-X-Y-Q-Y-M-S-G-F-T-A-R-W-K-Y-Y-F-I-W-
S-I-S-E-A-A-I-I-I-S-G-L-G-F-S-G-W-T-D-S-S-P-P-K-
P-K-W-D-R-A-K-N-V-D-I-L-G-V-E-L-A-K-S-A-V-Q-I-P-
L-V-W-N-I-Q-V-S-T-W-L-R-H-Y-V-Y-E-R-L-V-Q-K-G-K-
K-P-G-F-F-Q-L-L-A-T-Q-T-V-S-G-V-I-H-E-L-V-F-F-Y-
I-T-R-E-X-P-T-G-E-V-T-L-F-F-V-L-H-G-V-C-T-A-A-E-
I-A-A-K,
where X is any amino acid.

In some aspects, provided herein are MBOATs of Clade B. Examples of MBOATs of Clade B include, without limitation, A. thaliana At1g34490 (MBOAT3), At5g55320 (MBOAT6) and At5g55350 (MBOAT7). The polypeptides of MBOAT Clade B of A. thaliana share a consensus sequence that has 94.7% similarity and 45.7% identity between At1g34490 (MBOAT3), At5g55320 (MBOAT6) and At5g55350 (MBOAT7). In some aspects, a consensus sequence shared by A. thaliana MBOAT Clade B polypeptides is:

(SEQ ID NO: 119)
M-E-E-E-L-K-S-L-I-K-V-W-X-X-A-I-I-S-V-S-Y-C-Y-Y-I-
P-S-R-I-K-S-G-V-X-R-L-L-S-V-L-P-V-C-V-L-F-L-V-L-P-
L-F-F-S-F-S-I-F-S-S-T-T-A-F-F-L-S-X-L-A-N-F-K-L-I-
L-F-S-F-D-Q-G-P-L-F-P-L-P-S-N-L-F-R-F-I-C-F-T-C-F-
P-I-K-L-Q-Q-N-P-K-S-Q-N-H-L-P-K-W-V-F-P-V-K-I-A-I-
F-V-V-L-L-H-I-H-X-Y-K-Q-X-L-P-P-T-L-L-L-X-L-Y-P-L-
H-I-Y-L-L-L-E-I-L-L-T-I-L-K-I-L-L-T-I-I-L-X-C-D-L-
E-P-H-F-N-E-P-Y-L-A-T-S-L-Q-D-F-W-G-R-R-W-N-L-M-V-
S-A-I-L-R-P-A-V-Y-S-P-V-R-X-V-C-Q-R-X-M-X-S-D-W-A-
L-F-I-G-V-F-A-T-F-L-V-S-G-V-I-H-E-L-V-F-F-Y-I-T-R-
E-X-P-T-G-E-V-T-L-F-F-V-L-H-G-V-C-T-A-A-E-I-A-A-K-
R-T-X-X-V-R-R-W-X-V-S-P-M-V-S-R-L-I-T-V-G-F-V-V-V-
T-G-G-W-L-F-F-P-Q-L-X-R-S-N-M-I-E-R-X-A-N-E-A-S-L-
F-I-D-F-V-K-X-K-L-F-Y-F;
where X is any amino acid.

A consensus sequence shared by MBOAT Clade B proteins across multiple different species is also provided herein. The MBOAT Clade B proteins across multiple species share a consensus sequence provided herein that has 81.3% similarity and 31.3% identity across MBOAT Clade B proteins from Oryza sativa (rice), Zea mays (corn), Populus trichocarpa (poplar), Sorghum bicolor (sorghum), and Arabidopsis thaliana. In some aspects, a consensus sequence shared by MBOAT Clade B proteins across multiple different species is:

(SEQ ID NO: 120)
E-P-Q-F-D-X-P-Y-L-A-S-S-L-R-D-F-W-G-R-R-W-N-L-M-V-
S-A-I-L-R-P-S-V-Y-X-P-V-R-A-X-X-G-X-X-X-X-X-X-X-A-
X-A-X-G-V-L-A-T-F-L-V-S-G-L-M-H-E-L-M-F-Y-Y-I-X-R-
X-X-P-T-G-E-V-T-X-F-F-L-L-H-G-V-C-X-A-A-E,
where X is any amino acid.

In some aspects, provided herein are MBOATs of Clade C. An example of a MBOAT of Clade C includes, without limitation, A. thaliana At1g57600 (MBOAT4) (SEQ ID NO: 98). A consensus sequence shared by MBOAT Clade C proteins across multiple different species is also provided herein. The MBOAT Clade C proteins across multiple species share a consensus sequence provided herein has 96.4% similarity and 53.8% identity across MBOAT Clade C proteins from Oryza sativa (rice), Zea mays (corn), Populus trichocarpa (poplar), Sorghum bicolor (sorghum), and Arabidopsis thaliana. In some aspects, a consensus sequence shared by MBOAT Clade C proteins across multiple different species is:

(SEQ ID NO: 121)
N-D-L-S-D-A-Q-W-R-N-F-R-G-N-L-P-I-L-T-I-V-M-G-A-
F-L-M-L-A-N-X-L-R-Y-C-Y-X-L-K-G-R-G-X-A-L-L-W-L-
L-L-S-L-S-Y-L-C-Y-L-H-G-A-C-V-V-F-I-L-L-I-A-L-I-
N-Y-X-I-V-K-L-F-A-X-Y-K-Y-C-T-X-L-I-W-S-F-N-L-S-
V-L-I-L-N-R-V-Y-E-G-Y-S-F-S-L-F-G-Q-Q-L-A-F-L-D-
N-Y-R-G-T-F-R-W-H-I-C-F-N-F-V-V-L-R-M-I-S-F-G-C-
D-Y-C-W-S-I-X-S-S-H-F-D-X-K-K-H-M-Q-R-C-X-V-C-X-
S-G-K-T-C-Y-X-X-L-Q-E-R-G-L-S-X-D-K-Y-T-F-L-I-Y-
L-C-Y-L-T-Y-A-P-L-Y-I-A-G-P-I-V-S-Y-N-A-F-A-A-Q-
L-D-V-P-Q-K-N-Y-S-F-A-Q-I-S-W-Y-G-L-R-W-I-L-S-F-
L-L-M-E-G-M-T-H-F-F-H-Y-N-A-F-V-V-S-R-L-W-Q-X-L-
S-P-F-E-I-F-I-I-S-Y-G-V-L-N-F-M-W-L-K-F-F-L-I-W-
R-Y-F-R-F-W-S-L-V-G-G-V-E-T-P-E-N-M-P-R-C-I-N-N-
C-H-D-L-E-S-F-W-K-S-W-H-A-S-F-N-R-W-L-V-R-Y-L-Y-
I-P-L-G-G-S-Q-R-K-L-L-S-I-W-V-I-F-T-F-V-A-V-W-H-
D-L-E-W-K-L-I-S-W-A-W-L-T-C-L-F-F-I-P-E-I-L-V-K-
S,
where X is any amino acid.

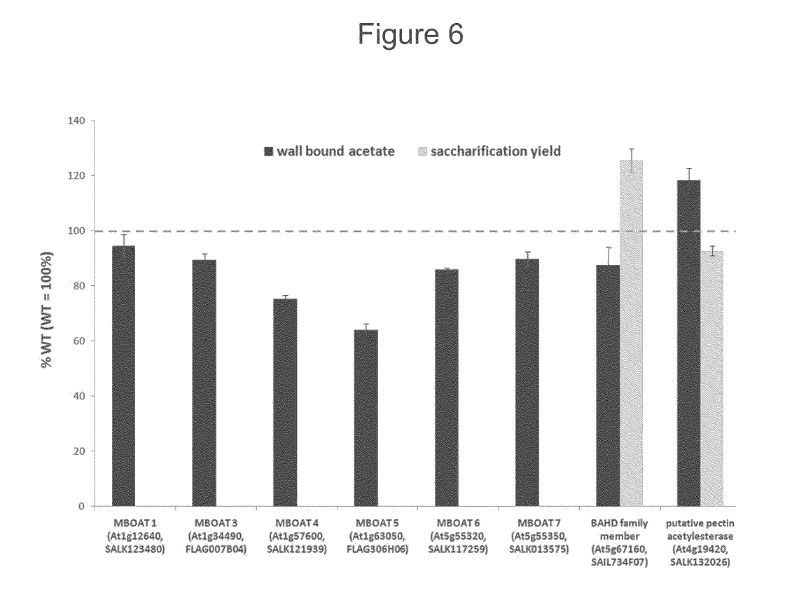

Analysis of A. thaliana tissue of rosette leaves and root material from plants having a T-DNA insertion in At1g12640, At1g34490, At1g57600, At1g63050, At5g55320, or At5g55350 has revealed that these lines exhibit a decrease in cell wall bound acetate between 6% and 36% compared to respective wild-type tissues (FIG. 6; Example 3).

The present disclosure also relates to polypeptides that are orthologs of A. thaliana At1g12640, At1g34490, At1g57600, At1g63050, At5g55320, or At5g55350. Methods for identification of polypeptides that are homologs and orthologs of a polypeptide of interest are well known to one of skill in the art, as described above.

Polypeptides of the disclosure include polypeptides containing the sequence of SEQ ID NO: 117 or SEQ ID NO: 118. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 117. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 118. Polypeptides of the disclosure also include polypeptides having at least 10, at least 12, at least 14, at least 16, at least 18, or at least 20 consecutive amino acids of SEQ ID NOs: 117 or 118.

Polypeptides of the disclosure include polypeptides containing the sequence of SEQ ID NO: 119 or SEQ ID NO: 120. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 119. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 120. Polypeptides of the disclosure also include polypeptides having at least 10, at least 12, at least 14, at least 16, at least 18, or at least 20 consecutive amino acids of SEQ ID NOs: 119 or 120.

Polypeptides of the disclosure include polypeptides containing the sequence of SEQ ID NO: 121. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 98. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 121. Polypeptides of the disclosure also include polypeptides having at least 10, at least 12, at least 14, at least 16, at least 18, or at least 20 consecutive amino acids of SEQ ID NOs: 98 or 121.

Polypeptides of the disclosure also include polypeptides that are orthologous to A. thaliana At1g12640, At1g34490, At1g57600, At1g63050, At5g55320, or At5g55350, including orthologous polypeptides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Examples of polypeptides that are orthologous to A. thaliana At1g12640, At1g34490, At1g57600, At1g63050, At5g55320, or At5g55350 include, without limitation the polypeptides orthologous to A. thaliana At1g12640, At1g34490, At1g57600, At1g63050, At5g55320, or At5g55350 provided in Table 2.

TABLE 2

| A. thaliana | |
|---|---|
| genes | Orthologs in Other Plant Species |
| BAHD family member- | rice: Os08g0112200 (gene); NP_001060828 (protein) |
| At5g67160 | sorghum: SORBIDRAFT_07g001180 (gene); XP_002444907 (protein) |
| | corn: LOC100191955 (gene); NP_001130851 (protein) |
| | poplar: POPTRDRAFT_645906 (gene); XP_002304178 (protein) |
| MBOAT1 - At1g12640/ | rice: Os02g0676000 (gene); NP_001047723 (protein) |
| MBOAT5 - At1g63050 | sorghum: SORBIDRAFT_04g031990 (gene); XP_002454488 (protein) |
| | poplar: POPTRDRAFT_799918 (gene); XP_002304469 (protein); |
| | OPTRDRAFT_815112 (gene); XP_002298063 (protein) |
| MBOAT3 - At1g34490 | rice: Os02g0454500 (gene); NP_001046772 (protein) |
| | sorghum: SORBIDRAFT_01g049060 (gene); XP_002468615 (protein) |
| | corn: LOC100280777 (gene); NP_001147171 (protein) |
| | poplar: POPTRDRAFT_548487 (gene); XP_002299545 (protein) |
| MBOAT4 - At1g57600 | rice: Os05g0144000 (gene); NP_001054621 (protein) |
| | sorghum: SORBIDRAFT_09g003400 (gene); XP_002440577 (protein) |
| | corn: LOC100282494 (gene); NP_001148875 (protein) |
| | poplar: POPTRDRAFT_823553 (gene); XP_002318916 (protein) |
| MBOAT6 - At5g55320/ | rice: Os04g0481900 (gene); NP_001053115 (protein) |
| MBOAT7 - At5g55350 | sorghum: SORBIDRAFT_06g004000 (gene); XP_002447562 (protein) |
| | corn: LOC100192487 (gene); NP_001131179 (protein) |
| | poplar: POPTRDRAFT_877603 (gene); XP_002314065 (protein) |
| Acetylesterase - | rice: Os02g0702400 (gene); NP_001047850 (protein) |
| At4g19420 | sorghum: SORBIDRAFT_04g030720 (gene); XP_002454426 (protein) |
| | corn: LOC100284816 (gene); NP_001151183 (protein) |
| | poplar: POPTRDRAFT_827492 (gene); XP_002328835 (protein) |

BAHD Polypeptides

The disclosure also relates to polypeptides that are members of the BAHD acyltransferase family. The BAHD superfamily represents a large group of plant specific acyl-CoA dependent acyltransferases, which contains many functionally diverse enzymes. The protein sequences are highly divergent sharing only 10-30% similarity on the amino acid level (Yu et al., Plant. Mol. Bio. 70(4): 421-442 (2009)). However, most members of the BAHD superfamily contain two highly conserved motifs—HXXXD (SEQ ID NO: 122) and DFGWG (SEQ ID NO: 123) (Yu et al. (2009) supra).

The disclosure also relates to the A. thaliana BAHD family member At5g67160 (SEQ ID NO: 106). Analysis of A. thaliana five week old cauline leaf tissue from a plant having a T-DNA insertion in At5g67160 has revealed that this line exhibits a decrease in cell wall bound acetate by 13% as compared to respective wild-type tissues (FIG. 6; Example 4).

Polypeptides of the disclosure also include polypeptides that are orthologs of A. thaliana At5g67160. Methods for identification of polypeptides that are homologs and orthologs of a polypeptide of interest are well known to one of skill in the art, as described above.

A consensus sequence shared by A. thaliana At5g67160 orthologs across multiple different species is also provided herein. The At5g67160 orthologs across multiple different species share a consensus sequence provided herein has 80% similarity and 20% identity across At5g67160 orthologs from Oryza sativa (rice), Zea mays (corn), Populus trichocarpa (poplar), Sorghum bicolor (sorghum), and Arabidopsis thaliana. In some aspects, a consensus sequence shared by A. thaliana At5g67160 orthologs across multiple species is:

(SEQ ID NO: 124)
E-C-F-F-X-F-X-A-E-S-V-R-K-L-K-A-K-A-N-A-E-M-A-A-
X-X-X-X-X-X-X-A-A-I-I-S-S-L-Q-A-L-L-A-H-I-W-R-A-
V-X-R-A-R-X-L-T-P-E-X-E-T-X-Y-X-L-V-I-G-C-R-A-R-
V-N-G-X-I-P-X-G-Y-V-G-N-A-V-V-X-G-I-A-X-L-T-A-G-
E-I-L-E-X-G-L-G-W-X-A-L-X-L-N-R-X-V-A-S-F-D-E-A-
X-M-R-A-X-L-A-X-W-V-R-X-P-X-F-X-X-X-X-X-X-X-X-G-
G-G-X-A-L-X-T-G-S-S-P-R-F-D-V-Y-G-N-D-F-G-W-G-R-
P-I-A-V-R-S-G-X-G-N-K-X-D-G-K-L-T-V-F-E-G-X-G-X-
A-G-S-M-S-L-E-V-C-L-A-P-X-A-L-X-K-L-V-A-D-X-E-F-
M-D-A-V;
where X is any amino acid.

Polypeptides of the disclosure also include polypeptides containing the sequence of SEQ ID NO: 124. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 106. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 124. Polypeptides of the disclosure also include polypeptides having at least 10, at least 12, at least 14, at least 16, at least 18, or at least 20 consecutive amino acids of SEQ ID NOs: 106 or 124.

Polypeptides of the disclosure further include polypeptides that are orthologous to A. thaliana At5g67160, including orthologous polypeptides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Examples of polypeptides that are orthologous to A. thaliana At5g67160 include, without limitation the polypeptides orthologous to A. thaliana At567160 provided in Table 2.

AXY9 Polypeptides

Polypeptides of the disclosure include polypeptides encoded by the nucleotide sequence of SEQ ID NO: 127. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the polypeptide encoded by the nucleotide sequence of SEQ ID NO: 127. Polypeptides of the disclosure also include polypeptides having at least 10, at least 12, at least 14, at least 16, at least 18, or at least 20 consecutive amino acids of the polypeptide encoded by the nucleotide sequence of SEQ ID NO: 127.

Polypeptides of the disclosure may further include polypeptides that are orthologous to A. thaliana AXY9. Such polypeptides may include, without limitation, orthologous AXY9 polypeptides from Aquilegia coerulea, Arabidopsis lyrata, Arabidopsis thaliana, Amborella trichopoda, Brachipodium distachyon, Brassica rapa, Citrus clementine, Carica papaya, Ceratodon purpureus, Capsella rubella, Cucumis sativus, Citrus sinensis, Eucalyptus grandis, Glycine max, Gossypium raimondii, Linum usitatissimum, Malus domestica, Manihot esculenta, Mimulus guttatus, Medicago truncatula, Nuphar advena, Oryza sativa, Phoenix dactilofera, Picea glauca, Physcomitrella patens, Prunus persica, Pinus pinaster, Pinus taeda, Populus trichocarpa, Panicum virgatum, Phaseolus vulgaris, Ricinus communis, Sorghum bicolor, Setaria italic, Solanum lycopersicum, Selaginella moellendorffii, Solanum tuberosum, Thellungiella halophile, Tropaeolum majus, Vitis vinifera, and Zea mays.

In some aspects, polypeptides that are orthologous to AXY9 may include, without limitation, AXY9 orthologs from corn, sorghum, miscanthus, sugarcane, poplar, spruce, pine, wheat, rice, soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), Miscanthus giganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Acetylesterase Polypeptides

The disclosure also relates to polypeptides that are acetylesterases. Acetylesterases are enzymes that catalyze the cleavage of an acetic ester.

The disclosure also relates to the A. thaliana pectin acetylesterase family member

At4g19420 (SEQ ID NO: 108). Analysis of A. thaliana five week old rosette leaf tissue from a plant having a T-DNA insertion in At4g19420 has revealed that this line exhibits an increase in cell wall bound acetate by 18% as compared to respective wild-type tissues, and a 8% decrease in glucose yield after dilute acid pre-treatment and enzymatic digest (FIG. 6; Example 5).

Polypeptides of the disclosure also include polypeptides that are orthologs of A. thaliana At4g19420. Methods for identification of polypeptides that are homologs and orthologs of a polypeptide of interest are well known to one of skill in the art, as described above. In one aspect, an acetylesterase polypeptide of the disclosure is the A. konjac polypeptide AkAE (SEQ ID NO: 112).

Polypeptides of the disclosure also include polypeptides containing the sequence of SEQ ID NO: 108. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 108. Polypeptides of the disclosure also include polypeptides having at least 10, at least 12, at least 14, at least 16, at least 18, or at least 20 consecutive amino acids of SEQ ID NO: 108.

Polypeptides of the disclosure also include polypeptides containing the sequence of SEQ ID NO: 112. Polypeptides of the disclosure also include polypeptides containing an amino acid sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 112. Polypeptides of the disclosure also include polypeptides having at least 10, at least 12, at least 14, at least 16, at least 18, or at least 20 consecutive amino acids of SEQ ID NO: 112.

The disclosure further relates to polypeptides that are orthologous to A. thaliana At4g19420, including orthologous polypeptides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Examples of polypeptides that are orthologous to A. thaliana At4g19420 include, without limitation, the polypeptides orthologous to A. thaliana At4g19420 provided in Table 2.

Polynucleotides of the Disclosure

The present disclosure further relates to decreasing the expression of polynucleotides that encode polypeptides that affect the acetylation of polysaccharides in plants. In some aspects, the disclosure relates to polynucleotides that encode polypeptides in the Trichome Birefringence (TBR)/Trichome Birefringence-Like (TBL) family. In some aspects, the disclosure relates to polynucleotides that encode polypeptides in the Membrane-Bound O-Acyl Transferase (MBOAT) family. In some aspects, the disclosure relates to polynucleotides that encode polypeptides in the BAHD acyltransferase family. In some aspects, the disclosure relates to polynucleotides that encode polypeptides that promote the O-acetylation of polysaccharides in plants. In some aspects, the disclosure relates to AXY9 polynucleotides and homologs thereof. In some aspects, the disclosure relates to polynucleotides that encode acetylesterases. Polynucleotides that encode a polypeptide are also referred to herein as “genes”. Methods for determining the relationship between a polypeptide and a polynucleotide that encodes the polypeptide are well known to one of skill in the art. Similarly, methods of determining the polypeptide sequence encoded by a polynucleotide sequence are well known to one of skill in the art.

As used herein, the terms “polynucleotide”, “nucleic acid sequence”, “nucleic acid”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications. As used herein, the symbols for nucleotides and polynucleotides are those recommended by the IUPAC-IUB Commission of Biochemical Nomenclature.

The present disclosure also relates to polynucleotides that affect the expression of a gene. In some aspects, polynucleotides that affect the expression of a gene inhibit gene expression. In some aspects, polynucleotides that inhibit gene expression have a sequence that is identical to the sequence of the gene to be affected. In some aspects, polynucleotides that inhibit gene expression have a sequence that is 75% or more, 80% or more, 85% or more, 90% or more, 95% or more, 96% or more, 97% or more, 98% or more, or 99% or more identical to the sequence of the gene to be affected. In some aspects, polynucleotides that inhibit gene expression have a sequence that is identical to a fragment of the sequence of the gene to be affected. In some aspects, polynucleotides that inhibit gene expression have a sequence that is 75% or more, 80% or more, 85% or more, 90% or more, 95% or more, 96% or more, 97% or more, 98% or more, or 99% or more identical to a fragment of the sequence of the gene to be affected. In some aspects, polynucleotides that inhibit gene expression have a sequence that is identical to a complement of the sequence of the gene to be affected. In some aspects, polynucleotides that inhibit gene expression have a sequence that is 75% or more, 80% or more, 85% or more, 90% or more, 95% or more, 96% or more, 97% or more, 98% or more, or 99% or more identical to a complement of the sequence of the gene to be affected. In some aspects, polynucleotides that inhibit gene expression have a sequence that is identical to a fragment of the complement of the sequence of the gene to be affected. In some aspects, polynucleotides that inhibit gene expression have a sequence that is 75% or more, 80% or more, 85% or more, 90% or more, 95% or more, 96% or more, 97% or more, 98% or more, or 99% or more identical to a fragment of the complement of the sequence of the gene to be affected.

In some aspects, polynucleotides of the disclosure include polynucleotides that have a nucleotide sequence that encodes a polypeptide sequence of a TBR/TBL family member. Polynucleotides that encode polypeptides of TBR/TBL family members include the A. thaliana polynucleotides SEQ ID NOs: 91 (TBR), 1 (TBL1), 3 (TBL2), 5 (TBL3), 7 (TBL4), 9 (TBL5), 11 (TBL6), 13 (TBL7), 15 (TBL8), 17 (TBL9), 19 (TBL10), 21 (TBL11), 23 (TBL12), 25 (TBL13), 27 (TBL14), 29 (TBL15), 31 (TBL16), 33 (TBL17) (YLS7), 35 (TBL18), 37 (TBL19), 39 (TBL20), 41 (TBL21), 43 (TBL22), 45 (TBL23), 47 (TBL24), 49 (TBL25), 51 (TBL26), 53 (TBL27), 55 (TBL28), 57 (TBL29) (ESK1), 59 (TBL30), 61 (TBL31), 63 (TBL32), 65 (TBL33), 67 (TBL34), 69 (TBL35), 71 (TBL36), 73 (TBL37), 75 (TBL38), 77 (TBL39), 79 (TBL40), 81 (TBL41), 83 (TBL42), 85 (TBL43), 87 (TBL44) (PMR5), and 89 (TBL45), and orthologous polynucleotides.

Methods for identifying orthologous sequences to a sequence of interest are well known to one of skill in the art, as described above. The disclosure relates to polynucleotides that encode polypeptides that are orthologous to A. thaliana TBR/TBL family polypeptides, including orthologous polynucleotides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Polynucleotides of the disclosure further include fragments of polynucleotides that encode polypeptides of the TBR/TBL family, polynucleotides that are complementary to polynucleotides that encode polypeptides of the TBR/TBL family, and fragments of polynucleotides that are complementary to polynucleotides that encode polypeptides of the TBR/TBL family.

Polynucleotides of the disclosure also include polynucleotides that have nucleotide sequences that encode polypeptides that contain the amino acid sequence of SEQ ID NOs: 113, 114, 115, or 116. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 113. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 114. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 115. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 116. Polynucleotides of the disclosure also include polynucleotides that encode polypeptides having at least 10, at least 12, at least 14, at least 16, at least 18, or at least 20 consecutive amino acids of SEQ ID NOs: 113, 114, 115 or 116.

Polynucleotides of the disclosure further include polynucleotides that have nucleotide sequences that encode the polypeptide sequence of A. thaliana TBL27 or orthologous polypeptides. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 54.

Polynucleotides of the disclosure further include polynucleotides that have nucleotide sequences that encode the polypeptide sequence of A. thaliana TBL25 or orthologous polypeptides. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 50.

Polynucleotides of the disclosure further include polynucleotides that have nucleotide sequences that encode the polypeptide sequence of A. konjac AkTBL25 or orthologous polypeptides. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 110. Polynucleotides of the disclosure also include polynucleotides that encode polypeptides having at least 10, at least 12, at least 14, at least 16, at least 18, or at least 20 consecutive amino acids of SEQ ID NOs: 54, 50, or 110.

Polynucleotides of the disclosure further include polynucleotides that have nucleotide sequences that encode polypeptides of MBOAT family members. Polynucleotides that encode polypeptides of MBOAT family members include the A. thaliana polynucleotides SEQ ID NOs: 93 (At1g12640), 95 (At1g34490), 97 (At1g57600), 99 (At1g63050), 101 (At5g55320), and 103 (At5g55350), and orthologous polynucleotides.

Methods for identifying orthologous sequences to a sequence of interest are well known to one of skill in the art, as described above. The disclosure relates to polynucleotides that encode polypeptides that are orthologous to A. thaliana MBOAT polypeptides, including orthologous polynucleotides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Polynucleotides of the disclosure further include fragments of polynucleotides that encode polypeptides of the MBOAT family, polynucleotides that are complementary to polynucleotides that encode polypeptides of the MBOAT family, and fragments of polynucleotides that are complementary to polynucleotides that encode polypeptides of the MBOAT family.

Polynucleotides of the disclosure further include polynucleotides that have nucleotide sequences that encode polypeptides that contain the amino acid sequence of SEQ ID NOs: 117, 118, 119, 120, or 121. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 117. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 118. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 119. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 120. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 121. Polynucleotides of the disclosure also include polynucleotides that encode polypeptides having at least 10, at least 12, at least 14, at least 16, at least 18, or at least 20 consecutive amino acids of SEQ ID NOs: 117, 118, 119, 120, or 121.

Polynucleotides of the disclosure further include polynucleotides that have nucleotide sequences that encode polypeptides sequences of BAHD family members. Polynucleotides that encode BAHD polypeptides include the A. thaliana polynucleotide SEQ ID NO: 105, and orthologous polynucleotides.

Methods for identifying orthologous sequences to a sequence of interest are well known to one of skill in the art, as described above. The disclosure relates to polynucleotides that encode polypeptides that are orthologous to A. thaliana BAHD polypeptides, including orthologous polynucleotides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Polynucleotides of the disclosure further include fragments of polynucleotides that encode polypeptides of the BAHD family, polynucleotides that are complementary to polynucleotides that encode polypeptides of the BAHD family, and fragments of polynucleotides that are complementary to polynucleotides that encode polypeptides of the BAHD family.

Polynucleotides of the disclosure include polynucleotides that have nucleotides sequences that encode polypeptides that contain the amino acid sequences of SEQ ID NOs: 122 and 123. Polynucleotides of the disclosure include polynucleotides that have nucleotides sequences that encode polypeptides that contain the amino acid sequences of SEQ ID NO: 106 or 124. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 106. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 124. Polynucleotides of the disclosure also include polynucleotides that encode polypeptides having at least 10, at least 12, at least 14, at least 16, at least 18, or at least 20 consecutive amino acids of SEQ ID NOs: 106 or 124.

Polynucleotides of the disclosure further include polynucleotides that have nucleotide sequences that encode polypeptide sequences of acetylesterase family members. Polynucleotides that encode acetylesterase polypeptides include the A. thaliana polynucleotide SEQ ID NO: 107, and orthologous polynucleotides. In one aspect, a polynucleotide that encodes an acetylesterase polypeptide provided herein is the A. konjac polynucleotide SEQ ID NO: 111

Methods for identifying orthologous sequences to a sequence of interest are well known to one of skill in the art, as described above. The disclosure relates to polynucleotides that encode polypeptides that are orthologous to A. thaliana acetylesterase polypeptides, including orthologous polynucleotides from corn (Zea mays), sorghum (Sorghum bicolor), sugarcane (Saccharum spp.), poplar (Populus trichocarpa), sitka spruce (Picea sitchensis), spruce (Picea spp.) pine (Pinaceae spp.), wheat (Triticum spp.), rice (Oryza sativa), soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), miscanthus (Miscanthus giganteus, Miscanthus sp.), sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Polynucleotides of the disclosure further include fragments of polynucleotides that encode acetylesterase polypeptides, polynucleotides that are complementary to polynucleotides that encode acetylesterase polypeptides, and fragments of polynucleotides that are complementary to polynucleotides that encode acetylesterase polypeptides.

Polynucleotides of the disclosure include polynucleotides that have nucleotides sequences that encode polypeptides that contain the amino acid sequences of SEQ ID NO: 108 or 112. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 108. Polynucleotides of the disclosure also include polynucleotides that encode polypeptides containing an amino acid sequence having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% similarity to the sequence of SEQ ID NO: 112. Polynucleotides of the disclosure also include polynucleotides that encode polypeptides having at least 10, at least 12, at least 14, at least 16, at least 18, or at least 20 consecutive amino acids of SEQ ID NOs: 108 or 112.

The disclosure also relates to the A. thaliana AXY9 polynucleotide (At3g03210) (SEQ ID NO: 127). Analysis of A. thaliana plants harboring AXY9 mutations revealed that these AXY9 mutant plants have reduced acetate content in multiple wall polymers as compared to a wild-type plant.

Polynucleotides of the disclosure include the polynucleotide sequence of SEQ ID NO: 127. Polynucleotides of the disclosure also include polynucleotides containing a nucleotide sequence having at least 10%, at least 15%, at least 20%, at least 25%, at least 30%, at least 35%, at least 40%, at least 45%, 50%, at least 55%, at least 60%, at least 65%, at least 70%, at least 75%, at least 80%, at least 85%, at least 90%, at least 95%, at least 98%, at least 99%, or 100% similarity to the polynucleotide sequence of SEQ ID NO: 127.

Polynucleotides of the disclosure may further include polynucleotides that are homologous to the sequence of SEQ ID NO: 127. Methods for identifying homologous nucleotide sequences to a nucleotide sequence of interest are well known to one of skill in the art, as described above. Such polynucleotides may include, without limitation, SEQ ID NO: 127 homologs from Aquilegia coerulea, Arabidopsis lyrata, Arabidopsis thaliana, Amborella trichopoda, Brachipodium distachyon, Brassica rapa, Citrus clementine, Carica papaya, Ceratodon purpureus, Capsella rubella, Cucumis sativus, Citrus sinensis, Eucalyptus grandis, Glycine max, Gossypium raimondii, Linum usitatissimum, Malus domestica, Manihot esculenta, Mimulus guttatus, Medicago truncatula, Nuphar advena, Oryza sativa, Phoenix dactilofera, Picea glauca, Physcomitrella patens, Prunus persica, Pinus pinaster, Pinus taeda, Populus trichocarpa, Panicum virgatum, Phaseolus vulgaris, Ricinus communis, Sorghum bicolor, Setaria italic, Solanum lycopersicum, Selaginella moellendorffii, Solanum tuberosum, Thellungiella halophile, Tropaeolum majus, Vitis vinifera, and Zea mays.

In some aspects, polynucleotides homologous to SEQ ID NO: 127 may include, without limitation, polynucleotides homologous to SEQ ID NO: 127 from corn, sorghum, miscanthus, sugarcane, poplar, spruce, pine, wheat, rice, soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), Miscanthus giganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

Plants

The present disclosure also relates to plants having a decreased level of polysaccharide O-acetylation. In some aspects, the disclosure relates to plants having a decreased level of O-acetylation of a cell wall polysaccharide. In some aspects, the disclosure relates to plants having a decreased level of O-acetylation of a hemicellulose. In some aspects, the disclosure relates to plants having a decreased level of O-acetylation of one or more of xylans, xyloglucans, glucuronoarabinoxylans, mannans, glucomannans, galactoglucomannans and arabinoxylans. As used herein, “decreased” level of acetylation refers to a decreased level of O-acetylation, as compared to the level of O-acetylation in a corresponding non-modified plant. As used herein, a “non-modified” plant refers to a plant that has not been modified in regards to the trait at issue (e.g. in this case, O-acetylation level). As used herein, a “decreased” level includes a “reduced” level.

The disclosure further relates to plants having a decreased level of O-acetylation of one or more polysaccharides of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less O-acetylation as compared to a corresponding non-modified plant. In some aspects, the disclosure relates to plants having a decreased level of O-acetylation of xyloglucan, of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less O-acetylation as compared to a corresponding non-modified plant. In some aspects, the disclosure relates to plants having a decreased level of O-acetylation of mannans of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less O-acetylation as compared to a corresponding non-modified plant. In some aspects, the disclosure relates to plants having a decreased level of O-acetylation of glucomannan of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less O-acetylation as compared to a corresponding non-modified plant. In some aspects, the disclosure relates to plants having a decreased level of O-acetylation of galactoglucomannan of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less O-acetylation as compared to a corresponding non-modified plant. In some aspects, the disclosure relates to plants having a decreased level of O-acetylation of xylan of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less O-acetylation as compared to a corresponding non-modified plant. In some aspects, the disclosure relates to plants having a decreased level of O-acetylation of arabinoxylan of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less O-acetylation as compared to a corresponding non-modified plant. In some aspects, the disclosure relates to plants having a decreased level of O-acetylation of glucuronoxylan of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less O-acetylation as compared to a corresponding non-modified plant. In some aspects, the disclosure relates to plants having a decreased level of O-acetylation of glucuronoarabinoxylan of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less O-acetylation as compared to a corresponding non-modified plant.

The present disclosure further relates to a plant having a decreased level of a polypeptide having O-acetyltransferase activity, as compared to the level of the polypeptide in a corresponding, non-modified plant. In some aspects, the disclosure relates to plants having a decreased level of a polypeptide having O-acetyltransferase activity of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less polypeptides as compared to a corresponding non-modified plant.

The present disclosure further relates to a plant having a decreased level of expression of a gene encoding a polypeptide having O-acetyltransferase activity, as compared to the level of expression of the polynucleotide in a corresponding non-modified plant. In some aspects, the disclosure relates to plants having a decreased level of a gene encoding a polypeptide having O-acetyltransferase activity of about 5% less, 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, or 100% less polynucleotides as compared to a corresponding non-modified plant.

The present disclosure further relates to a plant having an increased level of a polypeptide having acetylesterase activity, as compared to the level of the polypeptide in a corresponding, non-modified plant. In some aspects, the disclosure relates to plants having an increased level of a polypeptide having acetylesterase activity of about 5% more, 10% more, 15% more, 20% more, 25% more, 30% more, 35% more, 40% more, 45% more, 50% more, 55% more, 60% more, 65% more, 70% more, 75% more, 80% more, 85% more, 90% more, 95% more, or 100% more polypeptides as compared to a corresponding non-modified plant.

The present disclosure further relates to a plant having an increased level of expression of a gene encoding a polypeptide having acetylesterase activity, as compared to the level of expression of the polynucleotide in a corresponding non-modified plant. In some aspects, the disclosure relates to plants having an increased level of a gene encoding a polypeptide having acetylesterase activity of about 5% more, 10% more, 15% more, 20% more, 25% more, 30% more, 35% more, 40% more, 45% more, 50% more, 55% more, 60% more, 65% more, 70% more, 75% more, 80% more, 85% more, 90% more, 95% more, or 100% more polynucleotides as compared to a corresponding non-modified plant.

The present disclosure further relates to plants that have been modified to alter the level of one or more polypeptides that affect polysaccharide acetylation. In some aspects, the present disclosure relates to plants that have been modified to alter the level of two or more polypeptides that affect polysaccharide acetylation. In some aspects, the present disclosure relates to plants that have been modified to alter the level of three or more polypeptides that affect polysaccharide acetylation.

The present disclosure further relates to plants that have been modified to alter the expression of one or more genes that encode polypeptides that affect polysaccharide acetylation. In some aspects, the present disclosure relates to plants that have been modified to alter the expression of two or more genes that encode polypeptides that affect polysaccharide acetylation. In some aspects, the present disclosure relates to plants that have been modified to alter the expression of three or more genes that encode polypeptides that affect polysaccharide acetylation.

The present disclosure also includes offspring of plants that have been modified to have a decreased level of polysaccharide O-acetylation. The disclosure further includes seeds, cuttings, rhizomes, runners, plant cells, and tissues of plants that have been modified to have a decreased level of polysaccharide O-acetylation.

In some aspects, the present disclosure relates to plants that have been modified to alter the expression of an AXY9 polynucleotide. In some aspects, the present disclosure relates to plants that have been modified to alter the expression of the polynucleotide sequence set forth in SEQ ID NO: 127 or a homolog thereof. In some aspects, the present disclosure relates to plants that have altered expression of SEQ ID NO: 127 or a homolog thereof and at least one other gene involved in polysaccharide acetylation.

The present disclosure further relates to plants that have been modified to alter the level of one or more polypeptides that affect polysaccharide acetylation. In some aspects, the present disclosure relates to plants that have been modified to alter the level of an AXY9 polypeptide. In some aspects, the present disclosure relates to plants that have altered levels of an AXY9 polypeptide or an ortholog thereof and at least one other polypeptide involved in polysaccharide O-acetylation.

In some aspects, the present disclosure relates to offspring of plants that have been modified to alter the expression of an AXY9 polynucleotide. In some aspects, the present disclosure relates to offspring of plants that have been modified to alter the expression of the polynucleotide sequence set forth in SEQ ID NO: 127 or a homolog thereof.

Plant Types

The present disclosure also relates to various kinds of plants. Plants of the disclosure include both monocotyledonous and dicotyledonous plants. In some aspects, plants of the disclosure are used as lignocellulosic material for biofuel production and/or the production of commodity chemicals. In some aspects, plants of the disclosure are used for, without limitation food, cosmetic, or pharmaceutical production.

Plants of the disclosure may include, without limitation, Aquilegia coerulea, Arabidopsis lyrata, Arabidopsis thaliana, Amborella trichopoda, Brachipodium distachyon, Brassica rapa, Citrus clementine, Carica papaya, Ceratodon purpureus, Capsella rubella, Cucumis sativus, Citrus sinensis, Eucalyptus grandis, Glycine max, Gossypium raimondii, Linum usitatissimum, Malus domestica, Manihot esculenta, Mimulus guttatus, Medicago truncatula, Nuphar advena, Oryza sativa, Phoenix dactilofera, Picea glauca, Physcomitrella patens, Prunus persica, Pinus pinaster, Pinus taeda, Populus trichocarpa, Panicum virgatum, Phaseolus vulgaris, Ricinus communis, Sorghum bicolor, Setaria italic, Solanum lycopersicum, Selaginella moellendorffii, Solanum tuberosum, Thellungiella halophile, Tropaeolum majus, Vitis vinifera, and Zea mays.

Plants of the disclosure also include, without limitation, corn, sorghum, miscanthus, sugarcane, poplar, spruce, pine, wheat, rice, soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), Miscanthus giganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

The present disclosure also relates to plant cells that have been modified to reduce polysaccharide O-acetylation. Plant cells of the disclosure include cells from, without limitation, corn, sorghum, miscanthus, sugarcane, poplar, spruce, pine, wheat, rice, soy, cotton, barley, turf grass, tobacco, potato, bamboo, rape, sugar beet, sunflower, willow, eucalyptus, Amorphophallus spp., Amorphophallus konjac, switchgrass (Panicum virgatum), giant reed (Arundo donax), reed canarygrass (Phalaris arundinacea), Miscanthus giganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, Aquilegia coerulea, Arabidopsis lyrata, Arabidopsis thaliana, Amborella trichopoda, Brachipodium distachyon, Brassica rapa, Citrus clementine, Carica papaya, Ceratodon purpureus, Capsella rubella, Cucumis sativus, Citrus sinensis, Eucalyptus grandis, Glycine max, Gossypium raimondii, Linum usitatissimum, Malus domestica, Manihot esculenta, Mimulus guttatus, Medicago truncatula, Nuphar advena, Oryza sativa, Phoenix dactilofera, Picea glauca, Physcomitrella patens, Prunus persica, Pinus pinaster, Pinus taeda, Populus trichocarpa, Panicum virgatum, Phaseolus vulgaris, Ricinus communis, Sorghum bicolor, Setaria italic, Solanum lycopersicum, Selaginella moellendorffii, Solanum tuberosum, Thellungiella halophile, Tropaeolum majus, Vitis vinifera, and Zea mays, and Kentucky bluegrass.

Methods of Decreasing Gene Expression/Polypeptide Levels in a Plant

In some embodiments, the present disclosure relates to decreasing the expression of a gene in a plant. In certain embodiments, decreasing the expression of a gene in a plant results in reduced activity of the protein encoded by the gene. As used herein, “decreasing” the level of expression of a gene includes “reducing”, “inhibiting” and “suppressing” the expression of a gene. The level of expression of a gene may be assessed by measuring the level of mRNA encoded by the gene, and/or by measuring the level or activity of the polypeptide encoded by the gene.

Genes can be suppressed using any number of techniques well known in the art. For example, one method of suppression is sense suppression (also known as co-suppression). Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al, The Plant Cell 2:279-289 (1990); Flavell, Proc. Natl. Acad. Sci, USA 91:3490-3496 (1994); Kooter and Moi, Current Opin. Biol. 4:166-171 (1993); and U.S. Pat. Nos. 5,034,323, 5,231,020, and 5,283,184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity can exert a more effective repression of expression of the endogenous sequences. In some embodiments, sequences with substantially greater identity are used, e.g., at least about 80, at least about 95%, or 100% identity are used. As with antisense regulation, further discussed below, the effect can be designed and tested to apply to any other proteins within a similar family of genes exhibiting homology or substantial homology.

For sense suppression, the introduced sequence in the expression cassette, needing less than absolute identity, also need not be full length, relative to either the primary transcription product or fully processed rnRNA. Furthermore, the introduced sequence need not have the same intron or exon pattern, and identity of non-coding segments will be equally effective. In some embodiments, a sequence of the size ranges noted above for antisense regulation is used, i.e., 30-40, or at least about 20, 50, 100, 200, 500 or more nucleotides.

RNAi

Endogenous gene expression may also be suppressed by means of RNA interference (RNAi) (and indeed co-suppression can be considered a type of RNAi), which uses a double-stranded RNA having a sequence identical or similar to the sequence of the target gene. As used herein RNAi, includes the use of micro RNA, such as artificial miRNA to suppress expression of a gene.

RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA. Although complete details of the mechanism of RNAi are still unknown, it is considered that the introduced double-stranded RNA is initially cleaved into small fragments, which then serve as indexes of the target gene in some manner, thereby degrading the target gene. RNAi is known to be also effective in plants (see, e.g., Chuang, C. F. & Meyerowitz, E. M., Proc. Natl. Acad. Sd. USA 97: 4985 (2000); Waterhouse et al, Proc. Natl. Acad. Sci. USA 95:13959-13964 (1998); Tabara et al. Science 282:430-431 (1998); Matthew, Comp Fund Genom 5: 240-244 (2004); Lu, et al, Nucleic Acids Res. 32(21):171 (2004)).

Thus, in some embodiments, inhibition of gene expression is achieved using RNAi techniques. For example, to achieve suppression of the expression of a DNA encoding a protein using RNAi, a double-stranded RNA having the sequence of a DNA encoding the protein, or a substantially similar sequence thereof (including those engineered not to translate the protein) or fragment thereof, is introduced into a plant of interest. As used herein, RNAi and dsRNA both refer to gene-specific silencing that is induced by the introduction of a double-stranded RNA molecule, see e.g., U.S. Pat. Nos. 6,506,559 and 6,573,099, and includes reference to a molecule that has a region that is double-stranded, e.g., a short hairpin RNA molecule. The resulting plants may then be screened for a phenotype associated with the reduced expression of the target gene, e.g., reduced acetate, and/or by monitoring steady-state RNA levels for transcripts encoding the protein. Although the genes used for RNAi need not be completely identical to the target gene, they may be at least 70%, 80%, 90%, 91%, 92%, 93%, 94%, 95%, 96%, 97%, 98%, 99% or more identical to the target gene sequence. See, e.g., U.S. Patent Publication No. 2004/0029283. The constructs encoding an RNA molecule with a stem-loop structure that is unrelated to the target gene and that is positioned distally to a sequence specific for the gene of interest may also be used to inhibit target gene expression. See, e.g., U.S. Patent Publication No. 2003/0221211.

The RNAi polynucleotides may encompass the full-length target RNA or may correspond to a fragment of the target RNA. In some cases, the fragment will have fewer than 100, 200, 300, 400, or 500, nucleotides corresponding to the target sequence. In addition, in some aspects, these fragments are at least, e.g., 50, 100, 150, 200, or more nucleotides in length. Interfering RNAs may be designed based on short duplexes (i.e., short regions of double-stranded sequences). Typically, the short duplex is at least about 15, 20, or 25-50 nucleotides in length (e.g., each complementary sequence of the double stranded RNA is 15-50 nucleotides in length), often about 20-30 nucleotides, e.g., 20, 21, 22, 23, 24, 25, 26, 27, 28, 29, or 30 nucleotides in length. In some cases, fragments for use in RNAi will correspond to regions of a target protein that do not occur in other proteins in the organism or that have little similarity to other transcripts in the organism, e.g., selected by comparison to sequences in analyzing publicly-available sequence databases. Similarly, RNAi fragments may be selected for similarity or identity with a conserved sequence of a gene family of interest, such as those described herein, so that the RNAi targets multiple different gene transcripts containing the conserved sequence.

RNAi may be introduced into a cell as part of a larger DNA construct. Often, such constructs allow stable expression of the RNAi in cells after introduction, e.g., by integration of the construct into the host genome. Thus, expression vectors that continually express RNAi in cells transfected with the vectors may be employed for this disclosure. For example, vectors that express small hairpin or stem-loop structure RNAs, or precursors to microRNA, which get processed in vivo into small RNAi molecules capable of carrying out gene-specific silencing (Brummelkamp et al, Science 296:550-553 (2002), and Paddison, et al., Genes & Dev. 16:948-958 (2002)) can be used. Post-transcriptional gene silencing by double-stranded RNA is discussed in further detail by Hammond et al., Nature Rev Gen 2: 110-119 (2001), Fire et al., Nature 391: 806-811 (1998) and Timmons and Fire, Nature 395: 854 (1998).

Methods for selection and design of sequences that generate RNAi are well known in the art (e.g. U.S. Pat. No. 6,506,559; U.S. Pat. No. 6,511,824; and U.S. Pat. No. 6,489,127).

One of skill in the art will recognize that using technology based on specific nucleotide sequences (e.g., antisense or sense suppression technology), families of homologous genes can be suppressed with a single sense or antisense, discussed below, transcript. For instance, if a sense or antisense transcript is designed to have a sequence that is conserved among a family of genes, then multiple members of a gene family can be suppressed. Conversely, if the goal is to only suppress one member of a homologous gene family, then the sense or antisense transcript should be targeted to sequences with the most variation between family members.

The term “target gene” or “target sequences”, refers to a gene targeted for reduced expression.

In one format, one or more different genes can be inhibited using the same interfering RNA. For example, some or all genes in the TBR/TBL family in a plant may be targeted by using an RNAi that is designed to a conserved region of the gene of the TBR/TBL family. In another example, some or all genes in the TBL17-TBL27 clade in a plant may be targeted by using an RNAi that is designed to a conserved region of the gene of the TBL17-TBL27 family. In other aspects, individual gene may be targeted by using an RNAi that is specific that gene.

In some aspects, an RNAi having the sequence of uuuaucggugauuccauggcuagaaaucagcuugagucucuuuuaugc (SEQ ID NO: 125), or a fragment thereof. In some aspects, RNAi having the sequence of SEQ ID NO: 125 or a fragment thereof is used to repress the expression of one or more genes in the TBR/TBL family. Also provided herein is an RNAi having a sequence complementary to SEQ ID NO: 125, or complementary to a fragment of SEQ ID NO: 125.

Antisense and Ribozyme Suppression

A reduction of gene expression in a plant of a target gene may be obtained by introducing into plants antisense constructs based on a target gene polynucleotide sequences. For antisense suppression, a target sequence is arranged in reverse orientation relative to the promoter sequence in the expression vector. The introduced sequence need not be a full length cDNA or gene, and need not be identical to the target cDNA or a gene found in the plant variety to be transformed. Generally, however, where the introduced sequence is of shorter length, a higher degree of homology to the native target sequence is used to achieve for effective antisense suppression. In some aspects, the introduced antisense sequence in the vector will be at least 30 nucleotides in length, and improved antisense suppression will typically be observed as the length of the antisense sequence increases. In some aspects, the length of the antisense sequence in the vector will be greater than 100 nucleotides. Transcription of an antisense construct as described results in the production of RNA molecules that are the reverse complement of mRNA molecules transcribed from an endogenous target gene. Suppression of a target gene expression can also be achieved using a ribozyme. The production and use of ribozymes are disclosed in U.S. Pat. No. 4,987,071 and U.S. Pat. No. 5,543,508.

Mutagenesis

In some formats, random mutagenesis approaches may be used to disrupt or “knockout” the expression of a target gene using either chemical or insertional mutagenesis, or irradiation.

One method of mutagenesis and mutant identification is known as TILLING (for “Targeting Induced Local Lesions in Genomes”). In this method, mutations are induced in the seed of a plant of interest, for example, using ethane methyl sulfonate (EMS) treatment (Hoffman, Mutation Research 75(1): 63-129 (1980)) or fast neutron bombardment (Li et al., Plant Journal 27(3):235-242, (2001)). The resulting plants are grown and self-fertilized, and the progeny are assessed. For example, the plants may be assed using PCR to identify whether a mutated plant has a mutation in a target gene, e.g., that reduces expression of a target gene, or by evaluating whether the plant has reduced levels of wall acetate in a part of the plant that expressed the target gene. TILLING can identify mutations that may alter the expression of specific genes or the activity of proteins encoded by these genes (see Colbert et al. Plant Physiol 126:480-484 (2001); McCallum et al Nature Biotechnology 18:455-457 (2000)).

Another method for abolishing or decreasing the expression of a target gene is by insertion mutagenesis using the T-DNA of Agrobacterium tumefaciens. After generating the insertion mutants, the mutants can be screened to identify those containing the insertion in a target gene. Mutants containing a single mutation event at the desired gene may be crossed to generate homozygous plants for the mutation (Koncz et al. Methods in Arabidopsis Research. World Scientific (1992)).

Another method to disrupt a target gene is by use of the cre-lox system (for example, as described in U.S. Pat. No. 5,658,772).

In some aspects, the disclosure includes mutation of any gene orthologous to an A. thaliana gene provided herein as a gene that promotes polysaccharide O-acetylation. Examples of genes that may be disrupted by mutagenesis include, without limitation, NP_{—}001057240; NP_{—}001063036; NP_{—}001059311; NP_{—}001175123; XP_{—}002436768; XP_{—}002450061; XP_{—}002460152; XP_{—}002460153; XP_{—}002438121; XP_{—}002438125; XP_{—}002438123; XP_{—}002312510; XP_{—}002314730; NP_{—}001057237; NP_{—}001173972; NP_{—}001054930; NP_{—}001057758; XP_{—}002436766; XP_{—}002446633; XP_{—}002440799; NP_{—}001131156; NP_{—}001140539; XP_{—}002299487; XP_{—}002303634; XP_{—}002301401; XP_{—}002320186; XP_{—}002324017; XP_{—}002317022; Os08g0112200; SORBIDRAFT_{—}07g001180; LOC100191955; POPTRDRAFT_{—}645906; Os02g0676000; SORBIDRAFT_{—}04g031990; XP_{—}002454488; POPTRDRAFT_{—}799918; OPTRDRAFT_{—}815112; Os02g0454500; SORBIDRAFT_{—}01g049060; LOC100280777; POPTRDRAFT_{—}548487; Os05g0144000; SORBIDRAFT_{—}09g003400; LOC100282494; POPTRDRAFT_{—}823553; Os04g0481900; SORBIDRAFT_{—}06g004000; LOC100192487; POPTRDRAFT_{—}877603; and AkTBL25 (SEQ ID NO: 109).

Plants Having Multiple Target Genes Inhibited

Expression of two or more target genes may be inhibited in a plant in as described herein. As explained above, such plants can be generated by performing a molecular manipulation that targets multiple related gene targets in a plant, e.g., using an RNAi to a conserved region to inactivate all of the target genes. Such plants can also be obtained by breeding plants that each have individual mutations that inactivate different target genes to obtain progeny plants that are inactivated in all of the desired target genes. For example, to obtain a rice plant in which three target genes are inactivated, one of skill can target the genes using RNAi developed to a region that is conserved in all three of the rice target genes, or target the genes individually, and breed the resulting mutant plants.

In some aspects, AXY9 genes and homologs thereof are targeted for reduced expression in a plant. In some aspects, AXY9 genes and homologs thereof, as well as other genes involved in polysaccharide O-acetylation, are targeted for reduced expression in a plant.

Expression of Target Gene Inhibitors

Expression cassettes containing polynucleotides that encode target gene expression inhibitors, e.g., an antisense or siRNA, can be constructed using methods well known in the art. Constructs include regulatory elements, including promoters and other sequences for expression and selection of cells that express the construct. Typically, plant transformation vectors include one or more cloned plant coding sequences (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally- or developmentally-regulated, or cell- or tissue-specific expression), a transcription initiation start site, an RNA processing signal (such as intron splice sites), a transcription termination site, and/or a polyadenylation signal.

Examples of constitutive plant promoters which may be useful for expressing a target gene sequence include: the cauliflower mosaic virus (CaMV) 35S promoter, which confers constitutive, high-level expression in most plant tissues (see, e.g., Odel et al., Nature 313:810 (1985); the nopaline synthase promoter (An et al., Plant Physiol. 88:547 (1988)); and the octopine synthase promoter (Fromm et al., Plant Cell 1:977 (1989)).

Additional constitutive regulatory elements including those for efficient expression in monocots also are known in the art, for example, the pEmu promoter and promoters based on the rice Actin-1 5′ region (Last et al., Theor. Appl. Genet. 81:581 (1991); Mcelroy et al, Mol. Gen. Genet. 231:150 (1991); Mcelroy et al, Plant Cell 2:163 (1990)). Chimeric regulatory elements, which combine elements from different genes, also can be useful for ectopically expressing a nucleic acid molecule encoding an IND1 polynucleotide (Comai et al, Plant Mol. Biol. 15:373 (1990)).

Other examples of constitutive promoters include the 1′- or T-promoter derived from T-DNA of Agrobacterium tumafaciens (see, e.g., O'Grady, Plant Mol. Biol. 29:99-108 (1995)); actin promoters, such as the Arabidopsis actin gene promoter (see, e.g., Huang, Plant Mol. Biol. 33:125-139 (1997)); alcohol dehydrogenase (Adh) gene promoters (see, e.g., Millar, Plant Mol. Biol. 31:897-904 (1996)); ACTH from Arabidopsis (Huang et al., Plant Mol. Biol. 33:125-139 (1996)), CatS from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPcI from maize (GenBank No. X15596, Martinez et al. J. Mol. Biol. 208:551-565 (1989)), Gpc2 from maize (GenBank No. U45855, Manjunath et al, Plant Mol. Biol. 33:97-112 (1997)), other transcription initiation regions from various plant genes known to those of skill. See also Holtorf Plant Mol. Biol. 29:637-646 (1995).

A variety of plant gene promoters that regulate gene expression in response to various environmental, hormonal, chemical, developmental signals, and in a tissue-active manner are known in the art. Examples of environmental conditions that may affect transcription by inducible promoters include anaerobic conditions, elevated temperature, drought, or the presence of light. Examples of environmental promoters include drought-inducible promoter of maize; the cold, drought, and high salt inducible promoter from potato (Kirch (1997) Plant Mol. Biol. 33:897-909 (1997)). Plant promoters that are inducible upon exposure to plant hormones, such as auxins, may also be employed. For example, the invention can use the auxin response elements El promoter fragment (AuxREs) in the soybean (Glycine max L.) (Liu, Plant Physiol. 115:397-407 (1997)); the auxin-responsive Arabidopsis GST6 promoter (also responsive to salicylic acid and hydrogen peroxide) (Chen, Plant J. 10: 955-966 (1996)); the auxin-inducible parC promoter from tobacco (Sakai, 37:906-913 (1996)); a plant biotin response element (Streit, Mol. Plant. Microbe Interact. 10:933-937 (1997)); and, the promoter responsive to the stress hormone abscisic acid (Sheen, Science 274:1900-1902 (1996)).

Plant promoters which are inducible upon exposure to chemicals reagents that can be applied to the plant, such as herbicides or antibiotics, may also be used in vectors as described herein. For example, the maize In2 2 promoter, activated by benzenesulfonamide herbicide safeners, can be used; application of different herbicide safeners induces distinct gene expression patterns, including expression in the root, hydathodes, and the shoot apical meristem. Other promoters, e.g., a tetracycline inducible promoter; a salicylic acid responsive element promoter, promoters containing copper-inducible regulatory elements; promoters containing ecdysone inducible regulatory elements; heat shock inducible promoters, a nitrate-inducible promoter, or a light-inducible promoter may also be used.

In some aspects, the plant promoter may direct expression of a polynucleotide of the disclosure in a specific tissue (tissue-specific promoters), such as a leaf or a stem. Tissue specific promoters are transcriptional control elements that are only active in particular cells or tissues at specific times during plant development, such as in vegetative tissues or reproductive tissues. Examples of tissue-specific promoters include promoters that initiate transcription primarily in certain tissues, such as vegetative tissues, e.g., roots or leaves, or reproductive tissues, such as fruit, ovules, seeds, pollen, pistols, flowers, or any embryonic tissue. Other examples are promoters that direct expression specifically to cells and tissues with secondary cell wall deposition, such as xylem and fibers.

Plant expression vectors may also include RNA processing signals that may be positioned within, upstream or downstream of the coding sequence. In addition, the expression vectors may include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Plant expression vectors routinely also include dominant selectable marker genes to allow for the ready selection of transformants. Such genes include those encoding antibiotic resistance genes (e.g., resistance to hygromycin, kanamycin, bleomycin, G418, streptomycin or spectinomycin), herbicide resistance genes (e.g., phosphinothricin acetyltransferase), and genes encoding positive selection enzymes (e.g. mannose isomerase).

Once an expression cassette containing a polynucleotide encoding an inhibitor of the expression of a target gene, e.g., an antisense or siRNA, has been constructed, standard techniques may be used to introduce the polynucleotide into a plant in order to modify the target gene activity and accordingly, the level of acetylation in the plant or plant part in which the target gene is expressed. See protocols described in Ammirato et al., Handbook of Plant Cell Culture-Crop Species. Macmillan Publ. Co (1984); Shimamoto et al., Nature 338:274-276 (1989); Fromm et al. Bio/Technology 8:833-839 (1990); and Vasil et al., Bio/Technology 8:429-434 (1990).

Transformation and regeneration of plants is known in the art, and the selection of the most appropriate transformation technique will be determined by the practitioner. Suitable methods may include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence in a plant in a manner to cause stable or transient expression of the sequence. Examples of these methods in various plants include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are preferably selected using a dominant selectable marker incorporated into the transformation vector. Typically, such a marker will confer antibiotic or herbicide resistance on the transformed plants or the ability to grow on a specific substrate, and selection of transformants can be accomplished by exposing the plants to appropriate concentrations of the antibiotic, herbicide, or substrate.

Methods of Increasing Gene Expression/Polypeptide Levels in a Plant

The present disclosure also relates to increasing the expression of a gene in a plant. In certain embodiment, increasing the expression of a gene results in increased activity of the protein encoded by the gene. In some aspects, the expression of AXY9 or a homolog thereof is increased in a plant. The level of expression of a gene may be assessed by measuring the level of mRNA encoded by the gene, and/or by measuring the level or activity of the polypeptide encoded by the gene. Expression of genes may be increased using any number of techniques well known in the art.

Expression of a gene in a plant may be increased by introducing a copy of the gene of interest into a plant as part of a recombinant construct containing the gene of interest. The constructs typically includes a vector, such as a plasmid, a cosmid, a phage, a virus (e.g., a plant virus), a bacterial artificial chromosome (BAC), a yeast artificial chromosome (YAC), or the like, into which the gene of interest has been inserted. In one aspect, the construct further includes regulatory sequences, including, for example, a promoter, operably linked to the gene of interest. Large numbers of suitable vectors and promoters are known to those of skill in the art, and are commercially available.

General texts that describe molecular biological techniques useful herein, including the use and production of vectors, promoters and many other relevant topics, include Berger and Kimmel (1987)(supra), and Sambrook (1989)(supra). A number of expression vectors suitable for stable transformation of plant cells or for the establishment of transgenic plants have been described including those described in Weissbach and Weissbach, Methods for Plant Molecular Biology, Academic Press (1989) and Gelvin et al., Plant Molecular Biology Manual, Kluwer Academic Publishers (1990). Specific examples include those derived from a Ti plasmid of Agrobacterium tumefaciens, as well as those disclosed by Herrera-Estrella et al., Nature 303: 209 (1983), Bevan, Nucleic Acids Res. 12: 8711-8721 (1984), and Klee, Bio/Technology 3: 637-642 (1985) for dicotyledonous plants.

Alternatively, non-Ti vectors can be used to transfer the DNA into monocotyledonous plants and cells by using free DNA delivery techniques. Such methods can involve, for example, the use of liposomes, electroporation, microprojectile bombardment, silicon carbide whiskers, and viruses. By using these methods transgenic plants such as wheat, rice (Christou, Bio/Technology 9: 957-962 (1991) and corn (Gordon-Kamm, Plant Cell 2: 603-618 (1990) can be produced. An immature embryo can also be a good target tissue for monocots for direct DNA delivery techniques by using the particle gun (Weeks et al., Plant Physiol 102: 1077-1084 (1993); Vasil, Bio/Technology 10: 667-674 (1993); Wan and Lemeaux, Plant Physiol. 104: 37-48 (1994), and for Agrobacterium-mediated DNA transfer (Ishida et al. Nature Biotechnol 14: 745-750 (1996)).

Typically, plant transformation vectors include one or more cloned plant coding sequences (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally—or developmentally—regulated, or cell- or tissue-specific expression), a transcription initiation start site, an RNA processing signal (such as intron splice sites), a transcription termination site, and/or a polyadenylation signal.

Expression of a gene of interest may be increased in the absence of an recombinant construct by manipulating the activity or expression level of the endogenous gene by other means, such as, for example, by ectopically expressing a gene by T-DNA activation tagging (Ichikawa et al. Nature 390 698-701 (1997); Kakimoto et al. Science 274: 982-985 (1996)). This method entails transforming a plant with a gene tag containing multiple transcriptional enhancers and once the tag has inserted into the genome, expression of a flanking gene coding sequence becomes deregulated.

Methods of Generating a Plant with Reduced Polysaccharide O-Acetylation

Methods for generating a plant with reduced polysaccharide O-acetylation are described herein. In some aspects, plants having reduced polysaccharide O-acetylation may be generated by reducing the gene expression of a gene that promotes O-acetylation of polysaccharides in a plant or reducing the activity of a protein encoded by a gene that promotes O-acetylation of polysaccharides in a plant. In some aspects, plants having reduced polysaccharide O-acetylation may be generated by reducing the gene expression in a plant of one or more of: a gene orthologous to an A. thaliana gene of the TBR/TBL family (SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, and 91), a gene orthologous to A. thaliana TBL3 (SEQ ID NO: 5), a gene orthologous to an A. thaliana gene of the TBL17-TBL27 clade (SEQ ID NOs: 33, 35, 37, 39, 41, 43, 45, 47, 49, 51, and 53), a gene orthologous to A. thaliana TBL27 (SEQ ID NO: 53), a gene orthologous to A. thaliana TBL25 (SEQ ID NO: 49), a gene orthologous to A. thaliana At1g12640 (SEQ ID NO: 93), At1g34490 (SEQ ID NO: 95), At1g57600 (SEQ ID NO: 97), At1g63050 (SEQ ID NO: 99), At5g55320 (SEQ ID NO: 101), At5g55350 (SEQ ID NO: 103), or At5g67160 (SEQ ID NO: 105). In some aspects, plants having reduced polysaccharide O-acetylation may be generated by reducing the gene expression in a plant of an AXY9 gene or homolog thereof. In some aspects, plants having reduced polysaccharide O-acetylation may be generated by reducing the gene expression in a plant of the nucleotide sequence set forth in SEQ ID NO: 127 or a homolog thereof. Methods for determining sequences orthologous to a sequence of interest are provided above, and examples of orthologous sequences are described above and provided, without limitation, in Tables 1 and 2. Expression of a target gene may be reduced by any method provided herein for decreasing gene expression.

Plants having reduced polysaccharide O-acetylation maybe generated by increasing the gene expression in a plant of one or more genes orthologous to A. thaliana At4g19420 (SEQ ID NO: 107). In one aspect, plants having reduced polysaccharide O-acetylation maybe generated by increasing the gene expression in a plant of A. konjac putative acetylesterase SEQ ID NO: 111. Expression of a target gene may be increased by any method provided herein for increasing gene expression.

Methods of Evaluating Plants with Reduced O-Acetylation

After a plant has been potentially altered to reduce polysaccharide O-acetylation, one or more parts of the plants may be evaluated to determine the level of target gene expression in a part of the plant that expresses the target gene, e.g., by evaluating the level of mRNA or protein of the target gene, or determining the levels of acetate in the plants. These analyses can be performed using any number of methods known in the art.

Acetyl esters in plant cell wall material can be measured. For example, cell walls are prepared from plant material. Several methods are known, in the simplest method, the plant material is ground and extracted repeatedly with 96% and 70% ethanol. The resulting ‘alcohol insoluble residue’ is highly enriched in cell wall material. The sample is dried and resuspended in buffer at neutral pH. An aliquot of the sample is saponified by treatment with 0.1 M NaOH at 4° C. overnight or at room temperature for several hours. Following saponification, the sample is neutralized by adding 1 M HCl. Acetic acid in the saponified and neutralized sample can be determined in several different ways, e.g. by gas chromatography or HPLC on an appropriate column. A convenient method is to use an acetic acid determination kit, e.g., such as the kit by R-Biopharm, Germany, according to the manufacturer's instructions. The principle of the kit is that acetate is enzymatically consumed in a series of reactions leading to the formation of NADPH, which can be determined spectrophotometrically at 340 nm. The kit allows for correction for interference in the determination.

The procedure above can be used to determine total acetyl esters in the alcohol insoluble residue. To determine acetate esters in specific polymers, the alcohol insoluble residue can be sequentially extracted and/or digested with specific enzymes. The extracts and digests can be analyzed by the methods described above or by mass spectrometry.

Plants selected for reduced acetate levels may further be evaluated to further confirm that the plants provide for improved yield during a saccharification or fermentation process using material from the plant. For example, plant material from a plant with reduced acetate content can be compared to plant material from plants that that do not have reduced acetate content in a saccharification and/or fermentation process as described below.

Methods of Degrading Biomass/Making Fermentation Product

Plants that exhibit reduced polysaccharide O-acetylation can be used in a variety of methods. In some embodiments, biomass from plants having reduced polysaccharide O-acetylation is degraded into oligosaccharides and/or monosaccharides. In some embodiments, biomass from plants having reduced polysaccharide O-acetylation is degraded into oligosaccharides and/or monosaccharides, and the oligosaccharides and/or monosaccharides are fermented to produce a biofuel and/or commodity chemical.

Plant material from a plant having reduced O-acetylation may be subjected to any degradation procedure known in the art. In certain preferred embodiments, a plant having reduced O-acetylation is subjected to a chemical or enzymatic saccharification procedure.

A first step in a saccharification of biomass process is typically a “pretreatment” step. Many different pretreatment procedures may be used and are known in the art, including dilute acid or alkali treatment, steam explosion or ionic liquid treatments. As the beneficial effect of reduced acetate content will differ depending on the exact procedure used, several different pretreatment methods can be evaluated. For example, a dilute acid treatment method can be used. The pretreated plant material may then be subjected to enzymatic hydrolysis using a mixture of cell wall degrading enzymes.

Procedures for cell wall pretreatment and enzymatic digestion are well known to those skilled in the art. The yield or efficiency of the procedure can be readily determined by measuring the amount of reducing sugar released, using a standard method for sugar detection, e.g. the dinitrosalicylic acid method well known to those skilled in the art. Plants engineered in accordance with the disclosure to have reduced polysaccharide O-acetylation provide a higher sugar yield.

Plants having reduced polysaccharide O-acetylation may also be evaluated in comparison to non-modified plants to test for the effect of acetates and acetate-derived compounds on subsequent fermentation. For example, degraded biomass may be subjected to fermentation using an organism such as yeast or E. coli that can convert the biomass into compounds such as ethanol, butanol, alkanes, lipids, etc. In the simplest test, the yield of ethanol obtained with a given amount of starting plant material and a standard yeast fermentation can be determined. Yield can be determined not only with organisms that can ferment glucose, but also with organisms that have the ability to ferment pentoses and or other sugars derived from the biomass. In addition to determining the yield of product, e.g. ethanol, one can determine the growth rate of the organism. The plants of the disclosure that are engineered to have reduced O-acetylation and accordingly, to have reduced acetate, will exhibit a reduced inhibitory effect due to acetate and acetate-derived compounds in comparison to corresponding plants that have not been engineered to have reduced O-acetylation. The decreased inhibitory effect may result in higher final saccharification yields, in higher final yields of a fermentation reaction, or in faster fermentation, or combinations thereof.

Plants having reduced polysaccharide O-acetylation can be used in a variety of reactions, including fermentation reactions. Such reactions are well known in the art. For example, fermentation reactions noted above, e.g., a yeast or bacterial fermentation reaction, may employ plant material derived from a plant having reduced O-acetylation, to obtain ethanol, butanol, lipids, and the like. For example the plants with reduced O-acetylation may be used in industrial bioprocessing reactions that include fermentative bacteria, yeast, or filamentous fungi, such as Corynebacterium spp., Brevibacterium spp., Rhodococcus spp., Azotobacter spp., Citrobacter spp., Enterobacter spp., Clostridium spp., Klebsiella spp., Salmonella spp., Lactobacillus spp., Aspergillus spp., Saccharomyces spp., Zygosaccharomyces spp., Pichia spp., Kluyveromyces spp., Candida spp., Hansenula spp., Dunaliella spp., Debaryomyces spp., Mucor spp., Torulopsis spp., Methylobacteria spp., Bacillus spp., Escherichia spp., Pseudomonas spp., Serratia spp., Rhizobium spp., and Streptomyces spp., Zymomonas mobilis, acetic acid bacteria (family Acetobacteraceae), methylotrophic bacteria, Propionibacterium, Acetobacter, Arthrobacter, Ralstonia, Gluconobacter, Propionibacterium, and Rhodococcus.

Method of Altering Polysaccharide Chemical Properties

Provided herein are methods of reducing polysaccharide O-acetylation in plants. In some aspects, plants having reduced expression of the nucleotide sequence of SEQ ID NO: 127 or a homolog thereof have reduced acetylation of at least one polysaccharide. In some aspects, plants having reduced expression of the nucleotide sequence of SEQ ID NO: 127 or a homolog thereof have reduced acetylation of multiple polysaccharides. In some aspects, plants having reduced expression of the nucleotide sequence of SEQ ID NO: 127 or a homolog thereof have reduced xylan acetylation. In some aspects, plants having reduced expression of the nucleotide sequence of SEQ ID NO: 127 or a homolog thereof have reduced mannan acetylation. In some aspects, plants having reduced expression of the nucleotide sequence of SEQ ID NO: 127 or a homolog thereof have reductions in both xylan and mannan acetylation.

Xylan and mannan are non-limiting examples of polysaccharides that could have altered chemical properties by employing the methods of the present disclosure. Specific plant polysaccharides that may have altered chemical properties using the methods and compositions of the present disclosure may include, without limitation, xyloglucan, arabinoxylan, glucomannan, pectic polysaccharides, homogalacturonan, rhmanogalacturonan I, rhamnogalacturonan II, and lignin.

Method of Altering Glucomannan Chemical Properties

Methods for altering the physicochemical properties of glucomannan are provided herein. Physicochemical properties include, without limitation, gelling behavior in water, elasticity of glucomannan gels, and solubility in water. In some aspects, provided herein are methods for altering the gelation behavior of glucomannan. In some aspects, provided herein are methods for altering the elasticity of glucomannan gels.

Generally speaking, the higher the level of acetylation of glucomannan, the better the solubility of the glucomannan in aqueous solutions and the lower the viscosity of the glucomannan solution. In addition, higher degrees of acetylation of glucomannan are correlated with more elastic glucomannan gels. (Huang et al., Biomacromolecules 3(6): 1296-303 (2002)).

Accordingly, methods for altering the physicochemical properties of glucomannan in a plant by reducing the expression of a gene that promotes O-acetylation of glucomannan are provided. In some aspects, provided herein are methods for generating glucomannan having increased gelling properties by reducing the expression of a gene that promotes O-acetylation of glucomannan. In some aspects, provided herein are methods for generating glucomannan having decreased elasticity by reducing the expression of a gene that promotes O-acetylation of glucomannan. Expression of a gene that promotes O-acetylation of glucomannan may be reduced by any method provided herein.

Plants having glucomannan with altered physicochemical properties are provided herein. In some aspects, provided herein are plants having glucomannan with increased gelling properties and decreased elasticity, and having decreased expression of a gene that promotes O-acetylation of glucomannan. In some aspects, provided herein are A. konjac plants having decreased gene expression of AkTBL25 (SEQ ID NO: 109). In some aspects, provided herein are plants having decreased expression of a gene orthologous to A. thaliana TBL25. In some aspects, provided herein are A. konjac plants having increased expression of a gene that reduces the O-acetylation of glucomannan. In some aspects, provided herein are A. konjac plants having increased gene expression of AkAE (SEQ ID NO: 111). In some aspects, provided herein are plants having increased expression of a gene orthologous to A. thaliana At4g19420 (SEQ ID NO: 107).

In some aspects, AkAE may be produced recombinantly, and the expressed AkAE polypeptide (SEQ ID NO: 112) is used to reduce the O-acetylation of glucomannan. Methods of production of recombinant proteins are well known to one of skill in the art, and typically involve inserting the gene of interest into an expression vector such as a plasmid, and introducing the plasmid into an organism for production of the polypeptide, such as E. coli or yeast. In some aspects, recombinant AkAE polypeptide (SEQ ID NO: 112) is purified, and then incubated with glucomannan under conditions sufficient to reduce the O-acetylation of glucomannan.

### EXAMPLES

The following Examples are merely illustrative and are not meant to limit any aspects of the present disclosure in any way.

### Example 1

### Identification of a Putative Xyloglucan O-Acetyltransferase

An Arabidopsis mutant (axy4) had been identified that contained a significant reduction in the O-acetylation level of the major hemicellulose, xyloglucan, in dicots (FIGS. 8 and 9; and Lutz Neumetzler, Identification and characterization of Arabidopsis mutants associated with xyloglucan metabolism. Dissertation, Rhombos Verlag Berlin, Germany (2010)). However, the gene responsible for the effect was not known at the time. Recently a mutation in a potential candidate gene was identified by classical map-based cloning (Jander et al., Plant Physiology 129(2):440-450 (2002)).

To confirm that this mutation was responsible for the observed reduced O-acetylation effect, T-DNA insertion lines acquired from public stock centers were screened by xyloglucan oligosaccharide mass profiling (OLIMP) (Lerouxel et al., Plant Physiology 130(4):1754-1763 (2002)). This screen revealed four homozygous T-DNA mutants with insertions in the genetic locus At1g70230 (FIG. 1A) to have only non-acetylated xyloglucan in the tissue of 2 week old root material, as compared to the wild type which has a relative xyloglucan O-acetylation level of about 20% (FIG. 1B).

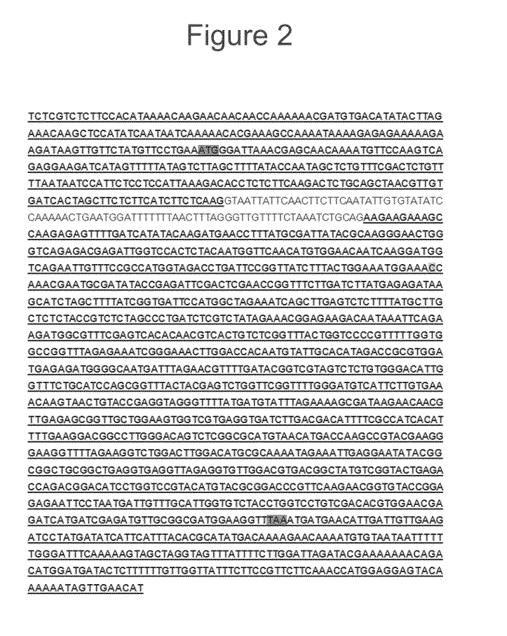

The genetic locus At1g70230 (FIG. 2) had been annotated as Trichome Birefringence-Like 27 (TBL27) and belongs to a gene family with 46 members (Bischoff et al. Plant Physiology 153(2):590-602 (2010)). Previous studies have not ascertained the function of the gene, but it was surmised that it is involved in cellulose deposition and might contain a pectin methylesterase inhibiting activity (Bischoff et al. (2010) supra),

Conversely, our data suggests that this gene (At1g70230) (SEQ ID NO: 53) encodes a xyloglucan O-acetyltransferase.

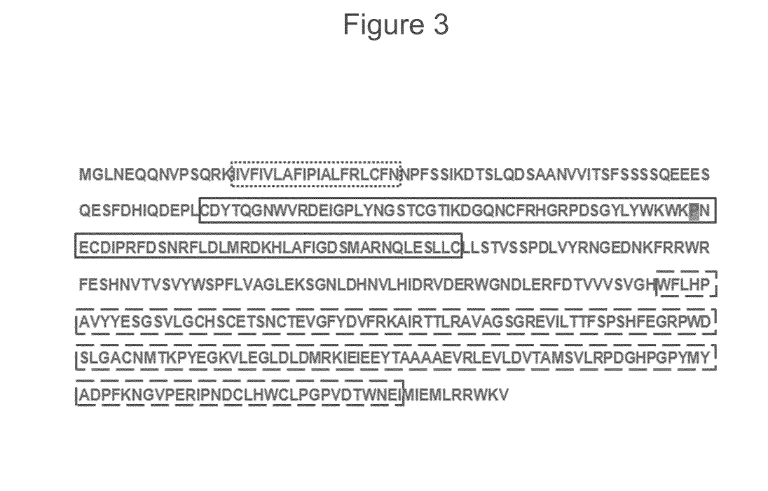

Like all TBL proteins, TBL27 contains one transmembrane domain at the N-terminus and a conserved, plant kingdom specific DUF231 domain at the C-terminus (FIG. 3 and FIG. 7, Bischoff et al. (2010) supra). Additionally, all proteins of this family share a highly conserved TBL domain. (FIG. 3). An alignment of the protein sequences of all 46 members of the TBL family revealed that all TBL proteins share a 62.7% similarity and 11.8% identity in this domain (FIG. 4). The core consensus sequence of the conserved TBL domain was determined as:

(SEQ ID NO: 113)
C[D/N/S/E]-[L/I/W/Y/V/F]-[F/Y/T/S/A]-X-G-X-W-
[V/I/F/T]-X-[D/R/N]-X-X-X-X-X-X-X-X-X-[P/T/G/S/]-
[L/Y/V/S/R/I/F]-[Y/F/S/H]-X-X-X-[S/Y/D/E/T/Q/K]-
[C/S]-X-X-X-[F/L/Y/E/W/T/H/A/I/Q]-
[I/V/L/H/Q/E/D/M]-X-X-X-[F/W/Q/K/V/L/T]-X-C-X-
[K/S/T/A/R/D/E/N/I/V/L/G/M]-[N/F/H/M/Y/Q/A]-
[G/K/N/Q]-[R/K/Q]-[P/D/R/L/S/K/D/T/G]-[D/N/H]-
X-X-[Y/F/V/E]-[L/Q/V/M/T/R/I/E/Q/K/S]-X-X-
[W/L/H/Y]-[R/K/E/S]-W-[Q/K/E/R/I]-P-X-X-C-
X-X-X-[L/I/A/M/]-[P/S/E/K/A]-[R/S/Q/E/V/I/L/K/T]-
[F/L/I/W]-X-[A/G/P/R/S/V]-X-X-[F/L/M/A/V]-
[L/W/M]-[E/K/V/G/Q/T/R/S/A/N/L]-
[K/M/R/S/L/I/N/V/E]-[L/I/S/M/H/V/W/Y/N/F]-
[R/Q/K/M]-X-[G/D/N/H]-[K/R/G/T]-[R/S/N/T/A/W/K/H]-
[L/V/I/M/W]-[M/V/G/A/N/L]-[F/L/I/Y/M]-[V/I/A]-
G-D-S-[L/I/M/V]-X-[R/E/Y/L/T/N/K]-[N/Q/E/G/S/T]-
[Q/M/F/H/T]-[W/F/L/M/V/Y]-[E/V/Q/I/]-S-[L/M/F]-
[L/V/F/M/I/A/S/T]-C-[L/I/S/V/M]-[L/A/I/V]-X-X-
X-[V/L/I/D/T/E/K/S/A]

### Example 2

### Identification of a Putative Glucomannan O-Acetyltransferase

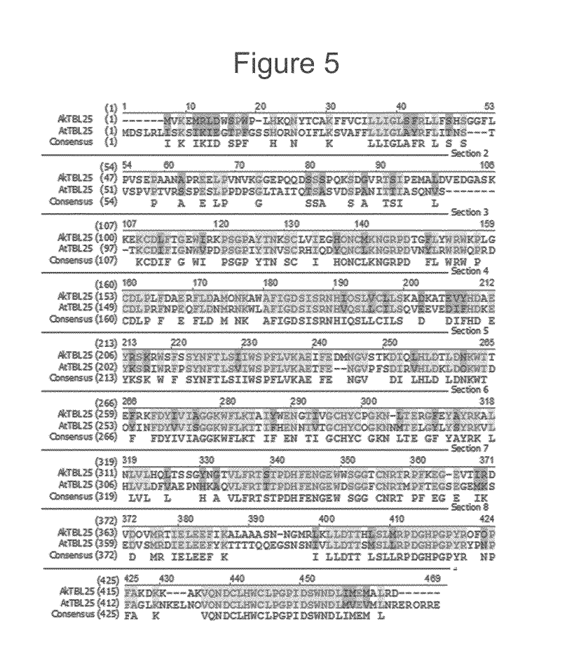

In an effort to identify genes involved in the biosynthesis of the hemicellulose glucomannan an mRNA deep sequencing approach of the developing corm of Amorphophallus konjac (also known as Voodoo Lily) was undertaken (Gille S.; Cheng, K.; Wilkerson, C.; Pauly, M., Deep Sequencing of Voodoo Lily (Amorphophallus konjac): An approach to identify relevant genes involved in the synthesis of the hemicellulose glucomannan. (2011), submitted). At a defined time point during its life cycle, A. konjac develops a corm which is highly enriched in O-acetylated glucomannan (up to 60% dry weight). Hence, it was hypothesized that genes involved in the biosynthesis of glucomannan, including its acetylation, are highly expressed in the developing corm. During this approach a cDNA library of the developing A. konjac corm was subjected to RNA deep sequencing and an EST database was established (Gille et al. (2011), supra). Since the results from the identification of the putative xyloglucan O-acetyltransferase (see above) indicated that members of the TBL family affect cell wall polymer O-acetylation, the established database was probed for TBL domain encoding EST's. Indeed, an ortholog of AtTBL25 (At1g01430) was found to be highly expressed in the Voodoo Lily EST database. An alignment of A. thaliana TBL25 and the putative A. konjac TBL25 revealed a similarity of 61.8% and an identity of 50.3% (FIG. 5). Therefore, TBL25 (At1g01430) may encode a glucomannan O-acetyltransferase in Voodoo Lily.

### Example 3

### Identification of MBOAT Proteins as Cell Wall Polysaccharide Acetyltransferases

In addition, the established Amorphophallus konjac EST database contained putative orthologs of a gene family annotated as membrane bound acyltransferases (MBOAT). This gene family contains 15 members in A. thaliana. The analyses of selected tissues from acquired T-DNA insertion lines for 6 MBOAT family members (At1g12640, At1g34490, At1g57600, At1g63050, At5g55320 and At5g55350) revealed that these lines exhibit a decrease in wall bound acetate between 6% and 36% compared to the respective wild type tissues (FIG. 6).

### Example 4

### Identification of BAHD Proteins as Cell Wall Polysaccharide Acetyltransferases

The established Amorphophallus konjac EST database was further probed for additional genes that might be involved in plant cell wall polymer O-acetylation. This search led to the identification of a BAHD family member (At5g67160). Analysis of a T-DNA insertion line (SAIL_{—}734F07) acquired from a public stock center revealed a 13% decrease in total wall bound acetate in 5 week old cauline leaves compared to wild type (FIG. 6). The same tissue was analyzed in a saccharification assay for its glucose yield after a dilute acid pre-treatment and digestion using a commercially available enzyme mixture. It was observed that the tissue from the mutant yielded in a 25% increase in saccharification (FIG. 6).

### Example 5

### Identification of a Correlation Between Cell Wall Bound Acetate Content and Saccharification Yield

The established A. konjac EST database was further probed for additional genes that may be involved in plant cell wall polymer O-acetylation. Specifically, the database was probed for genes that could be involved in removing O-acetyl substituents, such as acetylesterases. A putative acetylesterase gene was identified in the database: AkAE, having cDNA sequence of SEQ ID NO: 111 and polypeptide sequence of SEQ ID NO: 112. AkAE has a high homology to an A. thaliana gene previously putatively assigned as a pectin acetylesterase coding gene (At4g19420; SEQ ID NO: 107). Since A. konjac produces copious amounts of acetylated glucomannan, we conclude that AkAE is a glucomannan acetylesterase rather than a pectin acetylesterase.

In order to establish a correlation between wall bound acetate content and saccharification yield, an Arabidopsis mutant (SALK 132026) harboring a T-DNA insertion in this putative pectin acetylesterase (At4g19420) was analyzed. This line exhibited an 18% increase in wall bound acetate in 5 week old rosette leaves (FIG. 6). A saccharification assay on the same tissue showed an 8% decrease in glucose yield after dilute acid pre-treatment and enzymatic digest (FIG. 6). This indicates that cell wall polymer acetylation status has a direct impact on the saccharification yield.

Materials and Methods

Materials and methods for Examples 1-5 include:

Plant Material

Arabidopsis thaliana ecotype Columbia (Col-0) was used as a wild type control for the SALK and SAIL T-DNA insertion lines acquired from the public stock centers. Arabidopsis thaliana ecotype Wassilewskija (WS) was used as wild type control for FLAG T-DNA insertion lines acquired from the public stock center.

T-DNA insertion mutant lines were obtained and homozygous lines identified by PCR from the ABRC stock center at Ohio (Alonso et al., Science 301(5633):653-657 (2003)) and from Institut Jean-Pierre Bourgin at the Versailles Center of the National Institute for Agronomical Research (INRA) (Samson et al. Nucl. Acid Res. 30(1):94-97, (2002)). Plants were grown under standard conditions as described previously (Gille et al., PNAS 106(34):14699-14704 (2009)).

Preparation of Cell Wall Material

Plant material to be analyzed was harvested and flash frozen in liquid nitrogen. The frozen material was ground to a fine powder using a ball mill. The ground material was washed in 1 mL aqueous ethanol (70% v/v) followed by a wash in chloroform/methanol (1:1 v:v) and vacuum dried, yielding in an alcohol insoluble residue (AIR).

Oligosaccharide Mass Profiling (OLIMP)

The prepared AIR from tissues of interest was analyzed by Oligosaccharides mass profiling (OLIMP) using Matrix Assisted Laser Desorption Ionization—Time of Flight MS (MALDI-TOF MS) as described by Gunl et al. J. Visual Experiments, doi: 10.3791/2046 (2010).

Determination of Total Wall Bound Acetate Content

The total wall polymer bound acetate content of tissues of interest was determined using the Megazyme “Acetic Acid Kit” (catalog #K-ACET, Megazyme, Wicklow, Ireland). The assay was downscaled and adapted to a 96-well format. An amount of 1 mg AIR was solubilized in 100 μl water. The polymer bound acetate was released by adding 100 μl NaOH (1M) and incubation for 1 h at RT, shaking at 500 rpm. The samples were neutralized with 1M HCl, centrifuged for 10 min at 14,000 rpm, 100 of the supernatant containing released acetate was transferred to a UV capable 96-well flat bottom assay plate and diluted with 94 μl water. The kit content was used as follows. Solution 1 and Solution 2 were mixed in a ratio of 2.5:1 (30 μl+12 μl per sample) and 420 of the mixture were added to each sample, mixed and incubated at RT for 3 min. The absorption was read at 340 nm (A0). Solution 3 was diluted 1:10 in water; 120 was added to each sample, mixed and incubated at RT for 4 min. The absorption was read at 340 nm (A1). Solution 4 was diluted 1:10 in water, 120 was added each sample, mixed and incubated for 12 min at RT. The absorption was read at 340 nm (A2). The amount of acetate in the samples was calculated based on an acetic acid standard curve and according to the manufacturer's recommendations.

Saccharification Assay

To determine saccharification yields the AIR from tissues of interest was enzymatically destarched. About 10-15 mg AIR was re-suspended in 1.5 mL 0.1M Sodium acetate buffer (pH5.0) and incubated for 20 min at 80° C. The suspension was cooled down to room temperature (RT), 10 μL of 0.01 mg/mL Sodium azide, 10 μL of 50 μg/mL α-Amylase and 22 μL of Pullulanase M2 (catalog #E-PULBL, Megazyme, Wicklow, Ireland) were added. The suspension was incubated for 16 h at 37° C. shaking at 250 rpm. After incubation the digest was heated for 10 min at 100° C., centrifuged for 15 min at 14,000 rpm and the supernatant was discarded. The remaining pellet was re-suspended in 1.5 mL water, centrifuged for 15 min at 14,000 rpm and the supernatant was discarded. This step was repeated for a total of three times. Finally, the pellet was washed once with 1.5 mL acetone and vacuum dried, resulting in de-starched AIR. Prior to saccharification, the de-starched AIR was pretreated with dilute acid and heat. One mg of de-starched AIR was re-solubilized on 100 μL water, 900 μL of 2% (v/v) sulfuric acid added and the samples were heated for 45 min at 120° C. After incubation the samples were cooled down on ice and neutralized with 140 μL 5M sodium hydroxide. For saccharification 1M citrate buffer (pH 4.5) was added to a final concentration of 50 mM. To this mixture 1 μL of 10 mg/mL sodium azide and 2 μL of Accellerase 1500 (Genencor, USA) were added. The samples were incubated for 20 h at 50° C. shaking at 100 rpm, followed by 10 min at 100° C. The samples were centrifuged for 15 min at 14,000 rpm and the supernatant was recovered. The concentration of released glucose was measured using the YSI 2700 SELECT™ Biochemistry Analyzer (YSI Life Sciences, USA) and normalized to the weight of destarched AIR.

### Example 6

### Demonstration of O-Acetyltransferase Activity

O-acetyltransferase activity of AXY4 (TBL27) on xyloglucan may be demonstrated using the following approaches.

The sequence of the gene encoding Arabidopsis AXY4 (SEQ ID NO: 53) is cloned into an appropriate, commercially available vector for subsequent heterologous expressed in the yeast Pichia pastoris or in the bacteria E. coli. Protein expression vectors are well known to one of skill in the art. The expressed protein is purified via affinity chromatography using a suitable tag introduced onto the protein. The donor substrate for plant cell wall polymer acetylation was shown to be Acetyl-CoA (Pauly and Scheller, Planta 210(4):659-667 (2000)). Hence, radio-labeled Acetyl-CoA is incubated together with the expressed protein and de-acetylated xyloglucan oligosaccharides and/or non-acetylated xyloglucan polymer derived from the wall material of the identified Arabidopsis tbl27 (axy4) mutant. After incubation the products are precipitated, and incorporated ¹⁴C actetate is determined by scintillation counting (Pauly and Scheller, (2000) supra). The polymer bound acetic acid is released by mild alkali treatment, and subjected to HPLC analysis to ascertain that it was still acetate that is present on the polymers and that it was esterlinked (Pauly and Scheller, (2000) supra). Alternatively, the product of incubation (non-radioactive acetyl-CoA, heterologously expressed AXY4 protein, and deacetylated xyloglucan oligosaccharides/polymer incubated in a buffer) are structurally assessed by MALDI-TOF analysis (Lebouef et al, Anal. Biochem. 373 (1):9-17 (2008)) to indicate the degree of O-acetylation.

In the case that additional hitherto unidentified cofactors are required to acetylate xyloglucan, an in planta approach is used similar to the glycosyltransferase activity assay described by Jensen et al. 2008. The gene encoding for the putative O-acetyltransferase is cloned into a binary plant 35S overexpression vector and transformed into Agrobacterium tumefaciens. The gene under the control of the cauliflower mosaic virus 35S promoter is transformed via Agrobacterium into Nicotiana benthamiana leaves for overexpression. The transformed leaves are used for the preparation of microsomes as described previously (Jensen et al., The Plant Cell 20(5):1289-1302 (2008)). The derived microsomes are solubilized using detergent and incubated with radio-labeled Acetyl-CoA and non-acetylated XyGO and/or xyloglucan polymer derived from the identified tbl27 (axy4) mutants. The products are recovered and analyzed as described previously by Pauly and Scheller (2000) supra.

O-acetyltransferase activity analysis for other polypeptides of the disclosure and/or for other polysaccharides may be performed using the same methods as described above for A. thaliana AXY4/TBL27 and xyloglucan, substituting the other polypeptide and/or polysaccharide where appropriate. Other polypeptides that may be analyzed as above include, without limitation, any of A. thaliana TBR/TBL family polypeptides or their orthologs, and any of xylans and their derivatives and glucomannans and their derivatives. In one aspect, AkTBL25 is analyzed for O-acetyltransferase activity on glucomannan.

### Example 7

### Characterization of TBL3 Xylan/Mannan O-Acetyltransferase

The following example relates to the characterization of xylan/mannan O-acetylation in stem material from the Arabidopsis TBL3 knockout mutant.

Materials and Methods

Plant Material

The Arabidopsis thaliana TBL3 T-DNA insertion line (SALK_{—}078649C) was acquired from the public stock centers. A. thaliana ecotype Columbia (Col-0) was used as a wild type control.

The TBL3 mutant line was confirmed by PCR of flanking T-DNA, and sequencing to determine precise location of T-DNA

Plants were grown under standard conditions as described previously (Gille et al., PNAS 106(34):14699-14704 (2009)).

Preparation of Arabidopsis Stem Material

The alcohol insoluble residue of Arabidopsis stem material was ground in a PM 100 planetary ball mill for 7 hours with 5 minutes grinding and 5 minutes break interval. 25 mg ball milled material was dissolved in 0.75 ml DMSO-d6 doped with 10 μl deuterated 1-ethyl-3-methylimidazole acetate [Emim]OAc-d14.

The solution-state sample was measured by a Bruker AVANCE 600 MHz NMR spectrometer equipped with an inverse (proton coils closest to the sample) gradient 5-mm TXI 1H/13C/15N cryoprobe.

Analysis of O-Acetylation

The 2D one bond 13C-1H correlations (HSQC: Heteronuclear Single Quantum Coherence) in plant cell walls were determined by a Bruker standard pulse sequence ‘hsqcetgpsisp.2’. The experiment provides a phase-sensitive gradient edited 2D HSQC spectrum using adiabatic pulses for inversion and refocusing. The spectra were calibrated by the central DMSO solvent peak (δC 39.9 ppm, δH 2.49 ppm). The following parameters were applied in the NMR experiments: spectra width 16 ppm in F2 (1H) dimension with 2048 data points (TD1) and 240 ppm in F1 (13C) dimension with 256 data points (TD2); scan number (SN) of 128; interscan delay (D1) of 1.5 s.

All NMR data processing and analysis were performed using Bruker's Topspin 3.1 software.

Determination of Total Wall Bound Acetate Content

The total wall polymer bound acetate content of stem tissue was measured as described in the “Material and Methods” section for Examples 1-5.

Results

12 Tbl knockout mutations from 22 knockout lines were analyzed for wall acetate content. The TBL3 knockout line showed the strongest decrease in wall acetate content, and was thus selected for further analysis.

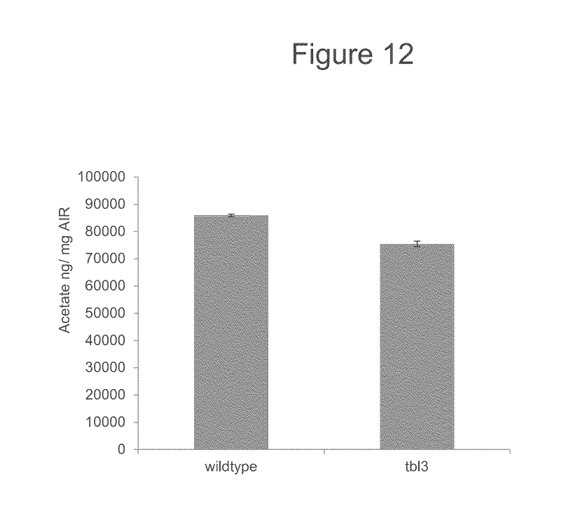

The TBL3 mutant line showed an approximately 10,000 Acetate ng/mg AIR as compared to the wild type Arabidopsis line (FIG. 12).

Further analysis of the TBL3 mutant line determined that amount of carbohydrates, lignin, and acetylation as compared to the wild type line (Tables 3-5). As shown in Table 5, the TBL3 mutant line showed a 6.53% reduction in xylan acetylation [Ac(Xyl)], a 12.21% reduction in mannan acetylation [Ac(Man)], and a 7.93% reduction in total acetylation.

TABLE 3

| Carbohydrates | Wild Type | TBL3 Mutant |
|---|---|---|
| Glc | 62.79% | 63.75% |
| Xyl | 23.76% | 23.64% |
| Man | 8.60% | 8.51% |
| GlcA | 2.32% | 1.98% |
| Gal | 0.67% | 0.44% |
| Ara | 1.41% | 1.16% |
| Rha | 0.46% | 0.51% |

TABLE 4

| | Wild Type | TBL3 Mutant |
|---|---|---|
| Lignin-Units | | |
| S | 26.70% | 25.75% |
| G | 67.09% | 65.77% |
| H | 6.21% | 8.48% |
| S/G | 0.39% | 0.39% |
| Lignin Side Chain | | |
| A | 79.78% | 83.08% |
| B | 7.93% | 7.10% |
| C | 12.29% | 9.82% |
| A (G/S) | 2.05% | 2.17% |

TABLE 5

| | Wild Type | TBL3 Mutant | Reduction |
|---|---|---|---|
| Ac(Xyl) | 60.12% | 56.20% | 6.53% |
| O2—Ac/O3—Ac (Xyl) | 1.12 | 0.70 | 37.37% |
| Ac(Man) | 54.95% | 48.24% | 12.21% |
| O2—Ac/O3—Ac (Man) | 1.06 | 1.26 | −19.49% |
| Total Ac(Xyl + Man) | 58.75% | 54.09% | 7.93% |

Based on these results, it is believed that the TBL3 protein is involved in xylan/mannan O-acetylation in Arabidopsis.

### Example 8

### Identification of a Gene Involved in O-Acetylation in Plants

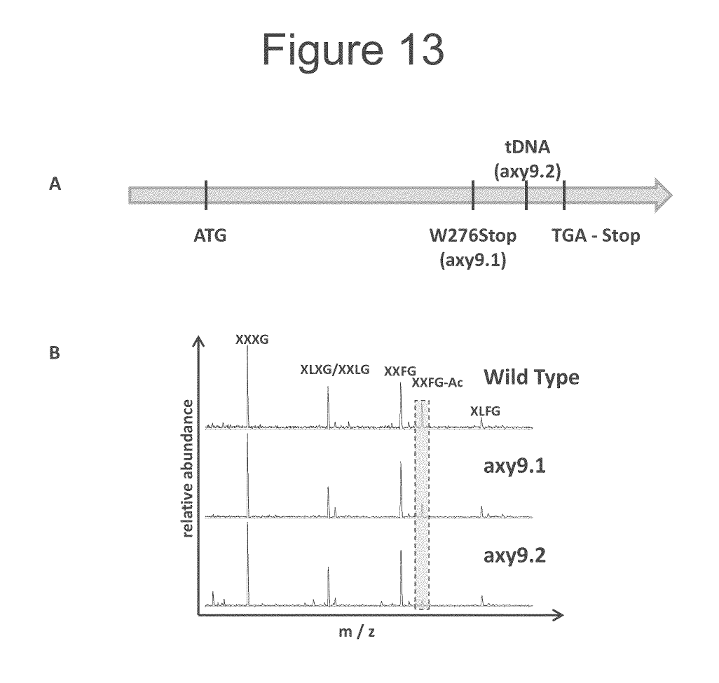

An Arabidopsis mutant (axy9) had been identified that contained a significant reduction in the O-acetylation level of the major hemicellulose, xyloglucan, in dicots (FIG. 13). However, the gene responsible for the effect was not known at the time. Recently a mutation in a potential candidate gene was identified by classical map-based cloning (Jander et al., Plant Physiology 129(2):440-450 (2002)). The mutant contained a W276Stop single base pair mutation that introduced a premature stop codon into the coding region of the candidate gene.

To confirm that this mutation was responsible for the observed reduced O-acetylation effect, a T-DNA insertion line acquired from public stock centers was screened by xyloglucan oligosaccharide mass profiling (OLIMP) (Lerouxel et al., Plant Physiology 130(4):1754-1763 (2002))(See Example 9 for Materials and Methods). This screen revealed that both the single base pair mutant and the T-DNA mutant with molecular defects in the genetic locus At3g03210 (FIG. 13A) have predominantly non-acetylated xyloglucans and reduced levels of O-acetylated xyloglucan as compared to wild-type plants (FIG. 13B).

The genetic locus At3g03210 had been annotated as encoding a protein of unknown function with no known role in any biological process (The Arabidopsis Information Resource Center). Our data suggests that this gene (At3g03210) encodes a protein that functions in promoting general polysaccharide O-acetylation. As a result of this discovery, the gene is referred to as AXY9. axy9 mutants exhibit reduced O-acetate content in plant biomass as compared to wild-type plants.

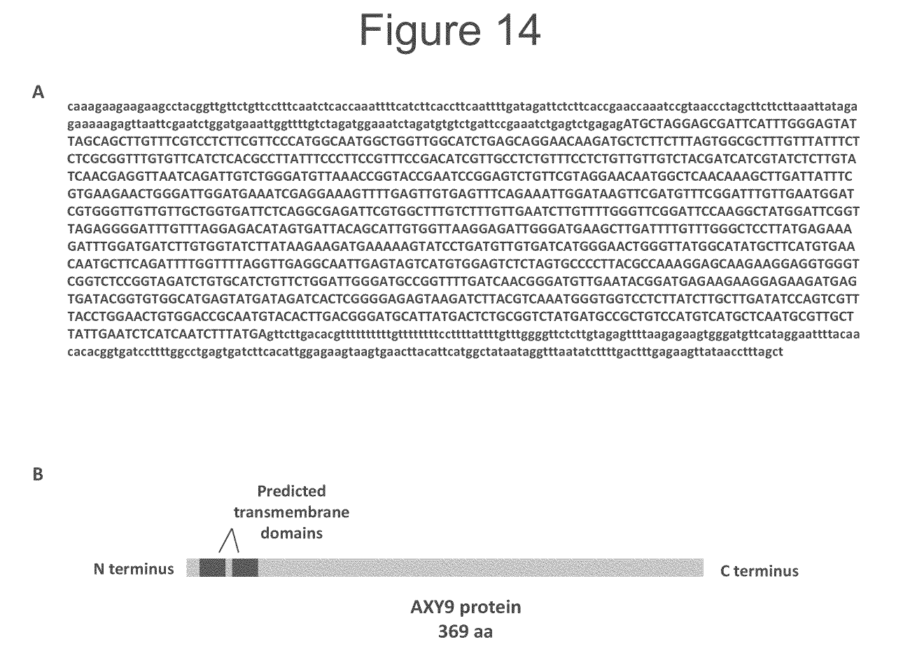

The AXY9 gene (SEQ ID NO: 127), the sequence of which is presented in FIG. 14A as SEQ ID NO: 127, encodes a protein that is 369 amino acids in length (FIG. 14B). Additionally, the AXY9 protein is predicted to have two transmembrane domains near the N-terminus of the protein (FIG. 14B).

### Example 9

### AXY9 Functions in Promoting O-acetylation in Plants

This example describes how plants with mutations in the AXY9 gene exhibit reductions in general O-acetylation content in a variety of plant tissues and organs.

Materials and Methods

The Materials and Methods described here for Example 9 were also used for the initial OLIMP profiling described in Example 8.

Plant Material

Arabidopsis thaliana ecotype Columbia (Col-0) was used as a wild type control for the axy9.1 and axy9.2 mutants. The T-DNA insertion mutant line (axy9.2) was obtained and homozygous lines identified by PCR from the ABRC stock center at Ohio (Alonso et al., Science 301(5633):653-657 (2003)). Plants were grown under standard conditions as described previously (Gille et al., PNAS 106(34):14699-14704 (2009)).

Preparation of Cell Wall Material

Plant material to be analyzed was harvested and flash frozen in liquid nitrogen. The frozen material was ground to a fine powder using a ball mill. The ground material was washed in 1 mL aqueous ethanol (70% v/v) followed by a wash in chloroform/methanol (1:1 v:v) and vacuum dried, yielding in an alcohol insoluble residue (AIR).

Oligosaccharide Mass Profiling (OLIMP)

The prepared AIR from tissues of interest was analyzed by Oligosaccharides mass profiling (OLIMP) using Matrix Assisted Laser Desorption Ionization—Time of Flight MS (MALDI-TOF MS) as described by Gunl et al. J. Visual Experiments, doi: 10.3791/2046 (2010).

Determination of Total Wall Bound Acetate Content

The total wall polymer bound acetate content of tissues of interest was determined using the Megazyme “Acetic Acid Kit” (catalog #K-ACET, Megazyme, Wicklow, Ireland). The assay was downscaled and adapted to a 96-well format. An amount of 1 mg AIR was solubilized in 100 μl water. The polymer bound acetate was released by adding 100 μl NaOH (1M) and incubation for 1 h at RT, shaking at 500 rpm. The samples were neutralized with 1M HCl, centrifuged for 10 min at 14,000 rpm, 100 of the supernatant containing released acetate was transferred to a UV capable 96-well flat bottom assay plate and diluted with 94 μl water. The kit content was used as follows. Solution 1 and Solution 2 were mixed in a ratio of 2.5:1 (30 μl+12 μl per sample) and 42 μl of the mixture were added to each sample, mixed and incubated at RT for 3 min. The absorption was read at 340 nm (A0). Solution 3 was diluted 1:10 in water; 12 μl was added to each sample, mixed and incubated at RT for 4 min. The absorption was read at 340 nm (A1). Solution 4 was diluted 1:10 in water, 12 μl was added each sample, mixed and incubated for 12 min at RT. The absorption was read at 340 nm (A2). The amount of acetate in the samples was calculated based on an acetic acid standard curve and according to the manufacturer's recommendations.

Results

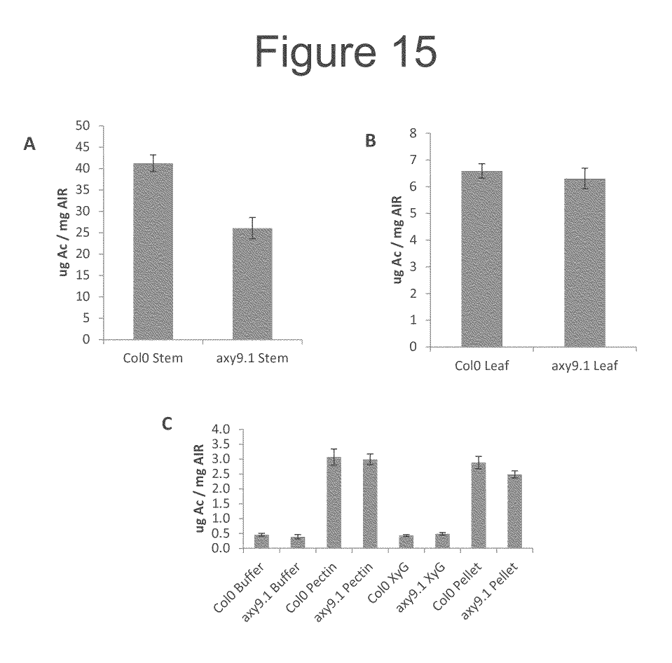

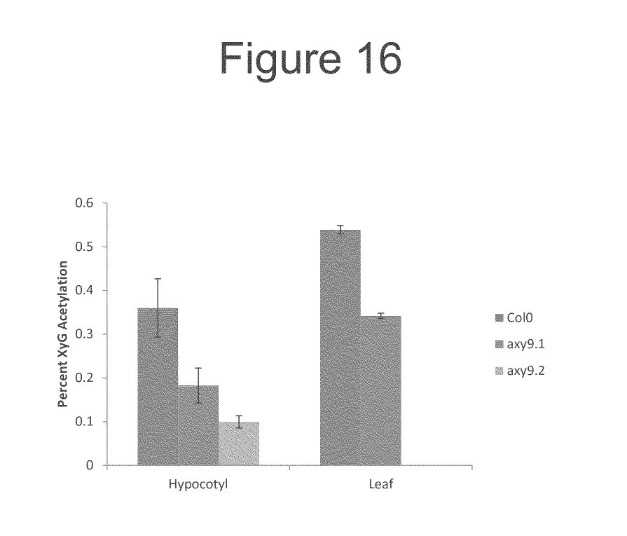

In order to further investigate the role of AXY9 in promoting O-acetylation in plants, the acetate content of axy9.1 mutants was determined in various plant tissues and compared to the acetate levels of wild-type plants. As can be seen in FIG. 3A, axy9.1 mutants had a reduced acetate content in stems as compared to wild-type plants. In leaves, wild type and axy9.1 had similar acetate content (FIG. 15B). FIG. 15C demonstrates that axy9.1 exhibited reduced acetate content only in the pellets of prepared leaf cell wall fractions as compared to wild-type plants.

MALDI-TOF MS analysis of xyloglucan oligosaccharides was used to assay the content of acetylated xyloglucan oligosaccharides in wild type, axy9.1 and axy9.2. As is shown in FIG. 4, both axy9.1 and axy9.2 had reduced xyloglucan acetylation compared to wild type in plant hypocotyls. The same decrease in xyloglucan acetylation was observed for axy9.1 in plant leaves relative to wild type acetylation levels.

### Example 10

### Analysis of Acetylated Sugar Content in Wild Type and axy9.1

The following example describes how axy9.1 mutants have reduced acetylated sugar content as compared to wild type plants.

Materials and Methods

Plant Material

The Arabidopsis thaliana wild-type (Col-0) and axy9.1 mutant were used as described above. Plants were grown under standard conditions as described previously (Gille et al., PNAS 106(34):14699-14704 (2009)).

Preparation of Arabidopsis Stem Material

The alcohol insoluble residue of Arabidopsis stem material was ground in a PM 100 planetary ball mill for 7 hours with 5 minutes grinding and 5 minutes break interval. 25 mg ball milled material was dissolved in 0.75 ml DMSO-d6 doped with 10 μl deuterated 1-ethyl-3-methylimidazole acetate [Emim]OAc-d14.

The solution-state sample was measured by a Bruker AVANCE 600 MHz NMR spectrometer equipped with an inverse (proton coils closest to the sample) gradient 5-mm TXI 1H/13C/15N cryoprobe.

Analysis of O-Acetylation

The 2D one bond 13C-1H correlations (HSQC: Heteronuclear Single Quantum Coherence) in plant cell walls were determined by a Bruker standard pulse sequence ‘hsqcetgpsisp.2’. The experiment provides a phase-sensitive gradient edited 2D HSQC spectrum using adiabatic pulses for inversion and refocusing. The spectra were calibrated by the central DMSO solvent peak (δC 39.9 ppm, δH 2.49 ppm). The following parameters were applied in the NMR experiments: spectra width 16 ppm in F2 (1H) dimension with 2048 data points (TD1) and 240 ppm in F1 (13C) dimension with 256 data points (TD2); scan number (SN) of 128; interscan delay (D1) of 1.5 s. All NMR data processing and analysis were performed using Bruker's Topspin 3.1 software.

Results

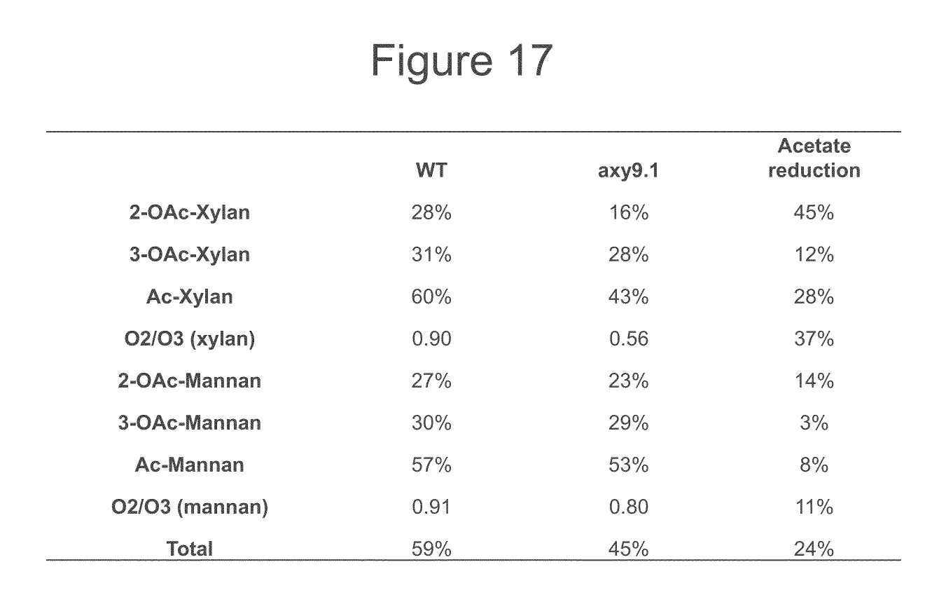

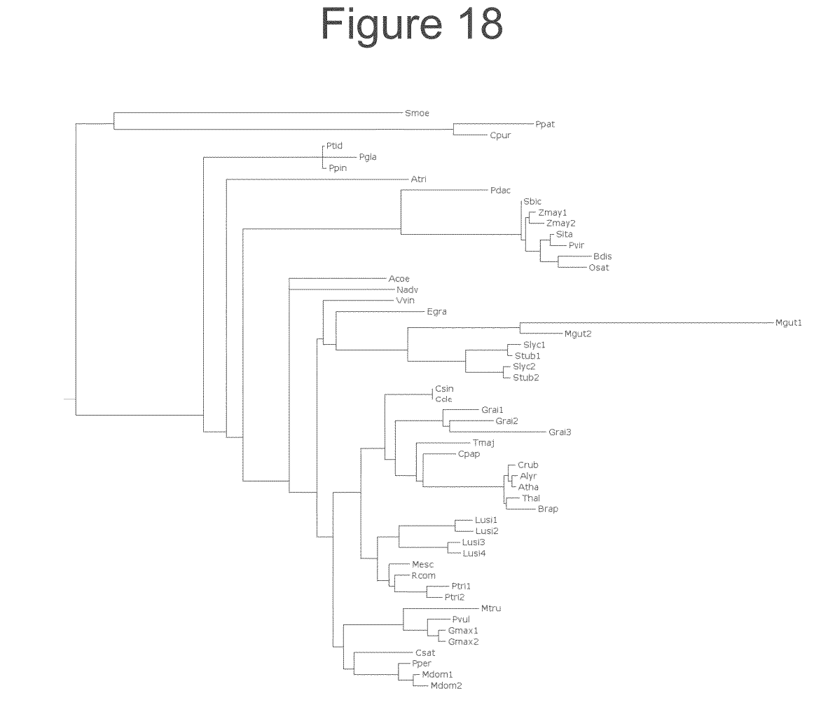

Quantification of xylan and mannan acetylation in stems of wild type and axy9.1 mutants was accomplished using NMR. It was observed that axy9.1 mutants exhibited an acetate reduction in each type of sugar analyzed (FIG. 17). The most severe reductions were seen in acetylated xylans: axy9.1 exhibited a 45% reduction in 2-OAc-xylan and a 37% reduction in O2/O3 (xylan) as compared to wild-type plants. Reductions in acetylated mannan content were also observed. For example, axy9.1 exhibited a 14% reduction in 2-OAc-mannan as compared to wild-type plants. Overall, axy9.1 exhibited a total acetate reduction of 24% when compared to the acetylated sugar contents of wild-type plants (FIG. 17).

Based on the results described herein, it is believed that AXY9 functions in promoting O-acetylation in plants of multiple hemicellulose including but not limited to xylan, mannan, and xyloglucan.

### Example 11

### Phylogenetic Analysis of AXY9 and its Homologs

This example describes a phylogenetic analysis of AXY9 and homologous sequences in other species.

A maximum likelihood tree of identified AXY9 putative orthologs was generated using standard phylogenetic analysis techniques described herein. FIG. 6 shows the maximum likelihood tree product of a phylogenetic analysis of AXY9. Putative orthologs of AXY9 were not identified outside of land plants. A single copy of AXY9 was found in most species, although some species were found to have multiple copies.

What is claimed:

1. A method of increasing the yield of fermentation product from a fermentation reaction, the method comprising:
A) providing a mutant or transgenic plant having reduced O-acetylation of one or more plant cell wall polysaccharides in said mutant or transgenic plant compared to the O-acetylation of one or more plant cell wall polysaccharides of a corresponding non-mutant or non-transgenic plant,
wherein said mutant or transgenic plant comprises a gene encoding a polypeptide having a polypeptide sequence selected from the group consisting of SEQ ID NOs: 113, 114, 115, 116, 117, 118, 119, 120, 121, and 124, and
wherein said mutant or transgenic plant has reduced expression of the gene or reduced activity of a protein encoded by the gene compared to the expression of the gene or activity of the protein encoded by the gene in the corresponding non-mutant or non-transgenic plant;
B) obtaining biomass from said mutant or transgenic plant;
C) subjecting the biomass to a degradation procedure, thereby yielding degraded biomass,
wherein said reduced O-acetylation of one or more plant cell wall polysaccharides in the mutant or transgenic plant increases the amount of degraded biomass compared to the amount of degraded biomass generated from the degradation of biomass obtained from the corresponding non-mutant or non-transgenic plant; and
D) incubating the degraded biomass with a fermentative organism under conditions suitable to yield a fermentation product,
wherein an increased yield of fermentation product from the fermentation reaction is obtained, as compared to the yield of fermentation product obtained from a fermentation reaction using degraded biomass from the corresponding non-mutant or non-transgenic plant.

2. The method claim 1, wherein the polysaccharide is glucomannan and the expression of a gene orthologous to SEQ ID NO: 49 is reduced.

3. The method of claim 1, wherein the mutant or transgenic plant is Amorphophallus konjac and the gene is SEQ ID NO: 109.

4. The method of claim 1, wherein the mutant or transgenic plant is a mutant plant.

5. The method of claim 4, wherein the reduced expression of the gene or reduced activity of the protein encoded by the gene in the mutant plant is a result of a mutation in the gene.

6. The method of claim 5, wherein the mutation in the gene was the result of TILLING or T-DNA insertion.

7. The method of claim 1, wherein the mutant or transgenic plant is a transgenic plant.

8. The method of claim 7, wherein the transgenic plant further comprises an RNAi-inducing vector or an antisense RNA construct.

9. The method of claim 8, wherein the reduced expression of the gene or reduced activity of the protein encoded by the gene in the transgenic plant is a result of the RNAi-inducing vector or the antisense RNA construct.

10. The method of claim 1, wherein the mutant or transgenic plant is selected from the group consisting of Zea mays, Oryza sativa, Sorghum bicolor, Populus trichocarpa, Picea sitchensis, Panicum virgatum, Miscanthus giganteus, Brachypodium distanchyon, and Amorphophallus konjac.

11. A method of increasing the yield of fermentation product from a fermentation reaction, the method comprising:
A) providing a mutant or transgenic plant having reduced O-acetylation of one or more plant cell wall polysaccharides in said mutant or transgenic plant compared to the O-acetylation of one or more plant cell wall polysaccharides of a corresponding non-mutant or non-transgenic plant,
wherein said mutant or transgenic plant comprises a gene comprising the nucleotide sequence of SEQ ID NO: 127 or a homolog thereof, and
wherein said mutant or transgenic plant has reduced expression of the gene or reduced activity of a protein encoded by the gene compared to the expression of the gene or activity of the protein encoded by the gene in the corresponding non-mutant or non-transgenic plant;
B) obtaining biomass from said mutant or transgenic plant;
C) subjecting the biomass to a degradation procedure, thereby yielding degraded biomass; and
D) incubating the degraded biomass with a fermentative organism under conditions suitable to yield a fermentation product,
wherein an increased yield of fermentation product from the fermentation reaction is obtained, as compared to the yield of fermentation product obtained from a fermentation reaction using degraded biomass from the corresponding non-mutant or non-transgenic plant.

12. The method claim 11, wherein the polysaccharide is selected from the group consisting of xylan and mannan, and wherein the expression of a gene homologous to SEQ ID NO: 127 is reduced.

13. The method of claim 11, wherein the polysaccharide is xylan and concomitantly mannan, and wherein the expression of a gene homologous to SEQ ID NO: 127 is reduced.

14. The method of claim 11, wherein the mutant or transgenic plant is a mutant plant.

15. The method of claim 15, wherein the reduced expression of the gene or reduced activity of the protein encoded by the gene in the mutant plant is a result of a mutation in the gene.

16. The method of claim 16, wherein the mutation in the gene is the result of a single base pair change or a T-DNA insertion.

17. The method of claim 11, wherein the mutant or transgenic plant is a transgenic plant.

18. The method of claim 18, wherein the transgenic plant further comprises an RNAi-inducing vector or an antisense RNA construct.

19. The method of claim 18, wherein the reduced expression of the gene or reduced activity of the protein encoded by the gene in the transgenic plant is a result of the RNAi-inducing vector or the antisense RNA construct.

20. The method of claim 11, wherein the mutant or transgenic plant is selected from the group consisting of Zea mays, Oryza sativa, Sorghum bicolor, Populus trichocarpa, Picea sitchensis, Panicum virgatum, Miscanthus giganteus, Brachypodium distanchyon, and Amorphophallus konjac.


SEQUENCE LISTING (127 sequences)

SEQ ID NO: 1 - DNA, 1671 bp, Arabidopsis thaliana
atggcgttggactccgttaagcatttgcctattcacggcgtctccgccttctcctccgtcaccgtcgaaatcaaaagctttttctccaccgtgaaaccaagaaaaacttcaactttcgtctacgctttcgtagtaactttcgttgccttgactgttttcttagcctttagtccttctcccatcacagtcgcactcgctccttccatttcttcctatgttctccctaatatcactgtctcgaattcatccaactcttcaccgtcgagtctcgattctaatttcaccacattgcgtacaccggcgccggaaaatctcactgcagttactaaaaacttaacctttgagtcccccgtcgcaaatgggacaacggacacaaatgctaaaaccatcactattcagttccagaccggtcatgcaaaggaaaacatatcgtgtcctgacaacaaaactgctcgagatttggacacacatggagcaaggaaagcacctctgtcggaagttctagctgtcaattcgactgcttctccgaagcgaaaacagagaaggaagtcgtcgctgagaaaggtgatcgagtctctgaagagttgtgagtttttcgagggagattgggtcaaagacgattcgtatccgctctacaaacccggttcgtgtaatctgatcgatgaacagtttaattgtatctcaaacggaagacctgacgtggatttccagaaattgaaatggaaaccaaagcaatgttctttaccaaggttaaatggaggtaaattgcttgagatgattagaggaagaaggctagtgtttgttggagattcgctgaatagaaacatgtgggagtctttggtttgcattcttaaaggatcagtgaaagacgagagtcaagtctttgaggctcatggaaggcatcaattccgttgggaggctgagtactctttcgtctttaaagattataattgcactgtggagttctttgcatcgcctttcttggttcaagaatgggaagttacagagaagaacgggacgaagaaggagactttgcggttagatttggtcgggaagtcatcggagcagtacaaaggagcggatattcttgtattcaataccggacattggtggactcatgagaaaacttccaaaggggaggattattatcaagaagggagcactgttcacccgaaactcgatgtggatgaagctttccgaaaagcattgacaacttggggtcgatgggttgataagaatgtgaatccaaagaagtcccttgttttcttccgtggttattccccgtcacatttcagcggcgggcaatggaatgcgggaggagcatgcgatgatgaaacagaaccgatcaagaacgagacttacctaactccttacatgttgaagatggagatacttgaaagagttctcaggggaatgaaaacaccagtcacgtatctcaacatcacgaggttaactgattacaggaaggacgctcacccatcgatataccggaaacaaaaactatccgcagaagaaagtaaatcaccattgttgtaccaagactgtagccactggtgtctccctggggtgcctgattcttggaacgaaattttctatgccgagttacttgtaaagcttgaccagcttggtggcaaaagacggcggaaagccctaaaagatcataggagttga

SEQ ID NO: 2 - protein, 556 aa, Arabidopsis thaliana
MALDSVKHLPIHGVSAFSSVTVEIKSFFSTVKPRKTSTFVYAFVVTFVALTVFLAFSPSPITVALAPSISSYVLPNITVSNSSNSSPSSLDSNFTTLRTPAPENLTAVTKNLTFESPVANGTTDTNAKTITIQFQTGHAKENISCPDNKTARDLDTHGARKAPLSEVLAVNSTASPKRKQRRKSSLRKVIESLKSCEFFEGDWVKDDSYPLYKPGSCNLIDEQFNCISNGRPDVDFQKLKWKPKQCSLPRLNGGKLLEMIRGRRLVFVGDSLNRNMWESLVCILKGSVKDESQVFEAHGRHQFRWEAEYSFVFKDYNCTVEFFASPFLVQEWEVTEKNGTKKETLRLDLVGKSSEQYKGADILVFNTGHWWTHEKTSKGEDYYQEGSTVHPKLDVDEAFRKALTTWGRWVDKNVNPKKSLVFFRGYSPSHFSGGQWNAGGACDDETEPIKNETYLTPYMLKMEILERVLRGMKTPVTYLNITRLTDYRKDAHPSIYRKQKLSAEESKSPLLYQDCSHWCLPGVPDSWNEIFYAELLVKLDQLGGKRRRKALKDHRS

SEQ ID NO: 3 - DNA, 1810 bp, Arabidopsis thaliana
ccaagtttccatttgaacaaactaagaagatggactcgtcgaagaaactcctgtttccagaccagattctgtcttctagaagaaacatattaaccagattcggtttaggaatcgctgcttctttccttctccttactcttctctcccttactagttcctccttcaatgtacccttcgtctctcctctgcttcaaggtctcaaaagctccaatctaaacaattcttcttctgtcaagcaagtgaacgagaagcctgaagtagtgaatctcaccgacaaggtcccagatgtcaaagttccaagctttgtggtcccagacgctggttcgaagaacacaactttgtctgaagaaagtaaagttccaagctttgattctggacaaagatctggagaaacagttaagaactcgagtctggctgaagagggtaatggttcagttgcagatgaccaaaatacccttgaagcaaatgcgacaacaagtgtgggaaacagctcgagcttggtgtctgatttgggaggaagatttgttgtacctgcaaacacaagcaaagagaatggctctgtgactgaagatcgaagcagaggttcgtatgaggattgtgatatctacgatggaagttgggttagagctgatgatgagacaatgccgtattacccacctggttcttgtccgtatatagacagagatttcaactgtcacgctaatggaagacctgatgatgcttatgttaaatggagatggcaaccaaatgggtgtgacattcccaggttgaatggaactgattttctggagaagctaagagggaagaagctggttttcgtgggagattcgataaaccggaacatgtgggagtctcttatttgcatccttagacatagtctcaaggacaagaaacgagtgtatgagatctctgggagaagagagttcaagaagaaagggttttatgctttcagattcgaggactataattgcacggtggatttcgttggctcgcccttttttgtaagggaatcaagtttcaaaggtgtgaatgggactacattggagactttaaggttggatatgatggataagacgacatccatgtatcgagatgctgatatactgattttcaacactggccattggtggactcatgacaaaacaaagctaggagaaaactattaccaagaaggtaacgttgtgtacccgaggctcaaggttctcgaagcttataaacgagctctcattacttgggcgaaatgggtcgataagaacatcgaccgcagtcaaacccacatagtatttagaggatattccgttacacacttcagaggagggccatggaactcaggtggacaatgccacaaagaaacagaaccgattttcaacacgagttacttagcaaagtatccatcgaagatgaaagctctcgagtatattctccgtgatacaatgaaaactccggtgatatatatgaacataagtcgactaacagatttcagaaaagatggtcatccttcgatatacagaatggtttacaggacggaaaaagagaaaagagaggccgtgagtcaccaagattgtagccattggtgcttgccgggtgtaccggacacatggaaccagctcttgtacgtatcactgttaaaggccgggctagcgtctaagtggtagtcgaaagtcctctgtctttgtattcttgtttactgttgtaaagtaaaaaatgtaccaaatgaaatctcctttatagaaaaagagattgtatcatgtcacatcacatttatcactggttggttactacggtatatagaaatatttttgtatgaggc

SEQ ID NO: 4 - protein, 541 aa, Arabidopsis thaliana
MDSSKKLLFPDQILSSRRNILTRFGLGIAASFLLLTLLSLTSSSFNVPFVSPLLQGLKSSNLNNSSSVKQVNEKPEVVNLTDKVPDVKVPSFVVPDAGSKNTTLSEESKVPSFDSGQRSGETVKNSSLAEEGNGSVADDQNTLEANATTSVGNSSSLVSDLGGRFVVPANTSKENGSVTEDRSRGSYEDCDIYDGSWVRADDETMPYYPPGSCPYIDRDFNCHANGRPDDAYVKWRWQPNGCDIPRLNGTDFLEKLRGKKLVFVGDSINRNMWESLICILRHSLKDKKRVYEISGRREFKKKGFYAFRFEDYNCTVDFVGSPFFVRESSFKGVNGTTLETLRLDMMDKTTSMYRDADILIFNTGHWWTHDKTKLGENYYQEGNVVYPRLKVLEAYKRALITWAKWVDKNIDRSQTHIVFRGYSVTHFRGGPWNSGGQCHKETEPIFNTSYLAKYPSKMKALEYILRDTMKTPVIYMNISRLTDFRKDGHPSIYRMVYRTEKEKREAVSHQDCSHWCLPGVPDTWNQLLYVSLLKAGLASKW

SEQ ID NO: 5 - DNA, 1482 bp, Arabidopsis thaliana
ataattcctaatttcgacacgttctttacataaattaccagatttgccactatgagcttcttgattcctaatagaggagtaggaggaaccaagattcctctctccatcatcgttctcgttctttgtggtttcatgttcttcattctcttatacactgaaagaatcagcttgttgtcttcttcttcctcttcgtcttcatctttcttcaagctaaagtcttgtccgaggaaagatgttagctcgaaacctaaggagaaaattcgaaaggaaagatcagagatattggaagtgcttgacgacaggttcgaattcgatcccgaagagtgtaacgttgcagcagggaaatgggtttacaatagctcaattgaaccactctacaccgatagatcatgtccatacatcgatcgtcaattctcttgtatgaaaaacggacagccagaaactgattatcttcggtgggaatggcagcctgatgactgtacaattccacgatttagtccgaagttagcaatgaacaaactcagaggaaaaagactactttttgtgggagattcgttgcaacgaagccaatgggaatcgttcgtgtgtttggtggaatcaataatacccgaaggagaaaaatcaatgaagcgtagccaaaaatactttgtatttaaagcaaaggaatacaatgcgactatagagttctattgggcaccgtatatcgtcgaatcaaacacagatatacctgtgatttcggatccaaagaaacggatagtgaaagtggactcagtgaaagatcgagctaagttttgggaaggagctgatattttggttttcaacacttatgtttggtggatgagtggtctccgcatgaaagctctgtggggttctttcggaaatggagagagtggtgcagaggcactagacacacaagtggcttacaggctaggactcaagacatgggctaattgggtagactccaccgttgaccctaataagaccagagtcttcttcaccaccatgtctcctactcatactagaagtgcggattggggaaagccaaacggcacgaagtgtttcaacgagacgaaaccgataaaagataagaagttttggggaacaggctctaacaagcagatgatgaaagtagtgtcaagcgtgataaagcacatgactacgcatgtgacagtcatcaacatcacgcagctctctgagtatcgcatcgacgctcacacatcagtttacaccgagacaggtggcaagatcctaaccgctgagcagagggctgatccaatgcatcacgctgattgtatacattggtgtttacctggactacccgatacatggaatcggatcctattggctcatttgtaaatcaagctaggaggaagctgagggttaaacgggaaaaacccaaatcctctgtttttctcctcttgtgttgtcggagatttgtttgtttcagtgtattgtgattgtcgtcaattttcataagatcaa

SEQ ID NO: 6 - protein, 434 aa, Arabidopsis thaliana
MSFLIPNRGVGGTKIPLSIIVLVLCGFMFFILLYTERISLLSSSSSSSSSFFKLKSCPRKDVSSKPKEKIRKERSEILEVLDDRFEFDPEECNVAAGKWVYNSSIEPLYTDRSCPYIDRQFSCMKNGQPETDYLRWEWQPDDCTIPRFSPKLAMNKLRGKRLLFVGDSLQRSQWESFVCLVESIIPEGEKSMKRSQKYFVFKAKEYNATIEFYWAPYIVESNTDIPVISDPKKRIVKVDSVKDRAKFWEGADILVFNTYVWWMSGLRMKALWGSFGNGESGAEALDTQVAYRLGLKTWANWVDSTVDPNKTRVFFTTMSPTHTRSADWGKPNGTKCFNETKPIKDKKFWGTGSNKQMMKVVSSVIKHMTTHVTVINITQLSEYRIDAHTSVYTETGGKILTAEQRADPMHHADCIHWCLPGLPDTWNRILLAHL

SEQ ID NO: 7 - DNA, 1374 bp, Arabidopsis thaliana
atggcggatctaaagaatctcttcctcatcacgaaacatccatcaacaacacagatcttcctaacgtctctcttcttcctttctctcttcctcctctcctcttcttctctctcagatttctctccatcactcatcgtctctagcttcacctctcgtcttctcacagcagcaaacttcttctcttctccttcttcttatacttcctctgcttcagatacaactatgtttctctcttctgtttctcccagaagaatcaacgagccaaagattgattcggagaccaaagaactcgcttcttgcgacatttttgatggaacttgggtctttgacgattctgaaccggtttatcttcctggttattgtcctttcgttgaagataaattcaactgcttcaagaatggtcgacctgattcggggtttctccgtcatcgatggcaacctcatggatgctcgattccaagattcgatgggaagaagatgttgaagatgctgagagggaaaagagttgtttttgttggtgattcattgaatagaaacatgtgggagtctctggtttgttcacttaggtcaacattggaagacaaaaacagagtttctaagattattggaaaacagagtaatcttcctaacgaaggcttttacggtttcaggttcaatgactttgaatgctctattgacttcatcaaatcaccattcctcgttcaagaatcagaagttgtagatgtatacgggaagagaagagagacactgagactcgatatgattcaaagatcgatgacgaagatatacaaaaacgcagacattgttatatttaacactggtcactggtggactcaccagaaaacctacgaagggaaaggttattaccaggaaggaaatagggtttacgaaagattagaagtgaaagaagcttatacaaaagctattcatacatgggctgactgggttgattccaatatcaacagtactaaaaccagagtcttcttcgttggttattcctcttcacattttagaaaaggggcgtggaatagcggaggacaatgcgatggagaaacaagaccgatacagaacgaaacgtacacaggagtgtatccatggatgatgaaagttgtagaatctgtgatctcggaaatgaaaacgcctgtgttttacatgaacattacgaagatgacttggtaccgaacagatggtcatccgtcggtttacagacagcccgccgacccccgaggaacttctcctgcagcgggaatgtaccaagattgtagtcattggtgcttacctggagttcctgattcatggaaccagcttctctacgctactctgctagtttcacatggttccttgcctgacaagtcacttggaagtctcttgtaa

SEQ ID NO: 8 - protein, 457 aa, Arabidopsis thaliana
MADLKNLFLITKHPSTTQIFLTSLFFLSLFLLSSSSLSDFSPSLIVSSFTSRLLTAANFFSSPSSYTSSASDTTMFLSSVSPRRINEPKIDSETKELASCDIFDGTWVFDDSEPVYLPGYCPFVEDKFNCFKNGRPDSGFLRHRWQPHGCSIPRFDGKKMLKMLRGKRVVFVGDSLNRNMWESLVCSLRSTLEDKNRVSKIIGKQSNLPNEGFYGFRFNDFECSIDFIKSPFLVQESEVVDVYGKRRETLRLDMIQRSMTKIYKNADIVIFNTGHWWTHQKTYEGKGYYQEGNRVYERLEVKEAYTKAIHTWADWVDSNINSTKTRVFFVGYSSSHFRKGAWNSGGQCDGETRPIQNETYTGVYPWMMKVVESVISEMKTPVFYMNITKMTWYRTDGHPSVYRQPADPRGTSPAAGMYQDCSHWCLPGVPDSWNQLLYATLLVSHGSLPDKSLGSLL

SEQ ID NO: 9 - DNA, 1757 bp, Arabidopsis thaliana
aacacatataatttgaccaaagtcgcatctcacggtgagatgagttcttcgtagctccaatggtggtggtggcttcacaatgtcgacttcttcttctttccctcgaaccatcgtctcctacactgtgacaacttcgctcttcatcgttatcttcctatgcagcgtcttcttcttcactagacgcactctcgaaccttctctctctccttaccacaccgccgacatccctctccccgccgtcgatctaccacctcctacgcctctcctccctcaaatcgagcctcacgatggtgacgtcaccgtggaaacgaatcctaaggaggtagaagattcgcgccgcggaggcgatgacgtcgcggtagagactgagctgaaactgaaagatgtggaagattcgcacacggagaaaaccgaggaggaggaagaaggtagaggagaatcgcccggtgaagtttccgtagaatctgtggaacatgcggtgattgagaagatgagaggctgtgatttgtacaagggatcgtgggttaagggcgatgatgagtatccattgtatcagcctggatcgtgtccttatgtggacgatgcttttgattgccagaggaatgggaggcgtgattctgactatcttaattggagatggaagcctgacggttgtgatcttccacggtttaatgctacagacttcttggtgaagctacgaggtaaaagtttgatgttggttggagattcaatgaatcgtaaccagtttgaatcaatgctttgcgttctacgagaaggtttatcggataagagtagaatgtatgaggttcatggccataatataacaaaagggagaggctactttgtattcaagtttgaagattataattgcacagtagagtttgtgaggtcacattttcttgtgagggaaggagttagagcaaatgctcaaggaaatactaatccgaccctttccattgatcgcattgacaagtcacatgctaaatggaagcgagctgacattcttgtctttaacactggacattggtgggttcatggaaagaccgccagagggaaaaactactacaaagaaggtgactacatctacccgaaatttgatgctactgaagcatacagaaggtcgttgaagacttgggcaaaatggatcgatcagaatgtgaatcccaaaaagcagcttgtattttatcgcggatactcctctgcccattttcggggaggtgagtgggactcgggagggtcatgcaacggggaggtagaacctgtgaagaagggttcaatcatcgattcctatcctttgaaaatgaagatagtccaggaagccataaaggagatgcaagttcctgtgattcttctaaacgtgacaaagctgactaatttccgcaaagacggacatccatcaatctacggtaagaccaacacggatggtaagaaagtgtcgacaaggcgacaggattgcagccactggtgcctgccgggagttcccgatgtttggaaccatcttatctacgcatctttgctccttcaaccacattcttaaagttgagctcatacatcaacaaaattagatagaaagagaagagcagaaacaaatgaccagtccagtcttcactgttgtagtagtgatgtcaaattttgtgtaatttattgcccttggagctgataagtagttttttgttactttcatgtagaggtgtgaacaaacggaatcaggatgaaattttcagggttttttaatggcaaacaatttttctcataat

SEQ ID NO: 10 - protein, 485 aa, Arabidopsis thaliana
MSTSSSFPRTIVSYTVTTSLFIVIFLCSVFFFTRRTLEPSLSPYHTADIPLPAVDLPPPTPLLPQIEPHDGDVTVETNPKEVEDSRRGGDDVAVETELKLKDVEDSHTEKTEEEEEGRGESPGEVSVESVEHAVIEKMRGCDLYKGSWVKGDDEYPLYQPGSCPYVDDAFDCQRNGRRDSDYLNWRWKPDGCDLPRFNATDFLVKLRGKSLMLVGDSMNRNQFESMLCVLREGLSDKSRMYEVHGHNITKGRGYFVFKFEDYNCTVEFVRSHFLVREGVRANAQGNTNPTLSIDRIDKSHAKWKRADILVFNTGHWWVHGKTARGKNYYKEGDYIYPKFDATEAYRRSLKTWAKWIDQNVNPKKQLVFYRGYSSAHFRGGEWDSGGSCNGEVEPVKKGSIIDSYPLKMKIVQEAIKEMQVPVILLNVTKLTNFRKDGHPSIYGKTNTDGKKVSTRRQDCSHWCLPGVPDVWNHLIYASLLLQPHS

SEQ ID NO: 11 - DNA, 1750 bp, Arabidopsis thaliana
gaaactgttttttcttaactaacttataacagttacagagaaaaaataaataaagagatagaagatggagagacaaagaagtttctctgtgaagtccacaagagtcttagcattcatcatcacaataatctcttccgccattgttttcttcactttcttctcctcttccttactcaaatctaactcttctctttacccaacaccagaagctaacttccagatcgatttaagccctattgccgccatttccgattcttcagtttctcctcaagcaagtccgattctgatctctacccatttcaattcacccgaaaacacttccgggtcttcaaaaatttctgtttttgagcagaagattagcggcgagagtttggttaaggaagttagggaaattgctaatttaacttcgattaaagtgattgaacttccgagcaacaatggtgaagataagaagacagagaagagaatcgaagaatgtgatgttacgaaagggaagtgggtttacgatagtgattatccattgtacacaaatgcgtcttgtccttttatcgatgaaggatttggttgtcaaagcaatggaagattggatcttaattacatgaattggagatgggagcctcaggattgtcatgctcccaggttcaatgctacaaagatgctggaaatgattagaggcaagaggctagtgttcgttggagattcaattaataggaaccaatgggaatctatgctttgtttgttgtttcaagctgttaaagatcccaagagagtatacgaaacgcacaaccgaagaatcaccaaagagaaagggaattatagcttccgtttcgtggattataaatgcactgtggaattctatgttactcatttcttggttcgtgagggtagagcaagaataggaaagaagcgaagagaaacacttagaatcgatgctatggatcgaacctcttccagatggaaaggtgctaatatcttagtgttcaatacagctcattggtggtctcattacaaaaccaaatcaggggtcaactactaccaagaaggagacctgattcatcccaaactcgatgtatcaaccgcttttaagaaagctttgcagacttggtcatcatgggttgacaaaaatgtcgatccgaagaaaactcgcgttttctttagaagcgctgcaccttctcatttcagtggaggagaatggaactcaggaggtcattgcagagaagccaacatgccgttgaaccaaaccttcaaaccaagttactcgagcaaaaagtccattgttgaggacgtactgaagcaaatgagaacaccggtaaccctcttgaacgttagcggtttatctcagtacagaattgacgcccatccttctatttacggaacaaaacccgaaaaccggcgttcaagagctgtccaagattgcagccattggtgtctccctggtgttcctgatacttggaaccacttcctttacctccatttgctccataaacgataaatacagttacaaactaatcaaagctccagttacgtgttcaaaagtttgtttaatgtaggcattgctcgatccgatttaaccaaccaaaccaaaaccattttggaaaacccactatgacaactgaactgaaaatacaaaaccaaattttcgttttcctgtgactcacgctggtcccgtgaatgtcaatctcttggttcattgtttaggaaaataactaaataaagttacttggaccatctcttggttcattgtctacaa

SEQ ID NO: 12 - protein, 475 aa, Arabidopsis thaliana
MERQRSFSVKSTRVLAFIITIISSAIVFFTFFSSSLLKSNSSLYPTPEANFQIDLSPIAAISDSSVSPQASPILISTHFNSPENTSGSSKISVFEQKISGESLVKEVREIANLTSIKVIELPSNNGEDKKTEKRIEECDVTKGKWVYDSDYPLYTNASCPFIDEGFGCQSNGRLDLNYMNWRWEPQDCHAPRFNATKMLEMIRGKRLVFVGDSINRNQWESMLCLLFQAVKDPKRVYETHNRRITKEKGNYSFRFVDYKCTVEFYVTHFLVREGRARIGKKRRETLRIDAMDRTSSRWKGANILVFNTAHWWSHYKTKSGVNYYQEGDLIHPKLDVSTAFKKALQTWSSWVDKNVDPKKTRVFFRSAAPSHFSGGEWNSGGHCREANMPLNQTFKPSYSSKKSIVEDVLKQMRTPVTLLNVSGLSQYRIDAHPSIYGTKPENRRSRAVQDCSHWCLPGVPDTWNHFLYLHLLHKR

SEQ ID NO: 13 - DNA, 1613 bp, Arabidopsis thaliana
atgagtaatttcaccaagagttcatcattcaatagaagggcattgagttccttagctatagaaagccctaggaactcatcttctagtgtcttcactagtcccattggctcagctttcgctagcccgagatcacagaactttggaggaagtccacgcccttctacgaaccggttaaaagagatctcatatttgtttcaggtgcttatcatcgctggaactattgtgtcgtttcttgttatcatcgctggaggttatttgtatgttgttcctagccttggtcaaacgttcttaggttacaatggggcattggagtttaacagctctgttgtgggtgatacagaatgtgatatcttcgatgggaattgggttgttgacgataactacccgttatataacgcttcggagtgtccatttgtagagaaagggttcaattgcttagggaatgggcgtggacacgatgagtatttgaagtggaggtggaagcctaagcattgtactgttccaagatttgaagtacgtgatgtattgaagagattaagagggaaaaggatagtttttgtgggagattcgatgagtaggacgcagtgggagtctttgatatgcatgttgatgacagggttggaagataaaaggagtgtttatgaagtcaatgggaacaatattacgaaaaggatacggtttttgggtgtgaggtttagctcctacaattttacagttgagttttaccgatcggttttcttagttcagcctgggaggctgcgttggcacgcaccgaaacgtgttaagtcaacgttaaagttagatgttttagatgttattaaccacgaatggagctcagctgattttctcatattcaatactggtcaatggtgggtgcctgggaagcttttcgaaacgggatgttactttcaggttggaaattcactgaggctcggaatgtcaattcctgcagcgtatagagtagcattagagacatgggcatcatggatagagagtacagttgatccaaacaaaactcgggttttgtttcgtacatttgagccatcgcattggagtgatcatagatcatgcaatgtgacaaagtatccagccccagacactgaaggaagagacaaaagcatattctctgaaatgatcaaagaagtagttaagaacatgacgattccggtatcaatattggatgtaacttcgatgtcagcgtttagaagcgatggtcatgtcggtttatggagcgataatcctttggtacctgattgtagtcattggtgtctacctggagtacctgatatctggaatgagatcttgctcttcttcctctttagacaaccagttcagtgaagtggataattgcaacatagaaaaaaagattaacgacccttgaagactgaatctctcggagctaattgttcatgtttcttctgagagctaaagacagataaaggggtcacaaatttttggtgaaagttaaaaatgattcttacttaattcattcttttattgtttcttctacgtccttcttctttttatttgtaatgtcacggcttgataactattcaacacgtaatgtcacggcttgatttatctgcaactgaattcaagacattttcttgccg

SEQ ID NO: 14 - protein, 445 aa, Arabidopsis thaliana
MSNFTKSSSFNRRALSSLAIESPRNSSSSVFTSPIGSAFASPRSQNFGGSPRPSTNRLKEISYLFQVLIIAGTIVSFLVIIAGGYLYVVPSLGQTFLGYNGALEFNSSVVGDTECDIFDGNWVVDDNYPLYNASECPFVEKGFNCLGNGRGHDEYLKWRWKPKHCTVPRFEVRDVLKRLRGKRIVFVGDSMSRTQWESLICMLMTGLEDKRSVYEVNGNNITKRIRFLGVRFSSYNFTVEFYRSVFLVQPGRLRWHAPKRVKSTLKLDVLDVINHEWSSADFLIFNTGQWWVPGKLFETGCYFQVGNSLRLGMSIPAAYRVALETWASWIESTVDPNKTRVLFRTFEPSHWSDHRSCNVTKYPAPDTEGRDKSIFSEMIKEVVKNMTIPVSILDVTSMSAFRSDGHVGLWSDNPLVPDCSHWCLPGVPDIWNEILLFFLFRQPVQ

SEQ ID NO: 15 - DNA, 1284 bp, Arabidopsis thaliana
atggatcatcatcaagaatcaaatcctctgaaggagattttctctctatcttcgtctcctttcttctcaacgttgaaaatcaagaaacatatatttgttgggatctctcttctcatcaccttcctaatattctctgttatcgtcgttgatcttgccggcttcgagcctcatctctgtttcggattcttattgtcgccccgaaccctaacgaaggaaagaggaaacgacgacgtatgcgactattcatacggaagatgggttcgtcgacgccgtgatgtggacgaaacctcttattatggagaagagtgtcggtttctggatcctggttttcgttgtctgaataatggaagaaaagattctggttttcgacaatggcgatggcaaccacatggctgcgatcttccacgatttaacgcaagtgattttctggagaggagtcggaatgggaggattgtgttcgtcggagattctatcggaagaaaccaatgggaatctcttctgtgtatgctttcacaagcagtgtctaataaatctgagatatatgaagttaatggaaaccctataagcaagcataagggtttcctttcgatgcgatttccggaacaaaacctaaccgttgaatatcatagaacaccgtttctggttgtggttggtcggcctcctgagaactcaccggtagatgtcaaaatgactgttagagtcgatgagtttaactggcagtccaaaaaatgggtcggctctgatgttctagtcttcaatacagggcattggtggaacgaagacaagacctttatcgcgggttgctattttcaggagggagggaaattgaacaagacgatgggagtaatggagggatttgagaagtctttaaagacatggaagtcatgggttttagagagactagattctgagaggagtcatgtcttcttcagaagcttctctcctgtacactacaggaatgggacatggaacttgggtggtttgtgtgatgcagatacagagccagagactgatatgaagaaaatggaacctgaccctatccacaacaattatatttctcaagcaatccaagaaatgagatatgaacatagtaaggttaagtttctgaacattacatatctgaccgagttcaggaaagatgctcatccttcgcggtatcgagaaccgggaactcctgaagatgctcctcaagactgcagtcactggtgcttacccggagtaccggacacatggaacgagattctctatgcgcagcttttggcaatgaattaccgaacaaagtga

SEQ ID NO: 16 - protein, 427 aa, Arabidopsis thaliana
MDHHQESNPLKEIFSLSSSPFFSTLKIKKHIFVGISLLITFLIFSVIVVDLAGFEPHLCFGFLLSPRTLTKERGNDDVCDYSYGRWVRRRRDVDETSYYGEECRFLDPGFRCLNNGRKDSGFRQWRWQPHGCDLPRFNASDFLERSRNGRIVFVGDSIGRNQWESLLCMLSQAVSNKSEIYEVNGNPISKHKGFLSMRFPEQNLTVEYHRTPFLVVVGRPPENSPVDVKMTVRVDEFNWQSKKWVGSDVLVFNTGHWWNEDKTFIAGCYFQEGGKLNKTMGVMEGFEKSLKTWKSWVLERLDSERSHVFFRSFSPVHYRNGTWNLGGLCDADTEPETDMKKMEPDPIHNNYISQAIQEMRYEHSKVKFLNITYLTEFRKDAHPSRYREPGTPEDAPQDCSHWCLPGVPDTWNEILYAQLLAMNYRTK

SEQ ID NO: 17 - DNA, 1242 bp, Arabidopsis thaliana
atggatcatcatcaactcttctctctctgctcattttcgtacatcttcaagatcaagaaacatctcttcgtcagcctctttttacttagcctcctcatattctcgaccgtggtcgtcgacgtcatgccctctcttcgtatcggattgttgtcatcgtcatcatcgcaaacagtaacaaaggagtgcgattactccaaaggaaaatgggttcgtcgagcatcatcatcatcatcatccgttaatggattgttttatggcgaagagtgtcgctttcttgactctggttttcgttgtcataagcatggaagaaaagattctggttatcttgattggcgatggcaaccacacggctgcgatcttccacgatttaacgcaagtgatttacttgagaggagtcggaacgggaggatagtgttcgtgggagattccatcggaagaaaccagtgggaatcccttatgtgcatgctctcacaagcaatacctaataaatctgagatctatgaagtaaatgggaaccctataactaaacacaagggctttctctccatgcgattccctcgggaaaacctcactgttgagtatcacagatcaccctttcttgtcgtgattggtcggccaccggataaatcaccgaaagagatcaaaaccacagtgagagttgatgagtttaactggcagtcgaaaagatgggtcgggtcagacgttctggtcttcaattcagggcattggtggaacgaagacaaaactgtgttaacgggatgctattttgaggagggaaggaaagtgaacaagacaatgggagtaatggaggcatttggaaagtctttgaagacatggaagtcatgggtcttggagaaacttgatcctgacaagagttatgtcttcttcagaagctactctccagtgcactacagaaatgggacatggaataccggtggtctatgtgatgcagagattgagcctgagactgataaaaggaagctggaacctgatgcgagtcacaatgaatatatctataaagtaattgaagaaatgagatatcgacatagtaaggttaagtttctgaacattacatatctgacagagtttaggaaagatggtcatatttctcggtatcgagagcaaggcacttctgttgatgttcctcaagactgtagccactggtgcctacccggtgtaccagatacatggaatgagatcctctatgcgcaactcttgtcaatgaactatagaacaaagtga

SEQ ID NO: 18 - protein, 413 aa, Arabidopsis thaliana
MDHHQLFSLCSFSYIFKIKKHLFVSLFLLSLLIFSTVVVDVMPSLRIGLLSSSSSQTVTKECDYSKGKWVRRASSSSSSVNGLFYGEECRFLDSGFRCHKHGRKDSGYLDWRWQPHGCDLPRFNASDLLERSRNGRIVFVGDSIGRNQWESLMCMLSQAIPNKSEIYEVNGNPITKHKGFLSMRFPRENLTVEYHRSPFLVVIGRPPDKSPKEIKTTVRVDEFNWQSKRWVGSDVLVFNSGHWWNEDKTVLTGCYFEEGRKVNKTMGVMEAFGKSLKTWKSWVLEKLDPDKSYVFFRSYSPVHYRNGTWNTGGLCDAEIEPETDKRKLEPDASHNEYIYKVIEEMRYRHSKVKFLNITYLTEFRKDGHISRYREQGTSVDVPQDCSHWCLPGVPDTWNEILYAQLLSMNYRTK

SEQ ID NO: 19 - DNA, 1041 bp, Arabidopsis thaliana
atgtctaagaactcaaacgttgaagagaatggaggagcaaagccaatctgtgaagctttgaggagattcaaaaggtcgaggcttgtgttcgagccgtctttaggagtgttggggtttttcttggttggggtctgtttggtttgcagttttttcttctttgattacagaagcgttgctaaaagctatggcctttcggataaatctgagagattcgtttggctcaagtttgacaatatcagcagcagcagcagtagtagtagtaatagttcaaagagagttgggtttctggaagagagtggaagtgggtgtgatgtgttcgatggagattgggtttgggacgagtcttatccattgtatcaatccaaagattgcagatttctcgatgaagggtttaggtgtagtgactttggaaggtctgatttgttctatactcagtggcgttggcaacctagacactgtaatttgcccagatttgatgcaaaactgatgctggagaaactccgggacaagcggcttgtgtttgttggcgattcaataggaagaaaccagtgggagtctctactttgtctactatcttctgcagtcaagaacgagagcttgatttatgaaataaatggaagtccgatcacaaagcacaaagggttcttggtgttcaaatttgaggagtacaattgcacagtggagtactacagatctccatttttggttcctcaaagcagaccaccgattggatcacctggaaaggtcaagacaagtttgaaactggacactatggattggacttcaagcaaatggagagatgctgacgttttagtgctcaatactggacactggtggaacgaggggaaaactactagaacgggatgttactttcaagaaggagaagaagtaaaattgaagatgaatgtagatgatgcttataagcgagctctgaataccgttgtgaaatggatacataccgagctcgattcaaacaaaactcaagtcttctttcgcactttcgcccccgtgcatttcaggtttgtacggtattactcttga

SEQ ID NO: 20 - protein, 346 aa, Arabidopsis thaliana
MSKNSNVEENGGAKPICEALRRFKRSRLVFEPSLGVLGFFLVGVCLVCSFFFFDYRSVAKSYGLSDKSERFVWLKFDNISSSSSSSSNSSKRVGFLEESGSGCDVFDGDWVWDESYPLYQSKDCRFLDEGFRCSDFGRSDLFYTQWRWQPRHCNLPRFDAKLMLEKLRDKRLVFVGDSIGRNQWESLLCLLSSAVKNESLIYEINGSPITKHKGFLVFKFEEYNCTVEYYRSPFLVPQSRPPIGSPGKVKTSLKLDTMDWTSSKWRDADVLVLNTGHWWNEGKTTRTGCYFQEGEEVKLKMNVDDAYKRALNTVVKWIHTELDSNKTQVFFRTFAPVHFRFVRYYS

SEQ ID NO: 21 - DNA, 1395 bp, Arabidopsis thaliana
atgtcgaagaacccatctcaggaagaagatgaagccatgccaatctctgaagtagtaataaagagattcaagcgtttaaggctcgtgttcgagccatcactaggagtcttgggttttttcttggtgggtttgtgtttagtcttcagcttcttctacctcgattacagaaccgtggctaaaactaaaagtcatgacttttcggatcaatcggagagattcctttggctcaaggagctcgatgggtttgaagttaacaacaccaaagttgggtttcttgaagagagtgggaatggttgtgatttgttcaatgggaaatgggtttgggacgaatcgtatcccttgtatcagtctaaagattgtacctttattgatgaaggattcagatgcactgagtttgggagacctgatttgttctatactaagtggagatggcaacctaaccattgtgatttgcccagatttgatgctaagttgatgttggagaagctaaggaacaagcggctagtgtttgtaggagactcaattggaagaaaccaatgggagtctcttctttgtatgctagcttctgcaatcagtaacaagaatttggtttatgaagtgaacaatagacccatcacaaagcacatgggcttctttgtgttcaggtttcatgactacaattgcactgttgaatactatagagcaccgtttttggttcttcaaagccggccacctgaaggatcaccagagaaggtaaagaccactcttaagctagagacgatggaatggactgctgataagtggagagatgcggatatattagtgtttaacacgggtcattggtggaactatgagaaaactattcgcggaggttgttactttcaagaaggagaaaaagtaagaatgagaatgaagatagagcatgcgtatagacgagctatgaagactgtaatgaaatggatacaagaagaggttgatgcaaacaagacacaggttttctttcggacatttgcccctgtgcatttcaggggaggagattggagaactggaggaacatgtcatatggagacactgcctgattttggagcttctctggttccagcagagacatgggatcatataaagctccttcaagatgtcttgtcttcgtctctttactactcgaacatatcagagacggttaaactgaaagtgttgaacataacagccatggctgctcaaagaaacgatggtcacccatcactgtactacctgggcttggctggtcctgcgccattccacaggcaagactgcagccattggtgtctccctggagttcctgattcgtggaatgagcttctttatgcattgtttctaaagcatgaaggctattcaagtccaagatcaaacaatagcgatacagacaactttacataa

SEQ ID NO: 22 - protein, 464 aa, Arabidopsis thaliana
MSKNPSQEEDEAMPISEVVIKRFKRLRLVFEPSLGVLGFFLVGLCLVFSFFYLDYRTVAKTKSHDFSDQSERFLWLKELDGFEVNNTKVGFLEESGNGCDLFNGKWVWDESYPLYQSKDCTFIDEGFRCTEFGRPDLFYTKWRWQPNHCDLPRFDAKLMLEKLRNKRLVFVGDSIGRNQWESLLCMLASAISNKNLVYEVNNRPITKHMGFFVFRFHDYNCTVEYYRAPFLVLQSRPPEGSPEKVKTTLKLETMEWTADKWRDADILVFNTGHWWNYEKTIRGGCYFQEGEKVRMRMKIEHAYRRAMKTVMKWIQEEVDANKTQVFFRTFAPVHFRGGDWRTGGTCHMETLPDFGASLVPAETWDHIKLLQDVLSSSLYYSNISETVKLKVLNITAMAAQRNDGHPSLYYLGLAGPAPFHRQDCSHWCLPGVPDSWNELLYALFLKHEGYSSPRSNNSDTDNFT

SEQ ID NO: 23 - DNA, 978 bp, Arabidopsis thaliana
atggagttgggatccagacgaatctacacgacgatgccgtcgaagctccgatcttcttcttcacttcttccacgtattctgctcctttctctcttattactccttttctattccttaattctccgccgtccgattacttcaaacattgcctctcctccgccgtgcgatctcttctccggcagatgggttttcaatccagaaactccgaaaccgttgtacgacgagacttgtcctttccacagaaacgcttggaattgcttacggaacaagcgagataatatggatgttatcaattcatggagatgggagcccaatggatgtggattgagtcgaattgacccgacccggtttctagggatgatgaggaacaagaatgttgggtttgttggggattctctgaatgagaatttcttggtttcgttcttgtgtatacttagagtggctgatccaagtgctattaagtggaagaagaaaaaagcttggcgtggagcttacttccctaagttcaatgtcactgttgcttatcacagagctgttcttctcgctaaatatcagtggcaggcgagatcttctgctgaagctaatcaagacggagtgaaaggaacttatcgagttgatgttgatgttcctgcaaatgagtggatcaacgtcactagcttctatgatgttctcatatttaactctggccattggtggggttacgataagtttcctaaagagacacctcttgtcttctaccgaaagggaaagccgataaatcctcctcttgatatattaccaggatttgaactagttcttcaaaatatggtttcttatatccaaagagaagtccctgcgaagacccttaagttctggcgtttgcaatcaccaaggcatttctatggtggtgattggaaccagaatggaagttgtttgctcgataagccactcgaagaaaaccaggaacaatggagtgaacaaagaagcaagaaagattaa

SEQ ID NO: 24 - protein, 325 aa, Arabidopsis thaliana
MELGSRRIYTTMPSKLRSSSSLLPRILLLSLLLLLFYSLILRRPITSNIASPPPCDLFSGRWVFNPETPKPLYDETCPFHRNAWNCLRNKRDNMDVINSWRWEPNGCGLSRIDPTRFLGMMRNKNVGFVGDSLNENFLVSFLCILRVADPSAIKWKKKKAWRGAYFPKFNVTVAYHRAVLLAKYQWQARSSAEANQDGVKGTYRVDVDVPANEWINVTSFYDVLIFNSGHWWGYDKFPKETPLVFYRKGKPINPPLDILPGFELVLQNMVSYIQREVPAKTLKFWRLQSPRHFYGGDWNQNGSCLLDKPLEENQEQWSEQRSKKD

SEQ ID NO: 25 - DNA, 1239 bp, Arabidopsis thaliana
atggctacaacttcacacaacaaaccctcactcttccctctactctcccttctctgtttcatctccattttcctcctcctctctctatccaaacgcgcttccctctcctctcccaaaactcaccgctccgccaccgtcttccctccaaaacccgacggttccctctcacctctctccgccacttgcgacttctccgaaggctcctggatctacgaccccaaccccagatccaccaggtacaccagtagttgtaaagaaatcttcaaaggatggaattgtattcgtaacaacaaaaccaatggtttcgagatatctaattggcgatggaaacctaaacactgtgatcttccttcgtttgatccactcaaatttcttcagtctcatcgcaataccaacatcggatttgtgggtgattcattgaataggaacatgtttgtatctttattctgtatgttaaagagtgtgacgggtgaattgaagaagtggcgacctgccggtgcagatcgcggattcacgttttcgcagtataatttgactatcgcgtatcatcgaacaaatctattggctcgttatggtagatggtcagccaatgctaagggtggtgagttagaatctcttggatttaaagagggctatagagttgatgttgatattccagacagctcgtgggcgaaggcttcgagtttccatgacattctcattttgaacactgggcattggtggtgggcgccatcaaaatttgaccctgtaaaatctcctatgcttttctttgaaggcggtcggccaatacttccacctatacctcctgctactggcctagaccgggttctaaacaatatggtaaattttgtggagaaaacaaagcgacctggagggatcatattcttccggacacaatcacctaggcactttgaaggaggtgactgggaccaaggtggtacttgtcaacgactgcaacctttgttacctggaaaagttgaggaatttttctcagtggggaacaacggaacaaatgtagaggttcgtctcgtgaatcaacacctctataactccctcaagagtagaagcgccttccacgttttggacataactcgaatgagcgagtacagagccgacgctcatccggcagcagctggtggaaagaatcacgatgactgcatgcattggtgcctcccgggccttaccgacacttggaatgatttattcgttgcaactcttcatacgatcaaagctttgtaa

SEQ ID NO: 26 - protein, 412 aa, Arabidopsis thaliana
MATTSHNKPSLFPLLSLLCFISIFLLLSLSKRASLSSPKTHRSATVFPPKPDGSLSPLSATCDFSEGSWIYDPNPRSTRYTSSCKEIFKGWNCIRNNKTNGFEISNWRWKPKHCDLPSFDPLKFLQSHRNTNIGFVGDSLNRNMFVSLFCMLKSVTGELKKWRPAGADRGFTFSQYNLTIAYHRTNLLARYGRWSANAKGGELESLGFKEGYRVDVDIPDSSWAKASSFHDILILNTGHWWWAPSKFDPVKSPMLFFEGGRPILPPIPPATGLDRVLNNMVNFVEKTKRPGGIIFFRTQSPRHFEGGDWDQGGTCQRLQPLLPGKVEEFFSVGNNGTNVEVRLVNQHLYNSLKSRSAFHVLDITRMSEYRADAHPAAAGGKNHDDCMHWCLPGLTDTWNDLFVATLHTIKAL

SEQ ID NO: 27 - DNA, 1227 bp, Arabidopsis thaliana
atgtttggtggaaaaagtcatatactcagagggagtgtatcactggccttgatcgtccttattcttttggtcattatactcctggtttctgaggaaaacccgcttcgtgattctctctttgaagtaaaacgtcagttttcatcttcttcttcttcttcttcttcagtttgtaactttgcaaagggaaaatgggttgaagaccggaagagaccattgtattctggttttgaatgtaaacaatggttatcatccatgtggtcatgtagaataatgggcagacccgatttctcatttgagggttaccgttggcagccagaaggttgcaacatgccacagtttgacagattcactttcttgacaagaatgcagaacaagacgatagcatttataggagactcattggggcggcaacagtttcaatccctaatgtgtatggccagtggtggtgaagatagcccagaggttcaaaacgtgggatgggagtacggtttggtgaaagccaaaggagctctccgtcctgatggttgggcttatcggttcccaaccacaaacacgacgatcttgtattattggtcagctagcttatcagatttggtacccatgaacaatacagatccacctagtcttactgcaatgcatctagatcgtccaccagcattcatgagaaactaccttcaccgtttcgatgtattggttctaaacactggtcaccattggaacagaggtaagattgaaggaaaccattgggtgatgcatgtaaatggaacacaggtcgaaggcgaatatctcaaagatatcagaaacgccaaggattttacaatacacagcgttgcgaagtggctcgatgcacagcttccattgcatccgcggttgaaagctttctttaggacaatttctccgaggcattttaaaaacggagattggaatacaggtggaaactgtaacaacacggttcctttgtctagaggcagcgaaatcacaggggatgatggatcgatcgatgcaacagttgagagtgctgtgaacgggacaaggatcaagattcttgacataactgcactttctgagctaagagacgaagctcatatctcagggtctaaactcaaaccccgaaaaccgaagaaggcaagtaacgtgacctcaactccaacgatcaacgattgcttgcattggtgcttaccagggatcccagatacttggaatgaacttttcattgctcagatttga

SEQ ID NO: 28 - protein, 408 aa, Arabidopsis thaliana
MFGGKSHILRGSVSLALIVLILLVIILLVSEENPLRDSLFEVKRQFSSSSSSSSSVCNFAKGKWVEDRKRPLYSGFECKQWLSSMWSCRIMGRPDFSFEGYRWQPEGCNMPQFDRFTFLTRMQNKTIAFIGDSLGRQQFQSLMCMASGGEDSPEVQNVGWEYGLVKAKGALRPDGWAYRFPTTNTTILYYWSASLSDLVPMNNTDPPSLTAMHLDRPPAFMRNYLHRFDVLVLNTGHHWNRGKIEGNHWVMHVNGTQVEGEYLKDIRNAKDFTIHSVAKWLDAQLPLHPRLKAFFRTISPRHFKNGDWNTGGNCNNTVPLSRGSEITGDDGSIDATVESAVNGTRIKILDITALSELRDEAHISGSKLKPRKPKKASNVTSTPTINDCLHWCLPGIPDTWNELFIAQI

SEQ ID NO: 29 - DNA, 1449 bp, Arabidopsis thaliana
atggtgcagattgtgccccacgtagcaaatgcacacatggtcaaaacttctagaaaaggactcttagcgcttgtcgtagttggagtagaagagtgttgcccgacgcgtcgatgtgatttgagagatccaaatttagtggtggattctaattgcttgctgtcaagatctacacatcaaagctcactcgttgaagattcaatcatcatcaatcatttccggatcttgaacttcaacaacagagcaagcatctttctttgtttgagaattggtgttatctgtagcactcaatctaagatgtatggtggaaaaagttacatactcaaagggagtgtatcactttccttgatcatcctcattcttttggtgacaacactcctggtttccgcaaaaaaccagcctcgcatttctctgattgaagtaaaaacttgtaacttggcaaagggggaatgggttgaagacaagaagcggccattgtattctggttttgaatgtaaacaatggttatcaaacatattttcctgcagggtaatgggcagacccgacttctcatttgagggttaccggtggcagccagaaggttgcaacataccagagtttaacagagtcaacttcttgagaagaatgcagaacaagactatagcatttataggagactccttgggaagggaacaatttcaatccctgatgtgtatggccactggaggtaaagagagtccggaggttcaaaacgtgggatcggagtatggtcttgtgatacctaaaggagctcctcgtcctggtggttgggcttataggtttccgaccacaaacacaacggttttgtcttattggtcagctagtttaacagatttggtaccgatgaacaatacagatccacctcatcttattgcaatgcatcttgaccgtccaccagcattcataagaaactaccttcaccgtttccatgtattggttctaaacaccggtcaccattggagcagagataagatagaaaaaaatcattgggtgatgcacgtaaatggaacacgtgtcgaaggcggatatttcaagaacgttgaaaacgctaaaatttttacaatacacagcctggtgaagtggcttgatgcacaacttccattgcatccacggttgaaagctttctttacgacgatttcccctaggcatgaaaagtgtaacaatactatccctttgtctagaggaagcaaaatcacaggcgaaggtggatcgctcgatacaatagttgagagtgctgtgaatgggacaagggtcaagattcttgacataactgcactttctaaactaagagatgaagctcatatcgcagggtgtaaactcaaaccgaaaaaggcgagtaacgtgacttcagctccaacgttcaacgattgcttgcattggtgcttacccgggatcccggatacttggaatgaacttctcatagcacagctttga

SEQ ID NO: 30 - protein, 482 aa, Arabidopsis thaliana
MVQIVPHVANAHMVKTSRKGLLALVVVGVEECCPTRRCDLRDPNLVVDSNCLLSRSTHQSSLVEDSIIINHFRILNFNNRASIFLCLRIGVICSTQSKMYGGKSYILKGSVSLSLIILILLVTTLLVSAKNQPRISLIEVKTCNLAKGEWVEDKKRPLYSGFECKQWLSNIFSCRVMGRPDFSFEGYRWQPEGCNIPEFNRVNFLRRMQNKTIAFIGDSLGREQFQSLMCMATGGKESPEVQNVGSEYGLVIPKGAPRPGGWAYRFPTTNTTVLSYWSASLTDLVPMNNTDPPHLIAMHLDRPPAFIRNYLHRFHVLVLNTGHHWSRDKIEKNHWVMHVNGTRVEGGYFKNVENAKIFTIHSLVKWLDAQLPLHPRLKAFFTTISPRHEKCNNTIPLSRGSKITGEGGSLDTIVESAVNGTRVKILDITALSKLRDEAHIAGCKLKPKKASNVTSAPTFNDCLHWCLPGIPDTWNELLIAQL

SEQ ID NO: 31 - DNA, 1656 bp, Arabidopsis thaliana
atgaacgaaactttctctgcaacgccaagaagcatgaccatccatagatataggatgaaaagaggagcccttagacgaagggctagggatatctctgttatgcttgttgtacttgtttgtgcaactgtagtcatatggacatgggatagaactccaacctcggcctttcttccacctgaaagtcactatctaaagctgcaatcagaagaaaaggttgagaaattacctactgcgctgaatactgaaactaaagacagctactcatcagctattccatttgtaaacaaagaggaaagcaaagaggattcatccgataataaagatacagaagaagaagaggagaaacaagtggaagaagttactgtaagtaatacgaatcgagggaagataccaacgattgaagaaaaaaagggggaacatgaagttattgcaagtgagccaaaatatcggaagacgccaactagagaagattttaaattagaaaaggtgaaacatgaggttgctgttggtgagggcgaagctacggagacaacacatattaaagagactaattcagatccaaagagtaatattcttgcaacagacgaagagaggactgatggtacttcaacagctcgcatcacgaatcaagcttgcaattatgcgaaagggaaatgggttgtggacaatcaccgtcctttgtattcaggatctcaatgcaaacagtggcttgcttcaatgtgggcatgcaggttgatgcaacgtacagactttgcctttgaaagtttaagatggcaacctaaagattgctctatggaagaatttgagggttccaagttcttgagaaggatgaagaacaaaaccctagcctttgttggagactcgttgggaagacaacagtttcagtctatgatgtgtatgatctcgggaggcaaagagagactcgatgtccttgatgtgggaccagaatttgggttcataactcccgaaggtggagctcgtcctggtggttgggcttacagattcccagaaactaacacaacggtcctataccactggtcatctacactgtgcgacatagaaccgctcaacatcactgaccccgcaactgaacacgctatgcatcttgatcgcccaccagcgtttttacgccaataccttcaaaagatcgacgtattggtaatgaacacaggtcaccattggaaccgcgggaagctcaacgggaacaaatgggtgatgcatgttaatggcgttcccaacactaacaggaagttagctgctcttgggaacgccaagaatttcacaatccacagcacagttagttgggttaactcacaactccctctccatcccggtctcaaggcattctacagaagcctttcaccgagacatttcgtgggtggggaatggaacaccggagggagctgcaataacacaaccccaatgtctatcgggaaagaagttttgcaagaggagtcaagcgactacagcgcgggtcgtgcagtgaaaggtacaggggttaagcttttagacataacagccttatctcatattagagacgaaggtcacatatcacggttcagtatctcggcttcacggggagttcaggattgcctccattggtgcttgcccggtgttcctgatacgtggaatgaaatcctttttgcaatgatctaa

SEQ ID NO: 32 - protein, 551 aa, Arabidopsis thaliana
MNETFSATPRSMTIHRYRMKRGALRRRARDISVMLVVLVCATVVIWTWDRTPTSAFLPPESHYLKLQSEEKVEKLPTALNTETKDSYSSAIPFVNKEESKEDSSDNKDTEEEEEKQVEEVTVSNTNRGKIPTIEEKKGEHEVIASEPKYRKTPTREDFKLEKVKHEVAVGEGEATETTHIKETNSDPKSNILATDEERTDGTSTARITNQACNYAKGKWVVDNHRPLYSGSQCKQWLASMWACRLMQRTDFAFESLRWQPKDCSMEEFEGSKFLRRMKNKTLAFVGDSLGRQQFQSMMCMISGGKERLDVLDVGPEFGFITPEGGARPGGWAYRFPETNTTVLYHWSSTLCDIEPLNITDPATEHAMHLDRPPAFLRQYLQKIDVLVMNTGHHWNRGKLNGNKWVMHVNGVPNTNRKLAALGNAKNFTIHSTVSWVNSQLPLHPGLKAFYRSLSPRHFVGGEWNTGGSCNNTTPMSIGKEVLQEESSDYSAGRAVKGTGVKLLDITALSHIRDEGHISRFSISASRGVQDCLHWCLPGVPDTWNEILFAMI

SEQ ID NO: 33 - DNA, 1506 bp, Arabidopsis thaliana
atgactttggcttctccacgagtttcaaattcaaagacgactgtgttgttgtttcctcgaaaagtttcttcaattgcatttgctattggtggattaacttctttcgtcatcttcgcttctctgcttcttttcacgtatcccattggttcctctgttactgactatttctacagaacagagaccactcaaaacgtccagtttcaccatagtatccatgatcctgatcgtaacccatctcctgtttctagctcagagtcaccaccagtattgacacaagatagtgatgataaagtgcttcccaagggatctcatgattcgaatgatgttagattaggtgaagaaacaaactcagggaagtcttcaaatgtttccattgatgaagaagctacacaagatcatgtagaaacagaatgtgatttatatcatggtaactggttttatgatccgatgggaccgttgtatacaaacaactcttgtcctcttttgacacagatgcaaaactgtcagggcaatggaagacctgacaaggggtatgaaaactggagatggaaaccgtctcagtgtgatcttccgcgttttgatgctaagaaatttcttgagctaatgagaggcaagacactagcttttataggcgattcagtagctcgtaatcagatggagtccatgatgtgtcttctttggcaggtagaaactccggttaaccgtgggaacaggaagatgcagagatggtatttcaggtcatcatcggtgatgatagctcggatgtggtcatcttggctggtccatcagttcaatgaaccgtttggttttgctacagatggtgtaactaaactcaagcttgaccaacccgatgagcgcataattgaagctcttccgaatttcgatgtggttgtactttcatcaggtcactggtttgcaaaacagtctgtctatattttaaatgaccagattgtgggaggccagttatggtggccagacaaatcaaaacccgaaaagattaacaacgtcgaagcatttgggatctctgtagaaacaatcataaaggcaatggctaaacacccaaactacacgggtctgaccatcttacgtacttggtcgcctgatcattatgaaggcggtgcgtggaacactggtggatcatgcacaggtaaagtagagccgcttcctcctgggaatttagttacaaacgggttcacagagataatgcacgagaagcaagcaacagggtttcaccgtgctgtcgcggatgataagctcgggaatagatcgaagaagctgaagctaatggatatcactgaggctttcgggtatcgccacgacggtcatcctggtccatacaggagtcctgacccgaaaaagatcacaaaacgtggaccagacggtcagcctcctccacaagactgtttgcattggtgcatgccagggccagtggacacatggaacgagatggttctggagattattaggagagattttgaaggcagacaaagttcaccatcatcttga

SEQ ID NO: 34 - protein, 501 aa, Arabidopsis thaliana
MTLASPRVSNSKTTVLLFPRKVSSIAFAIGGLTSFVIFASLLLFTYPIGSSVTDYFYRTETTQNVQFHHSIHDPDRNPSPVSSSESPPVLTQDSDDKVLPKGSHDSNDVRLGEETNSGKSSNVSIDEEATQDHVETECDLYHGNWFYDPMGPLYTNNSCPLLTQMQNCQGNGRPDKGYENWRWKPSQCDLPRFDAKKFLELMRGKTLAFIGDSVARNQMESMMCLLWQVETPVNRGNRKMQRWYFRSSSVMIARMWSSWLVHQFNEPFGFATDGVTKLKLDQPDERIIEALPNFDVVVLSSGHWFAKQSVYILNDQIVGGQLWWPDKSKPEKINNVEAFGISVETIIKAMAKHPNYTGLTILRTWSPDHYEGGAWNTGGSCTGKVEPLPPGNLVTNGFTEIMHEKQATGFHRAVADDKLGNRSKKLKLMDITEAFGYRHDGHPGPYRSPDPKKITKRGPDGQPPPQDCLHWCMPGPVDTWNEMVLEIIRRDFEGRQSSPSS

SEQ ID NO: 35 - DNA, 1602 bp, Arabidopsis thaliana
atggcttttggttctcctaggaataattctatattagctgcggctggatttcctcggaaagtgtctacagttgcaattgctattggtggtttggcatcattctttgttttcggattgttgcttcgtctttcgtatccgaatagttcttcagttggtggcatattttatgggatagggaatcctgaacaagttcatgtccctttatctttgagcaaccatactgtcaatatccttcagaagagtagtgacatcaatgcgtttgataaaaatctgacttctgattctagctctggcttacctgttgttgttagcaaaagtataccacctccagatttcagtagtgataggaagttggagacacctttgacacaagaaaaagaggatttggtttcttcggatataactgaaaagactgatgttcaatcaggtgaacgtgaaaccaatgtttccaaggctgaagacactccaagtgcatcatctcctccagatgatgtctctgaaactgcttcagcagaaccagagtgtgatttgtatcaaggtagttggttttatgatcctgggggacctctctacacgaacaactcttgccctgtcctgacgcagatgcagaattgccagggaaatggaagacctgacaagggatatgagaactggagatggaaaccgtctcagtgtgagcttcctcggtttgatgccagaaaatttcttgaacttatgaaaggcaagacactggcctttataggagattcagtagctcgtaatcagatggagtccatgttgtgccttctttggcaggttgaaacaccagtcaatcgtgggagccgaaagatgcagagatggtattttaagcaatcatcagtaatgattgcccgtatttggtcatcttggcttgttcatcagttcaacgaaaaattcgattatgctcctgaaggtgtcacaaaactaaagctggatcttcctgatgagcgcataatggaagctatcccgaaattcgacgtggttgtcctctcatcagggcactggtttgcaaagcagtctgtctacatattgaaagaagagatcgtgggaggccaattatggtggccagacaaatcaaaacccatgaaagtcaataacgtggatgcatttggaatatcagttgaaacaatccttaagtccatggctacacacccgaactacagcggtttgaccattgtgagaacattctcacctgatcattacgaaggtggtgcttggaacacaggtggatcatgcacggggaaagaagaacccatccttccagggaagctagtgaaaaacggattcacagagataatgcatgagaagcaagcgacaggatataaccaagccgtcgataaagtcgcggagaatttgaagctaaaactaaagctgatggatatcacagaggcatttggatatcgccatgatggtcatcctggtccattcagaagtcctgaccctaacaagatcactaaacggggaccagacggacggcctccacctcaggactgcttgcactggtgcatgccaggaccagtcgatacatggaacgaaatggtcttggagctcataaggagagacaggaaaagctcatcaacttga

SEQ ID NO: 36 - protein, 533 aa, Arabidopsis thaliana
MAFGSPRNNSILAAAGFPRKVSTVAIAIGGLASFFVFGLLLRLSYPNSSSVGGIFYGIGNPEQVHVPLSLSNHTVNILQKSSDINAFDKNLTSDSSSGLPVVVSKSIPPPDFSSDRKLETPLTQEKEDLVSSDITEKTDVQSGERETNVSKAEDTPSASSPPDDVSETASAEPECDLYQGSWFYDPGGPLYTNNSCPVLTQMQNCQGNGRPDKGYENWRWKPSQCELPRFDARKFLELMKGKTLAFIGDSVARNQMESMLCLLWQVETPVNRGSRKMQRWYFKQSSVMIARIWSSWLVHQFNEKFDYAPEGVTKLKLDLPDERIMEAIPKFDVVVLSSGHWFAKQSVYILKEEIVGGQLWWPDKSKPMKVNNVDAFGISVETILKSMATHPNYSGLTIVRTFSPDHYEGGAWNTGGSCTGKEEPILPGKLVKNGFTEIMHEKQATGYNQAVDKVAENLKLKLKLMDITEAFGYRHDGHPGPFRSPDPNKITKRGPDGRPPPQDCLHWCMPGPVDTWNEMVLELIRRDRKSSST

SEQ ID NO: 37 - DNA, 1281 bp, Arabidopsis thaliana
atggagcttgttcattctgcaacatttccatgtaaacaaaagctactcatcgcagtgaccattgctacatctcttttaaccatcatccctcttctctaccctttgcttgaagaccctaacttcttcctcaagcaacaacctccaagccaaagcagcatcatcaacctcgaaaacggtgttgtaacatcacatgacagctgcgacatattctcgggagagtgggttcctaaccctgaggcaccttactacaccaacacgacgtgctgggcgatacacgaacaccaaaattgcatgaagtttggaagacccgacacggatttcatcaaatggaaatggaaaccttacgggtgcgaagacggcttgccggtatttgacccggttaggtttcttgagatcgtgagaggtaaaaccatggcgtttgttggtgactcagttagcagaaaccacatgcagtctttgatctgccttctttctcaggttgaatatccgatggatgcttcggttaagaatgacgattatttcaaacgatggacatacgaaacatacaatttcacgatcgccgcattttggacaccacacttggttaagtccaaagaacccgaccagacccagcccaagcatattgatatttttgacctataccttgatgaggcagacgagagttggacagccgatataggagattttgacttcgtaatcatctcttctggccactggcattaccggccatcagtctactacgaaaaccggactatcaccggttgccattattgccaattaccgaacataaccgatctcacaatgttttatgggtatagaaaagcttttagaaccgcgtttaaagcgatcttggattcggaaagcttcaaaggcgtaatgtacttacgaagttttgcaccgtcacattttgaaggcgggttgtggaatgaaggaggagattgtttgaggaaacaaccttataggagcaatgagactcaagacgaaacgacaatgaagctacacaagattcagttggaagaattttggagagctgaagaagaagcgaagaagaaagggaaaaggttacgattactagatacgacccaagctatgtggcttagacccgacggtcacccaagccgctacggccatattccagaggccaatgtcacgttgtacaacgactgtgtccactggtgcttgcccggtcctatcgacaacctcaatgacttcttgctggctatgctaaagagagaagaagacaaaggattcttggcgcaagttcgaaaaatgttgtcctaa

SEQ ID NO: 38 - protein, 426 aa, Arabidopsis thaliana
MELVHSATFPCKQKLLIAVTIATSLLTIIPLLYPLLEDPNFFLKQQPPSQSSIINLENGVVTSHDSCDIFSGEWVPNPEAPYYTNTTCWAIHEHQNCMKFGRPDTDFIKWKWKPYGCEDGLPVFDPVRFLEIVRGKTMAFVGDSVSRNHMQSLICLLSQVEYPMDASVKNDDYFKRWTYETYNFTIAAFWTPHLVKSKEPDQTQPKHIDIFDLYLDEADESWTADIGDFDFVIISSGHWHYRPSVYYENRTITGCHYCQLPNITDLTMFYGYRKAFRTAFKAILDSESFKGVMYLRSFAPSHFEGGLWNEGGDCLRKQPYRSNETQDETTMKLHKIQLEEFWRAEEEAKKKGKRLRLLDTTQAMWLRPDGHPSRYGHIPEANVTLYNDCVHWCLPGPIDNLNDFLLAMLKREEDKGFLAQVRKMLS

SEQ ID NO: 39 - DNA, 1122 bp, Arabidopsis thaliana
atggaacttcctgcgacaaagttacgaataggactagtgatatttcctcttattctacttacgatagctccaatcctttatctcttttttggttatcctctctattattcaacatcaacatacaaacatttatcgaattcatcatcgtcgtcggcaatatcttctccatctaggtataaccattcatcatcatcatcagatagttataagacagaagattcagaaccatcgtcgtatgataatgattacgacgacacatatcatgatcccaagtcatcatctttgcataacaatgatcgtctatccatctcatcatcgaatggtcatcatcaagtaactcctaagaaagagcaccgccggaaaaagagaaaacgtaaatgcgatatattctccggcgagtggatcccaaaccctaaagccccgtactacacaaacacgacatgcagggcgatacacgaacaccaaaactgcatcaagtatggaagacctgatttaggtttcatgaaatggagatggaaacctaaagaatgtgatctccctctgtttgatccttacgagtttctggaaatagttagaggaacaagaatggcgttcgttggtgactccgttagcagaaaccacgttcagtctttgatctgcctactctctcgggtggaacatcctgagggtgactcacaacaggagtttaatttccaacgatggaaatacaaaacgtacaacttcacgatcgccacattttggacaacacatttggttcgcgctgaagaaacggaaaccggtccgacaggtcctaattcattctacaatctctacctcgacgaacctgaccctacttgggcttcccaaatcggtgagttcgattacataatcatctcatctggacaatggtttttccgaccactcttccttttcgacaaacaaaaaaggataggatgtttgtattgctacattcccggagtcagaaatgtcggggcacatttcgcgtatagaagagcattgagaacaacattcaagacaattcttggattagagaattttaaaggagaagtgtttctgaggacatttgctccttcacatttcgaaggtggagaatgggacaaaggagcaatgagacagagttag

SEQ ID NO: 40 - protein, 373 aa, Arabidopsis thaliana
MELPATKLRIGLVIFPLILLTIAPILYLFFGYPLYYSTSTYKHLSNSSSSSAISSPSRYNHSSSSSDSYKTEDSEPSSYDNDYDDTYHDPKSSSLHNNDRLSISSSNGHHQVTPKKEHRRKKRKRKCDIFSGEWIPNPKAPYYTNTTCRAIHEHQNCIKYGRPDLGFMKWRWKPKECDLPLFDPYEFLEIVRGTRMAFVGDSVSRNHVQSLICLLSRVEHPEGDSQQEFNFQRWKYKTYNFTIATFWTTHLVRAEETETGPTGPNSFYNLYLDEPDPTWASQIGEFDYIIISSGQWFFRPLFLFDKQKRIGCLYCYIPGVRNVGAHFAYRRALRTTFKTILGLENFKGEVFLRTFAPSHFEGGEWDKGAMRQS

SEQ ID NO: 41 - DNA, 1581 bp, Arabidopsis thaliana
atggagcttcctttgtttgcaatccttcaaagaacaccaagagttgcaattacattagcatttgttctatttgtcctcacaatagttcctgctttatatactctccttgccgatccaatcttacctccatcaatctcatctttcgagactgatcatacacgtctcagtcatttgaattcttcatcaaatcagattccttcacccgtgaatggttccattcccacgccaccctaccatacgccttcaaaacatcggaagtcttctttccatcggattcctaaacccaagaaaggtcccatttcctctccaaccgatcatatacctttaagacaacggagttcttcattcgatcagattccttcacccatgaatagtcccgttcccgctccaccacatcggaattcttcagctgatcagagtccttcacctgtgaacggtcccattcccgctccactcaatcacacgtccttaagacatctgaattcttcatctgatgatcattcatccccagtcaccacatcaccatcaaggacgcggatcagagatgatgagcaaatgtgtgacctttttaccggggagtgggtaccaaacgaagaagctccatactacaccaacacgacatgctgggcaatacatgagcaccagaactgcatgaaatatgggagacctgataccggattcatgagatggaggtggaagccggagagttgcgaccttcccatctttgatcctcaagagttcttggaaatggttagagggaaagccatggggtttgttggtgactccattagcagaaaccaagtccaatctctcttgtgccttctctctagggtggaataccctgaagacatttctccttcaccagatacggatttcaaagtatggaactacacatcctataacttcactcttcatgtcatgtggtcaccatttctagtgaaggctactaaacctgatccgaagtccaacttcttcagcctatacctcgacgaatatgacacgaagtggacgagtcagctcgaccaactcgactacctggttatatcatccggccactggttttcccggccggttattttctatgaaaaccaacaaatctctgggtgccaatactgtgcgttaccaaacacgactgaattgcctttgacctatggatacaggaaggctttaagaatatctctaaaagctataatcgagaatttcaagggtctggcatttttaaggagtttttctcctcaacatttcgaaggtggggcttggaacgaaggaggggactgtgtacggacacaaccttacagaagaaacgagactatacccgaggcagatctcaaggttcacgacattcaaagggaggaatttagagctgcagaagaagatgggatgaagaagagtggtttgagactgaagttaatggacacgacacaggcaatgctattgcgaccagatgggcatcctgggagatatggacatctacagaatccaaatgtgactttaagaaatgactgtattcattggtgcctgcctggacctatcgatactctgaatgatatattgctacaaatgatgaagacagataactag

SEQ ID NO: 42 - protein, 526 aa, Arabidopsis thaliana
MELPLFAILQRTPRVAITLAFVLFVLTIVPALYTLLADPILPPSISSFETDHTRLSHLNSSSNQIPSPVNGSIPTPPYHTPSKHRKSSFHRIPKPKKGPISSPTDHIPLRQRSSSFDQIPSPMNSPVPAPPHRNSSADQSPSPVNGPIPAPLNHTSLRHLNSSSDDHSSPVTTSPSRTRIRDDEQMCDLFTGEWVPNEEAPYYTNTTCWAIHEHQNCMKYGRPDTGFMRWRWKPESCDLPIFDPQEFLEMVRGKAMGFVGDSISRNQVQSLLCLLSRVEYPEDISPSPDTDFKVWNYTSYNFTLHVMWSPFLVKATKPDPKSNFFSLYLDEYDTKWTSQLDQLDYLVISSGHWFSRPVIFYENQQISGCQYCALPNTTELPLTYGYRKALRISLKAIIENFKGLAFLRSFSPQHFEGGAWNEGGDCVRTQPYRRNETIPEADLKVHDIQREEFRAAEEDGMKKSGLRLKLMDTTQAMLLRPDGHPGRYGHLQNPNVTLRNDCIHWCLPGPIDTLNDILLQMMKTDN

SEQ ID NO: 43 - DNA, 1245 bp, Arabidopsis thaliana
atgaaatcatcgtcatcgatttttcgtgaaacttcagagaaaaaatcagagagatggatgatgatgaacattggaagattttctccatttttcttgtcttccttttgtataactcttttcttcaccggtttctttgtttatcagaatccttttaaatccatcgctgatcaaaatgttctttctttccaacctcaaatcgatcctgagtgtgatttgttcaagggacattgggttccagacaaaagaggatctttatacacaaactcgagctgtgctacgattcccgactcgaagaactgcataaaacaagggagacccgacaaagatttcttgttctggagatggaaacccgatggttgcgatcttccgaggtttaatccaaaggcgtttctcagtatggttcgagggaagaagatgaattttattggtgattctgtggctaggaaccatatggaatctcttctttgcttgttgtcaatggaagaaactcctaaggacatctacaaggatggagaagatagaaacaggatttggtactttccgaagcacgattttactctctcgacctcgtggactaagtttcttgtggaggaacgagagagaagagatagtaacaatacaggtactggattgtttgatctagatatcggcaagatcgatgaagggtggtttaatggtctaccgaatacagacattgctattgtctcggctgctcattggttcttcagacctatatttatacatagaggagatgagacactgggttgcatttactgtaacttaccaaacatgactcagattagcccggaagaagggtttaagcttgtttactcagctgtccttaggcaaatcaatgagtgtgagatgtgtaagaaagatttagtgacggtactgaggactatttcacctgcgcatttcgagaatgggacttgggacaccggaggaacttgcagcaggacgagtcctttcggagaaaacaaaattgacctgcagagcaatgagatgaagatcaggaaatctcagattgaacagcttgaaggtataacaaaacgaggcaacaaagcaaagaagtttgcggtattggatgtgacaagggttatgcagatgagacctgatgggcatcccaatggttactggggaaataaatggatgaaaggttataatgattgcgtgcattggtgtttgcccgggccaatcgatgcgtggaacgactttttaatggcgataattagacagttaagatga

SEQ ID NO: 44 - protein, 414 aa, Arabidopsis thaliana
MKSSSSIFRETSEKKSERWMMMNIGRFSPFFLSSFCITLFFTGFFVYQNPFKSIADQNVLSFQPQIDPECDLFKGHWVPDKRGSLYTNSSCATIPDSKNCIKQGRPDKDFLFWRWKPDGCDLPRFNPKAFLSMVRGKKMNFIGDSVARNHMESLLCLLSMEETPKDIYKDGEDRNRIWYFPKHDFTLSTSWTKFLVEERERRDSNNTGTGLFDLDIGKIDEGWFNGLPNTDIAIVSAAHWFFRPIFIHRGDETLGCIYCNLPNMTQISPEEGFKLVYSAVLRQINECEMCKKDLVTVLRTISPAHFENGTWDTGGTCSRTSPFGENKIDLQSNEMKIRKSQIEQLEGITKRGNKAKKFAVLDVTRVMQMRPDGHPNGYWGNKWMKGYNDCVHWCLPGPIDAWNDFLMAIIRQLR

SEQ ID NO: 45 - DNA, 1299 bp, Arabidopsis thaliana
atgaagctaaaatgggaatcaatctctaatcttcaacaaaacacatatctcataaaactcgtcgctgcaacacttataacttgtctcgcttttcgtttcttcgtcttccgcttcggccaattttccccagttcaagtgtcggtgaccggaaactccaattcccagatttctccgacgtctgttatcttatcggataacgaagatcaaattcccgtcgatattgaggtggagaagtgtgatctatttactgggaaatggattaaagatcctttgggaccaatctacacgaatgagtcatgtggtatcgttgtagatgctcaccagaattgcatcactaatggtcgacctgattctggttttctcaattggaaatggaagcctaatgactgttccttgcctcggtttgattctctgagatttctgcagcttatgaggaataaatcttgggctataatcggtgactccattgctcgtaaccatgtcgagtctttgctttgtatgctatctacagttgaaaaacctgttgaagtgtatcacgatgagaactatagatcgaagcgttggcattttccttcgtataactttacggtatccaacatttggtcaccttttcttgttcaagctgatatctttgaagactcaaatggtgtctcatcagctgcagttcagcttcatctagataaactcgacaatacatggactgatctattcccgagccttgactatgcgatcatctcttcaggggaatggttcttgaaaactgcggtataccacgagaatgctaatcccgttggatgccatggctgcccggagagttcgaacatgacggatttaggattcgactatgcttacaatacgtcgttgcgccatgttatggacttcatagcgaaatctaaaactaaaggcatgatctttttccggacttcgatacctgatcattttgaagatggggaatggcataacggtgggacttgtaagaaaacagaaccggtgggtgaagaggcagtcgagatgaaagttctgaacaagatattgagagatgttgagattaatcaattcgagagagtggttacggagatgggtcaggagagtgagaatctaaagcttctggattttgctggcatgttgctgactcgacccgatggtcatccgggtccatatagagagtttaggccgtttgataaagataagaacgctacggtacagaacgattgtctgcattggtgcttgcctggtccgattgatcacttgaacgatgtcatattggagatcatagtgaatggtcgaaccggtaaataa

SEQ ID NO: 46 - protein, 432 aa, Arabidopsis thaliana
MKLKWESISNLQQNTYLIKLVAATLITCLAFRFFVFRFGQFSPVQVSVTGNSNSQISPTSVILSDNEDQIPVDIEVEKCDLFTGKWIKDPLGPIYTNESCGIVVDAHQNCITNGRPDSGFLNWKWKPNDCSLPRFDSLRFLQLMRNKSWAIIGDSIARNHVESLLCMLSTVEKPVEVYHDENYRSKRWHFPSYNFTVSNIWSPFLVQADIFEDSNGVSSAAVQLHLDKLDNTWTDLFPSLDYAIISSGEWFLKTAVYHENANPVGCHGCPESSNMTDLGFDYAYNTSLRHVMDFIAKSKTKGMIFFRTSIPDHFEDGEWHNGGTCKKTEPVGEEAVEMKVLNKILRDVEINQFERVVTEMGQESENLKLLDFAGMLLTRPDGHPGPYREFRPFDKDKNATVQNDCLHWCLPGPIDHLNDVILEIIVNGRTGK

SEQ ID NO: 47 - DNA, 1293 bp, Arabidopsis thaliana
atgaagttgaagttgtcatcaatctccatgaatcataagaatgtgcttctcatcaagctcatctccgctattctgatatcttttttcgcgttccgtctcttcattctccactccagcgagttttctccaatctttgcgtcggtcaccggaaaattcgaagctcggtttctgccgccggagattatcgtaccggaaaatgaagatctgattcctcaagatattgaagtagagaagtgtgatcttttcgccggaaaatggatcccagattcagtaggaccaatctacacaaacaaatcatgcggatcactcattgacggtcaccaaaattgtatcaccaatggccggcctgacttagattttctctactggaaatggaaacctcatgactgtttgttaccacgttttgaccctagaagatttcttcaacttatgaggcataaatcttgggcatttattggtgactccatatctcgtaaccacgtcgaatcactgctttgtatgctctctacgattgaagaaccggttgaagtataccatgacatggagtacaaatcaaaacgttggcatttccctttacataaccttactgtatccaacatatggtcgccatttcttgttcaagccgctattttcgaagattctaacggtgtctcgaccgcttctgttcagctacatcttgatagactcgatgagacatggaccagtctcatgcctagcttcgactatgcgatcatctcaaccggtaaatggtttcttaaatccgcaatttaccacgaaaatgcaaaactcgtcggttgccataactgtcaggaaaaacctcacatagaagagttaggattcgactatgcttataacgcatcgctgcataatgtcatggactttctagcagctgaagataacagtaaaggcacggttttctttcggacttcgactcccgatcatttccaaaacggggaatggcatagtggcgggacgtgtaagcaaacagagccggtgagcgatgaggagattgagattaaggatgtacataagatactaaaagatattgagattgatcagttcaagagggcggttagagagaagactaaccaggacggtgggaatctaaagcttttggatttcacccggatgttgctgacccgaccggacggtcatccgggtgagtaccgacagttcaggccgtttgataaagacaaaaatgcaaaggtacagaacgattgtttgcattggtgcttgcctggtccgtttgattacttgaatgatgttatattggagactatagtaaatggctga

SEQ ID NO: 48 - protein, 430 aa, Arabidopsis thaliana
MKLKLSSISMNHKNVLLIKLISAILISFFAFRLFILHSSEFSPIFASVTGKFEARFLPPEIIVPENEDLIPQDIEVEKCDLFAGKWIPDSVGPIYTNKSCGSLIDGHQNCITNGRPDLDFLYWKWKPHDCLLPRFDPRRFLQLMRHKSWAFIGDSISRNHVESLLCMLSTIEEPVEVYHDMEYKSKRWHFPLHNLTVSNIWSPFLVQAAIFEDSNGVSTASVQLHLDRLDETWTSLMPSFDYAIISTGKWFLKSAIYHENAKLVGCHNCQEKPHIEELGFDYAYNASLHNVMDFLAAEDNSKGTVFFRTSTPDHFQNGEWHSGGTCKQTEPVSDEEIEIKDVHKILKDIEIDQFKRAVREKTNQDGGNLKLLDFTRMLLTRPDGHPGEYRQFRPFDKDKNAKVQNDCLHWCLPGPFDYLNDVILETIVNG

SEQ ID NO: 49 - DNA, 1371 bp, Arabidopsis thaliana
atggattcattgagattaatatctaagagcatcaagattgaaggaaccccatttggttcctctcaccaacgcaatcaaattttcctcaaatccgtcgctttcttccttttgatcggtctcgcttaccgttttctcatcactaattccaccgtctctccggttcccaccgttcgatcctcgccggaatctctcccgccggatccttcaggtctcactgccatcactcaaacttcagcctctgtcgattccccagcaaacatcacaaccattgcttcacagaatgtttcaaccaagtgtgatatctttatcgggaattgggtaccagacccatcagggccaatctacaccaatgtctcttgccgtcatatccaagattaccaaaattgcttgaaaaacggaaggccagatgtgaattaccttcgatggagatggcaacctcgcgattgcgatctcccgaggtttaaccctgagcaattccttgacaatatgaggaacaaatggttggcttttatcggtgattccatctctcgtaaccatgtccaatccctcctctgcattctctctcaggtagaagaagtagaagatatcttccatgacaaggaatacaaatcaaggatctggagattcccttcttacaacttcacactctctgtcatttggtctcctttccttgtaaaagctgaaacctttgagaacggagttcctttctcagacattcgagtacacctcgacaagctagaccagaaatggactgatcagtacatcaatttcgactacgttgtcatctccggtggaaaatggttcctcaaaacaacaatcttccatgaaaacaacaccgtaaccggatgccattactgccaagggaagaacaatatgaccgaactcggttatctatactcgtaccgaaaggttcttcatttggttttagactttgtcgctgaaccgaaccataaagctcaggttctcttcagaacaacaacacctgatcatttcgagaatggagagtgggactctggtggtttctgtaaccgaacaatgccgtttacggaaggcagcgaaggagagatgaagagcgaggatgtatcgatgcgtgatatcgagcttgaagagttctataagactactactacacaacaagaaggttccaattcaaacattgtattacttgacacaacttcaatgtcgcttctccgaccagatggacatccgggaccgtaccggtatccaaatcctttcgctgggctgaagaacaaggagctgaatcaggttcagaacgattgtcttcattggtgcttacccggtccgattgattcatggaatgatctaatggtcgaggtgatgcttaaccgggaacggcaaagacgagaatag

SEQ ID NO: 50 - protein, 456 aa, Arabidopsis thaliana
MDSLRLISKSIKIEGTPFGSSHQRNQIFLKSVAFFLLIGLAYRFLITNSTVSPVPTVRSSPESLPPDPSGLTAITQTSASVDSPANITTIASQNVSTKCDIFIGNWVPDPSGPIYTNVSCRHIQDYQNCLKNGRPDVNYLRWRWQPRDCDLPRFNPEQFLDNMRNKWLAFIGDSISRNHVQSLLCILSQVEEVEDIFHDKEYKSRIWRFPSYNFTLSVIWSPFLVKAETFENGVPFSDIRVHLDKLDQKWTDQYINFDYVVISGGKWFLKTTIFHENNTVTGCHYCQGKNNMTELGYLYSYRKVLHLVLDFVAEPNHKAQVLFRTTTPDHFENGEWDSGGFCNRTMPFTEGSEGEMKSEDVSMRDIELEEFYKTTTTQQEGSNSNIVLLDTTSMSLLRPDGHPGPYRYPNPFAGLKNKELNQVQNDCLHWCLPGPIDSWNDLMVEVMLNRERQRRE

SEQ ID NO: 51 - DNA, 1329 bp, Arabidopsis thaliana
atggaacaacaattaaccttagtattgcttgatagcccaaaaggcgccaaatatgtagaaacctttgaagaagccgcttcttcttcttcttcttcttcttcttcttctgttccctcctgcactgatcaccgccatcgaaccttcgtcaaattcttcctttacttctctctcgtcgctcttgcttactatttcatcatctccagtctcgccgtctctccaattcccccaacattgccgcaatcttctcccggaaacgtttcatcagcaaagtgtgatcttttcaccggagattggataccagatccaacaggtcctctgtacacaaatgtcacttgtcgtcacattcaagattttcagaactgcctattgaatggacgaccagatgtgaattatctcttctggagatggaagcctcgtgattgtgatcttcctaggtttagtccatcgcagtttcttgcttcagtgaagaacaaatggtgggcttttatcggtgattccattgctcgtaatcatgtccagtctctcatctgcattctctctcaggtggaagaagtggaggaaatctatcacgataaggagttcagatccaagatatggagattcccttctcacaacttcacactatcagtcatttggtctcctttccttctcaaatccgaaacatctagcaactcggatattcagctttacctcgaccagcttgaccacaaatggactgtccaatacccgaaattcgactacgttgttatctctggaggcaaatggtttcttaaaacaacaattttccatgaaaacaacgtagtcacgggctgtcattactgccaaggaagaaacaacctaactgatctcggctatgattactcctaccgcaaaaccctaaaccttctccgtgacttcgtcctaaactcaacccacaaaccgctggttctgtttcgaacaacaacgcctgaccatttcgaaaacggagagtggaacactggtgggtattgcaacagaacgatgccgtttaaagaaggccaagcaaatatgaaaactgtagatgatgtgatgcgtgatgttgagcttgaggtgtttcagaaatttgggaaaggttttggcttaggttccaacatcaggctattagacacgactggaatgtctcttctccgtccagacgggcatccgggaccataccggcatccaaatccttttgctggagttaagaataagagcaatgttcagaatgattgtctgcattggtgcttacctggtccaattgattcatggaatgatgtgatggtggaaaccacgcttaaccgggaacgggaactatacgatttaaccggttaa

SEQ ID NO: 52 - protein, 442 aa, Arabidopsis thaliana
MEQQLTLVLLDSPKGAKYVETFEEAASSSSSSSSSSVPSCTDHRHRTFVKFFLYFSLVALAYYFIISSLAVSPIPPTLPQSSPGNVSSAKCDLFTGDWIPDPTGPLYTNVTCRHIQDFQNCLLNGRPDVNYLFWRWKPRDCDLPRFSPSQFLASVKNKWWAFIGDSIARNHVQSLICILSQVEEVEEIYHDKEFRSKIWRFPSHNFTLSVIWSPFLLKSETSSNSDIQLYLDQLDHKWTVQYPKFDYVVISGGKWFLKTTIFHENNVVTGCHYCQGRNNLTDLGYDYSYRKTLNLLRDFVLNSTHKPLVLFRTTTPDHFENGEWNTGGYCNRTMPFKEGQANMKTVDDVMRDVELEVFQKFGKGFGLGSNIRLLDTTGMSLLRPDGHPGPYRHPNPFAGVKNKSNVQNDCLHWCLPGPIDSWNDVMVETTLNRERELYDLTG

SEQ ID NO: 53 - DNA, 1251 bp, Arabidopsis thaliana
atgggattaaacgagcaacaaaatgttccaagtcagaggaagatcatagtttttatagtcttagcttttataccaatagctctgtttcgactctgttttaataatccattctcctccattaaagacacctctcttcaagactctgcagctaacgttgtgatcactagcttctcttcatcttctcaagaagaagaaagccaagagagttttgatcatatacaagatgaacctttatgcgattatacgcaagggaactgggtcagagacgagattggtccactctacaatggttcaacatgtggaacaatcaaggatggtcagaattgtttccgccatggtagacctgattccggttatctttactggaaatggaaaccaaacgaatgcgatataccgagattcgactcgaaccggtttcttgatcttatgagagataagcatctagcttttatcggtgattccatggctagaaatcagcttgagtctcttttatgcttgctctctaccgtctctagccctgatctcgtctatagaaacggagaagacaataaattcagaagatggcgtttcgagtcacacaacgtcactgtctcggtttactggtccccgtttttggtggccggtttagagaaatcgggaaacttggaccacaatgtattgcacatagaccgcgtggatgagagatggggcaatgatttagaacgttttgatacggtcgtagtctctgtgggacattggtttctgcatccagcggtttactacgagtctggttcggttttgggatgtcattcttgtgaaacaagtaactgtaccgaggtagggttttatgatgtatttagaaaagcgataagaacaacgttgagagcggttgctggaagtggtcgtgaggtgatcttgacgacattttcgccatcacattttgaaggacggccttgggacagtctcggcgcatgtaacatgaccaagccgtacgaagggaaggttttagaaggtctggacttggacatgcgcaaaatagaaattgaggaatatacggcggctgcggctgaggtgaggttagaggtgttggacgtgacggctatgtcggtactgagaccagacggacatcctggtccgtacatgtacgcggacccgttcaagaacggtgtaccggagagaattcctaatgattgtttgcattggtgtctacctggtcctgtcgacacgtggaacgagatcatgatcgagatgttgcggcgatggaaggtttaa

SEQ ID NO: 54 - protein, 416 aa, Arabidopsis thaliana
MGLNEQQNVPSQRKIIVFIVLAFIPIALFRLCFNNPFSSIKDTSLQDSAANVVITSFSSSSQEEESQESFDHIQDEPLCDYTQGNWVRDEIGPLYNGSTCGTIKDGQNCFRHGRPDSGYLYWKWKPNECDIPRFDSNRFLDLMRDKHLAFIGDSMARNQLESLLCLLSTVSSPDLVYRNGEDNKFRRWRFESHNVTVSVYWSPFLVAGLEKSGNLDHNVLHIDRVDERWGNDLERFDTVVVSVGHWFLHPAVYYESGSVLGCHSCETSNCTEVGFYDVFRKAIRTTLRAVAGSGREVILTTFSPSHFEGRPWDSLGACNMTKPYEGKVLEGLDLDMRKIEIEEYTAAAAEVRLEVLDVTAMSVLRPDGHPGPYMYADPFKNGVPERIPNDCLHWCLPGPVDTWNEIMIEMLRRWKV

SEQ ID NO: 55 - DNA, 1275 bp, Arabidopsis thaliana
atgcaaccttcaagaacaagagtttcattttttgaaacaggagagaccatgaagcaaagaaagaagagttatctctcaatcttcgtgatcttcttctctcttttcttcttcgggatcttcatgtacaatgacaacctcaaatcctccattgctgacttcacatcttcgaacccattctctagttcatttgtggagttgccgccggatgagtgtgatctgttcaccggtcaatgggttttcgacaacaagacatatccattgtataaagaagaagagtgtgagttcttgacggagcaagtgacttgtttaagaaacggaaggaaagattctttgtttcagaattggagatggcaacctagagactgttctttaccaaaattcaatgcaagagtgttgttagagaagctgaggaacaagagattgatgtttgtcggtgactcgttaaaccggaaccaatgggaatcaatggtttgtttggttcaatcagtgattcctcccggtagaaaaagcttaaaccagaccggttcactcactgttttcaaaatccaggactataacgcgacggtggagttttattgggcgccatttttggtggaatcaaattcagacgatccggaaaagcacagtataatcgaccgtataataatgccagagtccatcgagaagcatggagtcaactggataggcgttgactttcttgtcttcaatagttacatctggtggatgaatactgtctccatcaaagtcctacgtggatcgttcgatgatggggacacagagtatgatgagatcaaacggccaatagcgtacgagagggtgttgaggacattgggagattgggtggaccataacattgatcctctaagtacaactgttttcttcatgagcatgtctcctcttcacatcaagagctcagattgggctaatcctgagggtataaggtgtgctttagagacaacaccgatcttgaacatgtctttcaacgtggcttacgggcagttttccgcggtggggacggattacaggctgtttccggtggcggaaaacgttacacagtctctaaaagttcctattcatttcctcaacattactgcattgtctgagtatcgtaaagatgcacatacttcggtttacacgatcaagcaaggcaagttgctgacgcgggagcagcaaaacgatccagccaattttgctgattgcatacactggtgcttaccaggccttcctgatacatggaatgagtttctctatacacatattatttctcgaagatga

SEQ ID NO: 56 - protein, 424 aa, Arabidopsis thaliana
MQPSRTRVSFFETGETMKQRKKSYLSIFVIFFSLFFFGIFMYNDNLKSSIADFTSSNPFSSSFVELPPDECDLFTGQWVFDNKTYPLYKEEECEFLTEQVTCLRNGRKDSLFQNWRWQPRDCSLPKFNARVLLEKLRNKRLMFVGDSLNRNQWESMVCLVQSVIPPGRKSLNQTGSLTVFKIQDYNATVEFYWAPFLVESNSDDPEKHSIIDRIIMPESIEKHGVNWIGVDFLVFNSYIWWMNTVSIKVLRGSFDDGDTEYDEIKRPIAYERVLRTLGDWVDHNIDPLSTTVFFMSMSPLHIKSSDWANPEGIRCALETTPILNMSFNVAYGQFSAVGTDYRLFPVAENVTQSLKVPIHFLNITALSEYRKDAHTSVYTIKQGKLLTREQQNDPANFADCIHWCLPGLPDTWNEFLYTHIISRR

SEQ ID NO: 57 - DNA, 1464 bp, Arabidopsis thaliana
atgcaaccttggagaagaaaatttccattgttcgaaacaggagttacaatgaaacagaggaagaacagtaatctctcaatcttcgtcgtagtcttctctgttttcctcttcgggatcttcatgtacaatgaagacgtcaagtccatcgcagagtttcctttctccacctcaaaacctcacgacgtccacgacgaagcgacaccgatcacagagatcacaacactaccggttcaagagtcgatcaagaactcagacccaattcaggaatcgatcaaaaacgcagactcagttcaagattcagttaaagacgtagcagaaccagttcaagaggaggtatcgaaaacagaggaagttaaaaagattgagcttttcgctgcgacggaggatgaagaagacgtggaattgccgccggaggaatgcgatttgttcaccggagaatgggtttttgataacgagacgcatccattgtataaagaggatcaatgtgagttcttgacggcgcaagtcacttgcatgagaaatggaagaagagattctctgtatcagaactggagatggcaacctagagattgttctttaccaaagttcaaagcgaaattgctgttggagaagctaaggaacaagagaatgatgttcgttggagattccctaaaccggaaccaatgggaatcaatggtttgtttggttcaatcagtggttcctcccggtcgaaaaagcttgaacaaaaccggttctctctccgtcttccgagtcgaggattataatgcgacggtggagttttattgggcaccatttttggtggaatcgaattcagatgatccaaatatgcatagtatacttaaccgtataataatgcctgagtccattgaaaagcatggagtcaactggaaaggcgttgactttcttgttttcaacacttacatttggtggatgaacacattcgctatgaaagtcctacgaggatcgttcgataaaggagacaccgagtatgaggagatcgaacggccagtagcgtataggagagtgatgaggacttggggagattgggtggaacgaaatattgatcctctacgtaccactgtcttctttgctagcatgtctcctcttcacatcaagagcttggattgggagaatccagatgggatcaagtgtgcattagagacgacaccaatcctaaacatgtcaatgccattcagcgtaggaacagactacagactgttttcagtagcggaaaacgtcacgcattctcttaatgttccggtttactttctcaacattaccaaactctccgaataccggaaagatgctcacacttcggttcacacaatccgacaaggcaaaatgctgacgccggagcaacaagccgatcccaacacttacgccgattgtatccattggtgtcttcccggtttacctgacacgtggaatgagttcctttacacacgtatcatttcccgttcgtga

SEQ ID NO: 58 - protein, 487 aa, Arabidopsis thaliana
MQPWRRKFPLFETGVTMKQRKNSNLSIFVVVFSVFLFGIFMYNEDVKSIAEFPFSTSKPHDVHDEATPITEITTLPVQESIKNSDPIQESIKNADSVQDSVKDVAEPVQEEVSKTEEVKKIELFAATEDEEDVELPPEECDLFTGEWVFDNETHPLYKEDQCEFLTAQVTCMRNGRRDSLYQNWRWQPRDCSLPKFKAKLLLEKLRNKRMMFVGDSLNRNQWESMVCLVQSVVPPGRKSLNKTGSLSVFRVEDYNATVEFYWAPFLVESNSDDPNMHSILNRIIMPESIEKHGVNWKGVDFLVFNTYIWWMNTFAMKVLRGSFDKGDTEYEEIERPVAYRRVMRTWGDWVERNIDPLRTTVFFASMSPLHIKSLDWENPDGIKCALETTPILNMSMPFSVGTDYRLFSVAENVTHSLNVPVYFLNITKLSEYRKDAHTSVHTIRQGKMLTPEQQADPNTYADCIHWCLPGLPDTWNEFLYTRIISRS

SEQ ID NO: 59 - DNA, 1284 bp, Arabidopsis thaliana
atgaagcaaactgacggcagagaaaggaaagcttacctttctctgctatactttgcagtcattcttcttcccgtattccttctcggatgctatctgtacaacgagaagcagctgagggttggccagtttcaggaattcaatacacataacctgcaagaacacatcactccactacaacaaagcaaagaagataaagacaagaagactgatcttgttccattagaattctgtgatgttttcacaggaaaatgggttcttgataacgtcacacatcctttatacaaagaagatgaatgtgagtttctatcagagtgggtggcttgtacaagaaacgggaggccagactctaagtaccagaaatggagatggcaacctcaagattgctctttgccaaggttcgacagtaagctgctgctagagaaactcaggggaaagaaactaatgttcattggtgattcaatacattacaatcagtggcaatccatggtttgtatggttcaatccgtgattccctcaggcaaaaagaccttaaaacacacagcacaaatgtccatcttcaacatagaggagtacaatgcaaccatatcattttactgggcacctttcctagtcgaatcaaatgctgatcctccagacaagagagacgggaagaccgatccagtaatcattcctaattcaatctcgaaacacggcgagaactggaaagacgcagactacctcatattcaacacctacatatggtggactagacattccacgatcaaagttctaaaacaagaatctttcaacaaaggagactcaaaagagtacaatgaaatcggaatctatatagtatacaaacaagtgttatcaacatggacaaaatggctagaacaaaacatcaacccaagtcaaacatctatcttcttcagcagcatgtcaccaactcatatcagaagttcagattggggattcaatgaaggaagcaaatgtgaaaaagagacagaaccaatactaaacatgtcaaaaccaataaatgttggaacaaatcgaagactttacgaaatcgcactgaatgcgaccaaatctacaaaagtgccgattcatttcctcaacataacgactatgtcagagtatagaaaagatgggcatacctcattttacggttcgataaatgggaaactcatgacgccggagcagaagttagatccgagaacttttgctgattgttatcattggtgtcttccgggattgcctgattcatggaacgagttgctctctctgtatatcatctacaaaatttaa

SEQ ID NO: 60 - protein, 427 aa, Arabidopsis thaliana
MKQTDGRERKAYLSLLYFAVILLPVFLLGCYLYNEKQLRVGQFQEFNTHNLQEHITPLQQSKEDKDKKTDLVPLEFCDVFTGKWVLDNVTHPLYKEDECEFLSEWVACTRNGRPDSKYQKWRWQPQDCSLPRFDSKLLLEKLRGKKLMFIGDSIHYNQWQSMVCMVQSVIPSGKKTLKHTAQMSIFNIEEYNATISFYWAPFLVESNADPPDKRDGKTDPVIIPNSISKHGENWKDADYLIFNTYIWWTRHSTIKVLKQESFNKGDSKEYNEIGIYIVYKQVLSTWTKWLEQNINPSQTSIFFSSMSPTHIRSSDWGFNEGSKCEKETEPILNMSKPINVGTNRRLYEIALNATKSTKVPIHFLNITTMSEYRKDGHTSFYGSINGKLMTPEQKLDPRTFADCYHWCLPGLPDSWNELLSLYIIYKI

SEQ ID NO: 61 - DNA, 1242 bp, Arabidopsis thaliana
atgtctatacaaacaactgcagattcgaggatgattcaatccatcttccaagttgtgcttgttagtcttctagttctaggatccgtgagatggatacttgatgaactcaagagcaaagaaagtcgaatatccaaactatatggtttcagacagaaagaggcagtgtttgtcactaaagaggatcagcttgatgagagctgcaatgtctttgaaggacaatgggtttgggacaatgtctcttatcctctttatacagagaagagctgtccttatttagtcaaacaaacaacatgtcaacgaaatggacggcctgattcttattaccagaactggagatggaaaccgagctcatgcgacttgccaaggttcaatgctctgaaactgttggacgtgttgagaaacaaaagactaatgttcataggagactcagtgcagagaagcacgttcgagtcaatggtctgtatggtgcaatcagtaattcctgagaagaagaagtccttccataggattcctcctatgaagatcttcaaagctgaggaatacaatgcatccatcgagtattactgggcacctttcatcgtggaatcgatttcagatcatgcaactaatcatacagtacacaaaaggttggttaagctcgacgccattgaaaaacatagcaagagctgggaaggggttgatgttctagtctttgagagctatgtatggtggatgcatcaacctaagatcaatgcaacgtatggagataccagtgaagtccgagagtataatgtgacgactgcttataaaatggcgttagaaacatgggccaagtggtttaagacgaagatcaattccgagaagcaaaaagttttcttcacgagtatgtctccgactcatctatggagctgggagtggaatccagggagcgatggaacctgttatgatgagttatatccaatagacaaaagatcatattggggtacaggatcaaatcaagaaatcatgaagattgttggtgatgttctaagcagagttggagaaaatgtcacgttcctgaacattacgcaactgtcggaatacaggaaagatggacatacgactgtgtatggagaacggagagggaagctcttgacaaaggaacagagagctgatccgaaaaactatggagattgcatccactggtgtttgccaggagttcctgatacatggaatgagattctctatgcatatttgcttcgcagtcaccgcaatttcttctga

SEQ ID NO: 62 - protein, 413 aa, Arabidopsis thaliana
MSIQTTADSRMIQSIFQVVLVSLLVLGSVRWILDELKSKESRISKLYGFRQKEAVFVTKEDQLDESCNVFEGQWVWDNVSYPLYTEKSCPYLVKQTTCQRNGRPDSYYQNWRWKPSSCDLPRFNALKLLDVLRNKRLMFIGDSVQRSTFESMVCMVQSVIPEKKKSFHRIPPMKIFKAEEYNASIEYYWAPFIVESISDHATNHTVHKRLVKLDAIEKHSKSWEGVDVLVFESYVWWMHQPKINATYGDTSEVREYNVTTAYKMALETWAKWFKTKINSEKQKVFFTSMSPTHLWSWEWNPGSDGTCYDELYPIDKRSYWGTGSNQEIMKIVGDVLSRVGENVTFLNITQLSEYRKDGHTTVYGERRGKLLTKEQRADPKNYGDCIHWCLPGVPDTWNEILYAYLLRSHRNFF

SEQ ID NO: 63 - DNA, 1356 bp, Arabidopsis thaliana
atgacaacggcgttgttttcaccgtctaaccgtagaaaacgccgtctcacacacttcttcttcaccgtactcgctttcatcttactcgccgccttcatctacggccacgatttcatctctttctctcgtcgatctcttcactctccgacgatcgtccaccaatccgcaatcgtcgttgtagttgacgaacctcctccgcctccaccaacatctcctcctccgccttctcctccaccaccgtctcctccaccaccatctcctccaccgccatctcctccgccacctgcttttgcagtaggtaaaacgccggaaggatgcgatgtcttcaaagggaattgggtcaaagactggtcaacgcgtccactttacagagaatccgagtgtccgtacattcagccgcagctcacttgccgcacgcatggccgtcccgatagtgattatcagtcatggagatggcgacctgattcctgctcacttccttcgtttaatgcgacggtgatgttggagagcttaagagggaagaagatgatgttcgttggagattcgttgaacagaggaatgtatgtttctctcatctgtcttcttcattcacaaatcccagagaactccaaatccatggatactttcggatcactcaccgtcttctctctcaaggattacaatgcaacgattgagttctattgggcaccgtttcttctagaatcgaattcagataacgcaacagttcatagagtatcagatcgaattgtgaggaaaggatcgatcaacaaacacggtaggcattggcgaggggctgatatagttgtgttcaacacatacttgtggtggaggactggcttcaaaatgaagatcttggaaggttctttcaaggatgagaagaagagaatcgttgagatggagagtgaagatgcttatagaatggcgttgaagactatggttaaatgggtgaagaagaatatggatccattgaagacgagggttttctttgctaccatgtctcctactcattacaagggcgaggattggggaggcgaacaaggaaaaaattgttacaatcaaacgacaccgatccaagacatgaaccattggccatcagactgcagcaaaaccttaatgaaagtgataggagaggaattagaccaaagagcagagtttccggtgacggtacttaacattacacaactctctggctatcggaaagacgcgcacacgtcaatctacaagaagcaatggagcccattgacgaaggagcagctagcaaatccggctagctactcagactgtatacattggtgcttgcctggtcttcaagatacttggaacgagcttttctttgccaagttattttatccatga

SEQ ID NO: 64 - protein, 451 aa, Arabidopsis thaliana
MTTALFSPSNRRKRRLTHFFFTVLAFILLAAFIYGHDFISFSRRSLHSPTIVHQSAIVVVVDEPPPPPPTSPPPPSPPPPSPPPPSPPPPSPPPPAFAVGKTPEGCDVFKGNWVKDWSTRPLYRESECPYIQPQLTCRTHGRPDSDYQSWRWRPDSCSLPSFNATVMLESLRGKKMMFVGDSLNRGMYVSLICLLHSQIPENSKSMDTFGSLTVFSLKDYNATIEFYWAPFLLESNSDNATVHRVSDRIVRKGSINKHGRHWRGADIVVFNTYLWWRTGFKMKILEGSFKDEKKRIVEMESEDAYRMALKTMVKWVKKNMDPLKTRVFFATMSPTHYKGEDWGGEQGKNCYNQTTPIQDMNHWPSDCSKTLMKVIGEELDQRAEFPVTVLNITQLSGYRKDAHTSIYKKQWSPLTKEQLANPASYSDCIHWCLPGLQDTWNELFFAKLFYP

SEQ ID NO: 65 - DNA, 1278 bp, Arabidopsis thaliana
atgaaaccttcatcaccaatttcactcactagttcttcaattgctagaaaagctcgtttttctccttacctcttcacactcttagccttcattcttttcgtctctgtcctctatggtgaagattttatgtgcatctttggacagctcgagcctaacttcgtcctcccgccttctcgaacacccgaaaagaataagaaatcggagaagcttgcgtttgctattggtaaaacagaggaaagctgtgacgttttcagcgggaaatgggtgagagatgaagtctcccgaccactgtatgaggaatgggagtgtccgtacattcaacctcagcttacatgtcaagaacacggccgtcccgacaaagactaccagttttggcggtggcagcctaatcactgcgatcttccctcgttcaacgcgtcactgatgctggagacattaagagggaagaggatgatgtatgtaggagactcgttaaaccgtggaatgtttgtatcaatgatttgtcttcttcatcgtcttattcctgaagatcagaaatcaattaaaaccaatggttccctcactgtcttcactgccaaggagtataacgcgacgatagagttctattgggcaccgtttcttctagaatcgaattcagacgatgcaattgtccatagaatatcggatagggttgtgagaaaaggatcaattaacaaacatggtcgtcactggaaaggagtcgatataatcatcttcaacacttatctgtggtggatgactggattgaagatgaacatcttgcaaggatcgtttgatgataaagagaagaatatcgtagaggtttcgactgaagatgcgtaccggatgggtatgaagagtatgttgagatgggtgaagaacaacatggatcgtaagaaaactagggtcttcttcactagcatgtctccaacgcatgccaaggggatagattggggaggtgaaccgggtcagaattgctataatcagacaacacttatagaagatccaagctactggggatcagattgtaggaaaagcataatgaaagtgattggagaagtgtttggaagatcgaaaacaccgataacgttactgaacataacgcaaatgtcgaattacaggaaagatgcacatacgtcgatatacaagaagcagtggagtccacttacagcggagcagctggagaatccaacgagctatgcggattgtgtacattggtgcttgcctggccttcaagatacttggaatgagcttctctttgctaaacttttctatacttga

SEQ ID NO: 66 - protein, 425 aa, Arabidopsis thaliana
MKPSSPISLTSSSIARKARFSPYLFTLLAFILFVSVLYGEDFMCIFGQLEPNFVLPPSRTPEKNKKSEKLAFAIGKTEESCDVFSGKWVRDEVSRPLYEEWECPYIQPQLTCQEHGRPDKDYQFWRWQPNHCDLPSFNASLMLETLRGKRMMYVGDSLNRGMFVSMICLLHRLIPEDQKSIKTNGSLTVFTAKEYNATIEFYWAPFLLESNSDDAIVHRISDRVVRKGSINKHGRHWKGVDIIIFNTYLWWMTGLKMNILQGSFDDKEKNIVEVSTEDAYRMGMKSMLRWVKNNMDRKKTRVFFTSMSPTHAKGIDWGGEPGQNCYNQTTLIEDPSYWGSDCRKSIMKVIGEVFGRSKTPITLLNITQMSNYRKDAHTSIYKKQWSPLTAEQLENPTSYADCVHWCLPGLQDTWNELLFAKLFYT

SEQ ID NO: 67 - DNA, 1233 bp, Arabidopsis thaliana
atggcaaagcgacaactgttgatgctgggtattagaactagctttcatactatcgccgccgtgcttgtggccggattaatattcactgccgtgtttttgtccagaaacagtttgccaaaggaaaatcctcaaagccacggtgttacggatcgcggaggggacagtggcagagaatgtaatttgttcgaagggaaatgggtatttgataacgtatcataccctctttataaagaagaagattgcaagttcatgtctgaccagttggcttgtgagaagtttgggaggaaagatctgagttacaaattctggaggtggcaacctcacacgtgtgatcttcccaggtttaatggaacgaagttgctggagaggctaagaaataagaggatggtgtacgtaggagactctctgaacagaggccaatgggtatcaatggtgtgtatggttagttcggtaattacaaacccaaaggcgatgtatatgcacaacaatggctctaacctcatcacgttcaaagctctcgaatacaatgctacaatagactactattgggctcctttgctggtggaatccaactcggacgacccgacaaaccatagattccccgatcggatagttcggatccaatcaatagaaaaacatgcaaggcattggacgaactctgatatcattgttttcaactcttacttatggtggagaatgcctcatatcaagtcactgtggggatcgtttgagaaattagatggaatatacaaggaagtggagatggtgagagtttatgaaatggctttgcaaacactgtctcagtggttggaggttcacgttaaccccaatattaccaaacttttcttcatgtccatgtctcccacccatgaaagggctgaagaatggggaggaatacttaatcagaactgttatggagaagctagtctaatagataaagaaggatatactggaagagggtcagatccaaagatgatgagagtcttagaaaatgtgcttgacgggctaaaaaatcgaggattaaacatgcagatgataaacattacgcaactatcagagtatcgaaaagaaggtcatccttcgatctacagaaaacaatggggaacagtgaaggaaaatgaaatctcgaatccaagtagtaacgcagattgtatacattggtgtttgcctggagttcctgatgtttggaatgaactcctttatgcttatattcttgaccatcattctagttga

SEQ ID NO: 68 - protein, 410 aa, Arabidopsis thaliana
MAKRQLLMLGIRTSFHTIAAVLVAGLIFTAVFLSRNSLPKENPQSHGVTDRGGDSGRECNLFEGKWVFDNVSYPLYKEEDCKFMSDQLACEKFGRKDLSYKFWRWQPHTCDLPRFNGTKLLERLRNKRMVYVGDSLNRGQWVSMVCMVSSVITNPKAMYMHNNGSNLITFKALEYNATIDYYWAPLLVESNSDDPTNHRFPDRIVRIQSIEKHARHWTNSDIIVFNSYLWWRMPHIKSLWGSFEKLDGIYKEVEMVRVYEMALQTLSQWLEVHVNPNITKLFFMSMSPTHERAEEWGGILNQNCYGEASLIDKEGYTGRGSDPKMMRVLENVLDGLKNRGLNMQMINITQLSEYRKEGHPSIYRKQWGTVKENEISNPSSNADCIHWCLPGVPDVWNELLYAYILDHHSS

SEQ ID NO: 69 - DNA, 1648 bp, Arabidopsis thaliana
aaagaaacaaaaggatttatttataaaaaaaaaaaagagatgaagacagctcaagggatgcaatagagccaagaggaggagaaagatgggatctgctccaaacttgtaccagtcgcatcaaagcaagttttggtgaagaagaagagacgaggtcttcctcttactgtggtggtgacattttctctaaaaagaaagtctccgatatgtcgcagagatggagtagaaagaagagtagacttccattagcgggtctcctctttattctcgttgtcacctttatgattctcttcaacgagcgtagcattcagcagattcatcaccacgccgcgagtcacactcaaaatctccgagaaccttccacgttcgatttcgtcaagcctaatgttcctcggattaactacttgggagctcatgaggttttgggtagattcagcaaatgcaactcgacgaaagagtacagtgggaagaaaatcggatgcgttgacccgtttgaagaccacccgggtcaagtaacaaaggaggagcagaaatgtgatgtcttttctgggaaatgggtctttgataattcatcatcataccctttacacaaggaatctcagtgtccttacatgtccgaccagttggcttgtcagaagcatggtaggaaggatttggagtatcagcattggagatggcaacctcatgcctgcaacttgaagagatggaatgtgatagaaatgtgggagaagctgagaggaaagagattgatgtttgttggagactcgttaaacagaagccaatggatttcaatggtttgtctcttacggtctgtcattccacgtgacaagcagtctatgtctcctaacgctcacctcaccattttcagggctgaggactacaatgccacagtggagtttctctgggcaccgttgctcgtggagtcgaattctgatgaccctgttaatcacagattgagcgagcggattatccgacccgattctgttcttaaacatgcatcaaagtggcaacatgctgatattctaatcttcaacacctacttatggtggagacaagactctgtcaagctccgatggagcagtgaagaaaaagggtcatgcgaggaggtgaagagcgccgagggaatggagatggcaatggatagttggggtgattgggttgctaacaatgtcgatccaaacaaaaagcgagttttcttcgttacaatgtctcctacacatcaatggagccgagaatggaacccgggaagcgaaggaaactgctacggggagaagaaaccaatagaggaagagagttattggggaagtgggtcggacattccgacaatgaggatggtgaagagagttttggagagattgggaccaaaggtctcagttataaacatcactcagttgtctgagtatcgaaaagatggtcatccatcggtgtaccggaaattctgggaacctctaaatgaagaccggttgaaaaacccggcatcgtattctgactgtactcattggtgtgtacctggagttcctgatgtctggaatcaattgcttttccattttttgtgaaaatgaaatgaatataaccttaccttgatggccgaatatcacccataaattatataacggggtgatttcagctagctataaggatccaaatttgt

SEQ ID NO: 70 - protein, 449 aa, Arabidopsis thaliana
MSQRWSRKKSRLPLAGLLFILVVTFMILFNERSIQQIHHHAASHTQNLREPSTFDFVKPNVPRINYLGAHEVLDRFSKCNSTKEYSGKKIGWVDPFEDHPGQVTKEEQKCDVFSGKWVFDNSSSYPLHKESQCPYMSDQLACQKHGRKDLEYQHWRWQPHACNLKRWNAIEMWEKLRGKRLMFVGDSLNRGQWISMVCLLQSVIPRDKQSMSPNAHLTIFRAEDYNATVEFLWAPLLVESNSDDPVNHRLSERIIRPDSVLKHASKWQHADILIFNTYLWWRQDSVKLRWSSEEKGSCEEVKSAEGMEMAMDSWGDWVANNVDPNKKRVFFVTMSPTHQWSREWNPGSEGNCYGEKKPIEEESYWGSGSDIPTMRMVKRVLERLGPKVSVINITQLSEYRKDGHPSVYRKFWEPLNEDRLKNPASYSDCTHWCVPGVPDVWNQLLFHFL

SEQ ID NO: 71 - DNA, 1140 bp, Arabidopsis thaliana
atggctacctcagagactcgagtcttgttcttatctttgtgtctcatccttggcaaagtagttttgtctcagtttgatgagttatggctagtaggggacgatgatccactcaacgccttgcagacgaggcgtgaaagaagagaggagaggtgtgattactcggtggggaaatggacgttcgatgaaacgtatccgctctatgattcaagctgtccatatctgagttctgcactaagttgccaaagaaatggaagacccgactcttattaccagaaatggagatggatccctaaggcttgttcactaccaaggtttgacgcattgaaatttcttgggaaaatgagaggaaaaagaataatgctggttggagattcaatgatgagaaaccagtgggaatctcttgtctgcttagtacagtctgttcttcccactcatcgtaagaagctcacttacaatggtcctacgatgtctttccattctctggattttgagacatcaattgagttttgttgggctcctctgcttgtggaactcaagagaggagttgaccgtaaaagggtgttacatttggactcaatcgaagacaatgctagatattggcgtggtgtagatgttcttgtattcgattccgctcactggtggactcactctcagagatggagttcgtgggattattacatggatgggaataagatcttcaaagctatggacccaatggttgcttatgagagaggactaaccacatgggctaaatgggttgagataaatcttgatccatccaaaacaaaagtcattttccgcactgtctcaccgagagagagtggtcagatgtgctacaaccagaaacatcccttgccttctttatcttcttccaccaaaccccacgtgcctcaacagtcaagagtgttgaacaaagtgctaaggacaatgaaataccgagtgtatttatacgatatcacaacaatgtctgcgtatagaagagacggtcatccttcggtgttcaagagagcaatgcacgaggaagagaagcaccatcgtatcgctggaccttcatcagattgcagtcattggtgcttgcccggtgtcccggatatttggaatgagatgctttcttcaattatcttaaccaatgctgtatga

SEQ ID NO: 72 - protein, 379 aa, Arabidopsis thaliana
MATSETRVLFLSLCLILGKVVLSQFDELWLVGDDDPLNALQTRRERREERCDYSVGKWTFDETYPLYDSSCPYLSSALSCQRNGRPDSYYQKWRWIPKACSLPRFDALKFLGKMRGKRIMLVGDSMMRNQWESLVCLVQSVLPTHRKKLTYNGPTMSFHSLDFETSIEFCWAPLLVELKRGVDRKRVLHLDSIEDNARYWRGVDVLVFDSAHWWTHSQRWSSWDYYMDGNKIFKAMDPMVAYERGLTTWAKWVEINLDPSKTKVIFRTVSPRESGQMCYNQKHPLPSLSSSTKPHVPQQSRVLNKVLRTMKYRVYLYDITTMSAYRRDGHPSVFKRAMHEEEKHHRIAGPSSDCSHWCLPGVPDIWNEMLSSIILTNAV

SEQ ID NO: 73 - DNA, 1158 bp, Arabidopsis thaliana
atgggtttcaaactcaactctcttttccttcttctttcgcttctcatactcacaatcttgtccggagccgaccaagccttggcttacgtcaagaaaccacatgtgagccaacgcaacaaaacggcgttagcagccgtcgccggaagaggaggaggaaaagagatgttgaaagggaggaagcagacgagtggttgtaacttgtttcaagggagatgggttttcgatgcttcttaccctttctacgattcatccacgtgccctttcatcgacggtgagttcgactgtctcaaattcggccgaccagacaaacagttccttaagtactcttggcagcctgattcatgcaccgtcccaaggtttgatggggaagcgtttttgaagaaatggagagggaaacgagtgatgttcgtgggtgactcactgagtctaaacatgtgggaatcgttggcatgtatgatacattcgtcggttccaaacactaagaccacttttctcaagcgaaccccactctcctctctcactttccaggaatatgacgtcacactattcctataccgaacaccatacctagtggacatctccaaagaaagtgtcgggcgtgtgcttaaccttggagccattgaagatggagctgatgcttggaaaaatatggacctcctcgtcttcaattcttggcactggtggactcacacaggagtacagtctcaagggtgggattttataagagatgggtcttcactgatgagagacatggaccgccttgatgctttcaacaaaggactcaccacttggggtcaatgggttgatcaaaatgttaatgtttcgcaaacccgagtcttcttccaaggcatttctcccactcactacatgggaagggaatggaacgagccgaggaaaacttgcaacgggcagatgcaaccgttgaccggatcaacatacccaggtggttcacttcctgcagcaagcattgtgtctcgagtattaagcacgatgagaacgcccgtttacttactcgacatcacaactctatctcaactgagaaaagatgctcatccatctacatatggaggcgatggcggaacggattgcagtcactggtgccttcctggcttgccggatacttggaaccagcttctctatgcagctctttcgatgtga

SEQ ID NO: 74 - protein, 385 aa, Arabidopsis thaliana
MGFKLNSLFLLLSLLILTILSGADQALAYVKKPHVSQRNKTALAAVAGRGGGKEMLKGRKQTSGCNLFQGRWVFDASYPFYDSSTCPFIDGEFDCLKFGRPDKQFLKYSWQPDSCTVPRFDGEAFLKKWRGKRVMFVGDSLSLNMWESLACMIHSSVPNTKTTFLKRTPLSSLTFQEYDVTLFLYRTPYLVDISKESVGRVLNLGAIEDGADAWKNMDLLVFNSWHWWTHTGVQSQGWDFIRDGSSLMRDMDRLDAFNKGLTTWGQWVDQNVNVSQTRVFFQGISPTHYMGREWNEPRKTCNGQMQPLTGSTYPGGSLPAASIVSRVLSTMRTPVYLLDITTLSQLRKDAHPSTYGGDGGTDCSHWCLPGLPDTWNQLLYAALSM

SEQ ID NO: 75 - DNA, 1143 bp, Arabidopsis thaliana
atgggtttcaaactcatctctctcctccttctttttcttccacttctaacagtcacaatcttgtcgggagtcgagcaggcctttgcttccgacaaggcactcctcgtgaccggccgcaacatcacggctgacggtggaaggtcgtcactgagagggaagaagcagaggagagcatcaggatgtaacttgttccaaggccgatgggtgttcgatgcttcttaccctttctatgactcgtccaagtgtcctttcatcgacggcgagtttgactgtctcaagttcggccgaccagacaaacagttcctcaagtactcttggcaacctgaatcttgcaccatcccaaggttcgacggtggggcatttctgaggaaatacagaggaaaacgagtcatgttcgtcggagactcactgagtttaaacatgtgggaatcgttggcctgtatgatccatgcgtcggtaccaaacgccaagaccacttttctcaagcgtaccccactctctactctcactttccaggaatatggagtaacgttatatctatatagaacaccatacatagtagatatatccaaagagagagttgggcgtgtgcttaaccttggagctattgaaggaggtgctgatgcttggaaaaacatggacgttcttgtcttcaactcttggcattggtggactcacaaaggccaatctcaagggtgggattatattagagatgggtcgtctttggtgagagacatgaaccgtcttgacgctttctacaaaggactcagtacttgggctcgatgggttgatcaaaacgtcgatacggccaaaactcgagttttcttccaaggcatttctcccactcattacgagggaagggaatggaacgagccgaggaagacttgtagtgggcaaatgcagccgttgggtggatcaagttacccaagtggtcagcctccatcctcaggagtagtgtcgaaagtgttgagttcgatgaaaaagccagtgaccttgctcgatatcacaactttgtctcagttgagaaaagatgctcatccttcttcttatggtggcgatggaggaacagattgcagccattggtgcctcccagggttgcctgatacatggaaccaacttctctacgcagctcttacgatgtga

SEQ ID NO: 76 - protein, 380 aa, Arabidopsis thaliana
MGFKLISLLLLFLPLLTVTILSGVEQAFASDKALLVTGRNITADGGRSSLRGKKQRRASGCNLFQGRWVFDASYPFYDSSKCPFIDGEFDCLKFGRPDKQFLKYSWQPESCTIPRFDGGAFLRKYRGKRVMFVGDSLSLNMWESLACMIHASVPNAKTTFLKRTPLSTLTFQEYGVTLYLYRTPYIVDISKERVGRVLNLGAIEGGADAWKNMDVLVFNSWHWWTHKGQSQGWDYIRDGSSLVRDMNRLDAFYKGLSTWARWVDQNVDTAKTRVFFQGISPTHYEGREWNEPRKTCSGQMQPLGGSSYPSGQPPSSGVVSKVLSSMKKPVTLLDITTLSQLRKDAHPSSYGGDGGTDCSHWCLPGLPDTWNQLLYAALTM

SEQ ID NO: 77 - DNA, 1104 bp, Arabidopsis thaliana
atgggtttcacttcccgaggaaacccttcttttctcttcttcttcttcttcttcctctgtctctccacagtctctgcttacatcaacagcacaagcagcaataatgatgaagtaagaagagagctagcttctgggagatgtaattggtttagagggaattgggtttatgatgtaaagtacccactttatgatccttataaatgtccattcatagatccacagtttaactgtaagaagtatggtcgtcctgacaatgcttaccttaagtatcgatggcaaccatcttcttgctctctccccagattcaatgggttgtatttcttgaggaggatgagagggaagaagataatgttcgttggagattcactaagcacaaacatgtggcaatctcttgcttgtctaattcactcttgggttcctaacactcgttacactcttattcgccaaaagggtcttgcttcacttacctttgaggaatacggagtgacgttgttgctatacagaacacagttcttggtagatttgaatgtggagaaagttgggagagtgcttaagcttgattccattaagcaagggaatatgtggagaggcatggacgttttgattttcaactcgtggcattggtggacacacaccgaacacattcagccgtgggattacatggaagatgggaataggttgtacaaagacatgaaccggctcgttgctttctacaaaggaatgaccacttgggctcgatgggttaatgcttacgtcgatccttctaagactaaagtcttcttcaacggcgtgtctcctactcattacgagggaaaggattggggagagccaatgaactcatgtaggagtcaaacgcagccattttacggaaggaagtatccaggagggacaccaatggcttgggtgattctaaacaaagtgatgaggagattgaagaaaccggtccattggctcgatataaccggtctgtctcagcttcgtaaagatgctcatccttctgctttcagtgggaaccatcctggtaatgactgtagccattggtgtctccctggtttgcctgatacttggaacttgctcttttactctactcttttctcttcttaa

SEQ ID NO: 78 - protein, 367 aa, Arabidopsis thaliana
MGFTSRGNPSFLFFFFFFLCLSTVSAYINSTSSNNDEVRRELASGRCNWFRGNWVYDVKYPLYDPYKCPFIDPQFNCKKYGRPDNAYLKYRWQPSSCSLPRFNGLYFLRRMRGKKIMFVGDSLSTNMWQSLACLIHSWVPNTRYTLIRQKGLASLTFEEYGVTLLLYRTQFLVDLNVEKVGRVLKLDSIKQGNMWRGMDVLIFNSWHWWTHTEHIQPWDYMEDGNRLYKDMNRLVAFYKGMTTWARWVNAYVDPSKTKVFFNGVSPTHYEGKDWGEPMNSCRSQTQPFYGRKYPGGTPMAWVILNKVMRRLKKPVHWLDITGLSQLRKDAHPSAFSGNHPGNDCSHWCLPGLPDTWNLLFYSTLFSS

SEQ ID NO: 79 - DNA, 651 bp, Arabidopsis thaliana
atgactgtaaaaccaaaagaaaaggattatggagtgacgataaatttgtatagaacacagtttctagtagatgtggttcaagaaaaggcaggacgagtgcttgtgcttgactccatcaaacaagccgatgcttggcttggcatggatgttcttatcttcaactcgtggcactggtggactcacacctccggacttcagccctgggattatatgagggaaggaaaccaattgtacaaagacatgaacaggcttgtggcttattacaaaggacttaacacatgggctcgttggattaacaataacattgtcccttcacgtactcaagtcttctttcaaggtgtttctcctgtccactatgacggaagagagtggaacgaaccattgaagtcgtgcaatggtcaaactcagccgttcatgggacaaagatatccaggaggattgcccttaggttgggttgtggttaacaaagtgttgagccgaattagaaaaccggtccatcttctcgatctcactactctctcggagtatcgcaaagacgcacatccaagtctctacaatggtatctcaaaggatctagactgtagccattggtgcctccctggactccctgatacttggaacttacttctctactcatctcttacttcctag

SEQ ID NO: 80 - protein, 216 aa, Arabidopsis thaliana
MTVKPKEKDYGVTINLYRTQFLVDVVQEKAGRVLVLDSIKQADAWLGMDVLIFNSWHWWTHTSGLQPWDYMREGNQLYKDMNRLVAYYKGLNTWARWINNNIVPSRTQVFFQGVSPVHYDGREWNEPLKSCNGQTQPFMGQRYPGGLPLGWVVVNKVLSRIRKPVHLLDLTTLSEYRKDAHPSLYNGISKDLDCSHWCLPGLPDTWNLLLYSSLTS

SEQ ID NO: 81 - DNA, 762 bp, Arabidopsis thaliana
atgtttgttggtgattctcttagtctaaatcaatggcaatcgttggcatgtatgcttcattcctctgttccaaattctacgtatactttaaccacacaaggtagtatatcaacctacacattcaaggagtatggactggaattgaagcttgataggaatgtttatctagtagatatagttagggagaagattgggagagttttgaagcttgattcgatcaatgatggaaaaaattgggtagagatggatacattgatttttaatacttggcattggtggagtcggagaggtcctgcacaaccatgggatttgattcaaatagggactaacgttacaaaagacatggaccgtgtggccgcgtttgaaattgcgcttgggacttgggggaaatgggtcgataccgtcttaaatactaagaaaactagagtcttttttcaaggaatttctccatctcattacaaaggagttctgtggggcgaaccagcggcaaagagttgcgtgggacagaaggaaccacttttggggacaaaatatccaggaggattgccagcagaagtgggagttttgaagagagcactcgggaaaatatcgaaaccggtgacattgctagacattacgatgctttcgttgcttcgcaaagatgctcatccttcggtttacggtctaggcgggcgaaactcctccggtgactgcagccactggtgtctctctggggtaccggatacttggaatgagattctttacaattatatggttgagtaa

SEQ ID NO: 82 - protein, 253 aa, Arabidopsis thaliana
MFVGDSLSLNQWQSLACMLHSSVPNSTYTLTTQGSISTYTFKEYGLELKLDRNVYLVDIVREKIGRVLKLDSINDGKNWVEMDTLIFNTWHWWSRRGPAQPWDLIQIGTNVTKDMDRVAAFEIALGTWGKWVDTVLNTKKTRVFFQGISPSHYKGVLWGEPAAKSCVGQKEPLLGTKYPGGLPAEVGVLKRALGKISKPVTLLDITMLSLLRKDAHPSVYGLGGRNSSGDCSHWCLSGVPDTWNEILYNYMVE

SEQ ID NO: 83 - DNA, 1080 bp, Arabidopsis thaliana
atgaatttccatcaggtcctctttctccttcttcttatttttcttgttgatttatcagattatggtgtcttagcagataagaccaatgatggttacaagaacgcaacaaagtgtaacatttaccaaggacgttggatttatgacaattcttctaatcctctttatggaacatcaacttgtccgttcattggattggattgccagaagtttggtcggccggacaagaactaccttcattaccgatggcaacccaccggatgtgacatcccaagattcaacggacgagatttcttaacaagattcaaagggaaaaaaatactgtttgtgggagattcactaagcaacaatatgtgggtgtcactaagttgcatgcttcacgcggccgttcctaacgctaagtataccttccaactcaacaagggcctctcaactttcaccattccagaatatggaatatctgtaaatttcctgaaaaatgggttcctggtggatttggtatcggacaaaacaagaggattgattctgaaattagattcgatcagtaggggaaatcaatggttgggatctgatgtcgccatctttaacacattccactggtggtctcacactggccgtgccaaaacatgggattattttcaaacgggagataaaatagtgaaagagatgaatagaatggaggctttcaagattgctttgacaacttggtctaaatggattgatcacaatatcgatccttcaaaaactagggttttctatcaaggcgtctctcccgttcacttaaatggtggtgaatggggtaaaccggggaagacttgcttgggtgagacggtaccagtgcaaggaccaagttaccccggacgacctaatgaaggtgaagctatagtgaaaagtgtgatcggaaggatggctaaaccagtggagctccttgatgtgacggctatgacggagatgaggaaggacggtcacccttccatctatgccggtggaggcgatcgcttaaacgactgcagccattggtgcctccctggagtcccagacgcttggaaccagctcctttatacagctcttctcagccactag

SEQ ID NO: 84 - protein, 359 aa, Arabidopsis thaliana
MNFHQVLFLLLLIFLVDLSDYGVLADKTNDGYKNATKCNIYQGRWIYDNSSNPLYGTSTCPFIGLDCQKFGRPDKNYLHYRWQPTGCDIPRFNGRDFLTRFKGKKILFVGDSLSNNMWVSLSCMLHAAVPNAKYTFQLNKGLSTFTIPEYGISVNFLKNGFLVDLVSDKTRGLILKLDSISRGNQWLGSDVAIFNTFHWWSHTGRAKTWDYFQTGDKIVKEMNRMEAFKIALTTWSKWIDHNIDPSKTRVFYQGVSPVHLNGGEWGKPGKTCLGETVPVQGPSYPGRPNEGEAIVKSVIGRMAKPVELLDVTAMTEMRKDGHPSIYAGGGDRLNDCSHWCLPGVPDAWNQLLYTALLSH

SEQ ID NO: 85 - DNA, 1107 bp, Arabidopsis thaliana
atgatgagaggtgctgctcccaccggagtagtatcggtcatggttttaatgatacttgtgttgttaaaacaaatagagagtgctagtgcgaatggttcatcattagggctaccacctagaaagttttgtaatatttatcaaggaagttgggtctatgacaaatcttaccctctctatgattccaagaattgccctttcatcgagagacagtttaactgcaagtccaatggtcgccctgacagcgagtacctcaagtatcgatggcaaccttccggttgcaatctccccagattcaacggcttagattttctgggaaggataatgaaagggaagaaactgatgttcgtaggagattcactgagtctaaaccaatggcaatctctcacgtgtttgctccacaacgccgcaccaaaagccaattccacatccacacgttccccttctggcctctccgtcttctcattccccgcttacaattcttcgattatgttttcaagaaacgcgtttcttgtggatatagtcggagccccaccgaagcgagtgatgaagcttgactcaatctcgagtggttcgttgtggaaaaccgcagacgttttggtcttcaactcttggcattggtggcttcacacagaccgaaaacaaccatgggacgcaattatgtccgggaatgtaacggttaaagacatggaccggttagtggcatatgagaaagcaatgatgacttgggctaagtggattgatcaaaatattgatccttccaaaaccaaagtcttcttccaaggtatttctccggaccatggccgggcaagggaatggtcgaaacaaggtggcaaaggcagttgcataggagaaacaaagccgattatgggatcgagttacctggctgggccgcacgcagccgagatggtggtggcgaaagtgataaagacgatgaagaatcaggcgcggttaatggatgtgacattgatgtctcagctaaggaaagatggacatccttcagtatacggttttggcggtcacagaatggcggattgtagccattggtgtctctctggagtcccggatagctggaaccagctcttgtattctgagctgtttcattcttaa

SEQ ID NO: 86 - protein, 368 aa, Arabidopsis thaliana
MMRGAAPTGVVSVMVLMILVLLKQIESASANGSSLGLPPRKFCNIYQGSWVYDKSYPLYDSKNCPFIERQFNCKSNGRPDSEYLKYRWQPSGCNLPRFNGLDFLGRIMKGKKLMFVGDSLSLNQWQSLTCLLHNAAPKANSTSTRSPSGLSVFSFPAYNSSIMFSRNAFLVDIVGAPPKRVMKLDSISSGSLWKTADVLVFNSWHWWLHTDRKQPWDAIMSGNVTVKDMDRLVAYEKAMMTWAKWIDQNIDPSKTKVFFQGISPDHGRAREWSKQGGKGSCIGETKPIMGSSYLAGPHAAEMVVAKVIKTMKNQARLMDVTLMSQLRKDGHPSVYGFGGHRMADCSHWCLSGVPDSWNQLLYSELFHS

SEQ ID NO: 87 - DNA, 1209 bp, Arabidopsis thaliana
atgggttctcttcttcctcttcttggcatctcagtagtctccgccattttctttctggttcttcaacaaccagaacaatcttcttcagccattatactgagcttgaagaaacgccatggaagctcctctggtagtagtggtaaccagtacagttcaagcagaccatcagctggtttccaagggaacaggagcacgtgctctctcttcctcggcacgtgggttcgtgataactcttatcctctctataaaccggcggattgtcccggcgtcgttgagcctgagttcgattgtcagatgtacggtcgtcctgactctgactacctcaagtatcgatggcaacctcagaattgcaatttacccacgttcaatggtgctcagtttctgttgaaaatgaagggcaaaaccataatgtttgcgggtgattcattggggaagaatcaatgggagtctttgatctgccttattgtttcatctgcaccgtccactcggacagaaatgaccagaggcttgcctctctccaccttcagattcttggattatgggataacaatgtcattttacaaagctccgttcttggtggacatagatgctgttcaaggcaagcgtgtgttgaagctggatgagatctctggtaatgccaatgcttggcatgacgctgatctcctcatcttcaacactggtcactggtggagccacaccggatctatgcaaggatgggacttgattcaatcaggcaattcttattaccaagacatggaccgttttgtggcaatggagaaagcacttcgtacttgggcgtattgggtcgaaactcacgttgatagatcccgaacacaagtcttgttcctctccatttctccaacacacgacaacccgagtgactgggcggcatcatcgtcttcaggatccaagaactgctacggagaaacagaaccgatcacaggaacagcttatccagtgagctcctacacagatcagctaagatcagtgattgttgaagtgcttcacgggatgcacaatccggcgtttcttctcgacataacactcctctcttccctaagaaaagacggtcatccgtcagtatacagcggcctcattagcggttcacaaaggtctagaccagaccagtctgcagattgtagccattggtgtttgcctggtttacctgatacatggaaccagttgttgtatacgcttctcatctattag

SEQ ID NO: 88 - protein, 402 aa, Arabidopsis thaliana
MGSLLPLLGISVVSAIFFLVLQQPEQSSSAIILSLKKRHGSSSGSSGNQYSSSRPSAGFQGNRSTCSLFLGTWVRDNSYPLYKPADCPGVVEPEFDCQMYGRPDSDYLKYRWQPQNCNLPTFNGAQFLLKMKGKTIMFAGDSLGKNQWESLICLIVSSAPSTRTEMTRGLPLSTFRFLDYGITMSFYKAPFLVDIDAVQGKRVLKLDEISGNANAWHDADLLIFNTGHWWSHTGSMQGWDLIQSGNSYYQDMDRFVAMEKALRTWAYWVETHVDRSRTQVLFLSISPTHDNPSDWAASSSSGSKNCYGETEPITGTAYPVSSYTDQLRSVIVEVLHGMHNPAFLLDITLLSSLRKDGHPSVYSGLISGSQRSRPDQSADCSHWCLPGLPDTWNQLLYTLLIY

SEQ ID NO: 89 - DNA, 1197 bp, Arabidopsis thaliana
atggcggctgttcaatgtctcactttcctcttcttgtttcttctccaaaacgcgacgtcagcttcacctctccctctcttccggcggccgatccaaagcaaccacagcaacttcgtgaagcatcctcgccgaagtcaggtggttttcccggtgaatcatagtagttgtgatctgttcgccggcgagtgggtgcgggatgagacttacccactttaccggtctaaagaatgcggccgaggaatcatagatccgggattcgactgccagacttacggcagacctgactccgactatctcaagttccggtggaaacctttcaactgcaatgtccctagattcaatggagtgaagtttctacaagaaatgagagacaaaaccataatgttcgttggtgattcgttgggtagaaaccaatgggagtcgttgatatgtatgatctcttcatcagcaccgtccattaatactcacatcattcatgaagatcctctctctaccttcaagatccttgactacaacgtgaaggtatcattctatagagctccttatcttgtagacatcgacaaaattaacggaaagacaactcttaaactcgacgaaatctctgtggatgcatcgaatgcttggcgcacagccgacgttctattgttcaacactggtcactggtggagccacacaggttctttacgagggtgggagcagatggagacaggtgggagatattacggcgacatggataggttagtggcgttgagaaaaggactaggaacgtggtctagttgggtccttcgttatatcaattctcctctcactagagttttcttcctctcggtctcacccacacactacaacccaaatgagtggacttcaagatcaaaaacttcaacaataactcaaggaggtaaaagctgctacggacaaacgacaccgtttagtggaacaacgtatccaacaagttcatacgtgaaccagaagaaagtgatagacgacgtggttaaagagatgaagtctcatgtctcattgatggatataactatgctctctgctcttcgtgtcgacggtcatccttcgatatatagtggagatctcaacccttctttgaaaagaaatcctgaccgttcatctgattgtagccattggtgtcttccgggtcttcccgacacttggaaccaattgttttatgctgctttgttgtactga

SEQ ID NO: 90 - protein, 398 aa, Arabidopsis thaliana
MAAVQCLTFLFLFLLQNATSASPLPLFRRPIQSNHSNFVKHPRRSQVVFPVNHSSCDLFAGEWVRDETYPLYRSKECGRGIIDPGFDCQTYGRPDSDYLKFRWKPFNCNVPRFNGVKFLQEMRDKTIMFVGDSLGRNQWESLICMISSSAPSINTHIIHEDPLSTFKILDYNVKVSFYRAPYLVDIDKINGKTTLKLDEISVDASNAWRTADVLLFNTGHWWSHTGSLRGWEQMETGGRYYGDMDRLVALRKGLGTWSSWVLRYINSPLTRVFFLSVSPTHYNPNEWTSRSKTSTITQGGKSCYGQTTPFSGTTYPTSSYVNQKKVIDDVVKEMKSHVSLMDITMLSALRVDGHPSIYSGDLNPSLKRNPDRSSDCSHWCLPGLPDTWNQLFYAALLY

SEQ ID NO: 91 - DNA, 1827 bp, Arabidopsis thaliana
atggcgtcagacgccgttaagtatatgccaatccacggcggaggaacaacagcaacaaccgccgccgacatcaagagcttcttctccgctctaaaacccaaaaaaacctcaactttcgcttacgctttcgtcataacctttgtctcattcactctcttctttgcttttagtccttctccaaactcatcttctccttggttctctaacatcttcacttcctcctccacaaccacaacttcagacaacacttccggatctcagttctcttcaattttctcatatattcttcccaatgtcacttcaaccaaacccaccaatagatccagtgacgccacagattctctctctgttaacgccacttctccacctctcaattcgaattccaaaaacggaactttacaaacaccggcgccggaaactcacactccggttgctaaaaacacaacctttgagtctccgatcgtaaacggaaccaacccagatgcaaagaacaacacttcatctcatcctctgctttcagacaaatcttcaacgacgggatcgaataatcagtcacggaccaccgccgatactgaaaccgtgaatagaaaccaaacaacatctccggcgccgtcgaaagcacccgtgtcggtagatctgaagaccaattccagttccaattcatcaactgcttcgtctacaccgaaaaagcagactaaaaccgttgatttggtttcgtcggtgaagcaagagatcgagaaatggagtgaatctttgaagaattgtgagttcttcgacggtgaatggatcaaagatgattcgtatccgctttataaacctggttcgtgtaatcttatcgatgaacagttcaattgtattaccaacggaagaccagacaaagatttccagaaattgaagtggaaacctaagaaatgtagtttaccaaggttaaatggagctatattgttggagatgcttagaggaagaagacttgtctttgtgggtgattcactaaataggaacatgtgggaatctctggtttgtattctcaaaggatcagtgaaagatgagactaaagtctatgaggctagaggaagacatcatttccgtggggaggctgagtactcattcgtcttccaagattataactgcacggtggagttctttgtttcacccttcttggttcaagagtgggaaattgtagacaagaaaggaacaaagaaggagactttaaggttggatttggttgggaagtcctctgagcagtacaaaggagctgatgttattgtctttaataccggacattggtggactcatgagaaaacatccaaaggggaggattattatcaagaaggaagtaatgtttatcatgaactcgctgttcttgaagcttttcgtaaagctttgactacatggggtcgatgggttgagaagaatgtgaatccggcaaagtccctcgttttctttcggggctattccgcatcccatttcagtggtgggcaatggaactcaggaggagcatgcgatagcgaaacagaaccgatcaagaacgatacatacctaaccccgtacccgtctaaaatgaaagtgcttgagaaagtgttaagaggtatgaaaacacctgtcacttatctaaacatcacgagattaacggactacaggaaggacggtcacccgtcagtataccggaaacagagcttatcggaaaaagagaaaaagtcgccattgctgtaccaagactgcagccattggtgcctccccggggttccagactcttggaatgaaatcctctacgctgagctgatcgtcaaactcaatcagctcagccaaacacaacgaaaaacttaa

SEQ ID NO: 92 - protein, 608 aa, Arabidopsis thaliana
MASDAVKYMPIHGGGTTATTAADIKSFFSALKPKKTSTFAYAFVITFVSFTLFFAFSPSPNSSSPWFSNIFTSSSTTTTSDNTSGSQFSSIFSYILPNVTSTKPTNRSSDATDSLSVNATSPPLNSNSKNGTLQTPAPETHTPVAKNTTFESPIVNGTNPDAKNNTSSHPLLSDKSSTTGSNNQSRTTADTETVNRNQTTSPAPSKAPVSVDLKTNSSSNSSTASSTPKKQTKTVDLVSSVKQEIEKWSESLKNCEFFDGEWIKDDSYPLYKPGSCNLIDEQFNCITNGRPDKDFQKLKWKPKKCSLPRLNGAILLEMLRGRRLVFVGDSLNRNMWESLVCILKGSVKDETKVYEARGRHHFRGEAEYSFVFQDYNCTVEFFVSPFLVQEWEIVDKKGTKKETLRLDLVGKSSEQYKGADVIVFNTGHWWTHEKTSKGEDYYQEGSNVYHELAVLEAFRKALTTWGRWVEKNVNPAKSLVFFRGYSASHFSGGQWNSGGACDSETEPIKNDTYLTPYPSKMKVLEKVLRGMKTPVTYLNITRLTDYRKDGHPSVYRKQSLSEKEKKSPLLYQDCSHWCLPGVPDSWNEILYAELIVKLNQLSQTQRKT

SEQ ID NO: 93 - DNA, 1795 bp, Arabidopsis thaliana
acgacacgacaccaacaaccacacgacacgacacgaccgatctatagattcggcgagatcagaagaaagcttcccggagcaactcggtcgttgtgactcattccgagttaaaaaaaacgggttttcgacaccatggatatgagttcaatggctggttcaatcggagtttcggtagccgtactccgattcctcctctgtttcgttgccacgatccctgtttcattcgcttgtcgaatcgtcccgagtagactcggtaaacacttgtatgccgctgcttcaggtgctttcctctcttacctctcctttggcttctcctccaaccttcacttccttgttccgatgacgatcggatatgcttcaatggcgatttatagacccaagtgtggaatcatcactttcttcctcggtttcgcttatcttattggctgtcatgtgttttatatgagtggtgatgcgtggaaagaaggaggaatcgattctactggagcgttaatggtgttgacgctgaaagtcatctcatgttcaatgaattacaatgatgggatgttgaaggaggaaggtctacgtgaagctcagaagaaaaacagattgattcagatgccgtctttgattgagtactttggttactgcctttgttgtggtagccattttgctggtcctgtttatgaaatgaaagattatcttgaatggaccgaagggaaagggatttgggatactactgagaaaagaaagaagccatcgccttatggagctacaatccgagctattttgcaagctgcgatttgcatggctctgtatctctatttagtgcctcaatatccgttaactcggttcacagaaccagtgtatcaagaatggggattcttgagaaaatttagttaccaatacatggctggattcacggctcgttggaagtattacttcatctggtcaatttcagaggcttctattatcatctctggtttgggtttcagtggttggactgatgatgcttcaccaaagcccaaatgggaccgtgccaagaacgtagatattctcggtgttgaactagctaagagcgcggttcagattccacttgtgtggaacatacaagtcagcacgtggctccgtcactatgtgtatgagagacttgtgcagaacggaaagaaagcgggtttcttccagttactagctacacaaaccgtcagcgcggtttggcatggactgtatcctggatatatgatgttctttgttcagtcagctttgatgatcgcaggctcacgggttatttaccggtggcaacaagcgatcagtccgaaaatggcaatgctgagaaatataatggtcttcatcaacttcctttacactgttttggttctcaactactcagccgtcggtttcatggtgttaagcttgcacgaaacacttaccgcctacggaagcgtatattacattggaacaatcatacctgttggattgattctcctcagttacgttgtgcctgcaaaaccttcaagaccaaaaccgcgtaaagaagaataagcagttatcttcttctcttaacggtaagtaagtttcccgcgcttgccagcttcttcttcttcttctgtaacatttggaaacaaaccgatccggttcttgtttctctctgattttttagcaccgatattttttttgtatttgttgcttataaatcttatttttcacacttcttttttttaattagtattggatttgcaattatatagacaataagtataaatatgtaactgtaaattgcaaatgggaaaaaatagtagtgtttatgtttgatgtt

SEQ ID NO: 94 - protein, 462 aa, Arabidopsis thaliana
MDMSSMAGSIGVSVAVLRFLLCFVATIPVSFACRIVPSRLGKHLYAAASGAFLSYLSFGFSSNLHFLVPMTIGYASMAIYRPKCGIITFFLGFAYLIGCHVFYMSGDAWKEGGIDSTGALMVLTLKVISCSMNYNDGMLKEEGLREAQKKNRLIQMPSLIEYFGYCLCCGSHFAGPVYEMKDYLEWTEGKGIWDTTEKRKKPSPYGATIRAILQAAICMALYLYLVPQYPLTRFTEPVYQEWGFLRKFSYQYMAGFTARWKYYFIWSISEASIIISGLGFSGWTDDASPKPKWDRAKNVDILGVELAKSAVQIPLVWNIQVSTWLRHYVYERLVQNGKKAGFFQLLATQTVSAVWHGLYPGYMMFFVQSALMIAGSRVIYRWQQAISPKMAMLRNIMVFINFLYTVLVLNYSAVGFMVLSLHETLTAYGSVYYIGTIIPVGLILLSYVVPAKPSRPKPRKEE

SEQ ID NO: 95 - DNA, 1014 bp, Arabidopsis thaliana
atggaggaagaactcaagagcttcgtcaaagtatggggttctgcaataatctctgtctcttactgttactatataccatcgaagatcaaaagaggtgttcatcgattactctcggttcttcctgtctgtgttctgtttcttgttcttcctttgtttttcgtctttacgattttctcttccaccactgcgttttgcctctctatacttgccaattttaagctcatcctatttgccttcgacaaaggtcctcttttaccacttcccactaatctcttccgattcatatgctttacttgcttgcccatcaagcttcaaacaaaacctaactctcaaaatcatctacccaaatgggttttacctagtaaagttgcaatttttgtgctgttgttaaacattcgtagctataaaattttgttgcctccaattcttctactaggtctctatccattgcatctatacattgtgcttgacgttcttttaaccattgtcaacgctttgctaaccatcattcttaggtgcgaccttgagccacatttcaacgaaccatacttagccacgtctcttcaagacttctggggtcaccgttggaacctcatggtctcggctatttaccggccaggggtctactctccggtgcgttcggtatgccagcaccaaatgaggtctgattgggcaaggttcatggggtgtatgacgacatttttcgtttctggtttgattcacgagctggtatacttctacataaaccgtgagaagcctactttggaagttacttggttctttgtattacatggggtttgcacggcgatggaaatagccgtcaagaggaagatgcaatggtcgttgagtccgatgttgttacggttgatcaccgtgggttttttggttgtcacaggtgatttgctgtttttcggacagattgaaaggagcaacatgttggagagacgcgccaatgaagcctcgctgttcattgatttcgtaaaacgcaaggttttcaattacactgtttcgtaa

SEQ ID NO: 96 - protein, 337 aa, Arabidopsis thaliana
MEEELKSFVKVWGSAIISVSYCYYIPSKIKRGVHRLLSVLPVCVLFLVLPLFFVFTIFSSTTAFCLSILANFKLILFAFDKGPLLPLPTNLFRFICFTCLPIKLQTKPNSQNHLPKWVLPSKVAIFVLLLNIRSYKILLPPILLLGLYPLHLYIVLDVLLTIVNALLTIILRCDLEPHFNEPYLATSLQDFWGHRWNLMVSAIYRPGVYSPVRSVCQHQMRSDWARFMGCMTTFFVSGLIHELVYFYINREKPTLEVTWFFVLHGVCTAMEIAVKRKMQWSLSPMLLRLITVGFLVVTGDLLFFGQIERSNMLERRANEASLFIDFVKRKVFNYTVS

SEQ ID NO: 97 - DNA, 1950 bp, Arabidopsis thaliana
agtcgtcttcttcgtttggatctcgtcaccggagctacaccggcgcttcagtgcttgcttctatcgtcgactaatagaaccagaatccgataaaaaaaaaaaaaaagtgttgactcaatcaaagagaagtcatgaacactcacaataatcgatacgcaaaatggaaacagagagagcttgttcttatactcctatacgccatcgctttctatgcttatgtcgtatggagatcgcttcgtctctcccacgatcattacttcaagctccatggtttagcttctggctggctcattcctaatcgacgtaatgatgtttctgatgctcaatggaggaactttcgtggaaatttgccaattcttagctttgttttcgctgttttcactgtgattgcaaatggggttaggtcgtttttccatttaagagctaaaggaatggctatcttgtggctttcaatgtcgttaatatacttgatatacttacatggagcttgcgtcatttacattctctcgattgcaacagccaatttccttcttgttaaggtttttgcacggactagttactttccttacatgctgtggactttcaacatattttttctcttctgcaatcgcatttatgaaggatattctttctccattttcgggcaacagtttgaattcctggataactttcggggcacatttagatggcatatatgcttcaactttgttgttttgcgcatgttaagttttggatatgattatcactggagccaactggattctcattttgatcaggagaaacacgtaatgcgctgttccttatgtaagttggggaagacttgctacgtagtgcgacaggagagaggtctagcaagtgatagctgcagtttttctctatacctttgttatttggtatatgcacccttgtaccttgcggggccaattataagctttaatgcatttgcttctcagctagatatgccacagaatactctttcattcaaagatgttgcttggtatggagtgcgttggttattcagcttcttgctgattgaactcatgacacacttattttattacaatgccttcgtgatcagtggtctgtggagagaactatctcctgtggaaatatttattgtcggatacggagtactaaactttatgtggcttaagttccttcttttatggagatatttccgcttttggtctctggtgaatggcattgaaactgttgagaacatgccgaattgcattaacaattgctacagcctagaactattctggaaaacctggcatgcctcatttaataggtggcttatcaggtatatgtatattcctctaggtggatcacgaagaaaatttctgaatgtgtgggttgtatttacatttgttgccatgtggcatgatttggaatggaagcttctttcatgggcatggttaacatgtttattcttcatgccagaaatgttactgaaatcagcatccagtgcatataaggttgagagtgcttttggagaattcctccttcgtgaactcaaggcgttgtctggtgccgtcacaatcacatgcctcatgattgcaaatcttgctggctatgtcattggaccatctggtataaactggatggtctccagctttctcaaaagagaaggagtacctgttctgggcggcgtgttctttagcttgtacgtgggtacaaagctgatgttccacatccaagacttaagaagtgtggcgcatagcccgaagtagccaagggaaagagacaaagagttgtaatctcctcagttacaaccaagtaatttcattattgttacgttttgtaaaaaatttagtcggactaatcgagtgtatccttgctggaagaatggaaaattaaatcaacttttgcaaaggcttttggatagatgatcactgaccacctgcagttttcaaaagcacaatgaacgcttatgaaaggtatctagta

SEQ ID NO: 98 - protein, 533 aa, Arabidopsis thaliana
MNTHNNRYAKWKQRELVLILLYAIAFYAYVVWRSLRLSHDHYFKLHGLASGWLIPNRRNDVSDAQWRNFRGNLPILSFVFAVFTVIANGVRSFFHLRAKGMAILWLSMSLIYLIYLHGACVIYILSIATANFLLVKVFARTSYFPYMLWTFNIFFLFCNRIYEGYSFSIFGQQFEFLDNFRGTFRWHICFNFVVLRMLSFGYDYHWSQLDSHFDQEKHVMRCSLCKLGKTCYVVRQERGLASDSCSFSLYLCYLVYAPLYLAGPIISFNAFASQLDMPQNTLSFKDVAWYGVRWLFSFLLIELMTHLFYYNAFVISGLWRELSPVEIFIVGYGVLNFMWLKFLLLWRYFRFWSLVNGIETVENMPNCINNCYSLELFWKTWHASFNRWLIRYMYIPLGGSRRKFLNVWVVFTFVAMWHDLEWKLLSWAWLTCLFFMPEMLLKSASSAYKVESAFGEFLLRELKALSGAVTITCLMIANLAGYVIGPSGINWMVSSFLKREGVPVLGGVFFSLYVGTKLMFHIQDLRSVAHSPK

SEQ ID NO: 99 - DNA, 1688 bp, Arabidopsis thaliana
aaaagaccccatcagagagattccgattcagatgtccgaactgtgagagtcgtcgtcgtcgtcgtaactcagtccgagttgacacaatcttccacttcacgcaagatacaaccatggaattgcttgacatgaactcaatggctgcctcaatcggcgtctccgtcgccgttctccgtttcctcctctgtttcgtcgcaacgataccaatctcatttttatggcgattcatcccgagtcgactcggtaaacacatatactcagctgcttctggagctttcctctcttatctctcctttggcttctcctcaaatcttcacttccttgtcccaatgacgattggttacgcttcaatggcgatttatcgacccttgtctggattcattactttcttcctaggcttcgcttatctcattggctgtcatgtgttttatatgagtggtgatgcttggaaagaaggaggaattgattctactggagctttgatggtattaacactgaaagtgatttcgtgttcgataaactacaacgatggaatgttgaaagaagaaggtctacgtgaggctcagaagaagaaccgtttgattcagatgccttctcttattgagtactttggttattgcctctgttgtggaagccatttcgctggcccggttttcgaaatgaaagattatctcgaatggactgaagagaaaggaatttgggctgtttctgaaaaaggaaagagaccatcgccttatggagcaatgattcgagctgtgtttcaagctgcgatttgtatggctctctatctctatttagtacctcagtttccgttaactcggttcactgaaccagtgtaccaagaatggggattcttgaagagatttggttaccaatacatggcgggtttcacggctcgttggaagtattactttatatggtctatctcagaggcttctattattatctctggtttgggtttcagtggttggactgatgaaactcagacaaaggctaaatgggaccgcgctaagaatgtcgatattttgggggttgagcttgccaagagtgcggttcagattccgcttttctggaacatacaagtcagcacatggctccgtcactacgtatatgagagaattgtgaagcccgggaagaaagcgggtttcttccaattgctagctacgcaaaccgtcagtgctgtctggcatggactgtatcctggatacattatattctttgtgcaatcagcattgatgatcgatggttcgaaagctatttaccggtggcaacaagcaatacctccgaaaatggcaatgctgagaaatgttttggttctcatcaatttcctctacacagtagtggttctcaattactcatccgtcggtttcatggttttaagcttgcacgaaacactagtcgccttcaagagtgtatattacattggaacagttatacctatcgctgtgcttcttctcagctacttagttcctgtgaagcctgttagaccaaagaccagaaaagaagaataatgttgtctttttaaaaaatcaacaacattttggttcttttctttttttccacttggaccgttttatgtaaaacaagagaaatcaagatttgaggttttattcttcttctccttcccaattttcgaaaatgattttattttttctgatatatatctaagctagtccaaagtcaactcg

SEQ ID NO: 100 - protein, 465 aa, Arabidopsis thaliana
MELLDMNSMAASIGVSVAVLRFLLCFVATIPISFLWRFIPSRLGKHIYSAASGAFLSYLSFGFSSNLHFLVPMTIGYASMAIYRPLSGFITFFLGFAYLIGCHVFYMSGDAWKEGGIDSTGALMVLTLKVISCSINYNDGMLKEEGLREAQKKNRLIQMPSLIEYFGYCLCCGSHFAGPVFEMKDYLEWTEEKGIWAVSEKGKRPSPYGAMIRAVFQAAICMALYLYLVPQFPLTRFTEPVYQEWGFLKRFGYQYMAGFTARWKYYFIWSISEASIIISGLGFSGWTDETQTKAKWDRAKNVDILGVELAKSAVQIPLFWNIQVSTWLRHYVYERIVKPGKKAGFFQLLATQTVSAVWHGLYPGYIIFFVQSALMIDGSKAIYRWQQAIPPKMAMLRNVLVLINFLYTVVVLNYSSVGFMVLSLHETLVAFKSVYYIGTVIPIAVLLLSYLVPVKPVRPKTRKEE

SEQ ID NO: 101 - DNA, 1020 bp, Arabidopsis thaliana
atggaggaagaaatcaagagcttgatcaatgtagggtttttaacaattatctcagtatcttactgctactgcttaccaccaagaatcaaatctggtgttcttcgattactctctatttttccggtctgtgttttgttagttgttcttcctctgttcttctccttttcaattttcacttccaccacagcgtttttcttatcagctattgccaattcaagactcatcctcttttcctttgatcaaggtcctctttttccactaccttcaaatctattcagatttacctgctttacttgcttcccaatccagcgtcaacaaaaccctaaatctcaagatcatttgtccacgtatgtttttcccgttaaaattgcaatctttgttgtgttgttatatgtgcataacgacatacaaaaccttcctcgtacttttctattgtgtctccatccactgtatgtatatttgttacttgagattctcttaacgctccttagaattctaatgactatcattcttggttgtgacctagagccacattttcacgaaccatacttagccacatctcttcaagacttttggggtcgcaggtggaacctcatagtctcggcaagtcttcgggcaatcgtctacactcctgtgcggcgtgtctgccaacgagtaatgagctctgattatgcaatgttgattggtgtttttgcgacgtttgtaacctctggtgtggctcatgaagtggttttcttttatataacccgtgcgatgcctacaggggaagtcgctttattctttctcttacatggagtttgcacggtggcggaagtggcagcgaagaggacggcgtttgtacggaggtggccggtgagaccagtcgtatcttggatgttcacgatagcgtttgtaaatgtgaccgctggttggctgttttttcctcagttgattcggaacaacctgggggagagatgctccaatgaaatctccttgctcattgatttcttcagaagcaagttattttattttccccagtga

SEQ ID NO: 102 - protein, 339 aa, Arabidopsis thaliana
MEEEIKSLINVGFLTIISVSYCYCLPPRIKSGVLRLLSIFPVCVLLVVLPLFFSFSIFTSTTAFFLSAIANSRLILFSFDQGPLFPLPSNLFRFTCFTCFPIQRQQNPKSQDHLSTYVFPVKIAIFVVLLYVHNDIQNLPRTFLLCLHPLYVYLLLEILLTLLRILMTIILGCDLEPHFHEPYLATSLQDFWGRRWNLIVSASLRAIVYTPVRRVCQRVMSSDYAMLIGVFATFVTSGVAHEVVFFYITRAMPTGEVALFFLLHGVCTVAEVAAKRTAFVRRWPVRPVVSWMFTIAFVNVTAGWLFFPQLIRNNLGERCSNEISLLIDFFRSKLFYFPQ

SEQ ID NO: 103 - DNA, 1038 bp, Arabidopsis thaliana
atggaggaagaactcatgagcttaatcaaagtatgggtttatgcaataatctccatatcttactgttactacacatcaacaagaatcaaatctggtgtttttcgattactatctgttcttcctgtttgtgttctgtttcttgttctccctctgtttgtttcctctgttcacttttctggttccacagcatttttcctctcatggcttgccaatttcaaactaatcctcttctccttcgaccaaggtccacttttcccagttccctcaaatctctcccgattcgtctgcttcacttgcttccccatcaagcttcaacaaaaccctaaacctcaaaatcaaatgcctaaatggggtttcgcagttaaacttgccttctttggtgtgttgttgcatatgtatgaatacaaacaacatatgtctccgactgttctattggttctctattctctgcatatatacttggagtatgagattctcttagctcccttgaaagttctgcttagtatctctctttggtgcgacctcgagccgcatttcaatgaaccatacttatccacctctcttcaagacttctggggtcgtcgatggaacctcatggtcccggcgattctccggccggctgtctacctcccggtgcgacaaatggccggtcggaaaatgaactctgatcaggctttgttcttgggagtttttgcctcgttccttgtttccggtgtggttcacgagcttattttcttctattttacacgtgaatcgccgacaggtgaagtcactttgttctttgtattacatggagtttgcactgccgctgaatgcgctgcgaagaggacgaggttggtgcggcgatggaaggtgagtcagatggtttcacgactgctcacggtgggatttgttgttatgaccggtggttggttgtttttccctcaccttgcaaggagtggcatgatcgagagactagctgacgaagcctttttgtttattggtttcgtcaagcacaagtttttctacctttgtagaaaccaatcgctaaaatcgtag

SEQ ID NO: 104 - protein, 345 aa, Arabidopsis thaliana
MEEELMSLIKVWVYAIISISYCYYTSTRIKSGVFRLLSVLPVCVLFLVLPLFVSSVHFSGSTAFFLSWLANFKLILFSFDQGPLFPVPSNLSRFVCFTCFPIKLQQNPKPQNQMPKWGFAVKLAFFGVLLHMYEYKQHMSPTVLLVLYSLHIYLEYEILLAPLKVLLSISLWCDLEPHFNEPYLSTSLQDFWGRRWNLMVPAILRPAVYLPVRQMAGRKMNSDQALFLGVFASFLVSGVVHELIFFYFTRESPTGEVTLFFVLHGVCTAAECAAKRTRLVRRWKVSQMVSRLLTVGFVVMTGGWLFFPHLARSGMIERLADEAFLFIGFVKHKFFYLCRNQSLKS

SEQ ID NO: 105 - DNA, 1429 bp, Arabidopsis thaliana
aacccaaaacctcactgattaagaaaaaggtaaaacgatggaagaagaattggtggtaatctcgaagagcatagttaatcccagaagccttaagaaaccgacttctgtgaagaagattcagctgactccatgggatctctctcgccttcgttttggttatcttcaaagaggtcttctctttcacaaaattgaagtcaaacaattacaagcttcactctctgttgcacttgatcgtttctatcctttagctggtcgtcttgtgaagctaaagaacgacgacgatactgtctcgtttttcatcagctgcgatggttcaggtgtggagtttgttcacgcagtggctaaaaacatcgagctcagtgatgtccttgagttatccggttctgtccccgggtttttcgcttcctttttccctgcaacggggatcaagaactaccacggtgtctcaagatctctacttatggttcaagtgacagagatgaaggacggggttttcatcggttttggttacaactctaccgtggctgatgccacttcaatctggaagttcatcaatgcatggtcagagatttgctcgaaagattcttcaggatcccaaactttccaacgtcgtcttcaccttaaaggttggttctttgacgagattgattatccaatccatattccagaccctgaaacaaaaccaactagttatgtaacaacaccaaccaatctacaagagaagatgtttcatgttacaaaagagaatgttttgaaacttgacgctaaagccaacgacgaggctgatcaaaagatctcttctattcaagcggtgttagcatatatatggcgttcaatggttaaacacagtggcatgagccgagaggaagagactcattgcaggttacctataaacatgaggcaacggctaaatccgccactagaagaagagtgttttgggaatgtaagccaaaccgggatagctacggtcacggttggagagctgctggaccatggactaggctgggctgcaatgcaaatcaacaacatggagctgtcacaaactgatgaaaaagccaaagcatttgcagagaattgggtgaaaaatatcaagattccggtttctgttggtagcaaagatttagttgtcacaaactctcaccggttcgatgtgtactgtaatgatttcggttggggtaaaccgatagctgcaagagctggaccaccgtatctcaatggaagactcgttgttttcaaaggaattggagaagcaagtcttgattttcaggcttgtttactgcctcaggtagtggaaaaattagtcaaagatgctgaatttaacgaatatgtaagcattgtatgagatgatttggtttagcaatgtgaatttaactaagccaaacaccaattctctctcgtttttcaaattaatacaataataagcctatgc

SEQ ID NO: 106 - protein, 434 aa, Arabidopsis thaliana
MEEELVVISKSIVNPRSLKKPTSVKKIQLTPWDLSRLRFGYLQRGLLFHKIEVKQLQASLSVALDRFYPLAGRLVKLKNDDDTVSFFISCDGSGVEFVHAVAKNIELSDVLELSGSVPGFFASFFPATGIKNYHGVSRSLLMVQVTEMKDGVFIGFGYNSTVADATSIWKFINAWSEICSKDSSGSQTFQRRLHLKGWFFDEIDYPIHIPDPETKPTSYVTTPTNLQEKMFHVTKENVLKLDAKANDEADQKISSIQAVLAYIWRSMVKHSGMSREEETHCRLPINMRQRLNPPLEEECFGNVSQTGIATVTVGELLDHGLGWAAMQINNMELSQTDEKAKAFAENWVKNIKIPVSVGSKDLVVTNSHRFDVYCNDFGWGKPIAARAGPPYLNGRLVVFKGIGEASLDFQACLLPQVVEKLVKDAEFNEYVSIV

SEQ ID NO: 107 - DNA, 1194 bp, Arabidopsis thaliana
atgttcaagttgaagcaatggttgatttatttggtgtgttcgttagtaataatgaacacagaaggactgtttgtcaatattacatttgttcgaaacgcagtcgctaaaggggccgtttgtttagatggaagtccaccagcttatcatttggatagaggttctggaactggaatcaatagttggttgatacagcttgagggaggaggatggtgcaataatgtaacaaattgcgttagtcggatgcatactcgattaggttcatcgaagaaaatggtggagaaccttgctttctcagctattcttagcaataagaaacaatataatcctgatttttacaattggaatagagtgaaagttagatactgtgacggggcatcattcacaggagatgtagaagcagtgaaccctgctactaatcttcacttcagaggtgctcgagtttggttagccgttatgcaagagctgctagctaaaggcatgataaacgccgagaatgctgttttgtctggctgttctgctggcgggttagcttcgctgatgcattgtgatagtttccgtgctctattaccgatgggaaccaaagtaaaatgtctttcagatgctggtttttttctcaacacaagagacgtctcaggagttcaatacattaaaacatacttcgaagatgttgttactcttcatggatcagcaaagaacttgccgaggtcatgcacatcaagattaactcctgcaatgtgtttctttccgcaatatgtggctcgccagattagaactcctctgttcattcttaatgccgcttatgactcttggcagataaagaacattttggctccgcgagcagctgatccttacggaaaatggcaaagttgtcaactagacatcaagaattgccatccaagtcagatcaaagttatgcaagatttcaggttagagttcttgagtgcagtgataggtttagggagatcttcatcaagagggatgttcatagattcttgctacactcactgccaaaccgagacacaaacttcatggttctggcaagattctccaattctaaaccgaacgacaatagcaaaagctgttggagattgggtttatgacagaacattgtttcagaagatagattgtccttacccttgtaaccctacttgccaccacagggttttcactcctctagatgctcctccaatttaa

SEQ ID NO: 108 - protein, 397 aa, Arabidopsis thaliana
MFKLKQWLIYLVCSLVIMNTEGLFVNITFVRNAVAKGAVCLDGSPPAYHLDRGSGTGINSWLIQLEGGGWCNNVTNCVSRMHTRLGSSKKMVENLAFSAILSNKKQYNPDFYNWNRVKVRYCDGASFTGDVEAVNPATNLHFRGARVWLAVMQELLAKGMINAENAVLSGCSAGGLASLMHCDSFRALLPMGTKVKCLSDAGFFLNTRDVSGVQYIKTYFEDVVTLHGSAKNLPRSCTSRLTPAMCFFPQYVARQIRTPLFILNAAYDSWQIKNILAPRAADPYGKWQSCQLDIKNCHPSQIKVMQDFRLEFLSAVIGLGRSSSRGMFIDSCYTHCQTETQTSWFWQDSPILNRTTIAKAVGDWVYDRTLFQKIDCPYPCNPTCHHRVFTPLDAPPI

SEQ ID NO: 109 - DNA, 1353 bp, Amorphophallus konjac
atggtgaaggagatgaggctggattggagcccctggcctctgcacaagcagaactacacctgtgccaagttcttcgtctgcatcctcctgatagggctctcgttccgcctcctgttctcgcattccgggggtttcctccccgtgtcggagcccgctgccaacgccccgagggaggagcttcctgtcaatgtgaagggcggtgagccgcaacaagactcatcatctccacagaaaagtgatggagtacggacatcaattcctgagatggcattggatgtagaagatggagcatccaagaaagaaaaatgtgatctgtttactggagagtggatacgaaaaccatctgggccagcttacaccaacaagagttgccttgtgattgaaggtcatcaaaactgtatgaaaaatggacgacctgatacaggtttcctttattggagatggaaaccattgggctgtgatttgcctctgtttgatgcagagagatttcttgatgccatgcagaacaaagcctgggcattcattggtgactcaatttcacgtaatcacatccaatcgcttgtttgtcttctttcaaaggcggacaaggctaccgaggtctatcatgatgcagaatatagatccaaaagatggagtttctcctcctacaactttacactctccattatctggtcccccttcctggttaaagctgaaatctttgaagatatgaatggtgtgtccactaaagacatccagcttcatcttgacacccttgacaacaaatggaccactgaattcaggaagtttgactacatagtaatagccggtggaaaatggtttctcaaaactgctatatactgggagaatggcactattgtgggctgccattattgtccagggaagaatctaaccgaacgtgggttcgagtatgcatatcgcaaggcacttaatttggtgcttcatcagctcacttcatctggttacaatggaactgttcttttcaggacatccacaccagaccacttcgaaaatggcgagtggtggagcggggggacgtgcaaccggacacgaccatttaaagaaggagaggtaacaataagagatgtggaccaggtaatgaggacgattgagttagaggaattcattaaagctttggctgcagcatccaacaatggcatgcgtctgaagcttttggacacaactcatctttcactaatgaggccggatggacatcctggcccttacaggcagtttcaaccatttgcaaaggataagaaagccaaagttcagaatgactgcctccactggtgcttgcctggccccatagactcttggaatgatctaatcatggagatggctttaagagactga

SEQ ID NO: 110 - protein, 450 aa, Amorphophallus konjac
MVKEMRLDWSPWPLHKQNYTCAKFFVCILLIGLSFRLLFSHSGGFLPVSEPAANAPREELPVNVKGGEPQQDSSSPQKSDGVRTSIPEMALDVEDGASKKEKCDLFTGEWIRKPSGPAYTNKSCLVIEGHQNCMKNGRPDTGFLYWRWKPLGCDLPLFDAERFLDAMQNKAWAFIGDSISRNHIQSLVCLLSKADKATEVYHDAEYRSKRWSFSSYNFTLSIIWSPFLVKAEIFEDMNGVSTKDIQLHLDTLDNKWTTEFRKFDYIVIAGGKWFLKTAIYWENGTIVGCHYCPGKNLTERGFEYAYRKALNLVLHQLTSSGYNGTVLFRTSTPDHFENGEWWSGGTCNRTRPFKEGEVTIRDVDQVMRTIELEEFIKALAAASNNGMRLKLLDTTHLSLMRPDGHPGPYRQFQPFAKDKKAKVQNDCLHWCLPGPIDSWNDLIMEMALRD

SEQ ID NO: 111 - DNA, 1176 bp, Arabidopsis thaliana
atggcgttcagtagagcacatggatgtttgttcatctgtctctgcatactgatcttactgaaggctgatggttactttgtggacatcacttatgtcgagagtgccgtgtctaagggagcagtgtgcttggatgggagcccacctgcttatcatctctcccctggtttcgggtcaggggtgaacaactggctagttcagattgagggaggaggatggtgcaacaatgtcaccacctgtcttgctcggaagaacacccgcctagggtcttccaaggcgatggtgaagcagctagcattttcagggattttcagcaacaaacagtcaatgaacccagacttctataactggaacaaggtcaaagttcgttactgtgatggttcatcatttactggagatgtggagagagtagatcctgctaccaatcttcattttcgaggagcaagggtttttcttgctgtgatggaagatttactctcgaaagggatgagtcacgctgaaaatgctcttctttctggttgttcagctggagggttaacatctatattgcactgtgacaacttccgagctctgttgcctagtgccaatgttaaatgccttgcagatgctggttacttcatcagcgcgaaagacatcacaggagctgaacacataaaagatttctataatgatgttgtaacaacacatggttctgcaaagaatttgccctcatcatgcacttcaagactaaacccaggaatgtgcatgttcccacaatatgtagctcaagggatccgcacacctcttttcattttgaatgcagcatacgattcatggcagataaagaatatcgtggctccgggtgtagctgatccccatggaacttggcatgattgcaagcttgatataaagaagtgttcttcaagtcaaattggcgtaatgcaaggtttcagggaggaattcttgaatgctctggcatcagtgggaagctcttcatctagaggcttattcataaactcgtgttatgcccattgccagtcggagacgcaggagacatggctgagggctgattctcctaaactgggcggcataacgatcgcaaaggcagtcggagactggtattctggtcgggtcggcttccagaagatcgactgcccatatccgtgcgacagcacttgccacaatcgcatcttcgagtga

SEQ ID NO: 112 - protein, 391 aa, Arabidopsis thaliana
MAFSRAHGCLFICLCILILLKADGYFVDITYVESAVSKGAVCLDGSPPAYHLSPGFGSGVNNWLVQIEGGGWCNNVTTCLARKNTRLGSSKAMVKQLAFSGIFSNKQSMNPDFYNWNKVKVRYCDGSSFTGDVERVDPATNLHFRGARVFLAVMEDLLSKGMSHAENALLSGCSAGGLTSILHCDNFRALLPSANVKCLADAGYFISAKDITGAEHIKDFYNDVVTTHGSAKNLPSSCTSRLNPGMCMFPQYVAQGIRTPLFILNAAYDSWQIKNIVAPGVADPHGTWHDCKLDIKKCSSSQIGVMQGFREEFLNALASVGSSSSRGLFINSCYAHCQSETQETWLRADSPKLGGITIAKAVGDWYSGRVGFQKIDCPYPCDSTCHNRIFE

SEQ ID NO: 113 - protein, 105 aa, Arabidopsis thaliana
  VARIANT (2)...(2): Xaa = Asp or Asn or Ser or Glu
CXXXXGXWXXXXXXXXXXXXXXXXXXXXXXXXXXXXXXCXXXXXXXXXXXXXXXWXPXXCXXXXXXXXXXXXXXXXXXXXXXXXXGDSXXXXXXXSXXCXXXXXX

SEQ ID NO: 114 - protein, 108 aa, Artificial Sequence
  Synthetic Construct
CDLFXXXXXGXWVXDXXXXXXXXXPLYXXXXCXXXFIDXXXXCXKNGRPDXXYLKWRWQPXXXXXXXCXLPRXFDAXXFLERLRXGKRLMFVGDSLXRNQWESLVCLL

SEQ ID NO: 115 - protein, 92 aa, Arabidopsis thaliana
  VARIANT (3)...(3): Xaa = Leu or Tyr or Ile
CDXXGXWXXXXXXXXYXXXXCXXXXXXXXNCXXXGRPDXXXXXWXWXPXXCXXXXPXFXXXXFLXXXXXXXXXXXGDSXXRNXXXSXXCXLX

SEQ ID NO: 116 - protein, 105 aa, Artificial Sequence
  Synthetic Construct
CDLFXGXXXXEWVPDXXGPXXXXXXYYTNXTCXXIXXXQNCMKXGRPDXGYLXWRWKPXGCDXXXXXLPRFDXXRFLXLVRGKSLAFVGDSLARNQMXSLLCLLS

SEQ ID NO: 117 - protein, 462 aa, Arabidopsis thaliana
  VARIANT (1)...(462): Xaa = Any amino acid
LDMXSMAASIGVSVAVLRFLLCFVATIPISFXXRXIPSRLGKHIYAAASGAFLSYLSFGFSSNLHFLVPMTIGYASMAIYRPXXGXITFFLGFAYLIGCHVFYMSGDAWKEGGIDSTGALMVLTLKVISCSINYNDGMLKEEGLREAQKKNRLIQMPSLIEYFGYCLCCGSHFAGPVFEMKDYLEWTEXKGIWXXSEKXKKPSPYGAXIRAIXQAAICMALYLYLVPQFPLTRFTEPVYQEWGFLKKFXYQYMAGFTARWKYYFIWSISEASIIISGLGFSGWTDDXXXKXKWDRAKNVDILGVELAKSAVQIPLXWNIQVSTWLRHYVYERIVXXGKKAGFFQLLATQTVSAVWHGLYPGYIIFFVQSALMIXGSKXIYRWQQAIXPKMAMLRNILVXINFLYTVLVLNYSAVGFMVLSLHETLXAFXSVYYIGTIIPIALILLSYLVPXKPXRPKXRKEE

SEQ ID NO: 118 - protein, 382 aa, Artificial Sequence
  Synthetic Construct
MESMAAAIGVSVPVLRFLLCFVATIPVSFLWRFVPSAXGKHLYAALSGAFLSYLSFGFSSNLHFLVPMTLGYLSMLLFRPYCGIITFLXGFGYLIGCHVYYMSGDAWKEGGIDATGALMVLTLKVISCAINYNDGMLKEEGLREAQKKNRLIKLPSLIEYFGYCLCCGSHFAGPVYEMKDYLEWTERKGIWASSEKGPTPSPFGATIRALLQAAVCMALYLYLIPQFPLSRFSEPLYQEWGFWKRLXYQYMSGFTARWKYYFIWSISEAAIIISGLGFSGWTDSSPPKPKWDRAKNVDILGVELAKSAVQIPLVWNIQVSTWLRHYVYERLVQKGKKPGFFQLLATQTVSAVWHGLYPGYIIFFVQSALMIAGSRVIYRWQQ

SEQ ID NO: 119 - protein, 337 aa, Arabidopsis thaliana
  VARIANT (1)...(337): Xaa = Any amino acid
MEEELKSLIKVWXXAIISVSYCYYIPSRIKSGVXRLLSVLPVCVLFLVLPLFFSFSIFSSTTAFFLSXLANFKLILFSFDQGPLFPLPSNLFRFICFTCFPIKLQQNPKSQNHLPKWVFPVKIAIFVVLLHIHXYKQXLPPTLLLXLYPLHIYLLLEILLTILKILLTIILXCDLEPHFNEPYLATSLQDFWGRRWNLMVSAILRPAVYSPVRXVCQRXMXSDWALFIGVFATFLVSGVIHELVFFYITREXPTGEVTLFFVLHGVCTAAEIAAKRTXXVRRWXVSPMVSRLITVGFVVVTGGWLFFPQLXRSNMIERXANEASLFIDFVKXKLFYF

SEQ ID NO: 120 - protein, 96 aa, Artificial Sequence
  Synthetic Construct
EPQFDXPYLASSLRDFWGRRWNLMVSAILRPSVYXPVRAXXGXXXXXXXAXAXGVLATFLVSGLMHELMFYYIXRXXPTGEVTXFFLLHGVCXAAE

SEQ ID NO: 121 - protein, 385 aa, Arabidopsis thaliana
  VARIANT (1)...(385): Xaa = Any amino acid
NDLSDAQWRNFRGNLPILTIVMGAFLMLANXLRYCYXLKGRGXALLWLLLSLSYLCYLHGACVVFILLIALINYXIVKLFAXYKYCTXLIWSFNLSVLILNRVYEGYSFSLFGQQLAFLDNYRGTFRWHICFNFVVLRMISFGCDYCWSIXSSHFDXKKHMQRCXVCXSGKTCYXXLQERGLSXDKYTFLIYLCYLTYAPLYIAGPIVSYNAFAAQLDVPQKNYSFAQISWYGLRWILSFLLMEGMTHFFHYNAFVVSRLWQXLSPFEIFIISYGVLNFMWLKFFLIWRYFRFWSLVGGVETPENMPRCINNCHDLESFWKSWHASFNRWLVRYLYIPLGGSQRKLLSIWVIFTFVAVWHDLEWKLISWAWLTCLFFIPEILVKS

SEQ ID NO: 122 - protein, 5 aa, Artificial Sequence
  Synthetic Construct
HXXXD

SEQ ID NO: 123 - protein, 5 aa, Artificial Sequence
  Synthetic Construct
DFGWG

SEQ ID NO: 124 - protein, 220 aa, Artificial Sequence
  Synthetic Construct
ECFFXFXAESVRKLKAKANAEMAAXXXXXXXAAIISSLQALLAHIWRAVXRARXLTPEXETXYXLVIGCRARVNGXIPXGYVGNAVVXGIAXLTAGEILEXGLGWXALXLNRXVASFDEAXMRAXLAXWVRXPXFXXXXXXXXGGGXALXTGSSPRFDVYGNDFGWGRPIAVRSGXGNKXDGKLTVFEGXGXAGSMSLEVCLAPXALXKLVADXEFMDAV

SEQ ID NO: 125 - RNA, 48 nt, Artificial Sequence
  Synthetic Construct
uuuaucggugauuccauggcuagaaaucagcuugagucucuuuuaugc

SEQ ID NO: 126 - DNA, 1701 bp, Artificial Sequence
  Synthetic Construct
tctcgtctcttccacataaaacaagaacaacaaccaaaaaacgatgtgacatatacttagaaacaagctccatatcaataatcaaaaacacgaaagccaaaataaaagagagaaaaagaagataagttgttctatgttcctgaaatgggattaaacgagcaacaaaatgttccaagtcagaggaagatcatagtttttatagtcttagcttttataccaatagctctgtttcgactctgttttaataatccattctcctccattaaagacacctctcttcaagactctgcagctaacgttgtgatcactagcttctcttcatcttctcaaggtaattattcaacttcttcaatattgtgtatatccaaaaactgaatggatttttttaactttagggttgttttctaaatctgcagaagaagaaagccaagagagttttgatcatatacaagatgaacctttatgcgattatacgcaagggaactgggtcagagacgagattggtccactctacaatggttcaacatgtggaacaatcaaggatggtcagaattgtttccgccatggtagacctgattccggttatctttactggaaatggaaaccaaacgaatgcgatataccgagattcgactcgaaccggtttcttgatcttatgagagataagcatctagcttttatcggtgattccatggctagaaatcagcttgagtctcttttatgcttgctctctaccgtctctagccctgatctcgtctatagaaacggagaagacaataaattcagaagatggcgtttcgagtcacacaacgtcactgtctcggtttactggtccccgtttttggtggccggtttagagaaatcgggaaacttggaccacaatgtattgcacatagaccgcgtggatgagagatggggcaatgatttagaacgttttgatacggtcgtagtctctgtgggacattggtttctgcatccagcggtttactacgagtctggttcggttttgggatgtcattcttgtgaaacaagtaactgtaccgaggtagggttttatgatgtatttagaaaagcgataagaacaacgttgagagcggttgctggaagtggtcgtgaggtgatcttgacgacattttcgccatcacattttgaaggacggccttgggacagtctcggcgcatgtaacatgaccaagccgtacgaagggaaggttttagaaggtctggacttggacatgcgcaaaatagaaattgaggaatatacggcggctgcggctgaggtgaggttagaggtgttggacgtgacggctatgtcggtactgagaccagacggacatcctggtccgtacatgtacgcggacccgttcaagaacggtgtaccggagagaattcctaatgattgtttgcattggtgtctacctggtcctgtcgacacgtggaacgagatcatgatcgagatgttgcggcgatggaaggtttaaatgatgaacattgattgttgaagatcctatgatatcattcatttacacgcatatgacaaaagaacaaaatgtgtaataattttttgggatttcaaaaagtagctaggtagtttattttcttggattagatacgaaaaaaacagacatggatgatactcttttttgttggttatttcttccgttcttcaaaccatggaggagtacaaaaaatagttgaacat

SEQ ID NO: 127 - DNA, 1540 bp, Arabidopsis thaliana
caaagaagaagaagcctacggttgttctgttcctttcaatctcaccaaattttcatcttcaccttcaattttgatagattctcttcaccgaaccaaatccgtaaccctagcttcttcttaaattatagagaaaaagagttaattcgaatctggatgaaattggttttgtctagatggaaatctagatgtgtctgattccgaaatctgagtctgagagatgctaggagcgattcatttgggagtattagcagcttgtttcgtcctcttcgttcccatggcaatggctggttggcatctgagcaggaacaagatgctcttctttagtggcgctttgtttatttctctcgcggtttgtgttcatctcacgccttatttcccttccgtttccgacatcgttgcctctgtttcctctgttgttgtctacgatcatcgtatctcttgtatcaacgaggttaatcagattgtctgggatgttaaaccggtaccgaatccggagtctgttcgtaggaacaatggctcaacaaagcttgattatttcgtgaagaactgggattggatgaaatcgaggaaagttttgagttgtgagtttcagaaattggataagttcgatgtttcggatttgttgaatggatcgtgggttgttgttgctggtgattctcaggcgagattcgtggctttgtctttgttgaatcttgttttgggttcggattccaaggctatggattcggttagaggggatttgtttaggagacatagtgattacagcattgtggttaaggagattgggatgaagcttgattttgtttgggctccttatgagaaagatttggatgatcttgtggtatcttataagaagatgaaaaagtatcctgatgttgtgatcatgggaactgggttatggcatatgcttcatgtgaacaatgcttcagattttggttttaggttgaggcaattgagtagtcatgtggagtctctagtgccccttacgccaaaggagcaagaaggaggtgggtcggtctccggtagatctgtgcatctgttctggattgggatgccggttttgatcaacgggatgttgaatacggatgagaagaaggagaagatgagtgatacggtgtggcatgagtatgatagatcactcggggagagtaagatcttacgtcaaatgggtggtcctcttatcttgcttgatatccagtcgtttacctggaactgtggaccgcaatgtacacttgacgggatgcattatgactctgcggtctatgatgccgctgtccatgtcatgctcaatgcgttgcttattgaatctcatcaatctttatgagttcttgacacgttttttttttgttttttttccttttattttgtttggggttctcttgtagagttttaagagaagtgggatgttcataggaattttacaacacacggtgatccttttggcctgagtgatcttcacattggagaagtaagtgaacttacattcatggctataataggtttaatatcttttgactttgagaagttataacctttagct